(19) United States
(12) Patent Application Publication
(10) Pub. No.: US 2024/0294534 A1
(43) Pub. Date: Sep. 5, 2024

(54) IMIDAZOTRIAZINE DERIVATIVES AS IL-17 MODULATORS

(71) Applicant: UCB BIOPHARMA SRL, Brussels (BE)

(72) Inventors: Gareth Neil BRACE, Abingdon Oxfordshire (GB); Daniel Christopher BROOKINGS, Slough Berkshire (GB); Shuyu CHU, Slough Berkshire (GB); Rickki Lee CONNELLY, Abingdon Oxfordshire (GB); Anne Marie FOLEY, Slough Berkshire (GB); James Richard FROST, Slough Berkshire (GB); Ellen Olivia GALLIMORE, Slough Berkshire (GB); Paul GOLDSMITH, Slough Berkshire (GB); James Andrew JOHNSON, Slough Berkshire (GB); James Thomas REUBERSON, Slough Berkshire (GB); Robert STRAKER, Slough Berkshire (GB); Richard David TAYLOR, Slough Berkshire (GB); Luigi Piero STASI, Slough Berkshire (GB)

(21) Appl. No.: 18/572,274

(22) PCT Filed: Jun. 30, 2022
(86) PCT No.: PCT/EP2022/068165

§ 371 Date: Dec. 20, 2023

(30) Foreign Application Priority Data
Jul. 1, 2021 (GB) 2109520.3
Mar. 21, 2022 (GB) 2203876.4

(51) Int. Cl. C07D 487/04; A61K 31/53; A61K 31/5377; A61K 31/541

(52) U.S. Cl. CPC C07D 487/04; A61K 31/53; A61K 31/5377; A61K 31/541

(57) ABSTRACT

A series of substituted imidazo[1,2-b][1,2,4]triazine derivatives of formula (I), being potent modulators of human IL-17 activity, are accordingly of benefit in the treatment and/or prevention of various human ailments, including inflammatory and autoimmune disorders.

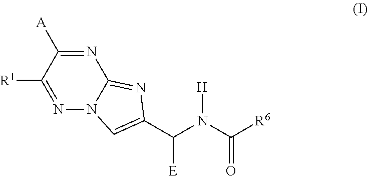

The present invention relates to heterocyclic compounds, and to their use in therapy. More particularly, this invention is concerned with pharmacologically active substituted imidazo[1,2-b][1,2,4]triazine derivatives. These compounds act as modulators of IL-17 activity, and are accordingly of benefit as pharmaceutical agents for the treatment and/or prevention of pathological conditions, including adverse inflammatory and autoimmune disorders.

IL-17A (originally named CTLA-8 and also known as IL-17) is a pro-inflammatory cytokine and the founder member of the IL-17 family (Rouvier et al., J. Immunol., 1993, 150, 5445-5456). Subsequently, five additional members of the family (IL-17B to IL-17F) have been identified, including the most closely related, IL-17F (ML-1), which shares approximately 55% amino acid sequence homology with IL-17A (Moseley et al., Cytokine Growth Factor Rev., 2003, 14, 155-174). IL-17A and IL-17F are expressed by the recently defined autoimmune related subset of T helper cells, Th17, that also express IL-21 and IL-22 signature cytokines (Korn et al., Ann. Rev. Immunol., 2009, 27, 485-517). IL-17A and IL-17F are expressed as homodimers, but may also be expressed as the IL-17A/F heterodimer (Wright et al., J. Immunol., 2008, 181, 2799-2805). IL-17A and F signal through the receptors IL-17R, IL-17RC or an IL-17RA/RC receptor complex (Gaffen, Cytokine, 2008, 43, 402-407). Both IL-17A and IL-17F have been associated with a number of autoimmune diseases.

The compounds in accordance with the present invention, being potent modulators of human IL-17 activity, are therefore beneficial in the treatment and/or prevention of various human ailments, including inflammatory and autoimmune disorders.

Furthermore, the compounds in accordance with the present invention may be beneficial as pharmacological standards for use in the development of new biological tests and in the search for new pharmacological agents. Thus, the compounds of this invention may be useful as radioligands in assays for detecting pharmacologically active compounds.

WO 2013/116682 and WO 2014/066726 relate to separate classes of chemical compounds that are stated to modulate the activity of IL-17 and to be useful in the treatment of medical conditions, including inflammatory diseases.

WO 2018/229079 and WO 2020/011731 describe spirocyclic molecules that are stated to act as modulators of IL-17 activity, and thus to be of benefit in the treatment of pathological conditions including adverse inflammatory and autoimmune disorders.

WO 2019/138017, WO 2020/260425, WO 2020/260426 and WO 2020/261141 describe various classes of fused bicyclic imidazole derivatives that are stated to act as modulators of IL-17 activity and thus to be of benefit in the treatment of pathological conditions including adverse inflammatory and autoimmune disorders. Fused bicyclic imidazole derivatives operating as modulators of IL-17 activity are also described in copending international patent applications PCT/EP2021/054519 and PCT/EP2021/054523 (both published on 2 Sep. 2021 as WO 2021/170627 and WO 2021/170631 respectively), in copending international patent applications PCT/EP2021/058937 and PCT/EP2021/058940 (both published on 14 Oct. 2021 as WO 2021/204800 and WO 2021/204801 respectively), in copending international patent applications PCT/EP2021/080250 and PCT/EP2021/080251 (both published on 12 May 2022 as WO 2022/096411 and WO 2022/096412 respectively), and in copending international patent application PCT/EP2021/084448 (published on 23 Jun. 2022 as WO 2022/128584).

WO 2020/120140 and WO 2020/120141 describe discrete classes of chemical compounds that are stated to act as modulators of IL-17 activity, and thus to be of benefit in the treatment of pathological conditions including adverse inflammatory and autoimmune disorders.

Heterocyclic compounds that are stated to inhibit IL-17A and to be useful as immunomodulators are described in WO 2019/223718, WO 2021/027721, WO 2021/027722, WO 2021/027724, WO 2021/027729 and WO 2021/098844.

Heterocyclic compounds stated to be capable of modulating IL-17 activity are also described in WO 2020/127685, WO 2020/146194 and WO 2020/182666.

None of the prior art available to date, however, discloses or suggests the precise structural class of substituted imidazo[1,2-b][1,2,4]triazine derivatives as provided by the present invention.

As well as being potent modulators of human IL-17 activity, the compounds in accordance with the present invention possess other notable advantages. In particular, the compounds of the invention display valuable metabolic stability, as determined in either microsomal or hepatocyte incubations. The compounds of the invention also display valuable permeability as determined by standard assays, e.g. the Caco-2 permeability assay.

The present invention provides a compound of formula (I) or an N-oxide thereof, or a pharmaceutically acceptable salt thereof:

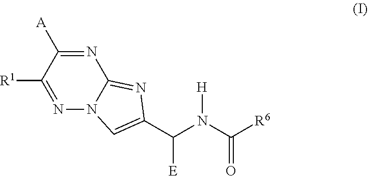

wherein
E represents a group of formula (Ea), (Eb), (Ec), (Ed) or (Ee):

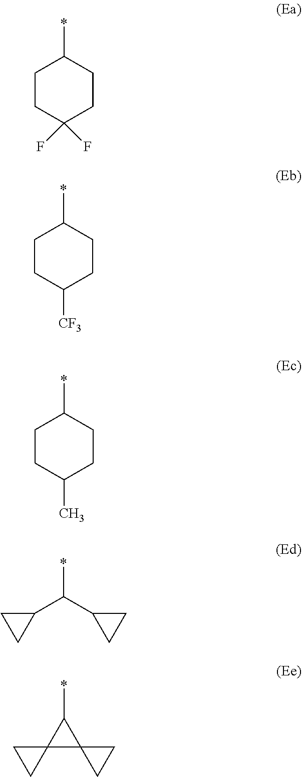

in which the asterisk (*) represents the point of attachment to the remainder of the molecule;
A represents a group of formula (Aa), (Ab), (Ac), (Ad) or (Ae):

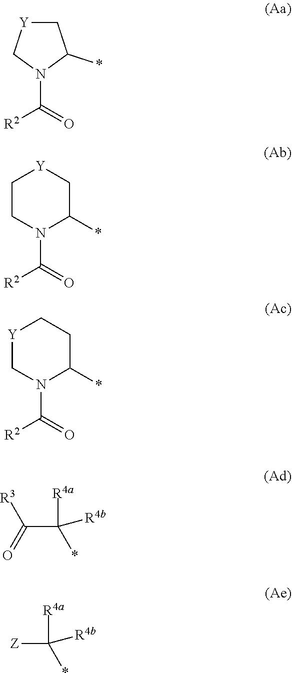

in which the asterisk (*) represents the point of attachment to the remainder of the molecule;
Y represents —O—, —N(R⁷)—, —C(R^{5a})(R^{5b})—, —S—, —S(O)—, —S(O)₂— or —S(O)(N—R⁸)—;
Z represents heteroaryl, which group may be optionally substituted by one or more substituents;
R¹ represents hydrogen, fluoro, chloro, methyl, difluoromethyl or trifluoromethyl;
R² represents —OR^{2a}; or R² represents C₃₋₉ cycloalkyl, C₄₋₁₂ bicycloalkyl, C₃₋₇ heterocycloalkyl or C₄₋₉ heterobicycloalkyl, any of which groups may be optionally substituted by one or more substituents;
R^{2a} represents C₁₋₆ alkyl; or R^{2a} represents C₃₋₉ cycloalkyl, which group may be optionally substituted by one or more substituents;
R³ represents —NR^{3a}R^{3b}; or R³ represents a group of formula (Wa):

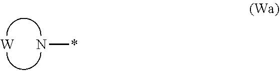

in which the asterisk (*) represents the point of attachment to the remainder of the molecule;
W represents the residue of an optionally substituted saturated monocyclic ring containing 3 to 6 carbon atoms, one nitrogen atom, and 0, 1, 2 or 3 additional heteroatoms independently selected from N, O and S, but containing no more than one O or S atom; or
W represents the residue of an optionally substituted saturated bicyclic ring system containing 4 to 10 carbon atoms, one nitrogen atom, and 0, 1, 2 or 3 additional heteroatoms independently selected from N, O and S, but containing no more than one O or S atom; or
W represents the residue of an optionally substituted saturated spirocyclic ring system containing 5 to 10 carbon atoms, one nitrogen atom, and 0, 1, 2 or 3 additional heteroatoms independently selected from N, O and S, but containing no more than one O or S atom;
R^{3a} represents hydrogen or C₁₋₆ alkyl;
R^{3b} represents C₁₋₆ alkyl, C₃₋₇ cycloalkyl, C₃₋₇ cycloalkyl(C₁₋₆)alkyl, C₄₋₁₂ bicycloalkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents;
R^{4a} represents hydrogen, fluoro or hydroxy; or R^{4a} represents C₁₋₆ alkyl, which group may be optionally substituted by one or more substituents; and
R^{4b} represents hydrogen, fluoro or C₁₋₆ alkyl; or
R^{4a} and R^{4b}, when taken together with the carbon atom to which they are both attached, represent C₃₋₉ cycloalkyl or C₃₋₇ heterocycloalkyl, either of which groups may be optionally substituted by one or more substituents;
R^{5a} represents hydrogen, fluoro, methyl, difluoromethyl or trifluoromethyl; and
R^{5b} represents hydrogen, fluoro, methyl or hydroxy; or
R^{5a} and R^{5b}, when taken together with the carbon atom to which they are both attached, represent cyclopropyl;
R⁶ represents —OR^{6a} or —NR^{6b}R^{6c}; or R⁶ represents C₁₋₆ alkyl, C₃₋₉ cycloalkyl, C₃₋₉ cycloalkyl(C₁₋₆)alkyl, aryl, aryl(C₁₋₆)alkyl, C₃₋₇ heterocycloalkyl, C₃₋₇ heterocycloalkyl-(C₁₋₆)alkyl, heteroaryl or heteroaryl(C₁₋₆)alkyl, any of which groups may be optionally substituted by one or more substituents;
R^{6a} represents C₁₋₆ alkyl, C₃₋₉ cycloalkyl or aryl(C₁₋₆)alkyl, any of which groups may be optionally substituted by one or more substituents;
R^{6b} represents hydrogen or C₁₋₆ alkyl; and
R^{6c} represents hydrogen or C₁₋₆ alkyl; or
R^{6b} and R^{6c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents;
R⁷ represents —COR^{7a}, —CO₂R^{7a} or —SO₂R^{7b}; or R⁷ represents hydrogen; or R⁷ represents C₁₋₆ alkyl or C₃₋₉ cycloalkyl, either of which groups may be optionally substituted by one or more fluorine atoms;
R^{7a} represents C₁₋₆ alkyl, optionally substituted by one or more fluorine atoms;
R^{7b} represents C₁₋₆ alkyl; and
R⁸ represents C₁₋₆ alkyl.

The present invention also provides a compound of formula (I) as defined above, or a pharmaceutically acceptable salt thereof.

The present invention also provides a compound of formula (I) as defined above or an N-oxide thereof, or a pharmaceutically acceptable salt thereof, for use in therapy.

The present invention also provides a compound of formula (I) as defined above or an N-oxide thereof, or a pharmaceutically acceptable salt thereof, for use in the treatment and/or prevention of disorders for which the administration of a modulator of IL-17 function is indicated.

The present invention also provides the use of a compound of formula (I) as defined above or an N-oxide thereof, or a pharmaceutically acceptable salt thereof, for the manufacture of a medicament for the treatment and/or prevention of disorders for which the administration of a modulator of IL-17 function is indicated.

The present invention also provides a method for the treatment and/or prevention of disorders for which the administration of a modulator of IL-17 function is indicated which comprises administering to a patient in need of such treatment an effective amount of a compound of formula (I) as defined above or an N-oxide thereof, or a pharmaceutically acceptable salt thereof.

Where any of the groups in the compounds of formula (I) above is stated to be optionally substituted, this group may be unsubstituted, or substituted by one or more substituents. Generally, such groups will be unsubstituted, or substituted by one, two, three or four substituents. Typically, such groups will be unsubstituted, or substituted by one, two or three substituents. Suitably, such groups will be unsubstituted, or substituted by one or two substituents.

For use in medicine, the salts of the compounds of formula (I) will be pharmaceutically acceptable salts. Other salts may, however, be useful in the preparation of the compounds of formula (I) or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. Suitable pharmaceutically acceptable salts of the compounds of formula (I) include acid addition salts which may, for example, be formed by mixing a solution of a compound of formula (I) with a solution of a pharmaceutically acceptable acid.

The present invention also includes within its scope co-crystals of the compounds of formula (I) above. The technical term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity (see Pharmaceutical Salts and Co-crystals, ed. J. Wouters & L. Quere, RSC Publishing, 2012).

Suitable alkyl groups which may be present on the compounds of use in the invention include straight-chained and branched C₁₋₆ alkyl groups, for example C₁₋₄ alkyl groups. Typical examples include methyl and ethyl groups, and straight-chained or branched propyl, butyl and pentyl groups. Particular alkyl groups include methyl, ethyl, n-propyl, isopropyl, n-butyl, sec-butyl, isobutyl, tert-butyl, 2,2-dimethylpropyl and 3-methylbutyl. Derived expressions such as “C₁₋₆ alkoxy”, “C₁₋₆ alkylthio”, “C₁₋₆ alkylsulphonyl” and “C₁₋₆ alkylamino” are to be construed accordingly.

The term “C₃₋₉ cycloalkyl” as used herein refers to monovalent groups of 3 to 9 carbon atoms derived from a saturated monocyclic hydrocarbon, and may comprise benzo-fused analogues thereof. Suitable C₃₋₉ cycloalkyl groups include cyclopropyl, cyclobutyl, benzocyclobutenyl, cyclopentyl, indanyl, cyclohexyl, cycloheptyl, cyclooctyl and cyclononanyl.

The term “C₄₋₁₂ bicycloalkyl” as used herein refers to monovalent groups of 4 to 12 carbon atoms derived from a saturated bicyclic hydrocarbon. Typical bicycloalkyl groups include bicyclo[1.1.1]pentanyl, bicyclo[3.1.0]hexanyl, bicyclo[4.1.0]heptanyl and bicyclo[2.2.2]octanyl.

The term “aryl” as used herein refers to monovalent carbocyclic aromatic groups derived from a single aromatic ring or multiple condensed aromatic rings. Suitable aryl groups include phenyl and naphthyl, preferably phenyl.

Suitable aryl(C₁₋₆)alkyl groups include benzyl, phenylethyl, phenylpropyl and naphthylmethyl.

The term “C₃₋₇ heterocycloalkyl” as used herein refers to saturated monocyclic rings containing 3 to 7 carbon atoms and at least one heteroatom selected from oxygen, sulphur and nitrogen, and may comprise benzo-fused analogues thereof. Suitable heterocycloalkyl groups include oxetanyl, azetidinyl, tetrahydrofuranyl, dihydrobenzo-furanyl, dihydrobenzothienyl, pyrrolidinyl, indolinyl, isoindolinyl, oxazolidinyl, thiazolidinyl, isothiazolidinyl, imidazolidinyl, tetrahydropyranyl, chromanyl, tetrahydro-thiopyranyl, piperidinyl, 1,2,3,4-tetrahydroquinolinyl, 1,2,3,4-tetrahydroisoquinolinyl, piperazinyl, 1,2,3,4-tetrahydroquinoxalinyl, hexahydro-[1,2,5]thiadiazolo[2,3-a]-pyrazinyl, homopiperazinyl, morpholinyl, benzoxazinyl, thiomorpholinyl, azepanyl, oxazepanyl, diazepanyl, thiadiazepanyl and azocanyl.

The term “C₄₋₉ heterobicycloalkyl” as used herein corresponds to C₄₋₉ bicycloalkyl wherein one or more of the carbon atoms have been replaced by one or more heteroatoms selected from oxygen, sulphur and nitrogen. Typical heterobicycloalkyl groups include 6-oxabicyclo[3.1.0]hexanyl, 3-azabicyclo[3.1.0]hexanyl, 2-oxa-5-azabicyclo[2.2.1]-heptanyl, 6-azabicyclo[3.2.0]heptanyl, 6-oxabicyclo[3.1.1]heptanyl, 3-azabicyclo[3.1.1]-heptanyl, 3-azabicyclo[4.1.0]heptanyl, 2-oxabicyclo[2.2.2]octanyl, quinuclidinyl, 2-oxa-5-azabicyclo[2.2.2]octanyl, 8-oxabicyclo[3.2.1]octanyl, 3-azabicyclo[3.2.1]octanyl, 8-azabicyclo[3.2.1]octanyl, 3-oxa-8-azabicyclo[3.2.1]octanyl, 3,8-diazabicyclo[3.2.1]-octanyl, 3,6-diazabicyclo[3.2.2]nonanyl, 3-oxa-7-azabicyclo[3.3.1]nonanyl, 3,7-dioxa-9-azabicyclo[3.3.1]nonanyl and 3,9-diazabicyclo[4.2.1]nonanyl.

The term “heteroaryl” as used herein refers to monovalent aromatic groups containing at least 5 atoms derived from a single ring or multiple condensed rings, wherein one or more carbon atoms have been replaced by one or more heteroatoms selected from oxygen, sulphur and nitrogen. Suitable heteroaryl groups include furyl, benzofuryl, dibenzofuryl, thienyl, benzothienyl, thieno[2,3-c]pyrazolyl, thieno[3,4-b]-[1,4]dioxinyl, dibenzothienyl, pyrrolyl, indolyl, pyrrolo[2,3-b]pyridinyl, pyrrolo[3,2-c]-pyridinyl, pyrrolo[3,4-b]pyridinyl, pyrazolyl, pyrazolo[1,5-a]pyridinyl, 4,5,6,7-tetrahydropyrazolo[1,5-a]pyridinyl, pyrazolo[3,4-d]pyrimidinyl, pyrazolo[1,5-a]-pyrazinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo[2,1-b]-thiazolyl, imidazo[1,2-a]pyridinyl, 5,6,7,8-tetrahydroimidazo[1,2-a]pyridinyl, imidazo-[4,5-b]pyridinyl, imidazo[1,2-b]pyridazinyl, purinyl, imidazo[1,2-a]pyrimidinyl, imidazo-[1,2-c]pyrimidinyl, imidazo[1,2-a]pyrazinyl, oxadiazolyl, thiadiazolyl, triazolyl, [1,2,4]-triazolo[1,5-a]pyridinyl, [1,2,4]triazolo[4,3-a]pyridinyl, [1,2,4]triazolo[4,3-a]pyrazinyl, 5,6,7,8-tetrahydro[1,2,4]triazolo[4,3-a]pyridinyl, [1,2,4]triazolo[1,5-a]pyrimidinyl, 6,8-dihydro-5H-[1,2,4]triazolo[4,3-a]pyrazinyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl and chromenyl groups.

The term “halogen” as used herein is intended to include fluorine, chlorine, bromine and iodine atoms, typically fluorine, chlorine or bromine.

Where the compounds of formula (I) have one or more asymmetric centres, they may accordingly exist as enantiomers. Where the compounds in accordance with the invention possess two or more asymmetric centres, they may additionally exist as diastereomers. The invention is to be understood to extend to the use of all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C═O)↔enol (CH═CHOH) tautomers or amide (NHC═O)↔hydroxyimine (N═COH) tautomers. Formula (I) and the formulae depicted hereinafter are intended to represent all individual tautomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium) or ³H (tritium) atom, preferably ¹H. Similarly, by way of example, each individual carbon atom present in formula (I), or in the formulae depicted hereinafter, may be present as a ¹²C, ¹³C or ¹⁴C atom, preferably ¹²C.

In a first embodiment, E represents a group of formula (Ea). In a second embodiment, E represents a group of formula (Eb). In a third embodiment, E represents a group of formula (Ec). In a fourth embodiment, E represents a group of formula (Ed). In a fifth embodiment, E represents a group of formula (Ee).

Typically, E represents a group of formula (Ea), (Eb) or (Ed).

Suitably, E represents a group of formula (Ea) or (Ed).

Generally, the present invention provides a compound of formula (IA-1), (IA-2), (IA-3), (IA-4) or (IA-5) or an N-oxide thereof, or a pharmaceutically acceptable salt thereof:

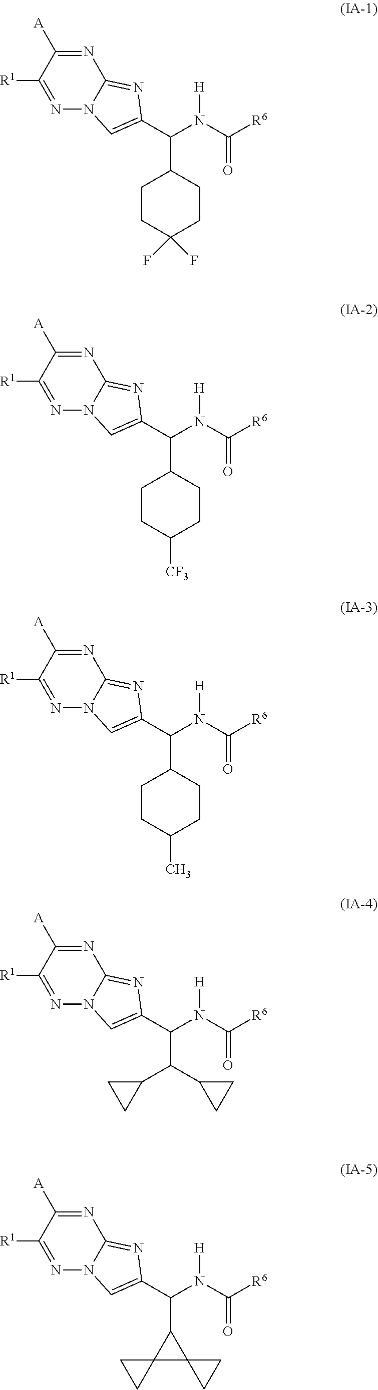

wherein A, R¹ and R⁶ are as defined above.

Typically, the present invention provides a compound of formula (IA-1), (IA-2) or (IA-4) as defined above or an N-oxide thereof, or a pharmaceutically acceptable salt thereof.

Suitably, the present invention provides a compound of formula (IA-1) or (IA-4) as defined above or an N-oxide thereof, or a pharmaceutically acceptable salt thereof.

In a first embodiment, A represents a group of formula (Aa). In a second embodiment, A represents a group of formula (Ab). In a third embodiment, A represents a group of formula (Ac). In a fourth embodiment, A represents a group of formula (Ad). In a fifth embodiment, A represents a group of formula (Ae).

Suitably, A represents a group of formula (Ab) or (Ad).

Generally, the present invention provides a compound of formula (IB-1), (IB-2), (IB-3), (IB-4) or (IB-5) or an N-oxide thereof, or a pharmaceutically acceptable salt thereof:

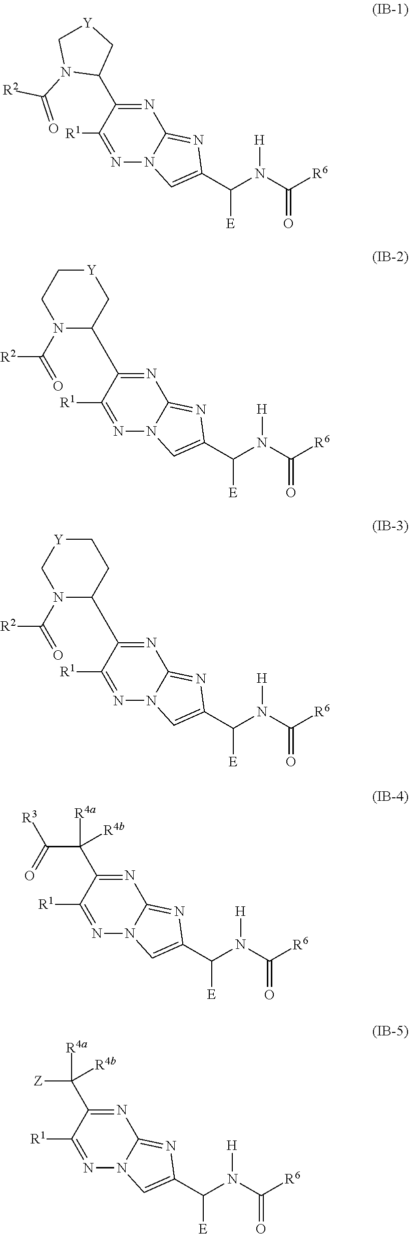

wherein E, Y, Z, R¹, R², R³, R^{4a}, R^{4b} and R⁶ are as defined above.

Suitably, the present invention provides a compound of formula (IB-2) or (IB-4) as defined above or an N-oxide thereof, or a pharmaceutically acceptable salt thereof.

In a first embodiment, Y represents —O—. In a second embodiment, Y represents —N(R⁷)—. In a third embodiment, Y represents —C(R^{5a})(R^{5b})—. In a fourth embodiment, Y represents —S—. In a fifth embodiment, Y represents —S(O)—. In a sixth embodiment, Y represents —S(O)₂—. In a seventh embodiment, Y represents —S(O)(N—R⁸)—.

Typically, Y represents —O—, —N(R⁷)—, —C(R^{5a})(R^{5b})— or —S(O)₂—, wherein R^{5a}, R^{5b} and R⁷ are as defined above.

Suitably, Y represents —O—, —C(R^{5a})(R^{5b})— or —S(O)₂—, wherein R^{5a} and R^{5b} are as defined above.

Generally, Z represents furyl, benzofuryl, dibenzofuryl, thienyl, benzothienyl, thieno[2,3-c]pyrazolyl, thieno[3,4-b][1,4]dioxinyl, dibenzothienyl, pyrrolyl, indolyl, pyrrolo[2,3-b]pyridinyl, pyrrolo[3,2-c]pyridinyl, pyrrolo[3,4-b]pyridinyl, pyrazolyl, pyrazolo[1,5-a]pyridinyl, 4,5,6,7-tetrahydropyrazolo[1,5-a]pyridinyl, pyrazolo[3,4-d]-pyrimidinyl, pyrazolo[1,5-a]pyrazinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo[2,1-b]thiazolyl, imidazo[1,2-a]pyridinyl, 5,6,7,8-tetrahydroimidazo[1,2-a]pyridinyl, imidazo[4,5-b]pyridinyl, imidazo[1,2-b]pyridazinyl, purinyl, imidazo[1,2-a]pyrimidinyl, imidazo[1,2-c]pyrimidinyl, imidazo[1,2-a]pyrazinyl, oxadiazolyl, thiadiazolyl, triazolyl, [1,2,4]triazolo[1,5-a]pyridinyl, [1,2,4]triazolo[4,3-a]-pyridinyl, [1,2,4]triazolo[4,3-a]pyrazinyl, 5,6,7,8-tetrahydro[1,2,4]triazolo[4,3-a]-pyridinyl, [1,2,4]triazolo[1,5-a]pyrimidinyl, 6,8-dihydro-5H-[1,2,4]triazolo[4,3-a]-pyrazinyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl or chromenyl, any of which groups may be optionally substituted by one or more substituents.

Appositely, Z represents pyrazolyl, pyrazolo[1,5-a]pyridinyl, isoxazolyl, isothiazolyl, imidazolyl, imidazo[1,2-a]pyridinyl, imidazo[1,2-a]pyrazinyl, oxadiazolyl, thiadiazolyl, triazolyl, [1,2,4]triazolo[1,5-a]pyridinyl, [1,2,4]triazolo[1,5-a]pyrazinyl, [1,2,4]triazolo[4,3-a]pyridinyl, tetrazolyl, pyridinyl, pyridazinyl, pyrimidinyl or pyrazinyl, any of which groups may be optionally substituted by one or more substituents.

Typically, Z represents imidazolyl, triazolyl, [1,2,4]triazolo[4,3-a]pyridinyl or tetrazolyl, any of which groups may be optionally substituted by one or more substituents.

Suitably, Z represents triazolyl, which group may be optionally substituted by one or more substituents.

Typical examples of optional substituents on Z include one, two or (where possible) three substituents independently selected from halogen, cyano, nitro, C₁₋₆ alkyl, difluoromethyl, difluoroethyl, trifluoro(C₁₋₆)alkyl, cyclopropyl, difluorocyclopropyl, difluorocyclobutyl, cyclopropylmethyl, difluorocyclopropylmethyl, fluorobicyclo[1.1.1]-pentanyl, cyanobicyclo[1.1.1]pentanyl, spiro[2.2]pentanyl, methylspiro[2.2]pentanyl, hydroxy, hydroxy(C₁₋₆)alkyl, oxo, C₁₋₆ alkoxy, difluoromethoxy, difluoroethoxy, trifluoromethoxy, trifluoroethoxy, phenoxy, methylenedioxy, difluoromethylenedioxy, C₁₋₆ alkylthio, C₁₋₆ alkylsulfinyl, C₁₋₆ alkylsulfonyl, amino, C₁₋₆ alkylamino, di(C₁₋₆)alkylamino, amino(C₁₋₆)alkyl, di(C₁₋₆)alkylamino(C₁₋₆)alkyl, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl and di(C₁₋₆)alkylsulfoximino.

Apposite examples of optional substituents on Z include one, two or (where possible) three substituents independently selected from halogen, cyano, C₁₋₆ alkyl, difluoromethyl, difluoroethyl, trifluoro(C₁₋₆)alkyl, cyclopropyl, difluorocyclopropyl, difluorocyclobutyl, cyclopropylmethyl, difluorocyclopropylmethyl, cyanobicyclo[1.1.1]-pentanyl and C₁₋₆ alkylamino.

Typical examples of particular substituents on Z include one, two or (where possible) three substituents independently selected from fluoro, chloro, bromo, cyano, nitro, methyl, ethyl, n-propyl, isopropyl, tert-butyl, difluoromethyl, difluoroethyl, trifluoromethyl, trifluoroethyl, trifluoropropyl, 2-methyl-3,3,3-trifluoropropyl, cyclopropyl, difluorocyclopropyl, difluorocyclobutyl, cyclopropylmethyl, difluorocyclopropylmethyl, fluorobicyclo[1.1.1]pentanyl, cyanobicyclo[1.1.1]pentanyl, spiro-[2.2]pentanyl, methylspiro[2.2]pentanyl, hydroxy, hydroxymethyl, hydroxyethyl, hydroxyisopropyl, oxo, methoxy, isopropoxy, difluoromethoxy, difluoroethoxy, trifluoromethoxy, trifluoroethoxy, phenoxy, methylenedioxy, difluoromethylenedioxy, methylthio, methylsulfinyl, methylsulfonyl, amino, methylamino, dimethylamino, aminomethyl, dimethylaminomethyl, acetylamino, methoxycarbonylamino, methylsulfonylamino, formyl, acetyl, carboxy, methoxycarbonyl, ethoxycarbonyl, aminocarbonyl, methylaminocarbonyl, dimethylaminocarbonyl, aminosulfonyl, methylaminosulfonyl, dimethylaminosulfonyl and dimethylsulfoximino.

Apposite examples of particular substituents on Z include one, two or (where possible) three substituents independently selected from fluoro, cyano, methyl, difluoro-methyl, difluoroethyl, trifluoroethyl, trifluoropropyl, 2-methyl-3,3,3-trifluoropropyl, cyclopropyl, difluorocyclopropyl, difluorocyclobutyl, cyclopropylmethyl, difluorocyclopropylmethyl, cyanobicyclo[1.1.1]pentanyl and methylamino.

Typical values of Z include trifluoroethylpyrazolyl, (methyl)(trifluoroethyl)-pyrazolyl, pyrazolo[1,5-a]pyridinyl, methylindazolyl, trifluoroethylisoxazolyl, (methyl)-(trifluoroethyl)isoxazolyl, trifluoroethylisothiazolyl, trifluoroethylimidazolyl, cyclopropylmethylimidazolyl, (methyl)(trifluoroethyl)imidazolyl, imidazo[1,2-a]-pyridinyl, difluoroethyltriazolyl, trifluoroethyltriazolyl, difluorocyclopropyltriazolyl, difluorocyclobutyltriazolyl, cyclopropylmethyltriazolyl, cyanobicyclo[1.1.1]pentanyltriazolyl, (fluoro)(trifluoroethyl)triazolyl, (methyl)(trifluoroethyl)triazolyl, (difluoro-methyl)(trifluoroethyl)triazolyl, (cyclopropylmethyl)(difluoromethyl)triazolyl, (methyl-amino)(trifluoroethyl)triazolyl, [1,2,4]triazolo[1,5-a]pyridinyl, fluoro[1,2,4]triazolo[4,3-a]pyridinyl, cyano[1,2,4]triazolo[4,3-a]pyridinyl, benzotriazolyl, trifluoroethyltetrazolyl, trifluoroethylpyridinyl, trifluoroethylpyridazinyl, trifluoroethylpyrimidinyl and trifluoroethylpyrazinyl.

Illustrative values of Z include cyclopropylmethylimidazolyl, difluoroethyltriazolyl, trifluoroethyltriazolyl, difluorocyclobutyltriazolyl, cyclopropylmethyltriazolyl, cyanobicyclo[1.1.1]pentanyltriazolyl, fluoro[1,2,4]triazolo[4,3-a]pyridinyl, cyano[1,2,4]-triazolo[4,3-a]pyridinyl and trifluoroethyltetrazolyl.

Suitably, Z represents a group of formula (Za), (Zb), (Zc), (Zd), (Ze), (Zf), (Zg), (Zh), (Zj), (Zk), (Zl), (Zm), (Zn), (Zp), (Zq), (Zr), (Zs), (Zt), (Zu), (Zv), (Zw), (Zx), (Zy), (Zz), (Zaa) or (Zab):

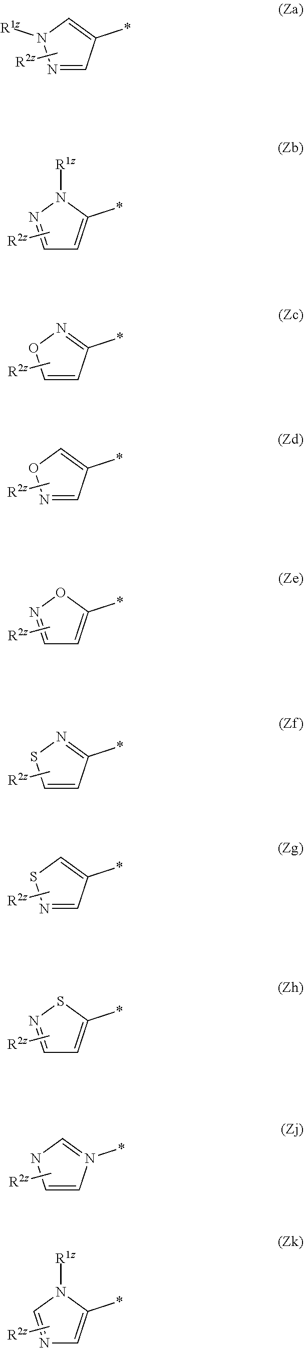

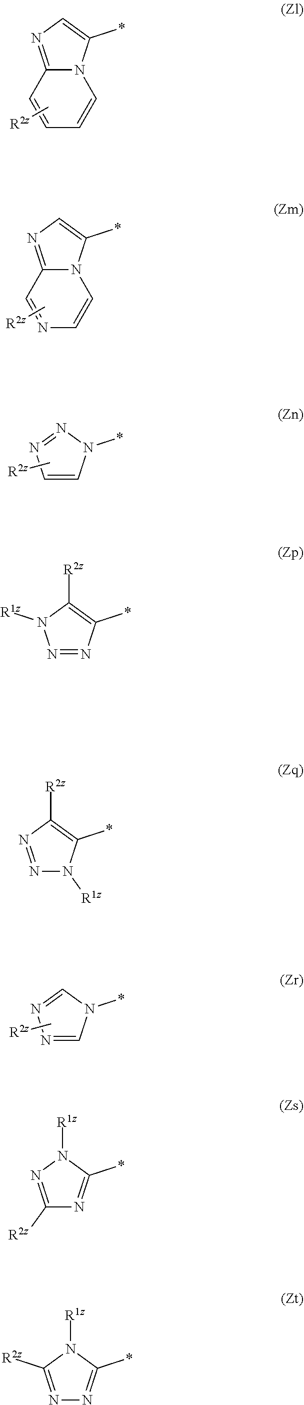

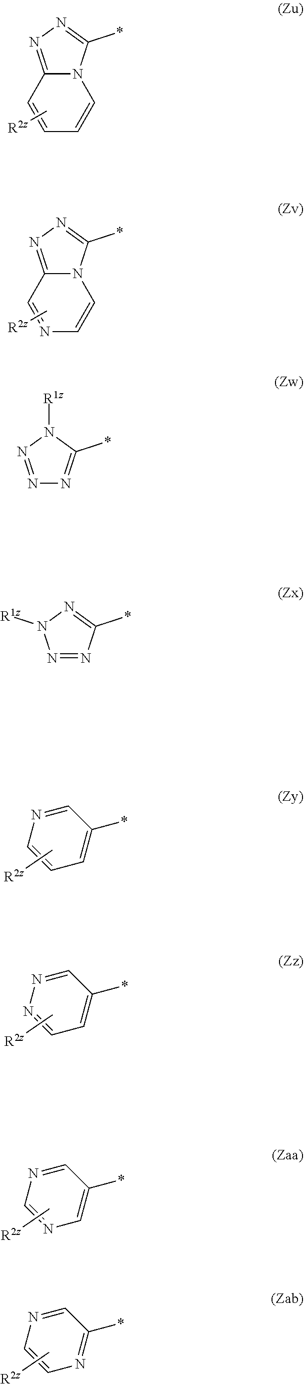

wherein
the asterisk (*) represents the point of attachment to the remainder of the molecule;
R^{1z} represents hydrogen, C₁₋₆ alkyl, difluoromethyl, difluoroethyl, trifluoro(C₁₋₆)-alkyl, cyclopropyl, difluorocyclopropyl, difluorocyclobutyl, cyclopropylmethyl, difluorocyclopropylmethyl, fluorobicyclo[1.1.1]pentanyl, cyanobicyclo[1.1.1]pentanyl, spiro[2.2]pentanyl, methylspiro[2.2]pentanyl, hydroxy(C₂₋₆)alkyl, C₁₋₆ alkylsulfonyl, amino(C₂₋₆)alkyl, di(C₁₋₆)alkylamino(C₁₋₆)alkyl, C₂₋₆ alkylcarbonyl, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl or di(C₁₋₆)alkylaminosulfonyl; and
R^{2z} represents hydrogen, halogen, cyano, nitro, C₁₋₆ alkyl, difluoromethyl, trifluoro(C₁₋₆)alkyl, cyclopropyl, difluorocyclopropyl, difluorocyclobutyl, cyclopropylmethyl, difluorocyclopropylmethyl, fluorobicyclo[1.1.1]pentanyl, cyanobicyclo[1.1.1]-pentanyl, spiro[2.2]pentanyl, methylspiro[2.2]pentanyl, hydroxy, hydroxy(C₁₋₆)alkyl, C₁₋₆ alkoxy, difluoromethoxy, difluoroethoxy, trifluoromethoxy, trifluoroethoxy, phenoxy, C₁₋₆ alkylthio, C₁₋₆ alkylsulfinyl, C₁₋₆ alkylsulfonyl, amino, C₁₋₆ alkylamino, di(C₁₋₆)alkylamino, amino(C₁₋₆)alkyl, di(C₁₋₆)alkylamino(C₁₋₆)alkyl, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl or di(C₁₋₆)alkylsulfoximino.

Particular values of Z include the groups of formula (Zk), (Zm), (Zp), (Zq), (Zt), (Zu), (Zv), (Zw) and (Zx) as defined above.

Typically, R^{1z} represents hydrogen, C₁₋₆ alkyl, difluoroethyl, trifluoro(C₁₋₆)alkyl, difluorocyclopropyl, difluorocyclobutyl, cyclopropylmethyl, difluorocyclopropylmethyl or cyanobicyclo[1.1.1]pentanyl.

Apposite values of R^{1z} include hydrogen, methyl, ethyl, n-propyl, isopropyl, tert-butyl, difluoromethyl, trifluoromethyl, difluoroethyl, trifluoroethyl, trifluoropropyl, 2-methyl-3,3,3-trifluoropropyl, cyclopropyl, difluorocyclopropyl, difluorocyclobutyl, cyclopropylmethyl, difluorocyclopropylmethyl, fluorobicyclo[1.1.1]pentanyl, cyanobicyclo[1.1.1]pentanyl, spiro[2.2]pentanyl, methylspiro[2.2]pentanyl, hydroxyethyl, hydroxyisopropyl, methylsulfonyl, aminoethyl, dimethylaminomethyl, acetyl, methoxycarbonyl, ethoxycarbonyl, aminocarbonyl, methylaminocarbonyl, dimethylaminocarbonyl, aminosulfonyl, methylaminosulfonyl and dimethylaminosulfonyl.

Typical values of R^{1z} include hydrogen, methyl, ethyl, n-propyl, isopropyl, tert-butyl, difluoroethyl, trifluoroethyl, trifluoropropyl, 2-methyl-3,3,3-trifluoropropyl, difluorocyclopropyl, difluorocyclobutyl, cyclopropylmethyl, difluorocyclopropylmethyl and cyanobicyclo[1.1.1]pentanyl.

Typically, R^{2z} represents hydrogen, halogen, cyano, C₁₋₆ alkyl, trifluoro(C₁₋₆)alkyl, cyclopropylmethyl, difluorocyclopropylmethyl or C₁₋₆ alkylamino.

Suitably, R^{2z} represents hydrogen, halogen or cyano. In a first embodiment, R^{2z} represents hydrogen. In a second embodiment, R^{2z} represents halogen, especially fluoro. In a third embodiment, R^{2z} represents cyano.

Apposite values of R^{2z} include hydrogen, fluoro, chloro, bromo, cyano, nitro, methyl, ethyl, n-propyl, isopropyl, tert-butyl, difluoromethyl, trifluoromethyl, trifluoroethyl, trifluoropropyl, 2-methyl-3,3,3-trifluoropropyl, cyclopropyl, difluorocyclopropyl, difluorocyclobutyl, cyclopropylmethyl, difluorocyclopropylmethyl, fluorobicyclo[1.1.1]-pentanyl, cyanobicyclo[1.1.1]pentanyl, spiro[2.2]pentanyl, methylspiro[2.2]pentanyl, hydroxy, hydroxymethyl, hydroxyethyl, hydroxyisopropyl, methoxy, isopropoxy, difluoromethoxy, difluoroethoxy, trifluoromethoxy, trifluoroethoxy, phenoxy, methylthio, methylsulfinyl, methylsulfonyl, amino, methylamino, ethylamino, dimethylamino, aminomethyl, dimethylaminomethyl, acetylamino, methoxycarbonylamino, methylsulfonylamino, formyl, acetyl, carboxy, methoxycarbonyl, ethoxycarbonyl, aminocarbonyl, methylaminocarbonyl, dimethylaminocarbonyl, aminosulfonyl, methylaminosulfonyl, dimethylaminosulfonyl and dimethylsulfoximino.

Typical values of R^{2z} include hydrogen, fluoro, cyano, methyl, difluoromethyl, trifluoroethyl, trifluoropropyl, 2-methyl-3,3,3-trifluoropropyl, cyclopropylmethyl, difluorocyclopropylmethyl and methylamino.

Suitable values of R^{2z} include hydrogen, fluoro and cyano.

In a first embodiment, R¹ represents hydrogen. In a second embodiment, R¹ represents fluoro. In a third embodiment, R¹ represents chloro. In a fourth embodiment, R¹ represents methyl. In a fifth embodiment, R¹ represents difluoromethyl. In a sixth embodiment, R¹ represents trifluoromethyl.

Typically, R¹ represents hydrogen, fluoro, chloro or methyl.

Generally, R¹ represents hydrogen or fluoro.

Suitably, R¹ represents hydrogen.

Typically, R² represents C₃₋₉ cycloalkyl, C₄₋₁₂ bicycloalkyl or C₃₋₇ heterocycloalkyl, any of which groups may be optionally substituted by one or more substituents.

Suitably, R² represents C₄₋₁₂ bicycloalkyl or C₃₋₇ heterocycloalkyl, either of which groups may be optionally substituted by one or more substituents.

Typical examples of R² include cyclobutyl, bicyclo[1.1.1]pentanyl, azetidinyl, pyrrolidinyl, tetrahydropyranyl and morpholinyl, any of which groups may be optionally substituted by one or more substituents.

Suitable examples of R² include bicyclo[1.1.1]pentanyl and tetrahydropyranyl, either of which groups may be optionally substituted by one or more substituents.

Typical examples of optional substituents on R² include one, two, three or four substituents independently selected from halogen.

Typical examples of particular substituents on R² include one, two, three or four substituents independently selected from fluoro.

Typical values of R² include difluorocyclobutyl, fluorobicyclo[1.1.1]pentanyl, difluoroazetidinyl, difluoropyrrolidinyl, tetrafluoropyrrolidinyl, difluorotetrahydropyranyl and tetrafluoromorpholinyl.

Suitable values of R² include fluorobicyclo[1.1.1]pentanyl and difluorotetrahydropyranyl.

In a first embodiment, R^{2a} represents C₁₋₆ alkyl. In a second embodiment, R^{2a} represents optionally substituted C₃₋₉ cycloalkyl.

Typically, R^{2a} represents C₁₋₆ alkyl; or R^{2a} represents cyclobutyl, which group may be optionally substituted by one or more substituents.

Typical examples of optional substituents on R^{2a} include one, two or three substituents independently selected from halogen, cyano, nitro, C₁₋₆ alkyl, trifluoro-methyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, amino(C₁₋₆)alkyl, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Suitable examples of optional substituents on R^{2a} include one, two or three substituents independently selected from halogen.

Typical examples of specific substituents on R^{2a} include one, two or three substituents independently selected from fluoro, chloro, bromo, cyano, nitro, methyl, ethyl, isopropyl, tert-butyl, trifluoromethylhydroxy, hydroxymethyl, oxo, methoxy, tert-butoxy, difluoromethoxy, trifluoromethoxy, methylthio, methylsulfinyl, methylsulfonyl, amino, aminomethyl, aminoethyl, methylamino, tert-butylamino, dimethylamino, acetylamino, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl and dimethylaminosulfonyl.

Suitable examples of specific substituents on R^{2a} include one, two or three substituents independently selected from fluoro.

Illustrative examples of specific values of R^{2a} include methyl, ethyl, n-propyl, isopropyl, n-butyl, tert-butyl, cyclobutyl and difluorocyclobutyl.

In a first embodiment, R³ represents —NR^{3a}R^{3b}. In a second embodiment, R³ represents a group of formula (Wa) as defined above.

In a first embodiment, R^{3a} represents hydrogen. In a second embodiment, R^{3a} represents C₁₋₆ alkyl, especially methyl or ethyl. In a first aspect of that embodiment, R^{3a} represents methyl. In a second aspect of that embodiment, R^{3a} represents ethyl.

Typically, R^{3b} represents C₁₋₆ alkyl or C₃₋₇ cycloalkyl(C₁₋₆)alkyl, either of which groups may be optionally substituted by one or more substituents.

Suitably, R^{3b} represents C₁₋₆ alkyl, which group may be optionally substituted by one or more substituents.

In a first embodiment, R^{3b} represents optionally substituted C₁₋₆ alkyl. In a second embodiment, R³⁶ represents optionally substituted C₃₋₇ cycloalkyl. In a third embodiment, R³⁶ represents optionally substituted C₃₋₇ cycloalkyl(C₁₋₆)alkyl. In a fourth embodiment, R³⁶ represents optionally substituted C₄₋₁₂ bicycloalkyl. In a fifth embodiment, R³⁶ represents optionally substituted aryl. In a sixth embodiment, R^{3b} represents optionally substituted aryl(C₁₋₆)alkyl. In a seventh embodiment, R^{3b} represents optionally substituted C₃₋₇ heterocycloalkyl. In an eighth embodiment, R^{3b} represents optionally substituted C₃₋₇ heterocycloalkyl(C₁₋₆)alkyl. In a ninth embodiment, R 3b represents optionally substituted heteroaryl. In a tenth embodiment, R^{3b} represents optionally substituted heteroaryl(C₁₋₆)alkyl.

Typical examples of R^{3b} include ethyl, propyl, isopropyl, 2-methylpropyl and cyclopropylmethyl, any of which groups may be optionally substituted by one or more substituents.

Suitable examples of R^{3b} include propyl, which group may be optionally substituted by one or more substituents.

Typical examples of optional substituents on R^{3b} include one, two or three substituents independently selected from halogen, cyano, nitro, C₁₋₆ alkyl, trifluoro-methyl, hydroxy, C₁₋₆ alkoxy, difluoromethoxy, difluoroethoxy, trifluoromethoxy, trifluoroethoxy, C₁₋₆ alkylthio, C₁₋₆ alkylsulfinyl, C₁₋₆ alkylsulfonyl, amino, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl and di(C₁₋₆)alkylsulfoximino.

Suitable examples of optional substituents on R^{3b} include one, two or three substituents independently selected from halogen.

Typical examples of particular substituents on R^{3b} include one, two or three substituents independently selected from fluoro, chloro, bromo, cyano, nitro, methyl, ethyl, trifluoromethyl, hydroxy, methoxy, isopropoxy, difluoromethoxy, difluoroethoxy, trifluoromethoxy, trifluoroethoxy, methylthio, methylsulfinyl, methylsulfonyl, ethylsulfonyl, amino, methylamino, dimethylamino, acetylamino, methoxycarbonylamino, methylsulfonylamino, formyl, acetyl, carboxy, methoxycarbonyl, ethoxycarbonyl, aminocarbonyl, methylaminocarbonyl, dimethylaminocarbonyl, aminosulfonyl, methylaminosulfonyl, dimethylaminosulfonyl and dimethylsulfoximino.

Suitable examples of particular substituents on R^{3b} include one, two or three substituents independently selected from fluoro.

Typical values of R^{3b} include difluoroethyl, trifluoroethyl, difluoropropyl, trifluoroisopropyl, methylaminocarbonyl-2-methylpropyl, (cyclopropyl)(trifluoromethyl)-methyl and difluorocyclopropylmethyl.

Suitable values of R^{3b} include difluoropropyl.

In a first embodiment, W represents the residue of an optionally substituted saturated monocyclic ring containing 3 to 6 carbon atoms, one nitrogen atom, and 0, 1, 2 or 3 additional heteroatoms independently selected from N, O and S, but containing no more than one O or S atom. In a first aspect of that embodiment, W represents the residue of an optionally substituted saturated monocyclic ring containing 3 or 4 carbon atoms, one nitrogen atom, and 0, 1, 2 or 3 additional heteroatoms independently selected from N, O and S, but containing no more than one O or S atom.

In a second embodiment, W represents the residue of an optionally substituted saturated bicyclic ring system containing 4 to 10 carbon atoms, one nitrogen atom, and 0, 1, 2 or 3 additional heteroatoms independently selected from N, O and S, but containing no more than one O or S atom. In a first aspect of that embodiment, W represents the residue of an optionally substituted saturated bicyclic ring system containing 5, 6 or 7 carbon atoms, one nitrogen atom, and 0, 1, 2 or 3 additional heteroatoms independently selected from N, O and S, but containing no more than one O or S atom.

In a third embodiment, W represents the residue of an optionally substituted saturated spirocyclic ring system containing 5 to 10 carbon atoms, one nitrogen atom, and 0, 1, 2 or 3 additional heteroatoms independently selected from N, O and S, but containing no more than one O or S atom. In a first aspect of that embodiment, W represents the residue of an optionally substituted saturated spirocyclic ring system containing 5, 6 or 7 carbon atoms, one nitrogen atom, and 0, 1, 2 or 3 additional heteroatoms independently selected from N, O and S, but containing no more than one O or S atom.

Suitably, W represents the residue of an optionally substituted saturated monocyclic ring containing 3 or 4 carbon atoms, one nitrogen atom, and 0 or 1 oxygen atom(s). In a first embodiment, W represents the residue of an optionally substituted saturated monocyclic ring containing 3 or 4 carbon atoms and one nitrogen atom. In a first aspect of that embodiment, W represents the residue of an optionally substituted saturated monocyclic ring containing 3 carbon atoms and one nitrogen atom. In a second aspect of that embodiment, W represents the residue of an optionally substituted saturated monocyclic ring containing 4 carbon atoms and one nitrogen atom. In a second embodiment, W represents the residue of an optionally substituted saturated monocyclic ring containing 4 carbon atoms, one nitrogen atom, and one oxygen atom.

In a first embodiment, the group of formula (Wa) represents a saturated monocyclic ring containing one nitrogen atom and no additional heteroatoms (i.e. it is an optionally substituted azetidin-1-yl, pyrrolidin-1-yl, piperidin-1-yl or hexahydroazepin-1-yl ring). In a second embodiment, the group of formula (Wa) represents a saturated monocyclic ring containing one nitrogen atom and one additional heteroatom selected from N, O and S. In a first aspect of that embodiment, the group of formula (Wa) is an optionally substituted morpholin-4-yl moiety. In a third embodiment, the group of formula (Wa) represents a saturated monocyclic ring containing one nitrogen atom and two additional heteroatoms selected from N, O and S, of which not more than one is O or

S. In a fourth embodiment, the group of formula (Wa) represents a saturated monocyclic ring containing one nitrogen atom and three additional heteroatoms selected from N, O and S, of which not more than one is O or S.

Typical values of the group of formula (Wa) include azetidin-1-yl, pyrrolidin-1-yl, oxazolidin-3-yl, thiazolidin-3-yl, isothiazolidin-2-yl, imidazolidin-1-yl, piperidin-1-yl, piperazin-1-yl, homopiperazin-1-yl, morpholin-4-yl, thiomorpholin-4-yl, azepan-1-yl, [1,4]oxazepan-4-yl, [1,4]diazepan-1-yl, [1,4]thiadiazepan-4-yl, azocan-1-yl, 3-azabicyclo-[3.1.0]hexan-3-yl, 2-oxa-5-azabicyclo[2.2.1]heptan-5-yl, 6-azabicyclo[3.2.0]heptan-6-yl, 3-azabicyclo[3.1.1]heptan-3-yl, 6-oxa-3-azabicyclo[3.1.1]heptan-3-yl, 3-azabicyclo-[4.1.0]heptan-3-yl, 2-oxa-5-azabicyclo[2.2.2]octan-5-yl, 3-azabicyclo[3.2.1]octan-3-yl, 8-azabicyclo[3.2.1]octan-8-yl, 3-oxa-8-azabicyclo[3.2.1]octan-8-yl, 3,8-diazabicyclo-[3.2.1]octan-3-yl, 3,8-diazabicyclo[3.2.1]octan-8-yl, 3,6-diazabicyclo[3.2.2]nonan-3-yl, 3,6-diazabicyclo[3.2.2]nonan-6-yl, 3-oxa-7-azabicyclo[3.3.1]nonan-7-yl, 3,7-dioxa-9-azabicyclo[3.3.1]nonan-9-yl, 3,9-diazabicyclo[4.2.1]nonan-3-yl, 3,9-diazabicyclo[4.2.1]-nonan-9-yl, 5-azaspiro[2.3]hexan-5-yl, 5-azaspiro[2.4]heptan-5-yl, 2-azaspiro[3.3]heptan-2-yl, 2-oxa-6-azaspiro[3.3]heptan-6-yl, 3-oxa-6-azaspiro[3.3]heptan-6-yl, 6-thia-2-azaspiro[3.3]heptan-2-yl, 2-oxa-6-azaspiro[3.4]octan-6-yl, 2-oxa-6-azaspiro[3.5]nonan-6-yl, 7-oxa-2-azaspiro[3.5]nonan-2-yl, 2-oxa-7-azaspiro[3.5]nonan-7-yl, 2,4,8-triazaspiro[4.5]-decan-2-yl, 2,4,8-triazaspiro[4.5]decan-4-yl and 2,4,8-triazaspiro[4.5]decan-8-yl, any of which groups may be optionally substituted by one or more substituents.

Suitable values of the group of formula (Wa) include azetidin-1-yl and pyrrolidin-1-yl, either of which groups may be optionally substituted by one or more substituents.

In a first embodiment, the group of formula (Wa) is unsubstituted. In a second embodiment, the group of formula (Wa) is substituted by one or more substituents, typically by one to six substituents, suitably by two to four substituents. In a first aspect of that embodiment, the group of formula (Wa) is substituted by one substituent. In a second aspect of that embodiment, the group of formula (Wa) is substituted by two substituents. In a third aspect of that embodiment, the group of formula (Wa) is substituted by three substituents. In a fourth aspect of that embodiment, the group of formula (Wa) is substituted by four substituents. In a fifth aspect of that embodiment, the group of formula (Wa) is substituted by five substituents. In a sixth aspect of that embodiment, the group of formula (Wa) is substituted by six substituents.

Typical examples of optional substituents on the group of formula (Wa) include halogen, C₁₋₆ alkyl, trifluoromethyl, hydroxy, hydroxy(C₁₋₆)alkyl, C₁₋₆ alkoxy, difluoromethoxy, trifluoromethoxy, C₁₋₆ alkoxy(C₁₋₆)alkyl, C₁₋₆ alkylthio, C₁₋₆ alkylsulfonyl, cyano, oxo, formyl, C₂₋₆ alkylcarbonyl, carboxy, carboxy(C₁₋₆)alkyl, C₂₋₆ alkoxycarbonyl, C₂₋₆ alkoxycarbonyl(C₁₋₆)alkyl, amino, amino(C₁₋₆)alkyl, C₁₋₆ alkylamino, di(C₁₋₆)alkylamino, C₂₋₆ alkylcarbonylamino, C₂₋₆ alkoxycarbonylamino, C₁₋₆ alkylsulfonylamino, aminocarbonyl, C₁₋₆ alkylaminocarbonyl and di(C₁₋₆)alkylaminocarbonyl.

Selected examples of optional substituents on the group of formula (Wa) include halogen and trifluoromethyl.

Suitable examples of optional substituents on the group of formula (Wa) include halogen.

Typical examples of particular substituents on the group of formula (Wa) include fluoro, chloro, bromo, methyl, ethyl, isopropyl, trifluoromethyl, hydroxy, hydroxymethyl, hydroxyethyl, methoxy, isopropoxy, difluoromethoxy, trifluoromethoxy, methoxymethyl, methylthio, ethylthio, methylsulfonyl, cyano, oxo, formyl, acetyl, ethylcarbonyl, tert-butylcarbonyl, carboxy, carboxymethyl, methoxycarbonyl, ethoxycarbonyl, tert-butoxycarbonyl, methoxycarbonylmethyl, ethoxycarbonylmethyl, amino, aminomethyl, methyl-amino, ethylamino, dimethylamino, acetylamino, tert-butoxycarbonylamino, methylsulfonylamino, aminocarbonyl, methylaminocarbonyl and dimethylaminocarbonyl.

Selected examples of particular substituents on the group of formula (Wa) include fluoro and trifluoromethyl.

Suitable examples of particular substituents on the group of formula (Wa) include fluoro.

Selected values of the group of formula (Wa) include trifluoromethylazetidin-1-yl and tetrafluoropyrrolidin-1-yl.

Generally, R^{4a} represents hydrogen or fluoro; or R^{4a} represents C₁₋₆ alkyl, which group may be optionally substituted by one or more substituents.

Typically, R^{4a} represents hydrogen; or R^{4a} represents C₁₋₆ alkyl, which group may be optionally substituted by one or more substituents.

Suitably, R^{4a} represents C₁₋₆ alkyl, which group may be optionally substituted by one or more substituents.

In a first embodiment, R^{4a} represents hydrogen. In a second embodiment, R^{4a} represents fluoro. In a third embodiment, R^{4a} represents hydroxy. In a fourth embodiment, R^{4a} represents C₁₋₆ alkyl, especially methyl or ethyl, which group may be optionally substituted by one or more substituents. In a first aspect of that embodiment, R^{4a} represents optionally substituted methyl. In a second aspect of that embodiment, R^{4a} represents optionally substituted ethyl.

Typical examples of optional substituents on R^{4a} include one, two or three substituents independently selected from halogen, cyano, nitro, hydroxy, C₁₋₆ alkoxy, difluoromethoxy, difluoroethoxy, trifluoromethoxy, trifluoroethoxy, C₁₋₆ alkylthio, C₁₋₆ alkylsulfinyl, C₁₋₆ alkylsulfonyl, amino, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di-(C₁₋₆)alkylaminocarbonyl, aminosulfonyl, C₁₋₆ alkylaminosulfonyl, di(C₁₋₆)alkylaminosulfonyl and di(C₁₋₆)alkylsulfoximino.

Suitable examples of optional substituents on R^{4a} include one, two or three substituents independently selected from halogen.

Typical examples of particular substituents on R^{4a} include one, two or three substituents independently selected from fluoro, chloro, bromo, cyano, nitro, hydroxy, methoxy, isopropoxy, difluoromethoxy, difluoroethoxy, trifluoromethoxy, trifluoroethoxy, methylthio, methylsulfinyl, methylsulfonyl, ethylsulfonyl, amino, methylamino, dimethylamino, acetylamino, methoxycarbonylamino, methylsulfonylamino, formyl, acetyl, carboxy, methoxycarbonyl, ethoxycarbonyl, aminocarbonyl, methylaminocarbonyl, dimethylaminocarbonyl, aminosulfonyl, methylaminosulfonyl, dimethylaminosulfonyl and dimethylsulfoximino.

Suitable examples of particular substituents on R^{4a} include one, two or three substituents independently selected from fluoro.

Illustrative values of R^{4a} include hydrogen, fluoro, hydroxy, methyl, difluoroethyl and trifluoroethyl.

In a first embodiment, R^{4b} represents hydrogen. In a second embodiment, R^{4b} represents fluoro. In a third embodiment, R^{4b} represents C₁₋₆ alkyl, especially methyl or ethyl. In a first aspect of that embodiment, R^{4b} represents methyl. In a second aspect of that embodiment, R^{4b} represents ethyl.

Typical values of R^{4b} include hydrogen and fluoro.

Alternatively, R^{4a} and R^{4b} may together form an optionally substituted cyclic moiety. Thus, R^{4a} and R^{4b}, when taken together with the carbon atom to which they are both attached, may represent C₃₋₇ cycloalkyl or C₃₋₇ heterocycloalkyl, either of which groups may be unsubstituted, or substituted by one or more substituents, typically by one or two substituents.

In a first embodiment, R^{4a} and R^{4b}, when taken together with the carbon atom to which they are both attached, may suitably represent C₃₋₇ cycloalkyl, which group may be unsubstituted, or substituted by one or more substituents, typically by one or two substituents. As a general illustration of that embodiment, R^{4a} and R^{4b}, when taken together with the carbon atom to which they are both attached, may suitably represent cyclopropyl, cyclobutyl, cyclopentyl or cyclohexyl, any of which groups may be unsubstituted, or substituted by one or more substituents, typically by one or two substituents. As a particular illustration of that embodiment, R^{4a} and R^{4b}, when taken together with the carbon atom to which they are both attached, may suitably represent cyclobutyl or cyclohexyl, either of which groups may be unsubstituted, or substituted by one or more substituents, typically by one or two substituents. In a first aspect of that embodiment, R^{4a} and R^{4b}, when taken together with the carbon atom to which they are both attached, may suitably represent a cyclopropyl ring, which may be unsubstituted, or substituted by one or more substituents, typically by one or two substituents. In a second aspect of that embodiment, R^{4a} and R^{4b}, when taken together with the carbon atom to which they are both attached, may suitably represent a cyclobutyl ring, which may be unsubstituted, or substituted by one or more substituents, typically by one or two substituents. In a third aspect of that embodiment, R^{4a} and R^{4b}, when taken together with the carbon atom to which they are both attached, may suitably represent a cyclopentyl ring, which may be unsubstituted, or substituted by one or more substituents, typically by one or two substituents. In a fourth aspect of that embodiment, R^{4a} and R^{4b}, when taken together with the carbon atom to which they are both attached, may suitably represent a cyclohexyl ring, which may be unsubstituted, or substituted by one or more substituents, typically by one or two substituents.

In a second embodiment, R^{4a} and R^{4b}, when taken together with the carbon atom to which they are both attached, may suitably represent C₃₋₇ heterocycloalkyl, which group may be unsubstituted, or substituted by one or more substituents, typically by one or two substituents. As a general illustration of that embodiment, R^{4a} and R^{4b}, when taken together with the carbon atom to which they are both attached, may suitably represent oxetanyl, pyrrolidinyl, tetrahydropyranyl or piperidinyl, any of which groups may be unsubstituted, or substituted by one or more substituents, typically by one or two substituents. As a particular illustration of that embodiment, R^{4a} and R^{4b}, when taken together with the carbon atom to which they are both attached, may suitably represent pyrrolidinyl, tetrahydropyranyl or piperidinyl, any of which groups may be unsubstituted, or substituted by one or more substituents, typically by one or two substituents. As a more particular illustration of that embodiment, R^{4a} and R^{4b}, when taken together with the carbon atom to which they are both attached, may suitably represent tetrahydropyranyl or piperidinyl, either of which groups may be unsubstituted, or substituted by one or more substituents, typically by one or two substituents. In a first aspect of that embodiment, R^{4a} and R^{4b}, when taken together with the carbon atom to which they are both attached, may suitably represent an oxetanyl ring, which may be unsubstituted, or substituted by one or more substituents, typically by one or two substituents. In a second aspect of that embodiment, R^{4a} and R^{4b}, when taken together with the carbon atom to which they are both attached, may suitably represent a pyrrolidinyl ring, which may be unsubstituted, or substituted by one or more substituents, typically by one or two substituents. In a third aspect of that embodiment, R^{4a} and R^{4b}, when taken together with the carbon atom to which they are both attached, may suitably represent a tetrahydropyranyl ring, which may be unsubstituted, or substituted by one or more substituents, typically by one or two substituents. In a fourth aspect of that embodiment, R^{4a} and R^{4b}, when taken together with the carbon atom to which they are both attached, may suitably represent a piperidinyl ring, which may be unsubstituted, or substituted by one or more substituents, typically by one or two substituents.

Typically, R^{4a} and R^{4b}, when taken together with the carbon atom to which they are both attached, may represent cyclopropyl, cyclobutyl, cyclopentyl, cyclohexyl, oxetanyl, pyrrolidinyl, tetrahydropyranyl or piperidinyl, any of which groups may be unsubstituted, or substituted by one or more substituents, typically by one or two substituents.

Appositely, R^{4a} and R^{4b}, when taken together with the carbon atom to which they are both attached, may represent cyclohexyl, tetrahydropyranyl or piperidinyl, any of which groups may be unsubstituted, or substituted by one or more substituents, typically by one or two substituents.

Suitably, R^{4a} and R^{4b}, when taken together with the carbon atom to which they are both attached, may represent cyclohexyl or tetrahydropyranyl, either of which groups may be unsubstituted, or substituted by one or more substituents, typically by one or two substituents.

Typical examples of optional substituents on the cyclic moiety formed by R^{4a} and R^{4b} include one, two or three substituents independently selected from C₁₋₆ alkyl, halogen, cyano, trifluoromethyl, trifluoroethyl, hydroxy, C₁₋₆ alkoxy, C₁₋₆ alkylthio, C₁₋₆ alkylsulfinyl, C₁₋₆ alkylsulfonyl, C₂₋₆ alkylcarbonyl, C₂₋₆ alkoxycarbonyl, amino, C₁₋₆ alkylamino and di(C₁₋₆)alkylamino. Additional examples include oxetanyl.

Apposite examples of optional substituents on the cyclic moiety formed by R^{4a} and R^{4b} include one, two or three substituents independently selected from halogen and oxetanyl.

Suitable examples of optional substituents on the cyclic moiety formed by R^{4a} and R^{4b} include one, two or three substituents independently selected from halogen.

Typical examples of particular substituents on the cyclic moiety formed by R^{4a} and R^{4b} include one, two or three substituents independently selected from methyl, fluoro, chloro, bromo, cyano, trifluoromethyl, trifluoroethyl, hydroxy, methoxy, methylthio, methylsulfinyl, methylsulfonyl, acetyl, methoxycarbonyl, ethoxycarbonyl, amino, methyl-amino and dimethylamino. Additional examples include oxetanyl.

Apposite examples of particular substituents on the cyclic moiety formed by R^{4a} and R^{4b} include one, two or three substituents independently selected from fluoro and oxetanyl.

Suitable examples of particular substituents on the cyclic moiety formed by R^{4a} and R^{4b} include one, two or three substituents independently selected from fluoro.

Typical examples of the cyclic moiety formed by R^{4a} and R^{4b} include cyclopropyl, difluorocyclobutyl, cyclopentyl, difluorocyclohexyl, oxetanyl, methoxycarbonyl-pyrrolidinyl, tetrahydropyranyl, piperidinyl and methoxycarbonylpiperidinyl. Additional examples include oxetanylpiperidinyl.

Selected examples of the cyclic moiety formed by R^{4a} and R^{4b} include difluoro-cyclohexyl, tetrahydropyranyl and oxetanylpiperidinyl.

Suitable examples of the cyclic moiety formed by R^{4a} and R^{4b} include difluoro-cyclohexyl and tetrahydropyranyl.

In a first embodiment, R^{5a} represents hydrogen. In a second embodiment, R^{5a} represents fluoro. In a third embodiment, R^{5a} represents methyl. In a fourth embodiment, R^{5a} represents difluoromethyl. In a fifth embodiment, R^{5a} represents trifluoromethyl.

Typically, R^{5a} represents hydrogen, fluoro, difluoromethyl or trifluoromethyl.

Appositely, R^{5a} represents hydrogen, methyl, difluoromethyl or trifluoromethyl.

Suitably, R^{5a} represents difluoromethyl or trifluoromethyl.

In a first embodiment, R^{5b} represents hydrogen. In a second embodiment, R^{5b} represents fluoro. In a third embodiment, R^{5b} represents methyl. In a fourth embodiment, R^{5b} represents hydroxy.

Typically, R^{5b} represents hydrogen, fluoro or hydroxy.

Suitably, R^{5b} represents fluoro or hydroxy, especially hydroxy.

Alternatively, R^{5a} and R^{5b} may together form a spiro linkage. Thus, R^{5a} and R^{5b}, when taken together with the carbon atom to which they are both attached, may represent cyclopropyl.

Typically, R⁶ represents —OR^{6a} or —NR^{6b}R^{6c}; or R⁶ represents C₁₋₆ alkyl, C₃₋₉ cycloalkyl, C₃₋₉ cycloalkyl(C₁₋₆)alkyl, aryl, aryl(C₁₋₆)alkyl, heteroaryl or heteroaryl-(C₁₋₆)alkyl, any of which groups may be optionally substituted by one or more substituents.

Appositely, R⁶ represents —OR^{6a} or —NR^{6b}R^{6c}; or R⁶ represents C₃₋₉ cycloalkyl, aryl or heteroaryl, any of which groups may be optionally substituted by one or more substituents.

Suitably, R⁶ represents —OR^{6a}; or R⁶ represents C₃₋₉ cycloalkyl or heteroaryl, either of which groups may be optionally substituted by one or more substituents.

In a first embodiment, R⁶ represents optionally substituted C₁₋₆ alkyl. In a second embodiment, R⁶ represents optionally substituted C₃₋₉ cycloalkyl. In a third embodiment, R⁶ represents optionally substituted C₃₋₉ cycloalkyl(C₁₋₆)alkyl. In a fourth embodiment, R⁶ represents optionally substituted aryl. In a fifth embodiment, R⁶ represents optionally substituted aryl(C₁₋₆)alkyl. In a sixth embodiment, R⁶ represents optionally substituted C₃₋₇ heterocycloalkyl. In a seventh embodiment, R⁶ represents optionally substituted C₃₋₇ heterocycloalkyl(C₁₋₆)alkyl. In an eighth embodiment, R⁶ represents optionally substituted heteroaryl. In a ninth embodiment, R⁶ represents optionally substituted heteroaryl(C₁₋₆)alkyl. In a tenth embodiment, R⁶ represents —OR^{6a}. In an eleventh embodiment, R⁶ represents —NR^{6a}R^{6b}.

Typical examples of R⁶ include —OR^{6a} or —NR^{6a}R^{6b}; and methyl, ethyl, propyl, 2-methylpropyl, butyl, cyclopropyl, cyclobutyl, cyclohexyl, cyclohexylmethyl, phenyl, benzyl, phenylethyl, pyrazolyl, isoxazolyl, oxadiazolyl, triazolyl, pyridinyl, triazolylmethyl, benzotriazolylmethyl or pyridinylmethyl, any of which groups may be optionally substituted by one or more substituents.

Apposite examples of R⁶ include —OR^{6a} or —NR^{6a}R^{6b}, and cyclopropyl, phenyl, pyrazolyl, isoxazolyl, oxadiazolyl or triazolyl, any of which groups may be optionally substituted by one or more substituents.

Selected examples of R⁶ include —OR^{6a}; and cyclopropyl, pyrazolyl, oxadiazolyl or triazolyl, any of which groups may be optionally substituted by one or more substituents.

Suitable examples of R⁶ include —OR^{6a}; and cyclopropyl, pyrazolyl or oxadiazolyl, any of which groups may be optionally substituted by one or more substituents.

Illustrative examples of R⁶ include pyrazolyl, isoxazolyl, oxadiazolyl and triazolyl, any of which groups may be optionally substituted by one or more substituents.

General examples of R⁶ include pyrazolyl, oxadiazolyl and triazolyl, any of which groups may be optionally substituted by one or more substituents.

Representative examples of R⁶ include pyrazolyl and oxadiazolyl, either of which groups may be optionally substituted by one or more substituents.

Particular examples of R⁶ include oxadiazolyl, which group may be optionally substituted by one or more substituents.

Typical examples of optional substituents on R⁶ include one, two or three substituents independently selected from halogen, cyano, nitro, C₁₋₆ alkyl, trifluoro-methyl, cyclopropyl, phenyl, fluorophenyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, amino(C₁₋₆)alkyl, C₁₋₆ alkylamino, di(C₁₋₆)alkylamino, pyrrolidinyl, tetrahydropyranyl, morpholinyl, piperazinyl, C₂₋₆ alkylcarbonylamino, C₂₋₆ alkylcarbonylamino-(C₁₋₆)alkyl, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl and di(C₁₋₆)alkylsulfoximinyl.

Apposite examples of optional substituents on R⁶ include one, two or three substituents independently selected from halogen, C₁₋₆ alkyl and cyclopropyl.

Suitable examples of optional substituents on R⁶ include one, two or three substituents independently selected from halogen and C₁₋₆ alkyl.

Typical examples of particular substituents on R⁶ include one, two or three substituents independently selected from fluoro, chloro, bromo, cyano, nitro, methyl, ethyl, isopropyl, tert-butyl, trifluoromethyl, cyclopropyl, phenyl, fluorophenyl, hydroxy, hydroxymethyl, oxo, methoxy, tert-butoxy, difluoromethoxy, trifluoromethoxy, methylthio, methylsulfinyl, methylsulfonyl, amino, aminomethyl, aminoethyl, methyl-amino, tert-butylamino, dimethylamino, pyrrolidinyl, tetrahydropyranyl, morpholinyl, piperazinyl, acetylamino, acetylaminoethyl, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl, dimethylaminosulfonyl and dimethylsulfoximinyl.

Apposite examples of particular substituents on R⁶ include one, two or three substituents independently selected from fluoro, methyl, ethyl, isopropyl and cyclopropyl.

Suitable examples of particular substituents on R⁶ include one, two or three substituents independently selected from fluoro, methyl and isopropyl.

Illustrative examples of particular values of R⁶ include methyl, difluoromethyl, methylsulfonylmethyl, aminomethyl, methylaminomethyl, difluoroethyl, carboxyethyl, difluoropropyl, 2-methylpropyl, butyl, fluorocyclopropyl, cyanocyclopropyl, methyl-cyclopropyl, ethylcyclopropyl, dimethylcyclopropyl, trifluoromethylcyclopropyl, phenyl-cyclopropyl, fluorophenylcyclopropyl, hydroxycyclopropyl, aminocyclopropyl, cyclobutyl, trifluoromethylcyclobutyl, cyclohexyl, cyclohexylmethyl, phenyl, fluorophenyl, chlorophenyl, cyanophenyl, methylphenyl, hydroxyphenyl, methylsulfonyl-phenyl, dimethylsulfoximinylphenyl, benzyl, fluorobenzyl, difluorobenzyl, chlorobenzyl, (chloro)(fluoro)benzyl, dichlorobenzyl, (chloro)(difluoro)benzyl, bromobenzyl, cyano-benzyl, methylbenzyl, dimethylbenzyl, trifluoromethylbenzyl, phenylbenzyl, hydroxy-benzyl, hydroxymethylbenzyl, benzoyl, methoxybenzyl, dimethoxybenzyl, trifluoro-methoxybenzyl, methylsulfonylbenzyl, aminomethylbenzyl, aminoethylbenzyl, dimethyl-aminobenzyl, pyrrolidinylbenzyl, (dimethyl)(pyrrolidinyl)benzyl, morpholinylbenzyl, (dimethyl)(morpholinyl)benzyl, piperazinylbenzyl, acetylaminoethylbenzyl, phenylethyl, chlorophenylethyl, methylpyrazolyl, ethylpyrazolyl, isopropylpyrazolyl, (methyl)-(tetrahydropyranyl)pyrazolyl, methylisoxazolyl, ethylisoxazolyl, methyloxadiazolyl, ethyloxadiazolyl, cyclopropyloxadiazolyl, isopropyltriazolyl, pyridinyl, triazolylmethyl, benzotriazolylmethyl, pyridinylmethyl and aminopyridinylmethyl.

Typical values of R⁶ include fluorocyclopropyl, methylpyrazolyl, ethylpyrazolyl, isopropylpyrazolyl, methylisoxazolyl, ethylisoxazolyl, methyloxadiazolyl, ethyloxadiazolyl, cyclopropyloxadiazolyl and isopropyltriazolyl.

Selected values of R⁶ include fluorocyclopropyl, methylpyrazolyl, isopropylpyrazolyl, methyloxadiazolyl and isopropyltriazolyl.

Suitable values of R⁶ include fluorocyclopropyl, isopropylpyrazolyl and methyloxadiazolyl.

In a first embodiment, R^{6a} represents optionally substituted C₁₋₆ alkyl. In a second embodiment, R^{6a} represents optionally substituted C₃₋₉ cycloalkyl. In a third embodiment, R^{6a} represents optionally substituted aryl(C₁₋₆)alkyl.

Typically, R^{6a} represents C₁₋₆ alkyl, cyclobutyl or benzyl, any of which groups may be optionally substituted by one or more substituents.

Typical examples of optional substituents on R^{6a} include one, two or three substituents independently selected from halogen, cyano, nitro, C₁₋₆ alkyl, trifluoro-methyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, amino(C₁₋₆)alkyl, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Suitable examples of optional substituents on R^{6a} include one, two or three substituents independently selected from halogen.

Typical examples of specific substituents on R^{6a} include one, two or three substituents independently selected from fluoro, chloro, bromo, cyano, nitro, methyl, ethyl, isopropyl, tert-butyl, trifluoromethylhydroxy, hydroxymethyl, oxo, methoxy, tert-butoxy, difluoromethoxy, trifluoromethoxy, methylthio, methylsulfinyl, methylsulfonyl, amino, aminomethyl, aminoethyl, methylamino, tert-butylamino, dimethylamino, acetylamino, methoxycarbonylamino, methylsulfonylamino, formyl, acetyl, carboxy, methoxycarbonyl, ethoxycarbonyl, tert-butoxycarbonyl, aminocarbonyl, methylaminocarbonyl, dimethylaminocarbonyl, aminosulfonyl, methylaminosulfonyl and dimethylaminosulfonyl.

Suitable examples of specific substituents on R^{6a} include one, two or three substituents independently selected from fluoro.

Illustrative examples of specific values of R^{6a} include methyl, ethyl, n-propyl, isopropyl, n-butyl, tert-butyl, cyclobutyl, difluorocyclobutyl and benzyl.

Typically, R^{6a} represents benzyl.

Typically, R^{6b} represents hydrogen or methyl.

In a first embodiment, R^{6b} represents hydrogen. In a second embodiment, R^{6b} represents C₁₋₆ alkyl, especially methyl.

Typically, R^{6c} represents hydrogen or methyl.

In a first embodiment, R^{6c} represents hydrogen. In a second embodiment, R^{6c} represents C₁₋₆ alkyl, especially methyl.

Alternatively, the moiety —NR^{6b}R^{6c} may suitably represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents.

Selected examples of suitable substituents on the heterocyclic moiety —NR^{6b}R^{6c} include C₁₋₆ alkyl, C₁₋₆ alkylsulfonyl, hydroxy, hydroxy(C₁₋₆)alkyl, amino(C₁₋₆)alkyl, cyano, oxo, C₂₋₆ alkylcarbonyl, carboxy, C₂₋₆ alkoxycarbonyl, amino, C₂₋₆ alkylcarbonylamino, C₂₋₆ alkylcarbonylamino(C₁₋₆)alkyl, C₂₋₆ alkoxycarbonylamino, C₁₋₆ alkylsulfonylamino and aminocarbonyl.

Selected examples of specific substituents on the heterocyclic moiety —NR^{6b}R^{6c} include methyl, methylsulfonyl, hydroxy, hydroxymethyl, aminomethyl, cyano, oxo, acetyl, carboxy, ethoxycarbonyl, amino, acetylamino, acetylaminomethyl, tert-butoxycarbonylamino, methylsulfonylamino and aminocarbonyl.

Generally, R⁷ represents —COR^{7a}, —CO₂R^{7a} or —SO₂R^{7b}; or R⁷ represents hydrogen; or R⁷ represents C₁₋₆ alkyl, which group may be optionally substituted by one or more fluorine atoms, generally by one, two or three fluorine atoms, typically by two fluorine atoms.

Suitably, R⁷ represents —CO₂R^{7a}.

In a first embodiment, R⁷ represents —COR^{7a}. In a second embodiment, R⁷ represents —CO₂R^{7a}. In a third embodiment, R⁷ represents —CO₂R^{7a}. In a fourth embodiment, R⁷ represents hydrogen. In a fifth embodiment, R⁷ represents C₁₋₆ alkyl, optionally substituted by one or more fluorine atoms, typically by one, two or three fluorine atoms. In one aspect of that embodiment, R⁷ represents unsubstituted C₁₋₆ alkyl, especially methyl or ethyl. In another aspect of that embodiment, R⁷ represents C₁₋₆ alkyl substituted by one, two or three fluorine atoms, typically by two fluorine atoms. Examples of that aspect include difluoroethyl. In a sixth embodiment, R⁷ represents C₃₋₉ cycloalkyl, optionally substituted by one or more fluorine atoms, typically by one, two or three fluorine atoms. In one aspect of that embodiment, R⁷ represents unsubstituted C₃₋₉ cycloalkyl, especially cyclopropyl, cyclobutyl, cyclopentyl or cyclohexyl. In another aspect of that embodiment, R⁷ represents C₃₋₉ cycloalkyl substituted by one, two or three fluorine atoms, typically by two fluorine atoms. Examples of that aspect include difluorocyclobutyl.

Typically, R^{7a} represents C₁₋₆ alkyl, optionally substituted by one, two or three fluorine atoms.

Suitably, R^{7a} represents C₁₋₆ alkyl or difluoro(C₁₋₆)alkyl.

In a first embodiment, R^{7a} represents C₁₋₆ alkyl, especially methyl or ethyl. In a first aspect of that embodiment, R^{7a} represents methyl. In a second aspect of that embodiment, R^{7a} represents ethyl. In a second embodiment, R^{7a} represents difluoro(C₁₋₆)-alkyl, especially difluoroethyl.

Particular values of R^{7a} include methyl and difluoroethyl.

Suitably, R⁷⁰ represents methyl or ethyl. In a first embodiment, R⁷⁰ represents methyl. In a second embodiment, R⁷⁰ represents ethyl.

Suitably, R⁸ represents methyl or ethyl. In a first embodiment, R⁸ represents methyl. In a second embodiment, R⁸ represents ethyl.

Various sub-classes of compounds according to the invention are represented by the compounds of formula (IIA-1), (IIA-2), (IIA-3), (IIB-1), (IIC-1) and (IIC-2) and N-oxides thereof, and pharmaceutically acceptable salts thereof:

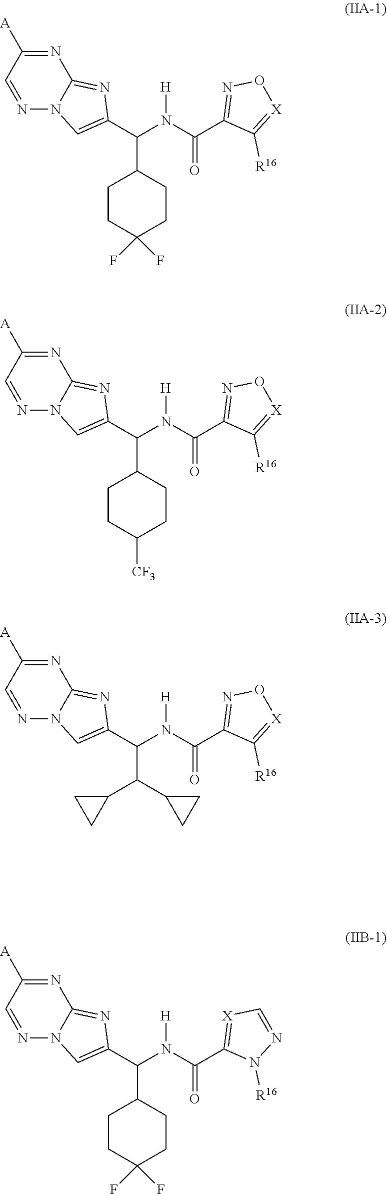

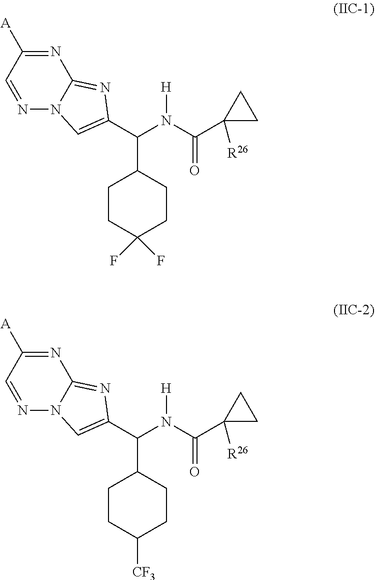

wherein
X represents CH or N;
R¹⁶ represents methyl, ethyl, isopropyl or cyclopropyl;
R²⁶ represents fluoro or trifluoromethyl; and
A is as defined above.

In a first embodiment, X represents CH. In a second embodiment, X represents N.

In a first embodiment, R¹⁶ represents methyl. In a second embodiment, R¹⁶ represents ethyl. In a third embodiment, R¹⁶ represents isopropyl. In a fourth embodiment, R¹⁶ represents cyclopropyl.

Typically, R¹⁶ represents methyl, ethyl or isopropyl.

Suitably, R¹⁶ represents methyl or isopropyl.

In a first embodiment, R²⁶ represents fluoro. In a second embodiment, R²⁶ represents trifluoromethyl.

Specific novel compounds in accordance with the present invention include each of the compounds whose preparation is described in the accompanying Examples, and pharmaceutically acceptable salts and solvates thereof.

The compounds in accordance with the present invention are beneficial in the treatment and/or prevention of various human ailments, including inflammatory and autoimmune disorders.

The compounds according to the present invention are useful in the treatment and/or prophylaxis of a pathological disorder that is mediated by a pro-inflammatory IL-17 cytokine or is associated with an increased level of a pro-inflammatory IL-17 cytokine. Generally, the pathological condition is selected from the group consisting of infections (viral, bacterial, fungal and parasitic), endotoxic shock associated with infection, arthritis, rheumatoid arthritis, psoriatic arthritis, systemic onset juvenile idiopathic arthritis (JIA), systemic lupus erythematosus (SLE), asthma, chronic obstructive airways disease (COAD), chronic obstructive pulmonary disease (COPD), acute lung injury, pelvic inflammatory disease, Alzheimer's Disease, Crohn's disease, inflammatory bowel disease, irritable bowel syndrome, ulcerative colitis, Castleman's disease, axial spondyloarthritis, ankylosing spondylitis and other spondyloarthropathies, dermatomyositis, myocarditis, uveitis, exophthalmos, autoimmune thyroiditis, Peyronie's Disease, coeliac disease, gall bladder disease, Pilonidal disease, peritonitis, psoriasis, atopic dermatitis, hidradenitis suppurativa, vasculitis, surgical adhesions, stroke, autoimmune diabetes, Type I Diabetes, lyme arthritis, meningoencephalitis, immune mediated inflammatory disorders of the central and peripheral nervous system such as multiple sclerosis and Guillain-Barr syndrome, other autoimmune disorders, pancreatitis, trauma (surgery), graft-versus-host disease, transplant rejection, fibrosing disorders including pulmonary fibrosis, liver fibrosis, renal fibrosis, scleroderma or systemic sclerosis, cancer (both solid tumours such as melanomas, hepatoblastomas, sarcomas, squamous cell carcinomas, transitional cell cancers, ovarian cancers and hematologic malignancies and in particular acute myelogenous leukaemia, chronic myelogenous leukemia, chronic lymphatic leukemia, gastric cancer and colon cancer), heart disease including ischaemic diseases such as myocardial infarction as well as atherosclerosis, intravascular coagulation, bone resorption, osteoporosis, periodontitis, hypochlorhydia and pain (particularly pain associated with inflammation).

WO 2009/089036 reveals that modulators of IL-17 activity may be administered to inhibit or reduce the severity of ocular inflammatory disorders, in particular ocular surface inflammatory disorders including Dry Eye Syndrome (DES). Consequently, the compounds in accordance with the present invention are useful in the treatment and/or prevention of an IL-17-mediated ocular inflammatory disorder, in particular an IL-17-mediated ocular surface inflammatory disorder including Dry Eye Syndrome. Ocular surface inflammatory disorders include Dry Eye Syndrome, penetrating keratoplasty, corneal transplantation, lamellar or partial thickness transplantation, selective endothelial transplantation, corneal neovascularization, keratoprosthesis surgery, corneal ocular surface inflammatory conditions, conjunctival scarring disorders, ocular autoimmune conditions, Pemphigoid syndrome, Stevens-Johnson syndrome, ocular allergy, severe allergic (atopic) eye disease, conjunctivitis and microbial keratitis. Particular categories of Dry Eye Syndrome include keratoconjunctivitis sicca (KCS), Sjögren syndrome, Sjögren syndrome-associated keratoconjunctivitis sicca, non-Sjögren syndrome-associated keratoconjunctivitis sicca, keratitis sicca, sicca syndrome, xerophthalmia, tear film disorder, decreased tear production, aqueous tear deficiency (ATD), meibomian gland dysfunction and evaporative loss.

Illustratively, the compounds of the present invention may be useful in the treatment and/or prophylaxis of a pathological disorder selected from the group consisting of arthritis, rheumatoid arthritis, psoriasis, psoriatic arthritis, systemic onset juvenile idiopathic arthritis (JIA), systemic lupus erythematosus (SLE), asthma, chronic obstructive airway disease, chronic obstructive pulmonary disease, atopic dermatitis, hidradenitis suppurativa, scleroderma, systemic sclerosis, lung fibrosis, inflammatory bowel diseases (including Crohn's disease and ulcerative colitis), axial spondyloarthritis, ankylosing spondylitis and other spondyloarthropathies, cancer and pain (particularly pain associated with inflammation).

Suitably, the compounds of the present invention are useful in the treatment and/or prophylaxis of psoriasis, psoriatic arthritis, hidradenitis suppurativa, axial spondyloarthritis or ankylosing spondylitis.

The present invention also provides a pharmaceutical composition which comprises a compound in accordance with the invention as described above, or a pharmaceutically acceptable salt thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds according to the present invention may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds according to the present invention may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds according to the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds according to the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds according to the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration the compounds according to the present invention may be formulated in an ointment such as petrolatum.

For rectal administration the compounds according to the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound according to the present invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

If desired, a compound in accordance with the present invention may be co-administered with another pharmaceutically active agent, e.g. an anti-inflammatory molecule.

The compounds of formula (I) above may be prepared by a process which comprises reacting a carboxylic acid of formula R⁶—CO₂H or a salt thereof (e.g. the lithium salt thereof) with a compound of formula (III):

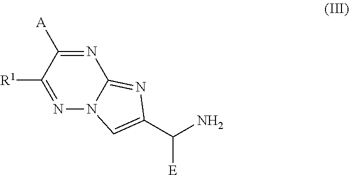

wherein A, E, R¹ and R⁶ are as defined above.

The reaction is conveniently accomplished in the presence of a coupling agent and a base. Suitable coupling agents include 1-[bis(dimethylamino)methylene]-1H-1,2,3-triazolo[4,5-b]pyridinium 3-oxid hexafluorophosphate (HATU); and 2,4,6-tripropyl-1,3,5,2,4,6-trioxatriphosphorinane-2,4,6-trioxide; and 2-chloro-1-methylpyridinium iodide. Suitable bases include organic amines, e.g. a trialkylamine such as N,N-diisopropylethylamine; or pyridine. The reaction is conveniently performed at ambient or elevated temperature in a suitable solvent, e.g. a cyclic ether such as tetrahydrofuran; or a dipolar aprotic solvent such as N,N-dimethylformamide or N,N-dimethylacetamide; or a chlorinated solvent such as dichloromethane; or an organic ester solvent such as ethyl acetate.

Alternatively, the reaction may conveniently be accomplished in the presence of a coupling agent such as N-(3-dimethylaminopropyl)-N′-ethylcarbodiimide (EDCI). The reaction is suitably performed at an appropriate temperature, e.g. a temperature in the region of 0° C., in a suitable solvent, e.g. an organic nitrile solvent such as acetonitrile.

Where R⁶ represents C₁₋₆ alkyl, e.g. methyl, the compounds of formula (I) above may be prepared by a process which comprises reacting a compound of formula R⁶—COCl, e.g. acetyl chloride, with a compound of formula (III) as defined above. The reaction is conveniently accomplished in the presence of a base. Suitable bases include organic amines, e.g. a trialkylamine such as N,N-diisopropylethylamine. The reaction is conveniently performed at ambient temperature in a suitable solvent, e.g. a cyclic ether such as tetrahydrofuran.

Where R⁶ represents —OR^{6a}, the compounds of formula (I) above may be prepared by a two-step process which comprises: (i) reacting a compound of formula R^{6a}—OH with N,N′-disuccinimidyl carbonate, ideally in the presence of a base, e.g. an organic amine such as triethylamine; and (ii) reacting the resulting material with a compound of formula (III) as defined above. Steps (i) and (ii) are conveniently performed at ambient temperature in a suitable solvent, e.g. a chlorinated solvent such as dichloromethane, or an organic nitrile solvent such as acetonitrile.

The intermediates of formula (III) above may be prepared by removal of the N-protecting group R^{p} from a compound of formula (IV):

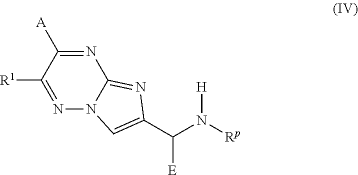

wherein A, E and R¹ are as defined above, and R^{p} represents a N-protecting group.

The N-protecting group R^{p} will suitably be tert-butoxycarbonyl (BOC), in which case the removal thereof may conveniently be effected by treatment with an acid, e.g. a mineral acid such as hydrochloric acid, or an organic acid such as trifluoroacetic acid.

Alternatively, the N-protecting group R^{p} may be benzyloxycarbonyl, in which case the removal thereof may conveniently be effected by catalytic hydrogenation, typically by treatment with hydrogen gas or ammonium formate in the presence of a hydrogenation catalyst, e.g. palladium on charcoal, or palladium hydroxide on charcoal. In a variant procedure, where the N-protecting group R^{p} is benzyloxycarbonyl, the removal thereof may be effected by treatment with hydrogen bromide and acetic acid.

In an alternative procedure, the compounds of formula (I) above wherein A represents a group of formula (Ad) may be prepared by a two-step process which comprises:
(i) saponifying a compound of formula (V):

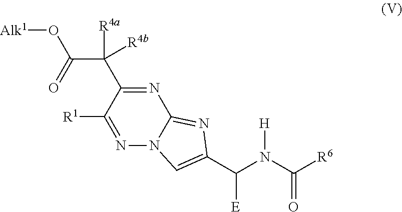

wherein E, R¹, R^{4a}, R^{4b} and R⁶ are as defined above, and Alk¹ represents C₁₋₄ alkyl, e.g. methyl, ethyl or tert-butyl; and
(ii) reaction of the carboxylic acid derivative thereby obtained with a compound of formula R³—H; under conditions analogous to those described above for the reaction between compound (III) and a carboxylic acid of formula R⁶—CO₂H.

Similarly, the intermediates of formula (IV) above wherein A represents a group of formula (Ad) may be prepared by a two-step process which comprises:
(i) saponifying a compound of formula (VI):

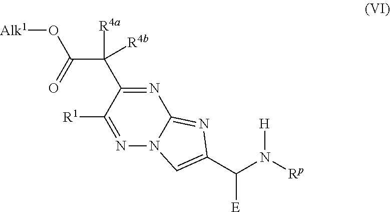

wherein E, R¹, R^{4a}, R^{4b}, Rₚ and Alk¹ are as defined above; and
(ii) reaction of the carboxylic acid derivative thereby obtained with a compound of formula R³—H; under conditions analogous to those described above for the reaction between compound (III) and a carboxylic acid of formula R⁶—CO₂H.

Where Alk¹ represents methyl or ethyl, the saponification reaction in step (i) will generally be effected by treatment with a base. Suitable bases include inorganic hydroxides, e.g. an alkali metal hydroxide such as lithium hydroxide or sodium hydroxide. The reaction is conveniently performed at ambient or elevated temperature in water and a suitable organic solvent, e.g. a cyclic ether such as tetrahydrofuran, or a C₁₋₄ alkanol such as methanol or ethanol, or a chlorinated solvent such as dichloromethane.

Alternatively, where Alk¹ represents tert-butyl, the saponification reaction in step (i) may generally be effected by treatment with an acid, e.g. an organic acid such as trifluoroacetic acid. The reaction is conveniently performed at ambient temperature in a suitable organic solvent, e.g. a chlorinated solvent such as dichloromethane.

The intermediates of formula (V) above may be prepared by reacting a carboxylic acid of formula R⁶—CO₂H with a compound of formula (VII):

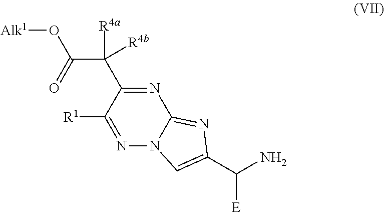

wherein E, R¹, R^{4a}, R^{4b}, R⁶ and Alk¹ are as defined above; under conditions analogous to those described above for the reaction between compound (III) and a carboxylic acid of formula R⁶—CO₂H.

The intermediates of formula (VII) above may be prepared by removal of the N-protecting group R^{p} from a compound of formula (VI) as defined above; under conditions analogous to those described above for the removal of the N-protecting group R^{p} from a compound of formula (IV).

The intermediates of formula (VI) above may be prepared by reacting a compound of formula (VIII) with a compound of formula (IX):

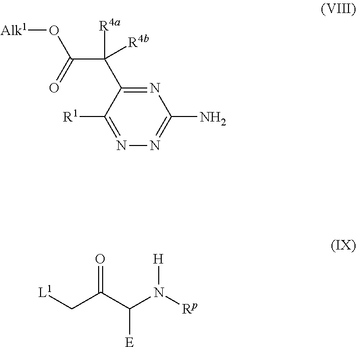

wherein E, R¹, R^{4a}, R^{4b}, Alk¹ and R^{p} are as defined above, and L¹ represents a suitable leaving group.

The leaving group L¹ is typically a halogen atom, e.g. bromo.

The reaction is typically accomplished in the presence of a base. Suitably, the base may be an inorganic base, e.g. a bicarbonate salt such as sodium bicarbonate; or an organic base such as pyridine. The reaction is conveniently effected at an elevated temperature in a suitable solvent, e.g. a C₁₋₄ alkanol such as ethanol or isopropanol, or a cyclic ether such as 1,4-dioxane.

In an alternative procedure, the compounds of formula (I) above wherein A represents a group of formula (Aa), (Ab) or (Ac) may be prepared by a process which comprises reacting a carboxylic acid of formula R²—CO₂H with a compound of formula (X):

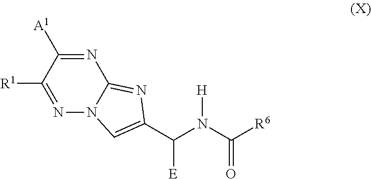

wherein
A¹ represents a group of formula (Aa-1), (Ab-1) or (Ac-1):

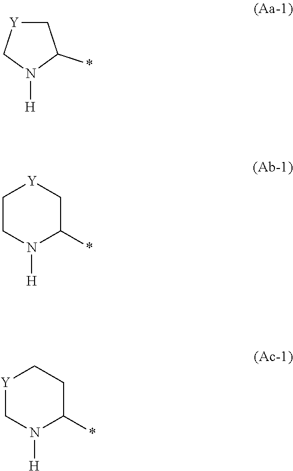

in which the asterisk (*) represents the point of attachment to the remainder of the molecule; and
E, Y, R¹, R² and R⁶ are as defined above;
   under conditions analogous to those described above for the reaction between compound (III) and a carboxylic acid of formula R⁶—CO₂H.

The intermediates of formula (X) above may be prepared by removal of the N-protecting group R^{z} from a compound of formula (XI):

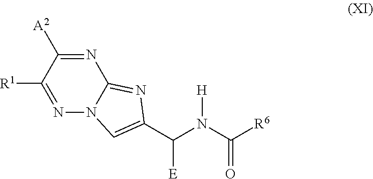

wherein
A² represents a group of formula (Aa-2), (Ab-2) or (Ac-2):

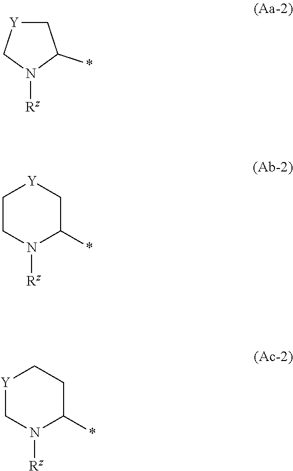

in which the asterisk (*) represents the point of attachment to the remainder of the molecule;
R^{z} represents a N-protecting group; and
E, Y, R¹ and R⁶ are as defined above.

The N-protecting group R^{z} will suitably be tert-butoxycarbonyl (BOC), in which case the removal thereof may conveniently be effected by treatment with an acid, e.g. a mineral acid such as hydrochloric acid, or an organic acid such as trifluoroacetic acid.

The intermediates of formula (XI) above may be prepared by a two-step procedure which comprises the following steps:
(i) removal of the N-protecting group R^{p} from a compound of formula (XII):

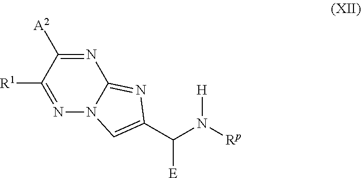

wherein E, A², R¹ and R^{p} are as defined above; under conditions analogous to those described above; and
(ii) reaction of the material thereby obtained with a carboxylic acid of formula R⁶—CO₂H, under conditions analogous to those described above for the reaction between compound (III) and a carboxylic acid of formula R⁶—CO₂H.

In an alternative method, the intermediates of formula (IV) above wherein A represents a group of formula (Aa), (Ab) or (Ac) may be prepared by a two-step procedure which comprises the following steps:
(i) removal of the N-protecting group R^{z} from a compound of formula (XII) as defined above; under conditions analogous to those described above; and
(ii) reaction of the material thereby obtained with a carboxylic acid of formula R²—CO₂H, under conditions analogous to those described above for the reaction between compound (III) and a carboxylic acid of formula R⁶—CO₂H.

The intermediates of formula (XII) above may be prepared by reacting a compound of formula A²—CO₂H with a compound of formula (XIII):

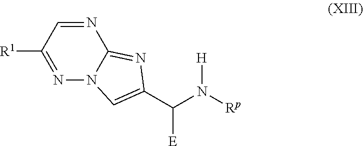

wherein E, A², R¹ and R^{p} are as defined above; in the presence of a transition metal catalyst.

Suitable transition metal catalysts of use in the reaction include [4,4′-bis(1,1-dimethylethyl)-2,2′-bipyridine-N1,N1′]bis-{3,5-difluoro-2-[5-(trifluoromethyl)-2-pyridinyl-N]phenyl-C}iridium(III) hexafluorophosphate; and tris[2-phenylpyridinato-C²,N]iridium(III). The reaction will generally be performed by exposing the reactants to a bright light source. A suitable bright light source will typically comprise the ‘integrated photoreactor’ described in ACS Cent. Sci., 2017, 3, 647-653; or the Penn Photoreactor M2 system. The reaction will conveniently be carried out at ambient temperature in a suitable solvent, e.g. a dipolar aprotic solvent such as N,N-dimethylformamide, or an organic disulfide such as dimethyl sulfoxide.

In an alternative method, the intermediates of formula (XI) above may be prepared by a three-step procedure which comprises the following steps:
(i) removal of the N-protecting group R^{p} from a compound of formula (XIII) as defined above; under conditions analogous to those described above;
(ii) reaction of the material thereby obtained with a carboxylic acid of formula R⁶—CO₂H or a salt thereof (e.g. the lithium salt thereof), under conditions analogous to those described above for the reaction between compound (III) and a carboxylic acid of formula R⁶—CO₂H or a salt thereof (e.g. the lithium salt thereof); and
(iii) reaction of the material thereby obtained with a compound of formula A²—CO₂H under conditions analogous to those described above for the reaction between compound (XIII) and a carboxylic acid of formula A²—CO₂H.

In another procedure, the compounds of formula (I) above wherein A represents a group of formula (Ad) may be prepared by a three-step process which comprises:
(i) removal of the N-protecting group R^{p} from a compound of formula (XIII) as defined above; under conditions analogous to those described above;
(ii) reaction of the material thereby obtained with a carboxylic acid of formula R⁶—CO₂H or a salt thereof (e.g. the lithium salt thereof), under conditions analogous to those described above for the reaction between compound (III) and a carboxylic acid of formula R⁶—CO₂H or a salt thereof (e.g. the lithium salt thereof); and
(iii) reaction of the material thereby obtained with a compound of formula A-CO₂H, wherein A represents a group of formula (Ad) as defined above, under conditions analogous to those described above for the reaction between compound (XIII) and a carboxylic acid of formula A²—CO₂H.

The intermediates of formula (XIII) above may be prepared by reacting a compound of formula (IX) as defined above with a compound of formula (XIV):

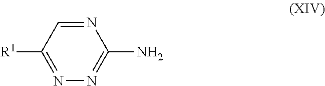

wherein R¹ is as defined above; under conditions analogous to those described above for the reaction between compounds (VIII) and (IX).

In an alternative procedure, the compounds of formula (I) wherein A represents a group of formula (Ae) and Z represents a group of formula (Zt) as defined above, in which R^{2z} is hydrogen, may be prepared by a process which comprises reacting a compound of formula R^{1z}—NH₂ and a trialkyl orthoformate HC(O-Alk¹)₃ with a compound of formula (XV):

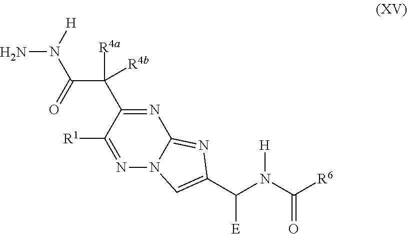

wherein E, R¹, R^{4a}, R^{4b}, R⁶, R^{1z} and Alk¹ are as defined above.

The reaction is conveniently performed at an elevated temperature in the presence of acetic acid. The reaction may typically be carried out in a suitable solvent, e.g. a cyclic ether such as 1,4-dioxane.

The intermediates of formula (XV) above may be prepared by reacting a compound of formula (V) as defined above with hydrazine hydrate.

The reaction is conveniently performed at an elevated temperature in a suitable solvent, e.g. a C₁₋₄ alkanol such as ethanol.

The intermediates of formula (IV) above wherein A represents a group of formula (Ae) and Z represents a group of formula (Zu) as defined above may be prepared by a three-step procedure which comprises the following steps:
(i) saponification of a compound of formula (VI) as defined above by treatment with a base;
(ii) reaction of the carboxylic acid derivative thereby obtained with a compound of formula (XVI):

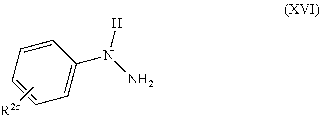

wherein R^{2z} is as defined above; under conditions analogous to those described above for the reaction between compound (III) and a carboxylic acid of formula R⁶—CO₂H; and
(iii) cyclisation of the resulting material by treatment with triphenylphosphine in the presence of a base.

The saponification reaction in step (i) will generally be effected by treatment with a base. Suitable bases include inorganic hydroxides, e.g. an alkali metal hydroxide such as lithium hydroxide.

Suitable bases of use in step (iii) include organic amines, e.g. a trialkylamine such as triethylamine. The reaction is conveniently performed at ambient temperature in the presence of hexachloroethane and a suitable solvent, e.g. a cyclic ether such as tetrahydrofuran.

In another procedure, the compounds of formula (I) wherein A represents a group of formula (Ae) and Z represents a group of formula (Zw) or (Zx) as defined above, in which R^{1z} is other than hydrogen, may be prepared by a two-step procedure which comprises the following steps:
(i) reacting an alkali metal azide with a compound of formula (XVII):

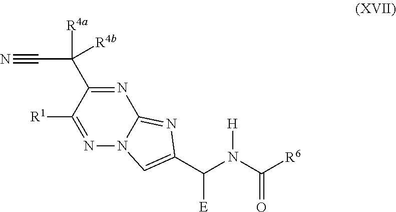

wherein E, R¹, R^{4a}, R^{4b} and R⁶ are as defined above; and
(ii) reacting the resulting material with a compound of formula R^{1z}-L³, wherein R^{1z} is as defined above (and is other than hydrogen), and L³ represents a suitable leaving group.

In step (i), the alkali metal azide is suitably sodium azide. The reaction is conveniently performed at an elevated temperature in the presence of ammonium chloride and a suitable solvent, e.g. a dipolar aprotic solvent such as N,N-dimethylformamide.

The leaving group L³ may suitably be a sulfonyloxy derivative, e.g. trifluoro-methanesulfonyloxy.

Step (ii) will generally be accomplished in the presence of a base. Suitable bases include alkali metal carbonates, e.g. potassium carbonate. The reaction is conveniently effected at an elevated temperature in a suitable solvent, e.g. a carbonyl-containing solvent such as acetone.

The intermediates of formula (XVII) above may be prepared by a two-step procedure which comprises the following steps:
(i) reacting a compound of formula (XIII) as defined above with ammonia; and
(ii) reacting the material thereby obtained with trifluoroacetic anhydride in the presence of pyridine.

Step (i) is conveniently performed at an elevated temperature in a suitable solvent, e.g. a C₁₋₄ alkanol such as methanol.

Step (ii) is conveniently carried out at ambient temperature in a suitable solvent, e.g. a cyclic ether such as 1,4-dioxane.

The intermediates of formula (IV) above wherein A represents a group of formula (Ae) and Z represents a group of formula (Zq) as defined above, in which R^{2z} is hydrogen, may be prepared by reacting an azide derivative of formula R^{1z}—N₃ with a compound of formula (XVIII):

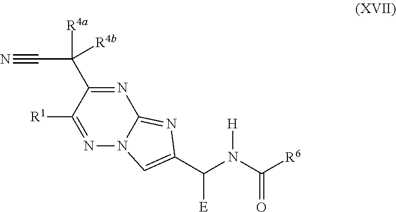

wherein E, R¹, R^{4a}, R^{4b}, R^{1z} and R^{p} are as defined above; in the presence of a transition metal catalyst.

Suitable transition metal catalysts of use in the above reaction include chloro-(pentamethylcyclopentadienyl)(cyclooctadiene)ruthenium (II).

The reaction is conveniently carried out at an elevated temperature in a suitable solvent or mixture of solvents. Typical solvents include alkyl ethers, e.g. tert-butyl methyl ether, or 1,2-dimethoxyethane; and cyclic ethers, e.g. tetrahydrofuran.

The intermediates of formula (XVIII) above may be prepared by reacting a compound of formula (XIX):

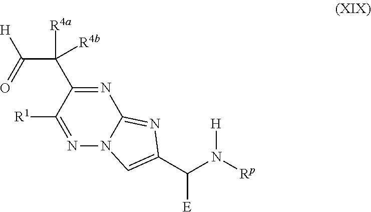

wherein E, R¹, R^{4a}, R^{4b} and R^{p} are as defined above; with dimethyl (1-diazo-2-oxo-propyl)phosphonate.

The reaction is generally performed in the presence of a base. Suitably, the base may be an alkali metal carbonate, e.g. potassium carbonate. The reaction is conveniently effected at ambient temperature in a suitable solvent or mixture of solvents. Typical solvents include C₁₋₄ alkanols, e.g. methanol; and chlorinated solvents, e.g. dichloromethane.

The intermediates of formula (XIX) above may be prepared by a two-step procedure which comprises the following steps:
(i) removal of the O-protecting group R^{s} from a compound of formula (XX):

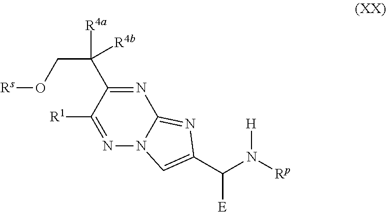

wherein E, R¹, R^{4a}, R^{4b} and R^{p} are as defined above, and R^{s} represents an O-protecting group; and
(ii) treatment of the compound thereby obtained with an oxidising agent.

The O-protecting group R^{s} will suitably be acetyl.

Where R^{s} represents acetyl, the removal thereof in step (i) above may conveniently be effected by treatment with a base. Suitably, the base may be an alkali metal carbonate, e.g. potassium carbonate. The reaction is conveniently effected at ambient temperature in a suitable solvent, e.g. a C₁₋₄ alkanol such as methanol.

Suitable oxidising agents of use in step (ii) above include 1,1,1-tris(acetyloxy)-1,1-dihydro-1,2-benziodoxol-3-(1H)-one (Dess-Martin periodinane). The reaction is conveniently effected at ambient temperature in a suitable solvent, e.g. a chlorinated solvent such as dichloromethane.

Alternatively, the oxidising agent of use in step (ii) above may comprise sulfur trioxide pyridine complex, in which case the reaction may conveniently be accomplished in the presence of a base. Suitably, the base may be an organic amine, e.g. N,N-diisopropylethylamine.

The intermediates of formula (XX) above may be prepared by reacting a compound of formula (IX) as defined above with a compound of formula (XXI):

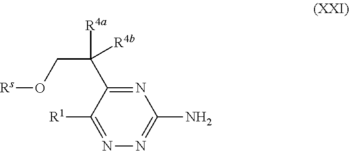

wherein R¹, R^{4a}, R^{4b} and R^{s} are as defined above; under conditions analogous to those described above for the reaction between compounds (VIII) and (IX).

Where they are not commercially available, the starting materials of formula (VIII), (IX), (XIV), (XVI) and (XXI) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art. By way of example, a compound comprising a N-BOC moiety (wherein BOC is an abbreviation for tert-butoxycarbonyl) may be converted into the corresponding compound comprising a N—H moiety by treatment with an acid, e.g. a mineral acid such as hydrochloric acid, or an organic acid such as trifluoroacetic acid.

A compound comprising a N—H functionality may be alkylated, e.g. methylated, by treatment with a suitable alkyl halide, e.g. iodomethane, typically in the presence of a base, e.g. an inorganic carbonate such as sodium carbonate.

A compound comprising a N—H functionality may be acylated, e.g. acetylated, by treatment with a suitable acyl halide, e.g. acetyl chloride, typically in the presence of a base, e.g. an organic base such as N,N-diisopropylethylamine or triethylamine. Similarly, a compound comprising a N—H functionality may be acylated, e.g. acetylated, by treatment with a suitable acyl anhydride, e.g. acetic anhydride, typically in the presence of a base, e.g. an organic base such as triethylamine.

Similarly, a compound comprising a N—H functionality may be converted into the corresponding compound comprising a N—S(O)₂Alk¹ functionality (wherein Alk¹ is as defined above) by treatment with the appropriate C₁₋₄ alkylsulfonyl chloride reagent, e.g. methylsulfonyl chloride, typically in the presence of a base, e.g. an organic base such as triethylamine.

Similarly, a compound comprising a N—H functionality may be converted into the corresponding compound comprising a carbamate or urea moiety respectively by treatment with the appropriate chloroformate or carbamoyl chloride reagent, typically in the presence of a base, e.g. an organic base such as triethylamine or N,N-diisopropylethylamine. Alternatively, a compound comprising a N—H functionality may be converted into the corresponding compound comprising a urea moiety by treatment with the appropriate amine-substituted (3-methylimidazol-3-ium-1-yl)methanone iodide derivative, typically in the presence of a base, e.g. an organic base such as triethylamine. Alternatively, a compound comprising a N—H functionality may be converted into the corresponding compound comprising a urea moiety N—C(O)N(H)Alk¹ (wherein Alk¹ is as defined above) by treatment with the appropriate isocyanate derivative Alk¹—N═C═O, typically in the presence of a base, e.g. an organic base such as triethylamine.

A compound comprising a N—H functionality may be converted into the corresponding compound comprising a N—C(H) functionality by treatment with the appropriate aldehyde or ketone in the presence of a reducing agent such as sodium triacetoxyborohydride or sodium cyanoborohydride.

A compound comprising a C₁₋₄ alkoxycarbonyl moiety —CO₂Alk¹ (wherein Alk¹ is as defined above) may be converted into the corresponding compound comprising a carboxylic acid (—CO₂H) moiety by treatment with a base, e.g. an alkali metal hydroxide salt such as lithium hydroxide. Alternatively, a compound comprising a tert-butoxycarbonyl moiety may be converted into the corresponding compound comprising a carboxylic acid (—CO₂H) moiety by treatment with trifluoroacetic acid.

A compound comprising a carboxylic acid (—CO₂H) moiety may be converted into the corresponding compound comprising an amide moiety by treatment with the appropriate amine, under conditions analogous to those described above for the reaction between compound (III) and a carboxylic acid of formula R⁶—CO₂H.

A compound comprising a C₁₋₄ alkoxycarbonyl moiety —CO₂Alk¹ (wherein Alk¹ is as defined above) may be converted into the corresponding compound comprising a hydroxymethyl (—CH₂OH) moiety by treatment with a reducing agent such as lithium aluminium hydride.

A compound comprising a C₁₋₄ alkylcarbonyloxy moiety —OC(O)Alk¹ (wherein Alk¹ is as defined above), e.g. acetoxy, may be converted into the corresponding compound comprising a hydroxy (—OH) moiety by treatment with a base, e.g. an alkali metal hydroxide salt such as sodium hydroxide.

A compound comprising a halogen atom, e.g. bromo, may be converted into the corresponding compound comprising an optionally substituted aryl, heterocycloalkenyl or heteroaryl moiety by treatment with the appropriately substituted aryl, heterocycloalkenyl or heteroaryl boronic acid or a cyclic ester thereof formed with an organic diol, e.g. pinacol, 1,3-propanediol or neopentyl glycol. The reaction is typically effected in the presence of a transition metal catalyst, and a base. The transition metal catalyst may be [1,1′-bis(diphenylphosphino)ferrocene]dichloropalladium(II). In the alternative, the transition metal catalyst may be tris(dibenzylideneacetone)dipalladium(0), which may advantageously be employed in conjunction with 2-dicyclohexylphosphino-2′,4′,6′-triisopropylbiphenyl (XPhos). Suitably, the base may be an inorganic base such as sodium carbonate or potassium carbonate.

A compound comprising a halogen atom, e.g. bromo, may be converted into the corresponding compound comprising an optionally substituted aryl or heteroaryl moiety via a two-step procedure which comprises: (i) reaction with bis(pinacolato)diboron; and (ii) reaction of the compound thereby obtained with an appropriately substituted bromoaryl or bromoheteroaryl derivative. Step (i) is conveniently effected in the presence of a transition metal catalyst such as [1,1′-bis(diphenylphosphino)ferrocene]-dichloropalladium(II), and potassium acetate. Step (ii) is conveniently effected in the presence of a transition metal catalyst such as [1,1′-bis(diphenylphosphino)ferrocene]-dichloropalladium(II), and a base, e.g. an inorganic base such as sodium carbonate or potassium carbonate.

A compound comprising a cyano (—CN) moiety may be converted into the corresponding compound comprising a 1-aminoethyl moiety by a two-step process which comprises: (i) reaction with methylmagnesium chloride, ideally in the presence of titanium(IV) isopropoxide; and (ii) treatment of the resulting material with a reducing agent such as sodium borohydride. If an excess of methylmagnesium chloride is employed in step (i), the corresponding compound comprising a 1-amino-1-methylethyl moiety may be obtained.

A compound comprising the moiety —S— may be converted into the corresponding compound comprising the moiety —S(O)(NH)— by treatment with (diacetoxyiodo)benzene and ammonium carbamate.

A compound comprising a C═C double bond may be converted into the corresponding compound comprising a CH—CH single bond by treatment with gaseous hydrogen in the presence of a hydrogenation catalyst, e.g. palladium on charcoal.

A compound comprising an aromatic nitrogen atom may be converted into the corresponding compound comprising an N-oxide moiety by treatment with a suitable oxidising agent, e.g. 3-chloroperbenzoic acid.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Greene's Protective Groups in Organic Synthesis, ed. P. G. M. Wuts, John Wiley & Sons, 5^{th} edition, 2014. The protecting groups may be removed at any convenient subsequent stage utilising methods known from the art.

The compounds in accordance with this invention potently inhibit IL-17 induced IL-6 release from human dermal fibroblasts. Thus, when tested in the HDF cell line assay described below, compounds of the present invention exhibit a pIC₅₀ value of 5.0 or more, generally of 6.0 or more, usually of 7.0 or more, typically of 7.2 or more, suitably of 7.5 or more, ideally of 7.8 or more, and preferably of 8.0 or more (pIC₅₀ equals-log₁₀[IC₅₀], in which IC₅₀ is expressed as a molar concentration, so the skilled person will appreciate that a higher pIC₅₀ figure denotes a more active compound).

Inhibition of IL-17A Induced IL-6 Release from Dermal Fibroblast Cell Line

The purpose of this assay is to test the neutralising ability to IL-17 proteins, in a human primary cell system. Stimulation of normal human dermal fibroblasts (HDF) with IL-17 alone produces only a very weak signal but in combination with certain other cytokines, such as TNFα, a synergistic effect can be seen in the production of inflammatory cytokines, i.e. IL-6.

HDFs were stimulated with IL-17A (50 pM) in combination with TNF-α (25 pM). The resultant IL-6 response was then measured using a homogenous time-resolved FRET kit from Cisbio. The kit utilises two monoclonal antibodies, one labelled with Eu-Cryptate (Donor) and the second with d2 or XL665 (Acceptor). The intensity of the signal is proportional to the concentration of IL-6 present in the sample (Ratio is calculated by 665/620×104).

The ability of a compound to inhibit IL-17 induced IL-6 release from human dermal fibroblasts is measured in this assay.

HDF cells (Sigma #106-05n) were cultured in complete media (DMEM+10% FCS+2 mM L-glutamine) and maintained in a tissue culture flask using standard techniques. Cells were harvested from the tissue culture flask on the morning of the assay using TrypLE (Invitrogen #12605036). The TrypLE was neutralised using complete medium (45 mL) and the cells were centrifuged at 300×g for 3 minutes. The cells were re-suspended in complete media (5 mL) counted and adjusted to a concentration of 3.125×10⁴ cells/mL before being added to the 384 well assay plate (Corning #3701) at 40 μL per well. The cells were left for a minimum of three hours, at 37° C./5% CO₂, to adhere to the plate.

Compounds were serially diluted in DMSO before receiving an aqueous dilution into a 384 well dilution plate (Greiner #781281), where 5 μL from the titration plate was transferred to 45 μL of complete media and mixed to give a solution containing 10% DMSO.

Mixtures of TNFα and IL-17 cytokine were prepared in complete media to final concentrations of TNFα 25 pM/IL-17A 50 pM, then 30 μL of the solution was added to a 384 well reagent plate (Greiner #781281).

10 μL from the aqueous dilution plate was transferred to the reagent plate containing 30 μL of the diluted cytokines, to give a 2.5% DMSO solution. The compounds were incubated with the cytokine mixtures for 5 h at 37° C. After the incubation, 10 μL was transferred to the assay plate, to give a 0.5% DMSO solution, then incubated for 18-20 h at 37° C./5% CO₂.

From the Cisbio IL-6 FRET kit (Cisbio #62IL6PEB) europium cryptate and Alexa 665 were diluted in reconstitution buffer and mixed 1:1, as per kit insert. To a white low volume 384 well plate (Greiner #784075) were added FRET reagents (10 μL), then supernatant (10 μL) was transferred from the assay plate to Greiner reagent plate. The mixture was incubated at room temperature for 3 h with gentle shaking (<400 rpm) before being read on a Synergy Neo 2 plate reader (Excitation: 330 nm; Emission: 615/645 nm).

When tested in the HDF cell line assay as described above, the compounds of the accompanying Examples were found to exhibit the following pIC₅₀ values.

| Example | pIC₅₀ |
|---|---|
| 1 | 8.1 |
| 2 | 6.8 |
| 3 | 7.1 |
| 4 | 6.2 |
| 5 | 8.4 |
| 6 | 7.0 |
| 7 | 8.2 |
| 8 | 6.9 |
| 9 | 8.2 |
| 10 | 6.3 |
| 11 | 7.3 |
| 12 | 5.1 |
| 13 | 7.3 |
| 14 | 7.0 |
| 15 | 6.9 |
| 16 | 7.9 |
| 17 | 7.9 |
| 18 | 8.6 |
| 19 | 7.0 |
| 20 | 7.8 |
| 21 | 6.4 |
| 22 | 8.2 |
| 23 | 6.8 |
| 24 | 8.0 |
| 25 | 6.5 |
| 26 | 8.0 |
| 27 | 6.6 |
| 28 | 7.4 |
| 29 | 6.2 |
| 30 | 7.9 |
| 31 | 7.8 |
| 32 | 7.2 |
| 33 | 6.9 |
| 34 | 8.4 |
| 35 | 6.9 |
| 36 | 7.9 |
| 37 | 6.1 |
| 38 | 6.9 |
| 39 | 8.3 |
| 40 | 7.1 |
| 41 | 6.1 |
| 42 | 7.9 |
| 43 | 7.6 |
| 44 | 6.9 |
| 45 | 8.4 |
| 46 | 7.9 |
| 47 | 6.2 |
| 48 | 8.2 |
| 49 | 6.2 |
| 50 | 7.9 |
| 51 | 6.6 |
| 52 | 8.1 |
| 53 | 6.3 |
| 54 | 8.1 |
| 55 | 7.2 |
| 56 | 7.8 |

The following Examples illustrate the preparation of compounds according to the invention.

### EXAMPLES

Abbreviations

| | |
|---|---|
| DCM: dichloromethane | THF: tetrahydrofuran |
| MeOH: methanol | EtOH: ethanol |
| DMSO: dimethyl sulfoxide | DIPEA: N,N-diisopropylethylamine |
| DMF: N,N-dimethylformamide | DMA: N,N-dimethylacetamide |
| EtOAc: ethyl acetate | TFA: trifluoroacetic acid |
| IPA: isopropyl alcohol | DMAP: 4-(dimethylamino)pyridine |
| TBME: tert-butyl methyl ether | LDA: lithium diisopropylamide |
| AcOH: acetic acid | TBAF: tetra-n-butylammonium fluoride |
| DMPU: 1,3-dimethyl-3,4,5,6-tetrahydro-2(1H)- | |
| pyrimidinone | |
| EDCI•HCl: N-(3-dimethylaminopropyl)-N′- | |
| ethylcarbodiimide hydrochloride | |
| TMEDA: N,N,N′,N′-tetramethylethylenediamine | |
| T3P ®: propylphosphonic anhydride solution | |
| HATU: 1-[bis(dimethylamino)methylene]-1H- | |
| 1,2,3-triazolo[4,5-b]pyridinium 3-oxid | |
| hexafluorophosphate | |
| {Ir[dF(CF₃)ppy]₂(dtbpy)}PF₆: [4,4′-bis(1,1- | |
| dimethylethyl)-2,2′-bipyridine-N1,N1′]bis- | |
| {3,5-difluoro-2-[5-(trifluoromethyl)-2-pyridinyl- | |
| N]phenyl-C}iridium(III) hexafluorophosphate | |
| h: hour | r.t.: room temperature |
| M: mass; molar | RT: retention time |
| HPLC: High Performance Liquid Chromatography | |
| LCMS: Liquid Chromatography Mass Spectrometry | |
| SFC: Supercritical Fluid Chromatography | |
| FCC: flash column chromatography | |

### Analytical and Separation Methods

### Method 1

Short pH10
Stationary Phase: Phenomenex Gemini NX-C18 2×20 mm, 3 μm
Mobile Phase A: 10 mM ammonium formate in water+0.1% ammonia solution
Mobile Phase B: acetonitrile+5% water+0.1% ammonia solution
Flow rate: 1 mL/minute
Gradient program:

| Time | A % | B % |
|---|---|---|
| 0.00 | 95.00 | 5.00 |
| 1.50 | 5.00 | 95.00 |
| 2.25 | 5.00 | 95.00 |
| 2.50 | 95.00 | 5.00 |

### Method 2

Stationary Phase: Phenomenex Kinetex-XB C18, 2.1 mm×100 mm, 1.7 μm
Column Temperature: 40° C.
Mobile Phase A: 0.1% formic acid in water
Mobile Phase B: 0.1% formic acid in acetonitrile
Flow rate: 0.6 mL/minute
Gradient program:

| Time | A % | B % |
|---|---|---|
| 0.00 | 95 | 5 |
| 5.30 | 0 | 100 |
| 5.80 | 0 | 100 |
| 5.82 | 95 | 5 |
| 7.00 | 95 | 5 |

### Method 3

Stationary Phase: Chiralpak AD-3, 100×6.4 mm, 3 μm
Column Temperature: 35° C.
Mobile Phase A: CO₂
Mobile Phase B: IPA (0.05% IP Am)
Flow rate: 3.4 mL/minute
Gradient program:

| Time | A % | B % |
|---|---|---|
| 0.00 | 95 | 5 |
| 2.00 | 60 | 40 |
| 3.00 | 60 | 40 |
| 3.60 | 95 | 5 |
| 4.00 | 95 | 5 |

### Method 4

Stationary Phase: Phenomenex Gemini NX-C18 2×50 mm, 3 μm
Column Temperature: 40° C.
Mobile Phase A: 10 mM ammonium formate in water+0.1% formic acid
Mobile Phase B: acetonitrile+5% water+0.1% formic acid
Flow rate: 1 mL/minute
Gradient program:

| Time | A % | B % |
|---|---|---|
| 0.00 | 95.00 | 5.00 |
| 1.80 | 5.00 | 95.00 |
| 2.10 | 5.00 | 95.00 |
| 2.30 | 95.00 | 5.00 |

### Method 5

Stationary Phase: Kinetex Core-Shell C18, 2.1×50 mm, 5 μm
Column Temperature: 40° C.
Mobile Phase A: water+0.1% formic acid
Mobile Phase B: acetonitrile+0.1% formic acid
Flow rate: 1.2 mL/minute
Gradient program:

| Time | A % | B % |
|---|---|---|
| 0.00 | 95 | 5 |
| 1.20 | 0 | 100 |
| 1.30 | 0 | 100 |
| 1.31 | 95 | 5 |

### Method 6

Stationary Phase: Phenomenex Gemini NX-C18 2×20 mm, 3 μm
Column Temperature: 40° C.
Mobile Phase A: 10 mM ammonium formate in water+0.1% formic acid
Mobile Phase B: acetonitrile+5% water+0.1% formic acid
Flow rate: 1 mL/minute
Gradient program:

| Time | A % | B % |
|---|---|---|
| 0.00 | 95.00 | 5.00 |
| 4.00 | 5.00 | 95.00 |
| 5.00 | 5.00 | 95.00 |
| 5.10 | 95.00 | 5.00 |

### Method 7

MSDXT, pH10
Stationary Phase: Waters Acquity UPLC BEH C18 2.1×50 mm, 1.7 μm
Mobile Phase A: 10 mM ammonium formate in water+0.1% ammonia solution
Mobile Phase B: acetonitrile+5% water+0.1% ammonia solution
Flow rate: 1.5 mL/minute
Gradient program:

| Time | A % | B % |
|---|---|---|
| 0.00 | 95.00 | 5.00 |
| 0.10 | 95.00 | 5.00 |
| 3.50 | 5.00 | 95.00 |
| 4.00 | 5.00 | 95.00 |
| 4.05 | 95.00 | 5.00 |

### Method 8

Long pH10
Stationary Phase: Phenomenex Gemini NX-C18 2×20 mm, 3 μm
Column Temperature: 40° C.
Mobile Phase A: 10 mM ammonium formate in water+0.1% ammonia solution
Mobile Phase B: acetonitrile+5% water+0.1% ammonia solution
Flow rate: 1 mL/minute
Gradient program:

| Time | A % | B % |
|---|---|---|
| 0.00 | 95.00 | 5.00 |
| 4.00 | 5.00 | 95.00 |
| 5.00 | 5.00 | 95.00 |
| 5.10 | 95.00 | 5.00 |

### Method 9

Purification was performed using SFC, on a Lux Cellulose-1 250×21.2 mm, 5 μm column, with a flow rate of 100 mL/minute, column temperature 40° C., eluting with an isocratic 7% MeOH (+0.1% NH₄OH) method (ABPR 60 bar), using a 14 minute run time on a Waters Prep 100 fractionlynx system, in tandem with a Waters SQD2 mass spectrometer.

### Method 10

Analysis was performed using a Lux Cellulose-C1 150×4.6 mm, 3 μm column, flow rate 3 mL/minute, column temperature 35° C., eluting with a gradient 3-40% MeOH (+0.1% NH₄OH) method (ABPR 120 bar), using a 6.5 minute run time on a Waters UPC² Acquity system, in tandem with a Waters QDa mass spectrometer.

### Method 11

Purification was performed using SFC, on a Lux Cellulose-4 250×21.2 mm, 5 μm column, flow rate 100 mL/minute, column temperature 40° C., eluting with a 10-25% MeOH (+0.1% NH₄OH) method (ABPR 60 bar), using a 10 minute run time on a Waters Prep 150 fractionlynx system, in tandem with a Waters QDa mass spectrometer.

### Method 12

Analysis was performed using a Lux Cellulose-C4 150×4.6 mm, 3 μm column, flow rate 3 mL/minute, column temperature 35° C., eluting with a gradient 3-40% MeOH (+0.1% NH₄OH) method (ABPR 120 bar), using a 6.5 minute run time on a Waters UPC² Acquity system, in tandem with a Waters QDa mass spectrometer.

### Method 13

Purification was performed using SFC, on a Lux Cellulose-2 250×21.2 mm, 5 μm column, flow rate 100 mL/minute, column temperature 40° C., eluting with a 3-40% MeOH (+0.1% NH₄OH) method (ABPR 60 bar), using a 7.5 minute run time on a Waters Prep 150 fractionlynx system, in tandem with a Waters QDa mass spectrometer.

### Method 14

Analysis was performed using a Lux Cellulose-C2 150×4.6 mm, 3 μm column, flow rate 3 mL/minute, column temperature 35° C., eluting with a gradient 3-40% MeOH (+0.1% NH₄OH) method (ABPR 120 bar), using a 6.5 minute run time on a Waters UPC² Acquity system, in tandem with a Waters QDa mass spectrometer.

### Method 15

SFC purification was performed using a Chiralpak IB 250×20 mm, 5 μm column, flow rate 100 mL/minute, column temperature 40° C., eluting with a 3-40% MeOH (+0.1% NH₄OH) method (ABPR 60 bar), using a 7.5 minute run time on a Waters Prep 150 fractionlynx system, in tandem with a Waters QDa mass spectrometer.

### Method 16

Analysis was performed using a Chiralpak IB, 150×4.6 mm, 3 μm column, flow rate 3 mL/minute, column temperature 35° C., eluting with a 3-40% MeOH (+0.1% NH₄OH) method (ABPR 120 bar), using a 6.5 minute run time on a Waters UPC² Acquity system, in tandem with a Waters QDa mass spectrometer.

### Method 17

Purification was performed using a Torus DEA 150×19 mm, 5 μm column, flow rate 100 mL/minute, column temperature 40° C., eluting with an isocratic 5% MeOH method (ABPR 60 bar), using a 5 minute run time on a Waters Prep 100 fractionlynx system, in tandem with a Waters SQD2 mass spectrometer.

### Method 18

Analysis was performed using a (R,R) Whelk-O1 150×4.6 mm, 3.5 μm column, flow rate 1.5 mL/minute, column temperature 30° C., eluting with a 50% EtOH:50% n-heptane (+0.1% diethylamine) isocratic method, using an 8 minute run time on a UV directed Agilent 1290 Infinity system.

### Method 19

SFC purification was performed using a Chiralpak, 250×20 mm, 5 μm column with a flow rate of 100 mL/minute, column temperature 40° C., eluting with a 3-40% MeOH (+0.1% NH₄OH) method (ABPR 60 bar), using a 6.5 minute run time on a Waters Prep 100 fractionlynx system, in tandem with a SQD2 mass spectrometer.

### Method 20

Chiral analysis was performed using a Chiralpal IC 150×4.6 mm, 3 μm column, flow rate 3 mL/minute, column temperature 35° C., eluting with a 3-40% MeOH (+0.1% NH₄OH) method (ABPR 120 bar), using a 6.5 minute run time on a Waters UPC² Acquity system, in tandem with a Waters QDa mass spectrometer.

### Method 21

SFC purification was performed using a Chiralpak, 250×20 mm, 5 μm column with a flow rate of 100 mL/minute, column temperature 40° C., eluting with a 3-40% MeOH (+0.1% NH₄OH) method (ABPR 60 bar), using a 7.5 minute run time on a Waters Prep 150 fractionlynx system, in tandem with a QDa mass spectrometer.

### Method 22

SFC purification was performed using a Lux Cellulose-4, 250×21.2 mm, 5 μm column, flow rate 100 mL/minute, column temperature 40° C., eluting with a 3-40% MeOH (+0.1% NH₄OH) method (ABPR 60 bar), using a 7.5 minute run time on a Waters Prep 100 fractionlynx system, in tandem with a SQD2 mass spectrometer.

### Method 23

SFC purification was performed using a Regis (R,R)-Whelk-01, 250×21.1 mm, 5 μm column with a flow rate of 100 mL/minute, column temperature 40° C., eluting with a 3-40% MeOH (+0.1% NH₄OH) method (ABPR 60 bar), using a 7.5 minute run time on a Waters Prep 100 fractionlynx system, in tandem with a SQD2 mass spectrometer.

### Method 24

Chiral analysis was performed using a Regis (R,R)-Whelk-01, 150×4.6 mm, 5 μm column, flow rate 3 mL/minute, column temperature 35° C., eluting with a 3-40% MeOH (+0.1% NH₄OH) method (ABPR 120 bar), using a 6.5 minute run time on a Waters UPC² Acquity system, in tandem with a Waters QDa mass spectrometer.

### Method 25

Chiral analysis was performed using a Cellulose-4, 4.6×250 mm, 5 μm column with a flow rate of 4 mL/minute, eluting with an isocratic gradient of 15% methanol:85% CO₂.

### Method 26

SFC purification was performed using a Regis (R,R)-Whelk-01, 250×21.1 mm, 5 μm column with a flow rate of 100 mL/minute, column temperature 40°, eluting with a 3-40% MeOH (no additive) method (ABPR 60 bar), using a 7.5 minute run time on a Waters Prep 100 fractionlynx system, in tandem with a SQD2 mass spectrometer.

### Method 27

Analysis was performed using a Regis (R,R)-Whelk-01, 150×4.6 mm, 5 μm column, flow rate 3 mL/minute, column temperature 35° C., eluting with a 3-40% MeOH (no additive) method (ABPR 120 bar), using a 6.5 minute run time on a Waters UPC² Acquity system, in tandem with a Waters QDa mass spectrometer.

### Method 28

SFC purification was performed using a ChiralpakIC, 250×20.0 mm, 5 μm column, flow rate 100 mL/minute, column temperature 40° C., eluting with a 10-25% methanol (no additive) gradient (60 bar), over 12 minutes on a Waters FractionLynx SFC Prep 100 in tandem with a SQD2 mass spectrometer.

### Method 29

Analysis was performed using a Chiralpak IC, 150×4.6 mm, 3 μm column, flow rate 3 mL/minute, column temperature 35° C., eluting with a 3-40% MeOH (no additive) method (ABPR 120 bar), using a 6.5 minute run time on a Waters UPC² Acquity system, in tandem with a Waters QDa mass spectrometer.

### Method 30

Stationary Phase: Phenomenex Gemini-NX C18 2.0×50 mm, 3 μm column
Column Temperature: 40° C.
Mobile Phase A: 10 mM ammonium formate in water+0.1% formic acid
Mobile Phase B: acetonitrile+5% water+0.1% formic acid
Flow rate: 1 mL/minute
Gradient program:

| Time | A % | B % |
|---|---|---|
| 0.00 | 95.00 | 5.00 |
| 5.00 | 5.00 | 95.00 |
| 5.40 | 5.00 | 95.00 |
| 5.42 | 95.00 | 5.00 |

### Method 31

Stationary Phase: Waters UPLC® BEH™ C18, Part No. 186005297, 2.1×50 mm,
Column Temperature: 40° C.
Mobile Phase A: water+0.1% formic acid
Mobile Phase B: acetonitrile+0.1% formic acid
Flow rate: 0.9 mL/minute
Gradient program:

| Time | A % | B % |
|---|---|---|
| 0.00 | 95.00 | 5.00 |
| 1.10 | 0.00 | 100 |
| 1.358 | 0.00 | 100 |
| 1.40 | 95.00 | 5.00 |

### Method 32

Preparative HPLC performed on a Gilson System using a Waters Sunfire C18 column (30×100 mm, 10 μm) at r.t. with a flow rate of 40 mL/minute, uv detection @ 215 nm, running a gradient of 30-95% acetonitrile+0.1% formic acid in water+0.1% formic acid.

### Method 33

Preparative Chiral LC carried out on a Gilson system with a 321/322 pump, GX-241 autosampler, 171/172 detector and prep FC fraction collector.
Column: Chiralcel OD-H, 20×250 mm, 5 μm
Column Temperature: r.t.
Flow Rate: 18 mL/minute
Gradient: 85% heptane: 15% EtOH

### Method 34

Chiral analysis carried out on a Waters 2795 with a Waters 2998 PDA detector. Purity and/or enantiomeric purity determined by UV (210-400 nm) and identity confirmed by MS.
Column: Chiralcel OD-H, 4.6×250 mm, 5 μm
Column Temperature: r.t.
Flow Rate: 1 mL/minute
Gradient: 70% heptane:30% EtOH

### Intermediate 1

### tert-Butyl 4-(trifluoromethyl)-4-(trimethylsilyloxy)piperidine-1-carboxylate

To a solution of tert-butyl 4-oxopiperidine-1-carboxylate (2.06 g, 10.3 mmol) in THF (20.7 mL) and DMPU (6.4 mL) at r.t. was added CsF (471 mg, 3.10 mmol) in one portion. The mixture was stirred for 5 minutes, then (trifluoromethyl)trimethylsilane (3.06 mL, 20.7 mmol) was added dropwise over 10 minutes via syringe. The reaction mixture was stirred at r.t. for 15 minutes, then diluted with EtOAc (40 mL) and water (20 mL). The layers were separated, and the aqueous layer was re-extracted with EtOAc (2×50 mL). The combined organic layers were washed with brine (2×40 mL), then dried (Na₂SO₄) and concentrated in vacuo. Purification by flash chromatography, eluting with EtOAc/isohexane (0-10% gradient), gave the title compound (3.12 g, 80%) as a pale yellow oil. δ_{H} (400 MHz, CDCl₃) 4.11-3.96 (m, 2H), 3.07-2.90 (m, 2H), 1.79-1.63 (m, 4H), 1.49 (s, 9H), 0.20 (s, 9H). Rr 0.59 (isohexane:EtOAc, 90:10), non-UV, KMnO₄.

### Intermediate 2

### 1-tert-Butoxycarbonyl-4-(trifluoromethyl)-4-(trimethylsilyloxy)piperidine-2-carboxylic acid

To a solution of Intermediate 1 (4.40 g, 13.0 mmol) and TMEDA (3.50 mL, 23.0 mmol) in diethyl ether (128 mL) at −78° C. was added sec-butyllithium (1.3M, 17.0 mL, 24.0 mmol) dropwise over 10 minutes. The mixture was stirred at −78° C. for 10 minutes, during which time a dull yellow colour resulted. After this time, the reaction mixture was warmed to −40° C. (replacement of dry ice/acetone bath with dry ice/acetonitrile bath) and stirred at −40° C. for 20 minutes. The reaction mixture was re-cooled to −78° C. and CO₂ was bubbled through the mixture for 30 minutes. After this time, the reaction mixture was warmed to r.t. and stirred for 1 h, then quenched by the addition of saturated aqueous NH₄Cl solution (100 mL) and H₂O (50 mL) (to solubilise the resulting precipitate). The layers were separated, then the aqueous layer was washed with diethyl ether (2×100 mL). The combined organic layers were dried (Na₂SO₄) and concentrated in vacuo. Purification by flash chromatography, eluting with EtOAc/isohexane (0-100% gradient), then DCM/MeOH (90:10), gave the title compound (assumed mixture of two diastereomers, indistinguishable by ¹H NMR) (2.58 g, 52%) as a thick yellow oil. δ_{H} (400 MHz, DMSO-d₆) 13.05-12.40 (br s, 1H), 4.25 (dd, J 8.3, 6.4 Hz, 1H), 3.74-3.58 (m, 1H), 3.39-3.19 (obscured m, 1H), 2.16 (dd, J 14.2, 8.4 Hz, 1H), 2.08 (dd, J 14.2, 6.5 Hz, 1H), 2.01-1.91 (m, 1H), 1.87-1.75 (m, 1H), 1.38 (s, 9H), 0.15 (s, 9H).

### Intermediate 3

### 1-tert-Butoxycarbonyl-4-hydroxy-4-(trifluoromethyl)piperidine-2-carboxylic acid

To a solution of Intermediate 2 (1.73 g, 4.49 mmol) in THF (45 mL) at r.t. was added TBAF (1M in THF, 6.73 mL, 6.73 mmol) dropwise over 2 minutes. The mixture was stirred at r.t. for 30 minutes, after which time TLC analysis showed the emergence of a different acid (100% EtOAc, baseline streaking, but more polar than the starting material). The mixture was concentrated in vacuo, then re-dissolved in diethyl ether (20 mL), and H₂O (20 mL) was added. The layers were separated, and the aqueous layer was re-extracted with diethyl ether (2×20 mL). The combined organic extracts were dried (Na₂SO₄) and concentrated in vacuo. To the crude material was added EtOAc (ca 1 mL, the minimum amount to solubilise), followed by isohexane (ca 5 mL). The resulting suspension was stirred at r.t. overnight, then filtered under suction using a Buchner funnel. The precipitate was washed with isohexane, then dried under vacuum, to give the title compound (assumed mixture of two diastereomers, indistinguishable by ¹H NMR) (1.13 g, 80%) as a beige solid. δ_{H} (400 MHz, DMSO-d₆) 13.07-12.34 (br s, 1H), 6.16 (s, 1H), 4.17 (dd, J 9.0, 6.1 Hz, 1H), 3.61-3.44 (m, 1H), 3.42-3.23 (obscured m, 1H), 2.09-1.93 (m, 2H), 1.88-1.76 (m, 1H), 1.75-1.63 (m, 1H), 1.38 (s, 9H).

### Intermediate 4

### O′-tert-Butyl O²-(1,3-dioxoisoindolin-2-yl) 4-hydroxy-4-(trifluoromethyl)piperidine-1,2-dicarboxylate

To a solution of Intermediate 3 (990 mg, 3.16 mmol) and N-hydroxyphthalimide (567 mg, 3.48 mmol) in DCM (15 mL) at r.t. was added EDCI·HCl (666 mg, 3.47 mmol) in one portion. The yellow mixture was stirred for 18 h, then concentrated in vacuo. Purification by flash chromatography, eluting with EtOAc/isohexane (0-60% gradient), gave the title compound (assumed mixture of two diastereomers, indistinguishable by ¹H NMR) (861 mg, 59%) as a white solid. δ_{H} (400 MHz, 373K, DMSO-d₆) 8.00-7.92 (m, 4H), 6.13 (s, 1H), 4.80 (dd, J 10.0, 5.9 Hz, 1H), 3.73 (dt, J 12.9, 6.2 Hz, 1H), 3.43 (ddd, J 13.4, 7.8, 5.3 Hz, 1H), 2.35 (ddd, J 14.0, 5.7, 1.2 Hz, 1H), 2.21 (dd, J 14.0, 10.1 Hz, 1H), 2.02-1.93 (m, 1H), 1.90-1.80 (m, 1H), 1.48 (s, 9H). LCMS (Method 1): [M+H−H₂O]⁺ m/z 476.2, RT 1.40 minutes.

### Intermediate 5

### 5-(Dicyclopropylmethyl)imidazolidine-2,4-dione

A stirred mixture of diammonium carbonate (47.10 g, 0.501 mol), 2,2-dicyclopropylacetaldehyde (95%, 26.10 g, 0.200 mol) and potassium cyanide (13.09 g, 0.201 mol) in a mixture of ethanol (140 mL) and water (140 mL) was heated at 60° C. for 18 h. To the cooled reaction mixture were added 2N HCl (200 mL), then 6N HCl (100 mL) partwise. Additional 2N HCl (60 mL) was added, and the mixture was stirred at r.t. for 1 h. Additional 2N HCl (50 mL) was added to the mixture and the solid was filtered off, then washed with water (2×200 mL) and dried, to afford the title compound (95% purity) (32.81 g, 80%) as a white solid. δ_{H} (500 MHz, DMSO-d₆) 10.58 (s, 1H), 8.04 (s, 1H), 4.05 (d, J 1.5 Hz, 1H), 0.91-0.81 (m, 1H), 0.82-0.73 (m, 1H), 0.52-0.36 (m, 3H), 0.36-0.23 (m, 3H), 0.23-0.17 (m, 1H), 0.13-0.07 (m, 1H), 0.06-0.00 (m, 1H). LCMS (Method 2): [M+H]⁺ m/z 195, RT 1.72 minutes.

### Intermediate 6

### 2-(Benzyloxycarbonylamino)-3,3-dicyclopropylpropanoic acid

To a stirred solution of Intermediate 5 (95%, 1.00 g, 4.89 mmol) in 1,4-dioxane (6 mL) was added 5M aqueous sodium hydroxide solution (6.0 mL, 30.0 mmol). The mixture was heated at 100° C. for 18 h, then 1,4-dioxane (6 mL) and water (6 mL) were added, and the mixture was heated at 120° C. for two days. To the cooled reaction mixture were added TBME (10 mL) and water (10 mL). The two-phase mixture was filtered. To the filtrate were added 6N HCl (6 mL) and TBME (10 mL). The undissolved solid was filtered off. To the filtrate was added TBME (10 mL). The layers were separated, and the aqueous layer was washed with TBME (3×10 mL). To the aqueous layer was added 5N aqueous NaOH solution (0.5 mL), and the pH of the solution was adjusted to pH 7 using 6N HCl/5M aqueous NaOH solution. To the aqueous solution (˜40 mL) were added THF (20 mL), then NaHCO₃ (1.01 g), then disodium carbonate (1.01 g, 9.53 mmol), then 1-(benzyloxycarbonyloxy)pyrrolidine-2,5-dione (0.90 g, 3.61 mmol) at r.t. The mixture was stirred at r.t. for 2.5 days, then TBME (20 mL) was added, followed by water (30 mL). The organic layer was separated off. To the aqueous layer was added water (10 mL). The aqueous layer was washed with TBME (10 mL), then filtered and washed with TBME (10 mL). The pH of the aqueous layer was adjusted to pH 3 using 6N HCl (˜4 mL). Crystals from a previous batch were seeded, then the flask was externally cooled and left to stand for 2 h. The contents were filtered off, then washed with water (2×10 mL) and dried, to afford the title compound (719 mg, 49%) as a white solid. δ_{H} (500 MHz, DMSO-d₆) 12.53 (s, 1H), 7.47 (d, J 8.9 Hz, 1H), 7.41-7.28 (m, 5H), 5.11-5.01 (m, 2H), 4.19 (dd, J 8.9, 4.4 Hz, 1H), 1.03-0.92 (m, 1H), 0.85-0.74 (m, 1H), 0.57-0.49 (m, 1H), 0.49-0.43 (m, 1H), 0.41-0.20 (m, 4H), 0.19-0.01 (m, 3H). LCMS (Method 2): [M+H]⁺ m/z 304, RT 3.13 minutes.

### Intermediates 7 & 8

### (2S)-2-(Benzyloxycarbonylamino)-3,3-dicyclopropylpropanoic acid (Intermediate 7)

### (2R)-2-(Benzyloxycarbonylamino)-3,3-dicyclopropyl propanoic acid (Intermediate 8)

Intermediate 6 (100% purity) (850 g, 2.80 mol) was subjected to separation by prep-SFC (column: Daicel Chiralpak AD, 250 mm×50 mm, 10 μm; mobile phase: [Neu-IPA]; B %: 45%-45%, 6 minutes), and the fractions were concentrated in vacuo at 45° C., to provide the title compounds (Peak 1, 324 g, 1.07 mol, 100% purity; and Peak 2, 351 g, 1.16 mol, 100% purity) as white solids. ¹H NMR and LCMS matched those for Intermediate 6. Chiral analysis (Method 3): Peak 1, RT 1.97 minutes; Peak 2, RT 2.29 minutes.

### Intermediate 9

### Benzyl N-{(1S)-1-(dicyclopropylmethyl)-3-[dimethyl(oxo)-λ⁶-sulfanylidene]-2-oxo-propyl}carbamate

To a suspension of trimethylsulfoxonium iodide (4.57 g, 20.4 mmol) in THF (42 mL) at r.t. was added potassium tert-butoxide (2.19 g, 19.1 mmol) portionwise over 2 minutes. A reflux condenser was fitted, and the mixture was heated at 70° C. for 2 h, then cooled to −5° C. (ice/water/salt bath), to provide an ylide. Meanwhile, a separate flask was charged with Intermediate 7 (2.00 g, 6.59 mmol), THF (22.8 mL), DIPEA (1.53 mL, 8.77 mmol) and HATU (3.20 g, 8.25 mmol) sequentially, and stirred at r.t. for 150 minutes. The resulting activated acid mixture was added to the ylide dropwise via cannula over 30 minutes, keeping the internal temperature of the now-turbid mixture below 0° C. Following the addition, the mixture was stirred for 5 minutes, then quenched with H₂O (15 mL) and saturated aqueous NaHCO₃ solution (15 mL), and stirred for 1 h. The reaction mixture was extracted with EtOAc (2×100 mL), and the combined organic extracts were washed with brine (150 mL), then dried (Na₂SO₄) and concentrated in vacuo. Purification by flash chromatography, eluting with EtOAc/isohexane (0-100% gradient), then DCM/MeOH (90:10), gave the title compound (2.13 g, 86%) as a white solid. δ_{H} (400 MHz, DMSO-d₆) 7.42-7.27 (m, 5H), 6.98 (d, J 9.5 Hz, 1H), 5.11-4.99 (m, 2H), 4.88 (s, 1H), 4.05-3.97 (m, 1H), 2.70 (s, 6H), 0.85-0.73 (m, 1H), 0.73-0.63 (m, 1H), 0.59-0.49 (m, 1H), 0.45-0.37 (m, 1H), 0.37-0.00 (m, 7H). LCMS (Method 1): [M+H]⁺ m/z 378.2, RT 1.18 minutes.

### Intermediate 10

### Benzyl N-[3-bromo-1-(dicyclopropylmethyl)-2-oxopropyl]carbamate

To a solution of Intermediate 9 (2.13 g, 5.64 mmol) in THF (26.1 mL) at 0° C. was added LiBr (495 mg, 5.64 mmol) in one portion. After about 2 minutes the LiBr had completely dissolved, and methanesulfonic acid (0.37 mL, 5.70 mmol) was added dropwise over ˜30 seconds. The mixture was stirred for 5 minutes, turning bright yellow and turbid, then warmed to r.t. (removal of the ice/water bath) and stirred for 30 minutes. The resulting mixture was heated at 65° C. for 2 h, then cooled to r.t. Saturated aqueous NaHCO₃ solution (20 mL) was added, and the reaction mixture was further diluted with H₂O (10 mL), then extracted with EtOAc (2×100 mL). The combined organic extracts were dried (Na₂SO₄) and concentrated in vacuo. Purification by flash chromatography, eluting with EtOAc/isohexane (0-20% gradient), gave the title compound (617 mg, 29%) as a white solid. δ_{H} (400 MHz, DMSO-d₆) 7.82 (d, J 8.4 Hz, 1H), 7.45-7.27 (m, 5H), 5.09 (d, J 12.6 Hz, 1H), 5.06 (d, J 12.6 Hz, 1H), 4.51 (s, 2H), 4.50 (dd, J 8.7, 4.9 Hz, 1H), 0.98-0.87 (m, 1H), 0.87-0.77 (m, 1H), 0.66 (td, J 9.6, 4.9 Hz, 1H), 0.53-0.44 (m, 1H), 0.42-0.14 (m, 5H), 0.11-0.02 (m, 1H), 0.02 to −0.06 (m 1H). LCMS (Method 1): [M+H]⁺ m/z 380.2, 382.2, RT 1.18 minutes.

### Intermediate 11

### Benzyl N-[(1S)-2,2-dicyclopropyl-1-(imidazo[1,2-b][1,2,4]triazin-6-yl)ethyl]carbamate

To a solution of Intermediate 10 (>99% e.e.) (984 mg, 2.59 mmol) and 1,2,4-triazin-3-amine (248 mg, 2.58 mmol) in IPA (18 mL) at r.t. was added NaHCO₃ (260 mg, 3.10 mmol) in one portion. The mixture was stirred at 80° C. for 24 h, then cooled to r.t. and concentrated in vacuo. Purification by flash chromatography, eluting with EtOAc/isohexane (0-100% gradient) gave the title compound (99% e.e.) (354 mg, 36%) as a brown solid. δ_{H} (400 MHz, DMSO-d₆) 8.63 (d, J 2.0 Hz, 1H), 8.53 (d, J 2.0 Hz, 1H), 8.21 (s, 1H), 7.76 (d, J 9.6 Hz, 1H), 7.43-7.20 (m, 5H), 5.13-5.04 (m, 3H), 0.85-0.66 (m, 3H), 0.43-0.29 (m, 2H), 0.29-0.17 (m, 2H), 0.16-0.06 (m, 2H), 0.03 to −0.05 (m, 1H), −0.19 to −0.28 (m, 1H). LCMS (Method 1): [M+H]⁺ m/z 378.2, RT 1.32 minutes.

### Intermediates 12 & 13

### tert-Butyl 2-{6-[(1S)-1-(benzyloxycarbonylamino)-2,2-dicyclopropylethyl]imidazo[1,2-b][1,2,4]triazin-3-yl}-4-hydroxy-4-(trifluoromethyl)piperidine-1-carboxylate (syn isomer) (Intermediate 12)

### tert-Butyl 2-{6-[(1S)-1-(benzyloxycarbonylamino)-2,2-dicyclopropylethyl]imidazo[1,2-b][1,2,4]triazin-3-yl}-4-hydroxy-4-(trifluoromethyl)piperidine-1-carboxylate (anti isomer) (Intermediate 13)

Into a screw-cap vial were introduced Intermediate 11 (356 mg, 0.94 mmol), Intermediate 4 (649 mg, 1.42 mmol), DMF (18 mL), {Ir[dF(CF₃)ppy]₂(dtbpy)}PF₆ (22.0 mg, 0.020 mmol) and TFA (0.11 mL, 1.50 mmol) sequentially. The vial was capped and the mixture was purged with N₂ for 5 minutes, then the cap was sealed with parafilm and the mixture was irradiated (450 nm) using an ‘integrated photoreactor’ (ACS Cent. Sci., 2017, 3, 647-653) (settings: Fan=1612 rpm; Stir=392 rpm; LED=100%) for 20 h. The mixture was diluted with EtOAc (20 mL) and washed with H₂O (2×10 mL). The combined organic layers were dried (Na₂SO₄) and concentrated in vacuo. The residue was purified by flash chromatography, eluting with EtOAc/isohexane (0-60% gradient), to give Intermediate 13 (minor, anti-configured, two stereoisomers) (56.0 mg, 9%) as a yellow foam, together with some additional impure material. Further purification of the additional material by flash chromatography, eluting with diethyl ether/isohexane (0-80% gradient), gave Intermediate 12 (syn-configured, two stereoisomers) (231 mg, 38%) as a white foam. LCMS (Method 1): [M+H]⁺ m/z 645.2, RT 1.59 minutes (major syn isomer), RT 1.55 minutes (minor anti isomer).

### Intermediate 14

### Benzyl N-[(1S)-2,2-dicyclopropyl-1-{3-[4-hydroxy-4-(trifluoromethyl)piperidin-2-yl]-imidazo[1,2-b][1,2,4]triazin-6-yl}ethyl]carbamate trifluoroacetic acid salt

Intermediate 12 (syn-configured, two stereoisomers) (231 mg, 0.36 mmol) was dissolved in DCM (2.6 mL) and TFA (0.41 mL, 5.40 mmol) was added. The solution was stirred at r.t. for 3 h, then neutralised with saturated aqueous NaHCO₃ solution (5 mL). To the mixture was added EtOAc (10 mL), and the layers were separated. The aqueous layer was re-extracted with EtOAc (2×10 mL), then the combined organic layers were dried (Na₂SO₄) and concentrated in vacuo, to give the title compound (mixture of two syn-configured stereoisomers) (236 mg, quantitative) as an orange oil which was utilised without further purification. LCMS (Method 1): [M+H]⁺ m/z 545.2, RT 1.36 minutes.

### Intermediate 15

### Benzyl N-[(1S)-2,2-dicyclopropyl-1-{3-[1-(3-fluorobicyclo[1.1.1]pentane-1-carbonyl)-4-hydroxy-4-(trifluoromethyl)piperidin-2-yl]imidazo[1,2-b][1,2,4]triazin-6-yl}ethyl]-carbamate

To a solution of Intermediate 14 (mixture of two syn-configured stereoisomers) (236 mg), 3-fluorobicyclo[1.1.1]pentane-1-carboxylic acid (51.0 mg, 0.39 mmol) and DIPEA (0.25 mL, 1.40 mmol) in DMF (5 mL) at r.t. was added HATU (169 mg, 0.43 mmol) in one portion. The mixture was stirred for 10 minutes, then H₂O (10 mL) was added. The mixture was extracted with EtOAc (2×20 mL) and the combined organic extracts were washed with brine (20 mL), then dried (Na₂SO₄) and concentrated in vacuo. Purification by flash chromatography, eluting with EtOAc/isohexane (0-80% gradient), gave the title compound (mixture of two stereoisomers) (104 mg, 44%) as a yellow foam. LCMS (Method 1): [M+H]⁺ m/z 657.2, RT 1.49 minutes.

### Intermediate 16

### [(2S,4R)-2-{16-[(1S)-1-Amino-2,2-dicyclopropylethyl]imidazo[1,2-b][1,2,4]triazin-3-yl}-4-hydroxy-4-(trifluoromethyl)piperidin-1-yl](3-fluorobicyclo[1.1.1]pentan-1-yl)-methanone

To a solution of Intermediate 15 (mixture of two stereoisomers) (50.0 mg, 0.076 mmol) in EtOH (2.2 mL) at r.t. were added 4N HCl in 1,4-dioxane (0.02 mL, 0.08 mmol) and 10% Pd/C (10 mg) sequentially. The vessel was evacuated and purged three times with H₂, then left to stir at r.t. for 210 minutes. The mixture was evacuated and placed under an atmosphere of Ar, and Na₂CO₃ (16.1 mg, 0.152 mmol) was added. The mixture was stirred for 5 minutes, then filtered through a pad of Celite® (10 g) under suction using EtOH (20 mL). The filtrate was concentrated in vacuo to give the title compound (mixture of two stereoisomers) (40.0 mg, quantitative) as a dark yellow solid which was utilised without further purification. LCMS (Method 1): [M+H]⁺ m/z 523.2, RT 1.24 minutes.

### Intermediate 17

### O⁴-tert-Butyl O³-(1,3-dioxoisoindolin-2-yl) morpholine-3,4-dicarboxylate

A solution of 4-(tert-butoxycarbonyl)morpholine-3-carboxylic acid (500 mg, 2.16 mmol), N-hydroxyphthalimide (440 mg, 2.70 mmol) and EDCI·HCl (520 mg, 2.70 mmol) in DCM (15 mL) was stirred at r.t. overnight. The reaction mixture was concentrated and the residue was purified by flash column chromatography, eluting with a gradient of 0-40% EtOAc in hexanes, to afford the title compound (812 mg, 99%) as an off-white solid. δ_{H} (400 MHz, DMSO-d₆) 8.02-7.92 (m, 4H), 4.99 (d, J 4.0 Hz, 1H), 4.31 (dt, J 12.2, 1.1 Hz, 1H), 3.92 (dd, J 11.6, 3.9 Hz, 1H), 3.83 (dd, J 12.3, 4.1 Hz, 1H), 3.75-3.66 (m, 1H), 3.51 (td, J 11.8, 3.1 Hz, 1H), 3.20 (td, J 12.7, 3.9 Hz, 1H), 1.47 (s, 9H).

### Intermediate 18

### Benzyl N—[(S)-(4,4-difluorocyclohexyl)(imidazo[1,2-b][1,2,4]triazin-6-yl)methyl]-carbamate

A mixture of benzyl N-[(1S)-3-bromo-1-(4,4-difluorocyclohexyl)-2-oxopropyl]-carbamate (300 mg, 0.74 mmol), 1,2,4-triazin-3-amine (70.0 mg, 0.74 mmol) and NaHCO₃ (75.0 mg, 0.89 mmol) in IPA (5 mL) was stirred at 80° C. overnight. The reaction mixture was concentrated and the residue was purified twice by flash column chromatography, eluting with a gradient of 0-60% EtOAc in hexanes, to afford the title compound (111 mg, 36%) as a brown solid. LCMS (Method 1) [M+H]⁺ m/z 402.2, RT 1.27 minutes.

### Intermediate 19

### tert-Butyl 3-{6-[(S)-benzyloxycarbonylamino(4,4-difluorocyclohexyl)methyl]imidazo-[1,2-b][1,2,4]triazin-3-yl}morpholine-4-carboxylate

A solution of Intermediate 17 (190 mg, 0.50 mmol), Intermediate 18 (135 mg, 0.34 mmol), {Ir[dF(CF₃)ppy]₂(dtbpy)}PF₆ (8.0 mg, 7.1 μmol) and TFA (40.0 μL, 0.53 mmol) in DMF (7 mL) was purged with nitrogen gas for 5 minutes. The reaction mixture was placed under 450 nm irradiation for 3 h, then diluted with EtOAc (20 mL) and washed with water (20 mL). The aqueous layer was extracted with EtOAc (20 mL). The combined organic layers were dried over Na₂SO₄, then filtered and concentrated. The residue was purified by flash column chromatography, eluting with a gradient of 0-40% EtOAc in hexanes, to afford the title compound (156 mg, 79%) as an off-white solid. LCMS (Method 1): [M+H]⁺ m/z 587.2, RT 1.47 minutes.

### Intermediate 20

### (S)-(4,4-Difluorocyclohexyl)(imidazo[1,2-b][1,2,4]triazin-6-yl)methanamine

To a solution of Intermediate 18 (900 mg, 2.02 mmol) in AcOH (6 mL) was added hydrogen bromide in AcOH (35%, 3.3 mL, 20.2 mmol). The solution was stirred at r.t. for 2 h. Diethyl ether (50 mL) was added, and the mixture was stirred at r.t. for 30 minutes. The resulting precipitate was collected by vacuum filtration. The sticky residue was washed with diethyl ether (2×50 mL), and transferred from filter paper to separating funnel by rinsing with water. The aqueous layer was washed with DCM (50 mL), then basified with saturated aqueous NaHCO₃ solution. The resulting material was extracted with DCM (3×50 mL). The combined organic extracts were washed with brine (50 mL) and dried over MgSO₄, then filtered and concentrated under reduced pressure, to give the title compound (435 mg, 78%). δ_{H} (500 MHz, CDCl₃) 8.42 (d, J 2.0 Hz, 1H), 8.33 (d, J 2.0 Hz, 1H), 7.90 (s, 1H), 4.07 (d, J 6.3 Hz, 1H), 2.38-1.87 (m, 6H), 1.82-1.58 (m, 3H), 1.56-1.36 (m, 2H). LCMS (Method 4): [M+H]⁺ 268.2, RT 1.26 minutes.

### Intermediate 21

### N—[(S)-(4,4-Difluorocyclohexyl)(imidazo[1,2-b][1,2,4]triazin-6-yl)methyl]-4-methyl-1,2,5-oxadiazole-3-carboxamide

To a solution of Intermediate 20 (435 mg, 1.58 mmol), 4-methyl-1,2,5-oxadiazole-3-carboxylic acid (222 mg, 1.74 mmol) and DIPEA (0.55 mL, 3.16 mmol) in anhydrous DMF (10 mL) was added HATU (720 mg, 1.89 mmol). The mixture was stirred at r.t. for 30 minutes, then diluted with EtOAc (25 mL) and washed with water (2×25 mL), followed by brine (10 mL). The organic layer was dried over MgSO₄, then filtered and concentrated under reduced pressure. The residue was purified by column chromatography, eluting with a gradient of EtOAc in heptane, to give the title compound (460 mg, 77%). δ_{H} (500 MHz, CDCl₃) 8.49 (d, J 1.9 Hz, 1H), 8.39 (d, J 1.9 Hz, 1H), 7.92 (s, 1H), 7.77 (d, J 9.0 Hz, 1H), 5.35-5.28 (m, 1H), 2.60 (s, 3H), 2.31-2.21 (m, 1H), 2.21-2.11 (m, 1H), 2.11-1.98 (m, 2H), 1.85-1.61 (m, 3H), 1.58-1.46 (m, 1H), 1.44-1.32 (m, 1H). LCMS (Method 4): [M+H]˜ 378.2, RT 2.09 minutes.

### Intermediate 22

### tert-Butyl 3-(6-{(S)-(4,4-difluorocyclohexyl)[(4-methyl-1,2,5-oxadiazole-3-carbonyl)-amino]methyl}imidazo[1,2-b][1,2,4]triazin-3-yl)morpholine-4-carboxylate

A solution of Intermediate 17 (1.34 g, 3.55 mmol), Intermediate 21 (940 mg, 2.37 mmol) and {Ir[dF(CF₃)ppy]2(dtbpy)}PF₆ (53 mg, 0.047 mmol) in anhydrous DMF (45 mL) was divided equally between two 40 mL vials. To each vial was added TFA (136 μL, 1.79 mmol). The solutions were purged for 10 minutes by bubbling through nitrogen with stirring, then sealed under nitrogen with parafilm and irradiated with a blue LED lamp (40 W, Kessil A160WE LED Aquarium Light; Tuna Blue; light set at maximum intensity and greater blue colour setting) with stirring at approximately 21° C. (fan-assisted temperature maintenance). The vials were positioned approximately 5 cm from the nearest light. After 24 h, the solutions were combined and diluted with EtOAc (100 mL), then washed with water (2×100 mL), followed by brine (50 mL). The organic fraction was dried over MgSO₄, then filtered and concentrated under reduced pressure. The residue was purified by column chromatography, eluting with a gradient of EtOAc in heptane, to give the title compound (950 mg, 66%). δ_{H} (400 MHz, CDCl₃) 8.34-8.27 (m, 1H), 7.90-7.84 (m, 1H), 7.80-7.71 (m, 1H), 5.35-5.12 (m, 2H), 4.80-4.63 (m, 1H), 4.04-3.80 (m, 3H), 3.67-3.54 (m, 1H), 3.54-3.08 (m, 1H), 2.63-2.56 (m, 3H), 2.32-1.99 (m, 4H), 1.86-1.59 (m, 3H), 1.59-1.30 (m, 11H). LCMS (Method 4): [M+H]⁺ 563.2, RT 2.36 minutes.

### Intermediate 23

### Benzyl N—{(S)-(4,4-difluorocyclohexyl)[3-(morpholin-3-yl)imidazo[1,2-b][1,2,4]triazin-6-yl]methyl}carbamate trifluoroacetic acid salt

Intermediate 19 (488 mg, 0.83 mmol) was dissolved in DCM (5.9 mL) and TFA (0.94 mL, 12.0 mmol) was added. The solution was stirred at r.t. for 160 minutes, then neutralised with saturated aqueous NaHCO₃ solution (10 mL). To the mixture was added EtOAc (10 mL), and the layers were separated. The aqueous layer was re-extracted with EtOAc (2×20 mL), then the combined organic layers were dried (Na₂SO₄) and concentrated in vacuo, to give the title compound (500 mg, quantitative) as an orange oil which was utilised without further purification. LCMS (Method 1): [M+H]˜ m/z 487.2, RT 1.22 minutes.

### Intermediate 24

### N—{(S)-(4,4-Difluorocyclohexyl)[3-(morpholin-3-yl)imidazo[1,2-b][1,2,4]triazin-6-yl]-methyl}-4-methyl-1,2,5-oxadiazole-3-carboxamide hydrochloride

To Intermediate 22 (1.00 g, 1.65 mmol) was added 4N HCl in 1,4-dioxane (15 mL) and the solution was stirred at r.t. for 30 minutes. The solvent was removed under reduced pressure to give the title compound (1.04 g, 99%). δ_{H} (400 MHz, CDCl₃) 10.57-10.41 (m, 1H), 9.97-9.82 (m, 1H), 9.57 (d, J 8.9 Hz, 1H), 9.01-8.91 (m, 1H), 8.45 (s, 1H), 5.29-5.19 (m, 1H), 4.92-4.83 (m, 1H), 4.43-4.28 (m, 1H, obs. by water), 4.07-3.98 (m, 1H), 3.88-3.72 (m, 2H), 3.43-3.34 (m, 1H), 3.32-3.19 (m, 1H), 2.48-2.45 (m, 3H), 2.28-2.15 (m, 1H), 2.13-1.95 (m, 2H), 1.95-1.60 (m, 4H), 1.50-1.37 (m, 1H), 1.37-1.24 (m, 1H). LCMS (Method 4): [M+H]⁺ 463.2, RT 1.79 minutes.

### Intermediate 25

### Benzyl N—[(S)-(4,4-difluorocyclohexyl){3-[4-(3-fluorobicyclo[1.1.1]pentane-1-carbonyl)-morpholin-3-yl]imidazo[1,2-b][1,2,4]triazin-6-yl}methyl]carbamate

To a solution of Intermediate 23 (500 mg, 0.83 mmol), 3-fluorobicyclo[1.1.1]-pentane-1-carboxylic acid (124 mg, 0.95 mmol) and DIPEA (0.60 mL, 3.50 mmol) in DMF (20 mL) at r.t. was added HATU (408 mg, 1.04 mmol) in one portion. The mixture was stirred for 45 minutes, then H₂O (30 mL) was added. The mixture was extracted with EtOAc (2×50 mL) and the combined organic extracts were washed with brine (100 mL), then dried (Na₂SO₄) and concentrated in vacuo. Purification by flash chromatography, eluting with EtOAc/isohexane (0-100% gradient), gave the title compound (444 mg, 89% over two steps) as an orange foam. LCMS (Method 1): [M+H]⁺ m/z 599.2, RT 1.36 minutes.

### Intermediate 26

### (3-{6-[(S)-Amino(4,4-difluorocyclohexyl)methyl]imidazo[1,2-b][1,2,4]triazin-3-yl}-morpholin-4-yl)(3-fluorobicyclo[1.1.1]pentan-1-yl)methanone

To a solution of Intermediate 25 (444 mg, 0.74 mmol) in EtOH (22 mL) at r.t. were added 4N HCl in 1,4-dioxane (0.23 mL, 0.92 mmol) and 10% Pd/C (85 mg) sequentially. The vessel was evacuated and purged three times with H₂, then left to stir at r.t. for ˜5 h. The mixture was filtered through a pad of Celite® (10 g) under suction using EtOH (60 mL). The filtrate was concentrated in vacuo to give the title compound (71% purity by LC-MS) (405 mg, quantitative) which was utilised without further purification. LCMS (Method 1): [M+H]⁺ m/z 465.2, RT 1.10 minutes.

### Intermediate 27

### tert-Butyl 3-{16-[(1S)-1-(benzyloxycarbonylamino)-2,2-dicyclopropylethyl]imidazo[1,2-b][1,2,4]triazin-3-yl}morpholine-4-carboxylate

Into a screw-cap vial were introduced Intermediate 11 (355 mg, 0.94 mmol), Intermediate 17 (531 mg, 1.41 mmol), DMF (18 mL), {Ir[dF(CF₃)ppy]2(dtbpy)}PF₆ (21.0 mg, 0.019 mmol) and TFA (0.11 mL, 1.50 mmol) sequentially. The vial was capped and the mixture was purged with N₂ for 5 minutes, then the cap was sealed with parafilm and the mixture was irradiated (450 nm) using an ‘integrated photoreactor’ (ACS Cent. Sci., 2017, 3, 647-653) (settings: Fan=1612 rpm; Stir=392 rpm; LED=100%) for 16 h. The mixture was diluted with EtOAc (40 mL) and washed with H₂O (2×20 mL). The combined organic fractions were dried (Na₂SO₄) and concentrated in vacuo. Purification by flash chromatography, eluting with EtOAc/isohexane (0-100% gradient), gave the title compound (mixture of two stereoisomers, 1:1 ratio) (278 mg, 52%) as an orange foam. LCMS (Method 1): [M+H]⁺ m/z 563.2, RT 1.45 minutes.

### Intermediate 28

### tert-Butyl 3-{6-[(1S)-1-amino-2,2-dicyclopropylethyl]imidazo[1,2-b][1,2,4]triazin-3-yl}-morpholine-4-carboxylate

To a solution of Intermediate 27 (278 mg, 0.49 mmol) in EtOH (22 mL) at r.t. were added 4N HCl in 1,4-dioxane (0.15 mL, 0.60 mmol) and 10% Pd/C (28 mg) sequentially. The vessel was evacuated and purged three times with H₂, then left to stir at r.t. for ˜3 h (95% conversion of starting material by LC-MS). The mixture was filtered through a pad of Celite® (10 g) under suction using EtOH (60 mL). The filtrate was concentrated in vacuo to give the title compound ( ) mixture of two stereoisomers, 1:1 ratio) (85% purity by LC-MS) (249 mg, quantitative) which was utilised without further purification. LCMS (Method 1): [M+H]⁺ m/z 429.2, RT 1.23 minutes.

### Intermediate 29

### tert-Butyl 3-(6-{(1S)-2,2-dicyclopropyl-1-[(4-methyl-1,2,5-oxadiazole-3-carbonyl)-amino]ethyl}imidazo[1,2-b][1,2,4]triazin-3-yl)morpholine-4-carboxylate

To a solution of Intermediate 28 (249 mg, 0.58 mmol), 4-methyl-1,2,5-oxadiazole-3-carboxylic acid (75.0 mg, 0.59 mmol) and DIPEA (0.40 mL, 2.30 mmol) in DMF (10 mL) at r.t. was added HATU (274 mg, 0.70 mmol) in one portion. The mixture was stirred for 30 minutes, then H₂O (25 mL) was added. The mixture was extracted with EtOAc (3×20 mL) and the combined organic extracts were washed with brine (40 mL), then dried (Na₂SO₄) and concentrated in vacuo. Purification by flash chromatography, eluting with EtOAc/isohexane (0-75% gradient), gave the title compound (mixture of two stereoisomers, 1:1 ratio) (175 mg, 56% over two steps) as an orange solid. LCMS (Method 1): [M+H]⁺ m/z 539.2, RT 1.43 minutes.

### Intermediate 30

### N-{(1S)-2,2-Dicyclopropyl-1-[3-(morpholin-3-yl)imidazo[1,2-b][1,2,4]triazin-6-yl]-ethyl}-4-methyl-1,2,5-oxadiazole-3-carboxamide trifluoroacetic acid salt

Intermediate 29 (175 mg, 0.32 mmol) was dissolved in DCM (1.9 mL) and TFA (0.37 mL, 4.90 mmol) was added. The solution was stirred at r.t. for 130 minutes, then neutralised with saturated aqueous NaHCO₃ solution (10 mL). To the mixture was added EtOAc (10 mL), and the layers were separated. The aqueous layer was re-extracted with EtOAc (2×20 mL). The combined organic layers were dried (Na₂SO₄), then concentrated in vacuo, to give the title compound (mixture of two stereoisomers, 1:1 ratio) (206 mg, quantitative) as an orange oil which was utilised without further purification. LCMS (Method 1): [M+H]⁺ m/z 439.2, RT 1.21 minutes.

### Intermediates 31 & 32

### tert-Butyl 2-{6-[(S)-benzyloxycarbonylamino(4,4-difluorocyclohexyl)methyl]imidazo-[1,2-b][1,2,4]triazin-3-yl}-4-hydroxy-4-(trifluoromethyl)piperidine-1-carboxylate (syn isomers) (Intermediate 31)

### tert-Butyl 2-{6-[(S)-benzyloxycarbonylamino(4,4-difluorocyclohexyl)methyl]imidazo-[1,2-b][1,2,4]triazin-3-yl}-4-hydroxy-4-(trifluoromethyl)piperidine-1-carboxylate (anti isomers) (Intermediate 32)

To a solution of Intermediate 4 (856 mg, 1.87 mmol), Intermediate 18 (500 mg, 1.25 mmol) and {Ir[dF(CF₃)ppy]₂(dtbpy)}PF₆ (28 mg, 0.025 mmol) in anhydrous DMF (20 mL) was added TFA (143 μL, 1.87 mmol). The solution was purged by bubbling through nitrogen with stirring for 10 minutes, then sealed under nitrogen with parafilm and irradiated at 450 nm in a Penn M2 photoreactor (LED 100%, stirring 50%, fan 50%) for 50 h. The solution was diluted with EtOAc (50 mL) and washed with water (2×50 mL), followed by brine (25 mL). The organic portion was dried over MgSO₄, then filtered and concentrated under reduced pressure. The residue was purified by column chromatography, eluting with a gradient of EtOAc in heptane, to give the title compounds (syn isomers, 390 mg, 44%; and anti isomers, 140 mg, 15%). LCMS (Method 5): [M+H]⁺ 669.2, RT 2.51 minutes (syn isomers). LCMS (Method 4): [M+H]⁺ 669.2, RT 2.45 minutes (anti isomers).

### Intermediate 33

### Benzyl N—[(S)-(4,4-difluorocyclohexyl){3-[4-hydroxy-4-(trifluoromethyl)piperidin-2-yl]imidazo[1,2-b][1,2,4]triazin-6-yl}methyl]carbamate hydrochloride

To Intermediate 31 (syn isomers) (390 mg, 0.55 mmol) was added 4N HCl in 1,4-dioxane (5 mL), and the solution was stirred at r.t. for 30 minutes. The solvent was removed under reduced pressure to give the title compound (mixture of syn stereoisomers) (415 mg, 89%). δ_{H} (400 MHz, DMSO-d₆) 10.58-9.89 (m, 1H), 9.71-9.48 (m, 1H), 8.97-8.90 (m, 1H), 8.30-8.15 (m, 1H), 7.97-7.86 (m, 1H), 7.42-7.16 (m, 5H), 5.19-5.11 (m, 1H), 5.11-4.97 (m, 2H), 4.85-4.70 (m, 1H), 3.43-3.27 (m, 1H), 2.64-2.56 (m, 1H), 2.47-2.38 (m, 1H), 2.28-2.15 (m, 1H), 2.15-1.86 (m, 5H), 1.86-1.55 (m, 4H), 1.48-1.32 (m, 1H), 1.32-1.20 (m, 1H). One exchangeable proton signal not observed. LCMS (Method 4): [M+H]⁺ 569.2, RT 1.95 minutes.

### Intermediate 34

### Benzyl N—[(S)-(4,4-difluorocyclohexyl){3-[1-(3-fluorobicyclo[1.1.1]pentane-1-carbonyl)-4-hydroxy-4-(trifluoromethyl)piperidin-2-yl]imidazo[1,2-b][1,2,4]triazin-6-yl}methyl]-carbamate

To a solution of 3-fluorobicyclo[1.1.1]pentane-1-carboxylic acid (70 mg, 0.57 mmol), DIPEA (255 μL, 1.46 mmol) and Intermediate 33 (415 mg, 0.49 mmol) in DCM (5 mL) was added HATU (222 mg, 0.58 mmol). The solution was stirred at r.t. for 2 h, then concentrated under reduced pressure. The residue was purified twice by column chromatography, eluting with a gradient of EtOAc in heptane, to give the title compound (mixture of syn stereoisomers) (240 mg, 67%). δ_{H} (400 MHz, DMSO-d₆) 8.79-8.69 (m, 1H), 8.17-8.05 (m, 1H), 7.90-7.78 (m, 1H), 7.43-7.16 (m, 5H), 5.99-5.56 (m, 2H), 5.11-4.97 (m, 2H), 4.82-4.68 (m, 1H), 4.53-4.04 (m, 1H), 3.80-3.65 (m, 1H), 2.65-2.54 (m, 1H), 2.49-2.41 (m, 3H), 2.32-2.09 (m, 3H), 2.09-1.90 (m, 3H), 1.88-1.51 (m, 7H), 1.44-1.20 (m, 2H). LCMS (Method 4): [M+H]⁺ 681.2, RT 2.39 minutes.

### Intermediate 35

### [2-{6-[(S)-Amino(4,4-difluorocyclohexyl)methyl]imidazo[1,2-b][1,2,4]triazin-3-yl}-4-hydroxy-4-(trifluoromethyl)piperidin-1-yl](3-fluorobicyclo[1.1.1]pentan-1-yl)methanone

To a solution of Intermediate 34 (240 mg, 0.33 mmol) in EtOH (10 mL) was added 10% Pd/C (55-65% wet) (5.0%, 70 mg, 0.033 mmol). The mixture was stirred at r.t. under a balloon of hydrogen for 4 h. The flask was flushed three times with nitrogen, then the mixture was filtered through Celite®, washing through with EtOH. The residue was concentrated under reduced pressure to give the title compound (mixture of syn stereoisomers) (180 mg, 90%). δ_{H} (400 MHz, DMSO-d₆) 8.76-8.65 (m, 1H), 8.18-8.08 (m, 1H), 5.98-5.54 (m, 2H), 4.56-3.65 (m, 4H), 2.49-2.06 (m, 6H), 2.06-1.47 (m, 12H), 1.47-1.26 (m, 2H). LCMS (Method 4): [M+H]⁺ 547.2, RT 1.85 minutes.

### Intermediate 36

### O⁴-tert-Butyl O³-(1,3-dioxoisoindolin-2-yl) 1,1-dioxo-1,4-thiazinane-3,4-dicarboxylate

To a suspension of 4-tert-butoxycarbonyl-1,1-dioxo-1,4-thiazinane-3-carboxylic acid (1.25 g, 4.48 mmol) in DCM (30 mL) at r.t. were added N-hydroxyphthalimide (1.03 g, 6.13 mmol), DMAP (56 mg, 0.45 mmol) and EDCI·HCl (1.32 g, 6.75 mmol) portionwise. The resultant white suspension was stirred and slowly resulted in a yellow solution, which was stirred for 18 h, then concentrated in vacuo. The resulting yellow syrup was partitioned between EtOAc (200 mL) and brine (300 mL). The aqueous phase was extracted with further EtOAc (2×200 mL). The combined organic extracts were washed with brine, then dried over anhydrous Na₂SO₄ and filtered. The volatiles were removed in vacuo. The resulting foamy pale solid was purified by column chromatography on silica, eluting with a gradient of 0-80% EtOAc in hexanes, to furnish the title compound (1.62 g, 85%) as a white solid. δ_{H} (400 MHz, DMSO-d₆) 8.23-7.88 (m, 4H), 6.13-5.80 (m, 1H), 4.65-4.30 (m, 1H), 3.94-3.73 (m, 1H), 3.70-3.36 (m, 3H), 3.29-3.07 (m, 1H), 1.46 (s, 9H).

### Intermediate 37

### (NE)-2-Methyl-N-{[trans-4-(trifluoromethyl)cyclohexyl]methylene}propane-2-sulfinamide

To a solution of trans-4-(trifluoromethyl)cyclohexanecarboxaldehyde (16.1 g, 74.2 mmol) in DCM (500 mL) was added (S)-(−)-2-methyl-2-propanesulfinamide (8.70 g, 70.0 mmol), followed by titanium(IV) ethoxide (52 mL, 223.2 mmol). The mixture was heated under reflux for 2 h, then cooled to r.t. and treated with water (50 mL). After vigorous stirring, the fine suspension was filtered through Celite®. The filtrate was passed through a hydrophobic frit and concentrated in vacuo to afford the title compound (18.8 g, 89%) as an off-white solid. [α]²⁵_{D}+145° (c 1.00, DCM). δ_{H} (300 MHz, CDCl₃) 8.00 (d, J 4.3 Hz, 1H), 2.57-2.40 (m, 1H), 2.17-1.96 (m, 5H), 1.54-1.28 (m, 4H), 1.21 (s, 9H). ¹⁹F {¹H} NMR (282 MHz, CDCl₃) δ −73.84 (s, 3F).

### Intermediate 38

### N—{(S)-Cyano[trans-4-(trifluoromethyl)cyclohexyl]methyl}-2-methylpropane-2-sulfinamide

To a solution of Intermediate 37 (18.8 g, 66.3 mmol) in DCM (180 mL) at 25° C. was added cesium fluoride (1.00 g, 6.58 mmol), followed by trimethylsilyl cyanide (18.0 mL, 134 mmol). The mixture was stirred for 17 h at 25° C. Saturated aqueous sodium bicarbonate solution (150 mL) was added, and the mixture was stirred for 1 h. The biphasic mixture was separated and the organic layers were concentrated in vacuo. The resulting pale yellow oil was purified by flash column chromatography (0-100% EtOAc/isohexane), then recrystallised from EtOAc:isohexane (1:2; 90 mL), to afford the title compound (single diastereoisomer) (7.20 g, 35%) as a white solid. δ_{H} (400 MHz, CDCl₃) 4.05 (dd, J 8.0, 6.4 Hz, 1H), 3.66 (d, J 8.0 Hz, 1H), 2.15-1.99 (m, 5H), 1.91-1.74 (m, 1H), 1.45-1.32 (m, 2H), 1.28 (s, 9H), 1.26-1.11 (m, 2H). ¹⁹F {¹H} NMR (282 MHz, CDCl₃) δ −73.80 (s, 3F).

### Intermediate 39

### (2S)-2-Amino-2-[trans-4-(trifluoromethyl)cyclohexyl]acetonitrile hydrochloride

To a solution of Intermediate 38 (7.00 g, 22.6 mmol) in MeOH (17 mL) was added HCl in 1,4-dioxane (4N, 12 mL), resulting in a white precipitate. After 4 h, the mixture was concentrated under reduced pressure. To the resulting oily slurry was added isohexane (100 mL). After vigorous stirring, the resultant white solid was filtered out, to afford the title compound (5.20 g, 95%). δ_{H} (300 MHz, DMSO-d₆) 9.15 (s, 3H), 4.57 (d, J 5.5 Hz, 1H), 2.39-2.20 (m, 1H), 2.11-1.79 (m, 5H), 1.40-1.01 (m, 4H).

### Intermediate 40

### (2S)-2-Amino-2-[trans-4-(trifluoromethyl)cyclohexyl]acetic acid hydrochloride

Acetic acid (35 mL) and HCl in H₂O (33%, 50 mL) were added to Intermediate 39 (5.20 g, 21.0 mmol). The mixture was heated under reflux for 4 h, then cooled with stirring to 20° C. After 18 h, the white precipitate was filtered off, and dried under a flow of air, to afford the title compound (4.40 g, 74%) as a white solid. [α]²⁵_{D}+22.1° (c 1.0, MeOH). δ_{H} (300 MHz, DMSO-d₆) 13.81 (s, 1H), 8.46 (s, 3H), 3.75 (d, J 4.3 Hz, 1H), 2.34-2.05 (m, 1H), 2.03-1.66 (m, 5H), 1.47-1.04 (m, 4H). ¹⁹F {¹H} NMR (282-MHz, DMSO-d₆) δ −72.33 (s, 3F).

### Intermediate 41

### (2S)-2-(Benzyloxycarbonylamino)-2-[4-(trifluoromethyl)cyclohexyl]acetic acid

To a suspension of Intermediate 40 (5.00 g, 19.1 mmol) and triethylamine (10 mL, 71.7 mmol) in DCM (100 mL) at 0° C. was added N-(benzyloxycarbonyloxy)succinimide (4.47 g, 17.6 mmol) in three portions. The reaction mixture was allowed to warm to r.t. overnight. The reaction mixture was diluted with DCM (10 mL), washed with 5% hydrochloric acid (2×15 mL) and water (15 mL), then dried (Na₂SO₄) and concentrated in vacuo, to give the title compound (6.34 g, 92%) as a white solid. δ_{H} (300 MHz, DMSO-d₆) 12.65 (s, 1H), 7.54 (d, J 8.4 Hz, 1H), 7.40-7.28 (m, 5H), 5.04 (s, 2H), 3.89 (dd, J 8.4, 5.9 Hz, 1H), 2.26-2.05 (m, 1H), 1.96-1.80 (m, 2H), 1.80-1.59 (m, 3H), 1.32-1.07 (m, 4H).

### Intermediate 42

### Benzyl N-{(1S)-3-[dimethyl(oxo)-26-sulfanylidene]-2-oxo-1-[4-(trifluoromethyl)-cyclohexyl]propyl}carbamate

Trimethylsulfoxonium iodide (70.00 g, 0.318 mol) was dissolved in THF (350 mL) and 1M potassium tert-butoxide in THF (300 mL, 0.300 mol) was added. The resulting mixture was stirred at 70° C. (external temperature) under nitrogen for 2 h, then cooled to −5° C. in an ice/salt bath under nitrogen, to provide an ylide. Meanwhile, in a separate flask, Intermediate 41 (37.00 g, 0.103 mol) was dissolved in THF (350 mL) and HATU (49.00 g, 0.129 mol) was added, followed by DIPEA (24 mL, 0.137 mol). The reaction mixture was stirred under nitrogen for 150 minutes, then added dropwise over ˜50 minutes to the cooled solution of the ylide, maintaining internal temperature below 1° C. The reaction mixture was stirred at −4° C. under nitrogen for 5 minutes, then quenched at −4° C. by addition of water (700 mL) and saturated aqueous NaHCO₃ solution (700 mL). The resulting suspension was extracted with TBME (3 L). The organic layer was washed with brine (500 mL) and concentrated to dryness under vacuum. The residue was suspended in TBME (500 mL) and water (50 mL), then heated to 50° C. and cooled to r.t., then filtered, then washed with TBME (100 mL) and heptane (100 mL). An additional three crops were obtained as more solid precipitated from solution in the filtrate each time. The four crops of solid were combined, then water (400 mL) was added. The mixture was sonicated to break up big lumps, then cooled to 0° C., filtered and washed with water (150 mL). the residue was dried in a vacuum oven overnight to afford the title compound (36.15 g, 81%) as a colourless solid. δ_{H} (400 MHz, DMSO-d₆) 7.42-7.27 (m, 5H), 7.06 (d, J 9.2 Hz, 1H), 5.01 (s, 2H), 4.87 (s, 1H), 3.67 (dd, J 9.1, 6.6 Hz, 1H), 3.43 (s, 6H), 2.20-2.03 (m, 1H), 1.92-1.78 (m, 2H), 1.76-1.54 (m, 3H), 1.26-0.99 (m, 4H). LCMS (Method 6): [M+H]˜ 434.2, RT 2.70 minutes.

### Intermediate 43

### Benzyl N-{(1S)-3-bromo-2-oxo-1-[4-(trifluoromethyl)cyclohexyl]propyl}carbamate

Intermediate 42 (28.08 g, 64.8 mmol) was dissolved in THF (300 mL) and cooled to 0° C. (external temperature) under nitrogen. LiBr (5.69 g, 64.8 mmol) was added, and the mixture was stirred until it fully dissolved (˜2 minutes). Methanesulfonic acid (4.2 mL, 64.7 mmol) was added and the mixture was stirred at 0° C. under nitrogen for 5 minutes, then allowed to warm to r.t. and stirred for 30 minutes under nitrogen. The resulting suspension was warmed to 58° C. (internal temperature; heating block at 65° C.) over approximately 30 minutes, and stirred under nitrogen at this temperature for 1 h. The reaction mixture was allowed to cool to r.t. and quenched by the addition of saturated aqueous NaHCO₃ solution (450 mL). The resulting mixture was diluted with water (200 mL) and extracted with EtOAc (600 mL). The organic layer was washed with saturated aqueous NaBr solution (400 mL), then dried (Na₂SO₄) and concentrated to dryness under vacuum. The residue was purified by FCC (750 g Biotage KP-Sil cartridge, wet loaded in 150 mL DCM, eluting with 10-20% EtOAc in heptane), and the product fractions were concentrated to dryness under vacuum, to afford the title compound (19.80 g, 68%) as a colourless solid. A mixed fraction was isolated and concentrated to dryness under vacuum, then re-purified by FCC (100 g Biotage Sfar Duo cartridge, wet loaded in 20 mL DCM, eluting with 10-20% EtOAc in heptane), and the product fractions were concentrated to dryness under vacuum, to afford a second crop of the title compound (4.90 g, 17%) as a colourless solid. δ_{H} (500 MHz, CDCl₃) 7.44-7.29 (m, 5H), 5.31 (d, J 8.7 Hz, 1H), 5.11 (s, 2H), 4.66 (dd, J 8.7, 4.6 Hz, 1H), 4.03 (q, J 13.3 Hz, 2H), 2.03-1.92 (m, 3H), 1.92-1.83 (m, 2H), 1.63 (d, J 13.0 Hz, 1H), 1.42-1.18 (m, 3H), 1.06 (qd, J 13.0, 3.2 Hz, 1H). LCMS (Method 4): [M+H]⁺ 436.0/438.0, RT 3.27 minutes.

### Intermediate 44

### Benzyl N—{(S)-(imidazo[1,2-b][1,2,4]triazin-6-yl)[4-(trifluoromethyl)cyclohexyl]methyl}carbamate

To a suspension of Intermediate 43 (5.09 g, 11.67 mmol) in EtOH (75 mL) at ambient temperature was added 3-amino-1,2,4-triazine (recrystallised from acetonitrile) (2.3 g, 23.22 mmol). The resultant suspension was heated to 80° C., whereupon the suspension became a red solution. After 1 h, the reaction mixture was cooled, and poured into a mixture of saturated aqueous NaHCO₃ solution (300 mL) and EtOAc (200 mL). The organic phase was separated. The aqueous phase was diluted with brine (200 mL) and extracted with further EtOAc (2×200 mL). The combined organic extracts were washed twice with brine, then dried over anhydrous Na₂SO₄ and filtered. The volatiles were removed in vacuo. The resulting brown solid was purified by column chromatography on silica, eluting with a gradient of EtOAc (0-80% in hexanes), to provide the title compound (1.8 g, 36%) as a reddish-brown crystalline solid. δ_{H} (400 MHz, DMSO-d₆) 8.64 (d, J 2.0 Hz, 1H), 8.55 (d, J 2.1 Hz, 1H), 8.20 (s, 1H), 7.82 (d, J 9.2 Hz, 1H), 7.55-7.04 (m, 5H), 5.03 (d, J 2.3 Hz, 2H), 4.69 (dd, J 9.2, 7.2 Hz, 1H), 2.19 (s, 1H), 1.85 (dd, J 16.6, 7.2 Hz, 4H), 1.59 (d, J 12.6 Hz, 1H), 1.30-0.95 (m, 4H).

### Intermediate 45

### tert-Butyl 3-(6-{(S)-benzyloxycarbonylamino[4-(trifluoromethyl)cyclohexyl]methyl}-imidazo[1,2-b][1,2,4]triazin-3-yl)-1,1-dioxo-1,4-thiazinane-4-carboxylate

To a solution of Intermediate 44 (517 mg, 1.19 mmol), Intermediate 36 (777 mg, 1.83 mmol) and {Ir[dF(CF₃)ppy]₂(dtbpy)}PF₆ (35 mg, 0.031 mmol) in anhydrous DMF (12 mL) under nitrogen was added TFA (140 μL, 1.85 mmol) dropwise. The mixture was purged with nitrogen for 5 minutes, then placed in a photoreactor and irradiated at 450 nm for 20 h at ambient temperature. The reaction mixture was poured into a mixture of EtOAc (50 mL) and saturated aqueous NaHCO₃ solution (150 mL). The organic phase was separated, and the aqueous phase was extracted with EtOAc (2×100 mL). The combined organic extracts were washed with brine (2×100 mL), then dried over anhydrous Na₂SO₄ and filtered. The solvents were removed in vacuo. The resulting brown residue was purified by column chromatography on silica (gradient elution 0-80% EtOAc in hexanes) to provide the title compound (402 mg, 51%) as a pale orange solid. 8H (400 MHz, DMSO-d₆) 8.81 (d, J 4.0 Hz, 1H), 8.15 (s, 1H), 7.65 (br s, 1H), 7.33 (m, 5H), 6.10-5.93 (m, 1H), 5.17-4.92 (m, 2H), 4.68 (t, J 8.1 Hz, 1H), 4.53-4.33 (m, 1H), 4.20-3.96 (m, 1H), 3.92-3.63 (m, 2H), 3.36 (td, J 12.9, 11.6, 4.2 Hz, 1H), 3.17-3.01 (m, 1H), 2.27-2.07 (m, 1H), 1.85 (m, 4H), 1.64 (d, J 12.4 Hz, 1H), 1.44 (m, 10H), 1.30-0.93 (m, 3H). LCMS (Method 1): [M+H]⁺ 667, RT 1.50 minutes.

### Intermediate 46

### Benzyl N—{(S)-[3-(1,1-dioxo-1,4-thiazinan-3-yl)imidazo[1,2-b][1,2,4]triazin-6-yl][4-(trifluoromethyl)cyclohexyl]methyl}carbamate

To a solution of Intermediate 45 (483 mg, 0.72 mmol) in DCM (6 mL), cooled on an ice/water bath, was slowly added TFA (2 mL). The golden-yellow solution was stirred at ambient temperature for 18 h. The volatiles were removed in vacuo and the residue was azeotroped with toluene (2×30 mL). The residue was dissolved in DCM (30 mL) and washed with saturated aqueous NaHCO₃ solution. The aqueous phase was extracted with DCM (20 mL). The combined organic phases were dried over anhydrous Na₂SO₄ and filtered, then the solvents were removed in vacuo, to give the title compound (450 mg, quantitative) as a golden-yellow glassy solid. LCMS (Method 1): [M+H]⁺ 567, RT 1.33 minutes.

### Intermediate 47

### [3-(6-{(S)-Amino[4-(trifluoromethyl)cyclohexyl]methyl}imidazo[1,2-b][1,2,4]triazin-3-yl)-1,1-dioxo-1,4-thiazinan-4-yl](3-fluorobicyclo[1.1.1]pentan-1-yl)methanone

To Example 12 (120 mg, 0.177 mmol) was added HBr (33% solution in AcOH) (5 mL, 30.38 mmol). The resultant yellow suspension was sonicated briefly to ensure solution, and the reaction mixture was stirred at ambient temperature. After 10 minutes, diethyl ether (20 mL) was added. The mixture was stirred at ambient temperature for 5 minutes, then the solid was collected on a frit, washed with additional diethyl ether and dried under suction. The solid was dissolved in MeOH (10 mL) and loaded onto a 2 g SCX column, eluting with MeOH (30 mL), followed by 7N ammonia in MeOH (30 mL). The yellow methanolic ammonia portion was concentrated in vacuo to yield the title compound (59 mg, 52%) as a yellow film. LCMS (Method 1): [M+H]⁺ 545, RT 1.18 minutes.

### Intermediate 48

### Ethyl 1-acetyl-4,4-difluorocyclohexanecarboxylate

To a solution of ethyl 4,4-difluorocyclohexanecarboxylate (11 g, 57.2 mmol) in THF (57 mL) at −78° C. was added LDA (2.0 mol/L, 31 mL, 63 mmol) dropwise over 30 minutes. The reaction mixture was stirred for a further 30 minutes, then acetic anhydride (8.1 mL, 85.8 mmol) was added. The reaction mixture was allowed to warm slowly to r.t., and stirred for 3 h. To the reaction mixture were added water (200 mL) and hexane (200 mL). The organic layer was separated, and the aqueous layer was extracted with hexane (2×100 mL). The combined organic extracts were dried over Na₂SO₄, passed through a phase separator and concentrated in vacuo. The crude material was purified by column chromatography (Biotage SFAR HC DUO, 350 g, Isolera, 0-20% EtOAc in isohexane) to give the title compound (6.37 g, 48%) as a yellow oil. δ_{H} (300 MHz, CDCl₃) 4.36-4.17 (m, 2H), 2.45-2.21 (m, 2H), 2.20 (s, 3H), 2.15-1.74 (m, 6H), 1.30 (td, J 7.1, 4.7 Hz, 3H).

### Intermediate 49

### Ethyl 1-(2,2-dibromoacetyl)-4,4-difluorocyclohexanecarboxylate

To a solution of Intermediate 48 (5.5 g, 23.48 mmol) in DCM (23 mL) was added bromine (2.4 mL, 46.96 mmol) dropwise. The reaction mixture was stirred at r.t. for 18 h, then diluted with DCM (200 mL) and washed with saturated aqueous Na₂CO₃ solution (200 mL), saturated aqueous sodium thiosulfate solution (20 mL) and brine (200 mL). The organic layer was dried over Na₂SO₄, then passed through a phase separator and concentrated in vacuo. The crude material was purified by column chromatography (Biotage SFAR HC DUO, 250 g, Isolera, 0-20% EtOAc in isohexane) to give the title compound (7.85 g, 85%) as a colourless oil. δ_{H} (300 MHz, CDCl₃) 6.26 (s, 1H), 4.28 (q, J 7.1 Hz, 2H), 2.45-2.30 (m, 2H), 2.27-1.79 (m, 6H), 1.33 (t, J 7.2 Hz, 3H).

### Intermediate 50

### Ethyl 1-(3-amino-1,2,4-triazin-5-yl)-4,4-difluorocyclohexanecarboxylate

To a vial containing a solution of Intermediate 49 (7 g, 17.86 mmol) in THF (18 mL) was added morpholine (6.2 mL, 71.42 mmol) dropwise. The reaction mixture was stirred at 65° C. overnight. The resulting suspension was filtered through a pad of Celite®, washing with THF (100 mL). The filtrate was concentrated in vacuo to give an orange oil. To a separate vial containing aminoguanidine bicarbonate (2.48 g, 17.86 mmol) in MeOH (18 mL) was added AcOH (4.1 mL, 71.423 mmol). The suspension was stirred at r.t. for 18 h, then added to a solution of the foregoing orange oil in MeOH (18 mL). The reaction mixture was stirred at reflux temperature for 4 h, then concentrated in vacuo.

The crude residue was dissolved in EtOAc (200 mL) and washed with water (200 mL). The aqueous layer was extracted with further EtOAc (2×200 mL). The combined organic extracts were dried and concentrated in vacuo. The crude material was triturated with diethyl ether (3×50 mL), and the solid was filtered and dried in vacuo, to give the title compound (1.52 g, 30%) as a colourless amorphous solid. δ_{H} (300 MHz, DMSO-d₆) 8.71 (s, 1H), 7.30 (s, 2H), 4.15 (q, J 7.1 Hz, 2H), 2.42-2.08 (m, 5H), 2.08-1.72 (m, 5H), 1.14 (t, J 7.1 Hz, 3H). LCMS (Method 8): [M+H]⁺ 287.2, RT 1.37 minutes.

### Intermediate 51

### Ethyl 1-{6-[(S)-benzyloxycarbonylamino(4,4-difluorocyclohexyl)methyl]imidazo[1,2-b]-[1,2,4]triazin-3-yl}-4,4-difluorocyclohexanecarboxylate

To a vial containing Intermediate 50 (490 mg, 1.71 mmol), benzyl N-[(1S)-3-bromo-1-(4,4-difluorocyclohexyl)-2-oxopropyl]carbamate (1.04 g, 2.57 mmol) and NaHCO₃ (288 mg, 3.43 mmol) was added IPA (10 mL). The reaction mixture was stirred at 80° C. for 18 h. Additional NaHCO₃ (70 mg, 0.86 mmol) was added, and heating at 80° C. was continued for 5 h. The reaction mixture was concentrated in vacuo. The residue was diluted with EtOAc (20 mL) and washed with water (20 mL), then the aqueous layer was further extracted with EtOAc (2×20 mL). The combined organic extracts were concentrated in vacuo. The crude material was purified by column chromatography (Biotage SFAR HC DUO, 25 g, Isolera, 0-40% EtOAc/hexanes) to give the title compound (590 mg, 58%). δ_{H} (300 MHz, CDCl₃) 8.62 (d, J 12.7 Hz, 1H), 7.87 (s, 1H), 7.51-7.04 (m, 5H), 6.42-6.22 (m, 1H), 5.22-5.00 (m, 4H), 4.25 (qd, J 7.1, 3.5 Hz, 2H), 2.63 (d, J 14.1 Hz, 2H), 2.57-2.27 (m, 3H), 2.29-1.90 (m, 6H), 1.96-1.35 (m, 4H), 1.35-0.98 (m, 3H). LCMS (Method 1): [M+H]⁺ 592.2, RT 1.53 minutes.

### Intermediate 52

### 1-{6-[(S)-Benzyloxycarbonylamino(4,4-difluorocyclohexyl)methyl]imidazo[1,2-b]-[1,2,4]triazin-3-yl}-4,4-difluorocyclohexanecarboxylic acid sodium salt

To a stirred solution of Intermediate 51 (200 mg, 0.34 mmol) in THF (4 mL), EtOH (4 mL) and water (2 mL) at r.t. was added aqueous NaOH solution (2 mol/L, 0.5 mL, 1 mmol). The reaction mixture was stirred at r.t. for 1 h, then concentrated in vacuo (water bath; 30° C.), to give the title compound (200 mg, quantitative) as a yellow oil which was utilised quickly without further purification. LCMS (Method 1): [M+H]⁺ 564.2, RT 1.10 minutes.

### Intermediate 53

### Benzyl N—[(S)-(4,4-difluorocyclohexyl){3-[1-(2,2-difluoropropylcarbamoyl)-4,4-difluoro-cyclohexyl]imidazo[1,2-b][1,2,4]triazin-6-yl}methyl]carbamate

To a solution containing crude Intermediate 52 (200 mg, 0.34 mmol) suspended in DMF (2 mL) were added DIPEA (0.15 mL, 0.86 mmol) and 2,2-difluoropropylamine hydrochloride (50 mg, 0.36 mmol), then HATU (155.6 mg, 0.4092 mmol) was added portionwise. The reaction mixture was stirred at r.t. for 18 h, then diluted with EtOAc (20 mL) and water (5 mL). The aqueous layer was extracted with EtOAc (3×25 mL). The combined organic extracts were concentrated in vacuo. The resulting yellow oil was purified by column chromatography (Biotage SFAR HC DUO, 25 g, Isolera, 0-100% EtOAc/hexanes) to give the title compound (impure mixture) (200 mg, 91%) as a clear oil which was utilised without further purification. LCMS (Method 3): [M+H]⁺ 641.2, RT 1.46 minutes.

### Intermediate 54

### 1-{16-[(S)-Amino(4,4-difluorocyclohexyl)methyl]imidazo[1,2-b][1,2,4]triazin-3-yl}-N-(2,2-difluoropropyl)-4,4-difluorocyclohexanecarboxamide

To a solution containing crude Intermediate 53 (200 mg, 0.31 mmol) in EtOH (3 mL), evacuated and placed under N₂, was added Pd/C (10 mass %) (20 mg, 0.019 mmol). The reaction flask was placed under an atmosphere of H₂ and stirred at r.t. for 1 h. The reaction mixture was filtered through a pad of Celite®, and concentrated in vacuo, to give the title compound (160 mg, quantitative) as a crude brown oil which was utilised without further purification. LCMS (Method 1): [M+H]⁺ 507.2, RT 1.24 minutes.

### Intermediate 55

### Methyl 4-acetyltetrahydropyran-4-carboxylate

To a vigorously stirring solution of methyl tetrahydro-2H-pyran-4-carboxylate (0.91 mL, 6.94 mmol) in THF (15 mL) at −75° C. (internal temperature) was added 2M LDA in THF (3.9 mL, 7.86 mmol) over 10 minutes. After 50 minutes, acetic anhydride (0.98 mL, 10.4 mmol) was added to the reaction mixture over 5 minutes. The reaction mixture was stirred at r.t. for 75 minutes, then cooled to 0° C. Heptane (10 mL) and water (5 mL) were added. The biphasic mixture was transferred to a separating funnel, and further diluted with heptane (30 mL) and water (30 mL). The phases were separated, and the aqueous phase was re-extracted with heptane (2×30 mL). The combined organic extracts were dried (phase separator) and concentrated in vacuo. The resulting crude yellow oil was purified by automated FCC (Isolera 4, Sfar Duo 100 g, 0-100% EtOAc in heptane) to afford the title compound (0.85 g, 59%) as a yellow oil. δ_{H} (500 MHz, CDCl₃) 3.81-3.72 (m, 5H), 3.63-3.53 (m, 2H), 2.19-2.10 (m, 5H), 2.02-1.94 (m, 2H).

### Intermediate 56

### Methyl 4-(2,2-dibromoacetyl)tetrahydropyran-4-carboxylate

To a stirred solution of Intermediate 55 (0.84 g, 4.06 mmol) in DCM (6 mL) at r.t. was added bromine (0.52 mL, 10.1 mmol). After 6 h, the mixture was diluted with DCM (30 mL) and washed with saturated aqueous NaHCO₃ solution (30 mL). The aqueous phase was re-extracted with DCM (30 mL), and the combined organic extracts were washed with saturated aqueous Na₂S₂O₃ solution (30 mL). The organic phase was dried (phase separator), then concentrated in vacuo, to give the title compound (1.46 g, 94%) as a yellow-orange oil. δ_{H} (500 MHz, CDCl₃) 6.24 (s, 1H), 3.85-3.74 (m, 5H), 3.70-3.57 (m, 2H), 2.29-2.18 (m, 2H), 2.17-2.06 (m, 2H).

### Intermediate 57

### Methyl 4-[2,2-di(morpholin-4-yl)acetyl]tetrahydropyran-4-carboxylate

To a stirred solution of Intermediate 56 (1.44 g, 3.77 mmol) in THF (15 mL) at r.t. was added morpholine (1.8 mL, 15.2 mmol). The resultant solution was warmed to 55° C. over 45 minutes, and heated at 55° C. for a further 18 h. Additional morpholine (0.9 mL, 7.6 mmol) was added, and heating was continued at 55° C. for 3 h. The reaction mixture was cooled to r.t. and stirred overnight, then the suspension was filtered, washed with THF (20 mL) and concentrated in vacuo. The oily orange residue was azeotroped with heptane (3×30 mL), then dried in vacuo at r.t. for 3 h, to afford the title compound (˜65% purity by ¹H NMR) (1.59 g, 77%) as an orange oil, which was utilised without further purification. δ_{H} (500 MHz, CDCl₃) 3.89-3.84 (m, 2H), 3.80 (s, 1H), 3.78 (s, 3H), 3.78-3.53 (m, 8H), 3.50-3.43 (m, 2H), 2.74-2.67 (m, 4H), 2.59-2.51 (m, 4H), 2.24-2.17 (m, 2H), 2.01-1.92 (m, 2H).

### Intermediate 58

### Methyl 4-(3-amino-1,2,4-triazin-5-yl)tetrahydropyran-4-carboxylate

A round-bottomed flask was charged with Intermediate 57 (65%, 1.55 g, 2.83 mmol), MeOH (10 mL), aminoguanidine hydrochloride (0.32 g, 2.93 mmol) and AcOH (0.33 mL, 5.77 mmol). The mixture was stirred at r.t. for 30 minutes, then heated at 65° C. for 24 h, then cooled to r.t. and left standing at r.t. overnight. The reaction mixture was concentrated in vacuo. The crude residue was diluted with EtOAc (50 mL), H₂O (50 mL) and saturated aqueous Na₂CO₃ solution (10 mL), then the phases were separated. The aqueous phase was re-extracted with EtOAc (2×50 mL). The combined organic extracts were dried (phase separator) and concentrated in vacuo. The residue was purified using automated chromatography (Isolera 4, Sfar Duo 50 g, 0-10% MeOH in DCM as eluant) to afford the title compound (˜65% purity by ¹H NMR) (650 mg, 63%) as a yellow-orange solid, which was utilised without further purification. δ_{H} (400 MHz, DMSO-d₆) 8.69 (s, 1H), 7.37-7.18 (m, 2H), 3.72-3.62 (m, 5H), 3.57-3.44 (m, 2H), 2.27-2.16 (m, 2H), 2.14-2.04 (m, 2H).

### Intermediate 59

### Methyl 4-{16-[(S)-benzyloxycarbonylamino(4,4-difluorocyclohexyl)methyl]imidazo[1,2-b][1,2,4]triazin-3-yl}tetrahydropyran-4-carboxylate

A suspension of Intermediate 58 (65%, 0.65 g, 1.76 mmol), benzyl N-[(1S)-3-bromo-1-(4,4-difluorocyclohexyl)-2-oxopropyl]carbamate (1.08 g, 2.62 mmol) and NaHCO₃ (0.29 g, 3.48 mmol) in IPA (8.38 mL) was placed under nitrogen and heated at 65° C. for 18 h. After cooling, the mixture was diluted with water (50 mL) and saturated aqueous NaHCO₃ solution (50 mL), and extracted with EtOAc (3×50 mL). The combined organic extracts were washed with brine (50 mL). The phases were separated, then the organic phase was dried (hydrophobic frit) and concentrated in vacuo. The crude residue was purified by automated FCC (Isolera 4, Sfar Duo 100 g, 0-100% EtOAc in heptane) to afford the title compound (0.64 g, 57%) as a red-brown oil, which was utilised without further purification. δ_{H} (400 MHz, DMSO-d₆) 8.83 (s, 1H), 8.17 (s, 1H), 7.86 (d, J 9.3 Hz, 1H), 7.42-7.25 (m, 5H), 5.05 (d, J 12.6 Hz, 1H), 5.01 (d, J 12.5 Hz, 1H), 4.80-4.72 (m, 1H), 3.78-3.64 (m, 5H), 3.63-3.47 (m, 2H), 2.41-1.49 (m, 11H), 1.46-1.20 (m, 2H).

### Intermediate 60

### Benzyl N—[(S)-(4,4-difluorocyclohexyl){3-[4-(2,2-difluoropropylcarbamoyl)-tetrahydropyran-4-yl]imidazo[1,2-b][1,2,4]triazin-6-yl}methyl]carbamate

To a solution of Intermediate 59 (200 mg, 0.353 mmol) in DCM (3 mL) at 20° C. was added a 3M solution of NaOH in MeOH (118 μL, 0.353 mmol). The reaction mixture was stirred at r.t. for 30 minutes, then additional 3M solution of NaOH in MeOH (118 μL, 0.353 mmol) was added. The reaction mixture was stirred at r.t. for a further 25 minutes. This procedure was repeated three further times, then the reaction mixture was concentrated to dryness under a stream of nitrogen. The resulting carboxylic acid sodium salt (195 mg) was taken up in DCM (3.8 mL), then 2,2-difluoropropan-1-amine hydrochloride (1:1) (52 mg, 0.392 mmol), DIPEA (137 μL, 0.785 mmol) and HATU (119 mg, 0.314 mmol) were added portionwise. The reaction mixture was stirred at r.t. for 64 h, then DMF (3.8 mL) and additional 2,2-difluoropropan-1-amine hydrochloride (1:1) (52 mg, 0.392 mmol), DIPEA (137 μL, 0.785 mmol) and HATU (119 mg, 0.314 mmol) were added in quick succession. The reaction mixture was stirred at r.t. for 10 minutes, then diluted with EtOAc (10 mL), saturated aqueous NaHCO₃ solution (5 mL) and water (5 mL). The biphasic mixture was stirred at r.t. for 20 minutes. The layers were separated, then the organic phase was dried (hydrophobic frit) and concentrated in vacuo. The crude residue was purified by automated FCC (Isolera 4, Sfar Duo 50 g, 0-100% EtOAc in heptane) to afford a mixture of the title compound (47%) and an inseparable impurity identified as benzyl N—{(S)-(4,4-difluorocyclohexyl)[3-(tetrahydropyran-4-yl)imidazo-[1,2-b][1,2,4]triazin-6-yl]methyl}carbamate (53%) (117 mg, 34%) as a yellow oil, which was utilised without further purification. LCMS (Method 8): [M+H]⁺ m/z 607, RT 3.90 minutes.

### Intermediate 61

### 4-{16-[(S)-Amino(4,4-difluorocyclohexyl)methyl]imidazo[1,2-b][1,2,4]triazin-3-yl}-N-(2,2-difluoropropyl)tetrahydropyran-4-carboxamide

To a stirred solution of Intermediate 60 (46%, 107 mg, 0.0811 mmol) in EtOH (5 mL) under nitrogen (three cycles of vacuum/nitrogen gas) was added 10% Pd/C (50% wet) (5.0%, 35 mg, 0.016 mmol) in a single portion. The reaction mixture was stirred under hydrogen (three cycles of vacuum/nitrogen gas, followed by three cycles of vacuum/hydrogen gas) for 3 h. The reaction mixture was filtered through Celite®, washing through with additional MeOH. The combined filtrates were concentrated in vacuo, then purified using automated chromatography (Isolera 4, Sfar KP Amino D, 11 g, 0-10% MeOH in DCM) to afford the title compound (89.0% purity) (33 mg, 77%) as a yellow oil, which was utilised without further purification. LCMS (Method 8): [M+H]+m/z 473, RT 2.93 minutes.

### Intermediate 62

### Benzyl N—[(S)-(4,4-difluorocyclohexyl)(3-{1-[(2S)-5,5-difluorotetrahydropyran-2-carbonyl]-4-hydroxy-4-(trifluoromethyl)piperidin-2-yl}imidazo[1,2-b][1,2,4]triazin-6-yl)-methyl]carbamate

To a solution of Intermediate 33 (mixture of two syn stereoisomers in 1:1 ratio) (310 mg, 0.51 mmol), Intermediate 79 (94.0 mg, 0.57 mmol) and DIPEA (0.36 mL, 2.10 mmol) in DMF (15 mL) at r.t. was added HATU (242 mg, 0.62 mmol) in one portion. The mixture was stirred for 10 minutes, then H₂O (20 mL) was added. The mixture was extracted with EtOAc (3×20 mL). The combined organic extracts were washed with brine (40 mL), then dried (Na₂SO₄) and concentrated in vacuo. Purification by flash chromatography, eluting with EtOAc/isohexane (0-100% gradient), afforded the title compound (mixture of two syn stereoisomers in 1:1 ratio, not distinguishable by LCMS) (246 mg, 67%) as a yellow foam. LCMS (Method 1): [M+H]⁺ m/z 717.2, RT 1.46 minutes.

### Intermediate 63

### [2-{6-[(S)-Amino(4,4-difluorocyclohexyl)methyl]imidazo[1,2-b][1,2,4]triazin-3-yl}-4-hydroxy-4-(trifluoromethyl)piperidin-1-yl][(2S)-5,5-difluorotetrahydropyran-2-yl]-methanone

To a solution of Intermediate 62 (mixture of two syn stereoisomers in 1:1 ratio) (246 mg, 0.34 mmol) in EtOH (10 mL) at r.t. was added 10% Pd/C (25 mg). The vessel was evacuated and purged three times with H₂, then stirred at r.t. for approximately 255 minutes. The mixture was filtered through a pad of Celite® (10 g) under suction, washing through with EtOH (60 mL). The filtrate was concentrated in vacuo. Purification by flash chromatography (KP-NH column), eluting with EtOAc/isohexane (0-100% gradient), then DCM:MeOH (90:10), afforded the title compound (mixture of two stereoisomers in 1:1 ratio, not distinguishable by LCMS) (85% purity) (162 mg, 69%) as a yellow foam. LCMS (Method 1): [M+H]⁺ m/z 583.2, RT 1.21 minutes.

### Intermediate 64

### tert-Butyl 4-(difluoromethyl)-4-(trimethylsilyloxy)piperidine-1-carboxylate

To a solution of tert-butyl 4-oxopiperidine-1-carboxylate (2.06 g, 10.3 mmol) in THF (20.7 mL) and DMPU (6.4 mL) at r.t. was added CsF (471 mg, 3.10 mmol) in one portion. The mixture was stirred for 5 minutes, then (difluoromethyl)trimethylsilane (2.82 mL, 20.7 mmol) was added dropwise over 2 minutes via syringe. The reaction mixture was stirred overnight at 70° C., then diluted with EtOAc (20 mL) and H₂O (20 mL). The layers were separated, and the aqueous layer was re-extracted with EtOAc (2×20 mL). The combined organic layers were washed with brine (2×40 mL), then dried (Na₂SO₄) and concentrated in vacuo. Purification by flash chromatography, eluting with EtOAc/isohexane (0-10% gradient), afforded the title compound (2.32 g, 69%) as a white solid. R_{f} 0.50 (isohexane:EtOAc, 90:10), non-UV, KMnO₄. δ_{H} (400 MHz, CDCl₃) 5.49 (t, J 56.0 Hz, 1H), 4.09-3.90 (m, 2H), 3.11-2.94 (m, 2H), 1.67-1.53 (m, 4H), 1.49 (s, 9H), 0.18 (s, 9H).

### Intermediate 65

### 1-(tert-Butoxycarbonyl)-4-(difluoromethyl)-4-(trimethylsilyloxy)piperidine-2-carboxylic acid

To a solution of Intermediate 64 (2.95 g, 9.12 mmol) and TMEDA (2.47 mL, 16.4 mmol) in diethyl ether (91 mL) at −78° C. was added sec-butyllithium (1.3M in cyclo-hexane, 12.0 mL, 17.0 mmol) dropwise over 10 minutes. The mixture was stirred at −78° C. for 10 minutes, during which time a bright orange colour resulted. After this time, the reaction mixture was warmed to −40° C. (replacement of dry ice/acetone bath with dry ice/acetonitrile bath) and stirred at −40° C. for 20 minutes. The reaction mixture was re-cooled to −78° C. and CO₂ was bubbled through the mixture for 30 minutes. After this time, the reaction mixture was warmed to r.t. and stirred for 1 h, then quenched by the addition of saturated aqueous NH₄Cl solution (30 mL) and H₂O (5 mL) (to solubilise the resulting precipitate). The layers were separated, then the aqueous layer was washed with diethyl ether (2×20 mL) and EtOAc (2×20 mL). The combined organic layers were dried (Na₂SO₄) and concentrated in vacuo. Purification by flash chromatography, eluting with EtOAc/isohexane (0-100% gradient), then DCM:MeOH (90:10), afforded the title compound (mixture of two diastereomers, not distinguishable by ¹H NMR) (1.97 g, 59%) as a thick yellow oil. δ_{H} (400 MHz, CDCl₃) 5.58 (t, J 55.8 Hz, 1H), 4.43 (dd, J 9.0, 5.9 Hz, 1H), 3.76-3.64 (m, 1H), 3.44 (ddd, J 13.6, 8.6, 4.8 Hz, 1H), 2.16 (dd, J 14.1, 9.0 Hz, 1H), 2.10-2.01 (obscured dd, 1H), 1.98-1.88 (m, 1H), 1.82-1.72 (m, 1H), 1.49 (s, 9H), 0.18 (s, 9H). The carboxylic acid proton signal was not observed.

### Intermediate 66

### 1-(tert-Butoxycarbonyl)-4-(difluoromethyl)-4-hydroxypiperidine-2-carboxylic acid

To a solution of Intermediate 65 (1.97 g, 5.36 mmol) in THF (50 mL) at r.t. was added TBAF (1M in THF, 8.04 mL, 8.04 mmol) dropwise over 2 minutes. The mixture was stirred at r.t. for 30 minutes, then concentrated to dryness and loaded onto SiO₂. Purification by flash chromatography, eluting with EtOAc/isohexane (0-100% gradient), then DCM:MeOH (90:10), afforded the title compound (mixture of two diastereomers, not distinguishable by ¹H NMR) (1.54 g, 97%) as a beige foam. δ_{H} (400 MHz, 373K, DMSO-d₆) 12.69-11.65 (br s, 1H), 5.72 (t, J 56.0 Hz, 1H), 5.34-5.97 (br s, 1H), 4.27 (dd, J 8.6, 6.3 Hz, 1H), 3.66-3.56 (m, 1H), 3.35-3.25 (m, 1H), 2.01-1.88 (m, 2H), 1.85-1.76 (m, 1H), 1.64-1.54 (m, 1H), 1.41 (s, 9H).

### Intermediate 67

### O¹-tert-Butyl O²-(1,3-dioxoisoindolin-2-yl) 4-(difluoromethyl)-4-hydroxypiperidine-1,2-dicarboxylate

To a solution of Intermediate 66 (800 mg, 2.71 mmol) and N-hydroxyphthalimide (486 mg, 2.98 mmol) in DCM (15 mL) at r.t. was added EDCI·HCl (571 mg, 2.98 mmol) in one portion. The yellow mixture was stirred for 18 h, then concentrated in vacuo. Purification by flash chromatography, eluting with EtOAc/isohexane (0-60% gradient), gave the title compound (mixture of two diastereomers, not distinguishable by LCMS or ¹H NMR) (566 mg, 47%) as a white foam. δ_{H} (400 MHz, DMSO-d₆) 8.00-7.93 (m, 4H), 5.83 (t, J 55.9 Hz, 1H), 5.48-5.33 (br s, 1H), 4.78 (dd, J 9.9, 6.0 Hz, 1H), 3.73 (dt, J 13.6, 6.0 Hz, 1H), 3.34 (ddd, J 13.6, 8.3, 5.1 Hz, 1H), 2.21 (dd, J 14.2, 5.9 Hz, 1H), 2.12 (dd, J 14.0, 9.9 Hz, 1H), 1.88 (dt, J 14.2, 5.7 Hz, 1H), 1.75-1.65 (m, 1H), 1.48 (s, 9H). LCMS (Method 1): [M+H-BOC]+m/z 341.0, RT 1.30 minutes.

### Intermediates 68 & 69

### tert-Butyl 2-{6-[(S)-benzyloxycarbonylamino(4,4-difluorocyclohexyl)methyl]imidazo-[1,2-b][1,2,4]triazin-3-yl}-4-(difluoromethyl)-4-hydroxypiperidine-1-carboxylate (syn isomer) (Intermediate 68)

### tert-Butyl 2-{6-[(S)-benzyloxycarbonylamino(4,4-difluorocyclohexyl)methyl]imidazo-[1,2-b][1,2,4]triazin-3-yl}-4-(difluoromethyl)-4-hydroxy piperidine-1-carboxylate (anti isomer) (Intermediate 69)

Into a screw-cap vial were introduced Intermediate 18 (345 mg, 0.86 mmol), Intermediate 67 (568 mg, 1.29 mmol), DMF (17 mL), {Ir[dF(CF₃)ppy]₂(dtbpy)}PF₆ (20.0 mg, 0.018 mmol) and TFA (0.10 mL, 1.30 mmol) sequentially. The vial was capped and the mixture was purged with N₂ for 5 minutes, then the cap was sealed with parafilm and the mixture was irradiated (450 nm) using an ‘integrated photoreactor’ (AC'S Cent. Sci., 2017, 3, 647-653) (settings: Fan=1612 rpm; Stir=392 rpm; LED=100%) for 20 h. The mixture was diluted with EtOAc (20 mL) and washed with H₂O (2×10 mL). The combined organic layers were dried (Na₂SO₄) and concentrated in vacuo. The residue was purified by flash chromatography, eluting with EtOAc/isohexane (0-70% gradient), to afford Intermediate 68 (syn-configured, major) (two stereoisomers in 1:1 ratio, not distinguishable by LCMS or ¹H NMR) (192 mg, 34%) and Intermediate 69 (anti-configured, minor) (two stereoisomers in 1:1 ratio, not distinguishable by LCMS or ¹H NMR) (56.0 mg, 10%) as yellow foams. LCMS (Method 1): [M+H]⁺ m/z 651.2, RT 1.46 minutes (minor anti diastereomer), RT 1.50 minutes (major syn diastereomer).

### Intermediate 70

### Benzyl N—[(S)-(4,4-difluorocyclohexyl){3-[4-(difluoromethyl)-4-hydroxypiperidin-2-yl]imidazo[1,2-b][1,2,4]triazin-6-yl}methyl]carbamate hydrochloride (syn isomer)

To Intermediate 68 (mixture of two syn stereoisomers in 1:1 ratio) (192 mg, 0.30 mmol) was added 4N HCl in 1,4-dioxane (2.70 mL, 11.0 mmol) dropwise. The mixture was stirred at r.t. for 30 minutes, then carefully neutralised with saturated aqueous NaHCO₃ solution. EtOAc (20 mL) was added. and the layers were separated. The aqueous layer was extracted with EtOAc (2×20 mL). The combined organic extracts were dried (Na₂SO₄), then concentrated in vacuo, to afford the title compound (mixture of two syn stereoisomers in 1:1 ratio, not distinguishable by LCMS or ¹H NMR) (169 mg, 98%) as a yellow foam, which was utilised without further purification. LCMS (Method 1): [M+H]⁺ m/z 551.2, RT 1.23 minutes.

### Intermediate 71

### Benzyl N—[(S)-(4,4-difluorocyclohexyl)(3-{4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)-methyl]carbamate (syn isomer)

To a solution of Intermediate 70 (mixture of two syn stereoisomers in 1:1 ratio) (169 mg, 0.29 mmol), Intermediate 79 (53.0 mg, 0.32 mmol) and DIPEA (0.20 mL, 1.20 mmol) in DMF (10 mL) at r.t. was added HATU (136 mg, 0.35 mmol) in one portion. The mixture was stirred for 45 minutes, then H₂O (20 mL) was added. The mixture was extracted with EtOAc (3×20 mL). The combined organic extracts were washed with brine (40 mL), then dried (Na₂SO₄) and concentrated in vacuo. Purification by flash chromatography, eluting with EtOAc/isohexane (0-100% gradient), afforded the title compound (mixture of two syn stereoisomers in 1:1 ratio, not distinguishable by LCMS) (177 mg, 88%) as a yellow foam. LCMS (Method 1): [M+H]⁺ m/z 699.2, RT 1.42 minutes.

### Intermediate 72

### [2-{6-[(S)-Amino(4,4-difluorocyclohexyl)methyl]imidazo[1,2-b][1,2,4]triazin-3-yl}-4-(difluoromethyl)-4-hydroxypiperidin-1-yl][(25)-5,5-difluorotetrahydropyran-2-yl]-methanone (syn isomer)

To a solution of Intermediate 71 (mixture of two syn stereoisomers in 1:1 ratio) (177 mg, 0.25 mmol) in EtOH (10 mL) at r.t. was added 10% Pd/C (17 mg). The vessel was evacuated and purged three times with H₂, then stirred at r.t. for approximately 90 minutes. The mixture was filtered through a pad of Celite® (10 g) under suction, washing through with EtOH (60 mL). The filtrate was concentrated in vacuo. Purification by flash chromatography (KP-NH column), eluting with EtOAc/isohexane (0-100% gradient), then DCM:MeOH (90:10), afforded the title compound (mixture of two stereoisomers in 1:1 ratio, not distinguishable by LCMS) (124 mg, 88%) as a yellow foam. LCMS (Method 1): [M+H]⁺ m/z 565.2, RT 1.14 minutes.

### Intermediate 73

### tert-Butyl 3-(6-{(S)-benzyloxycarbonylamino[4-(trifluoromethyl)cyclohexyl]methyl}-imidazo[1,2-b][1,2,4]triazin-3-yl)morpholine-4-carboxylate

Into a screw-cap vial were introduced Intermediate 44 (99% e.e.) (400 mg, 0.92 mmol), Intermediate 17 (521 mg, 1.38 mmol), DMF (18 mL), {Ir[dF(CF₃)ppy]₂(dtbpy)}-PF₆ (21.0 mg, 0.019 mmol) and TFA (0.11 mL, 1.50 mmol) sequentially. The vial was capped and the mixture was purged with N₂ for 5 minutes, then the cap was sealed with parafilm and the mixture was irradiated (450 nm) using the Hepatochem ‘PhotoRedOx Box’ photoreactor (U.S. Pat. No. 10,906,022) for 48 h. Next, the mixture was transferred to an ‘integrated photoreactor’ (ACS Cent. Sci., 2017, 3, 647-653) (settings: Fan=1612 rpm; Stir=392 rpm; LED=100%) and irradiated for a further 5 h. The mixture was diluted with EtOAc (40 mL) and washed with H₂O (2×20 mL). The combined organic layers were dried (Na₂SO₄) and concentrated in vacuo. The residue was purified by flash chromatography, eluting with EtOAc/isohexane (0-100% gradient), to afford the title compound (mixture of two stereoisomers in 1:1 ratio, not distinguishable by LCMS or ¹H NMR) (400 mg, 70%) as an orange foam. LCMS (Method 1): [M+H]⁺ m/z 619.2, RT 1.48 minutes.

### Intermediate 74

### tert-Butyl 3-(6-{(S)-amino[4-(trifluoromethyl)cyclohexyl]methyl}imidazo[1,2-b][1,2,4]-triazin-3-yl)morpholine-4-carboxylate

To a solution of Intermediate 73 (two stereoisomers in 1:1 ratio) (400 mg, 0.65 mmol) in EtOH (22 mL) at r.t. were added 4N HCl in 1,4-dioxane (0.20 mL, 0.80 mmol) and 10% Pd/C (40 mg) sequentially. The vessel was evacuated and purged three times with H₂, then stirred at r.t. for approximately 330 minutes. The mixture was filtered through a pad of Celite® (10 g) under suction, washing through with EtOH (60 mL). The filtrate was concentrated in vacuo. Purification by flash chromatography (KP-NH column), eluting with EtOAc/isohexane (0-100% gradient), then DCM:MeOH (90:10), afforded the title compound (mixture of two stereoisomers in 1:1 ratio, not distinguishable by LCMS or ¹H NMR) (85% purity) (240 mg, 65%) as an orange foam. LCMS (Method 1): [M+H]⁺ m/z 485.2, RT 1.31 minutes.

### Intermediate 75

### tert-Butyl 3-(6-{(S)-[(4-methyl-1,2,5-oxadiazole-3-carbonyl)amino][4-(trifluoromethyl)-cyclohexyl]methyl}imidazo[1,2-b][1,2,4]triazin-3-yl)morpholine-4-carboxylate

To a solution of Intermediate 74 (two stereoisomers in 1:1 ratio) (85% purity) (240 mg, 0.50 mmol), 4-methyl-1,2,5-oxadiazole-3-carboxylic acid (64.0 mg, 0.50 mmol) and DIPEA (0.34 mL, 2.00 mmol) in DMF (10 mL) at r.t. was added HATU (233 mg, 0.59 mmol) in one portion. The mixture was stirred for 50 minutes, then H₂O (25 mL) was added. The mixture was extracted with EtOAc (3×20 mL). The combined organic extracts were washed with brine (40 mL), then dried (Na₂SO₄) and concentrated in vacuo.

Purification by flash chromatography, eluting with EtOAc/isohexane (0-75% gradient), afforded the title compound (mixture of two stereoisomers in 1:1 ratio, not distinguishable by LCMS or ¹H NMR) (159 mg, 54%) as an orange solid. LCMS (Method 1): [M+H]⁺ m/z 595.2, RT 1.50 minutes.

### Intermediate 76

### 4-Methyl-N—{(S)-[3-(morpholin-3-yl)imidazo[1,2-b][1,2,4]triazin-6-yl][4-(trifluoro-methyl)cyclohexyl]methyl}-1,2,5-oxadiazole-3-carboxamide trifluoroacetic acid salt

Intermediate 75 (two stereoisomers in 1:1 ratio) (159 mg, 0.27 mmol) was dissolved in DCM (1.9 mL), and TFA (0.30 mL, 4.00 mmol) was added. The solution was stirred at r.t. for 165 minutes, then neutralised with saturated aqueous NaHCO₃ solution (10 mL). To the mixture was added EtOAc (10 mL), and the layers were separated. The aqueous layer was re-extracted with EtOAc (2×20 mL). The combined organic layers were dried (Na₂SO₄), then concentrated in vacuo, to afford the title compound (mixture of two stereoisomers in 1:1 ratio, not distinguishable by LCMS or ¹H NMR) (187 mg, quantitative) as an orange foam, which was utilised without further purification. LCMS (Method 1): [M+H]⁺ m/z 495.2, RT 1.25 minutes.

### Intermediates 77 & 78

### [(3R)-4,4-Dimethyl-2-oxotetrahydrofuran-3-yl](2R)-5,5-difluorotetrahydropyran-2-carboxylate (Intermediate 77)

### [(3R)-4,4-Dimethyl-2-oxotetrahydrofuran-3-yl](2S)-5,5-difluorotetrahydropyran-2-carboxylate (Intermediate 78)

5,5-Difluorooxane-2-carboxylic acid (5.00 g, 28.6 mmol), (R)-(−)-pantolactone (4.00 g, 30.4 mmol) and DMAP (176 mg, 1.43 mmol) were dissolved in DCM (145 mL), and EDCI·HCl (6.35 g, 31.5 mmol) was added. The reaction mixture was stirred at r.t. overnight, then diluted with DCM and water, and passed through a phase separator. The organic phase was dried over Na₂SO₄ and concentrated in vacuo. Purification by flash column chromatography on silica, eluting with 0-5% EtOAc/DCM, gave the title compounds (Peak 1, 3.34 g, 42%; and Peak 2, 2.91 g, 36%).

Peak 1: δ_{H} (400 MHz, DMSO-d₆) 5.65 (s, 1H), 4.55-4.47 (m, 1H), 4.16 (d, J 8.6 Hz, 1H), 4.08 (d, J 8.6 Hz, 1H), 4.02-3.90 (m, 1H), 3.83-3.68 (m, 1H), 2.29-2.06 (m, 3H), 1.93-1.78 (m, 1H), 1.13 (s, 3H), 1.00 (s, 3H).

Peak 2: δ_{H} (400 MHz, DMSO-d₆) 5.67 (s, 1H), 4.51 (dd, J 9.9, 2.9 Hz, 1H), 4.16 (d, J 8.6 Hz, 1H), 4.08 (d, J 8.6 Hz, 1H), 4.02-3.90 (m, 1H), 3.84-3.69 (m, 1H), 2.29-2.07 (m, 3H), 1.95-1.81 (m, 1H), 1.12 (s, 3H), 1.00 (s, 3H).

### Intermediate 79

### (2S)-5,5-Difluorotetrahydropyran-2-carboxylic acid

Intermediate 78 (2.91 g, 10.4 mmol) was dissolved in a mixture of THF (20 mL), water (2 mL) and MeOH (2 mL), and treated with lithium hydroxide monohydrate (4.38 g, 104 mmol). The reaction mixture was heated at 50° C. for 1 h, then cooled to r.t. The mixture was adjusted to pH 2 with concentrated HCl, then extracted three times with EtOAc. The combined organic phase was washed twice with saturated aqueous NaHCO₃ solution, and the resulting aqueous layer was adjusted to pH 2 with concentrated HCl. Extraction three times with EtOAc, and concentration of the organic phase in vacuo, gave the title compound (1.10 g, 63%). δ_{H} (400 MHz, DMSO-d₆) 12.92 (s, 1H), 4.20-4.13 (m, 1H), 3.97-3.85 (m, 1H), 3.75-3.60 (m, 1H), 2.22-1.96 (m, 3H), 1.86-1.70 (m, 1H).

### Intermediates 80 & 81

### tert-Butyl 2-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-(difluoromethyl)-4-hydroxy-piperidine-1-carboxylate (syn isomers) (Intermediate 80)

### tert-Butyl 2-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-(difluoromethyl)-4-hydroxy-piperidine-1-carboxylate (anti isomers) (Intermediate 81)

Into a screw-cap vial were introduced Intermediate 21 (500 mg, 1.33 mmol), Intermediate 67 (875 mg, 1.99 mmol), DMF (10.5 mL), {Ir[dF(CF₃)ppy]₂(dtbpy)}PF₆ (30 mg, 0.026 mmol) and TFA (0.12 mL, 1.9 mmol) sequentially. The vial was capped and the mixture was purged with N₂ for 5 minutes, then the cap was sealed with parafilm and the mixture was irradiated (450 nm) using an ‘integrated photoreactor’ (AC'S Cent. Sci., 2017, 3, 647-653) (settings: Fan=1612 rpm; Stir=392 rpm; LED=100%) for 20 h. The mixture was diluted with DCM (20 mL) and water (20 mL), then washed with brine (2×10 mL). The combined organic layers were phase-separated with a hydrophobic frit, and concentrated in vacuo. Purification by flash chromatography, eluting with EtOAc/isohexane (0-100% gradient), gave Intermediate 80 (major syn isomers, 370 mg, 45%) and Intermediate 81 (minor anti isomers, 102 mg, 12%) as yellow foams.

Intermediate 80: LCMS (Method 1): [M+H]⁺ m/z 527.2, RT 1.47 minutes.

Intermediate 81: LCMS (Method 1): [M+H]⁺ m/z 527.2, RT 1.43 minutes.

### Intermediate 82

### N—[(S)-(4,4-Difluorocyclohexyl){3-[4-(difluoromethyl)-4-hydroxypiperidin-2-yl]-imidazo[1,2-b][1,2,4]triazin-6-yl}methyl]-4-methyl-1,2,5-oxadiazole-3-carboxamide hydrochloride (syn isomers)

To Intermediate 80 (mixture of two syn stereoisomers in 1:1 ratio) (337 mg, 0.54 mmol) was added 4N HCl in 1,4-dioxane (4.90 mL, 20.0 mmol) dropwise. The mixture was stirred at r.t. for 25 minutes, then carefully neutralised with saturated aqueous NaHCO₃ solution. EtOAc (30 mL) was added, and the layers were separated. The aqueous layer was extracted with EtOAc (2×30 mL). The combined organic layers were dried (Na₂SO₄), then concentrated in vacuo, to afford the title compound (mixture of two syn stereoisomers in 1:1 ratio, not distinguishable by LCMS or ¹H NMR) (303 mg, quantitative) as a yellow foam. LCMS (Method 1): [M+H]⁺ m/z 527.2, RT 1.17 minutes.

### Intermediate 83

### N—[(S)-(4,4-Difluorocyclohexyl){3-[4-(difluoromethyl)-4-hydroxypiperidin-2-yl]-imidazo[1,2-b][1,2,4]triazin-6-yl}methyl]-4-methyl-1,2,5-oxadiazole-3-carboxamide hydrochloride (anti isomers)

To Intermediate 81 (mixture of two anti stereoisomers in 1:1 ratio) (104 mg, 0.17 mmol) was added 4N HCl in 1,4-dioxane (1.51 mL, 6.04 mmol) dropwise. The mixture was stirred at r.t. for 70 minutes, then carefully neutralised with saturated aqueous NaHCO₃ solution. EtOAc (10 mL) was added, and the layers were separated. The aqueous layer was extracted with EtOAc (2×10 mL). The combined organic layers were dried (Na₂SO₄), and concentrated in vacuo, to afford the title compound (mixture of two anti stereoisomers in 1:1 ratio, not distinguishable by LCMS or ¹H NMR) (93.4 mg, quantitative) as a yellow foam. LCMS (Method 1): [M+H]⁺ m/z 527.2, RT 1.19 minutes.

### Intermediates 84 & 85

### tert-Butyl 2-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-hydroxy-4-(trifluoromethyl)-piperidine-1-carboxylate (syn isomers) (Intermediate 84)

### tert-Butyl 2-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-hydroxy-4-(trifluoromethyl)-piperidine-1-carboxylate (anti isomers) (Intermediate 85)

Into a screw-cap vial were introduced Intermediate 21 (210 mg, 0.56 mmol), Intermediate 4 (383 mg, 0.84 mmol), DMF (10.5 mL), {Ir[dF(CF₃)ppy]₂(dtbpy)}PF₆ (13.0 mg, 0.012 mmol) and TFA (0.06 mL, 0.80 mmol) sequentially. The vial was capped and the mixture was purged with N₂ for 5 minutes, then the cap was sealed with parafilm and the mixture was irradiated (450 nm) using an ‘integrated photoreactor’ (ACS (′ent. Sci., 2017, 3, 647-653) (settings: Fan=1612 rpm; Stir=392 rpm; LED=100%) for 40 h. The mixture was diluted with EtOAc (20 mL) and washed with H₂O (2×10 mL). The combined organic layers were dried (Na₂SO₄) and concentrated in vacuo.

Purification by flash chromatography, eluting with EtOAc/isohexane (0-80% gradient), gave Intermediate 84 (major syn isomers, 250 mg, 70%) and Intermediate 85 (minor anti isomers, 43.0 mg, 12%) as yellow foams.

Intermediate 84: LCMS (Method 1): [M+H]⁺ m/z 645.2, RT 1.52 minutes.

Intermediate 85: LCMS (Method 1): [M+H]⁺ m/z 645.2, RT 1.49 minutes.

### Intermediate 86

### N—[(S)-(4,4-Difluorocyclohexyl){3-[4-hydroxy-4-(trifluoromethyl)piperidin-2-yl]-imidazo[1,2-b][1,2,4]triazin-6-yl}methyl]-4-methyl-1,2,5-oxadiazole-3-carboxamide hydrochloride (anti isomers)

To Intermediate 85 (mixture of two anti stereoisomers in 1:1 ratio) (43.0 mg, 0.07 mmol) was added 4N HCl in 1,4-dioxane (0.61 mL, 2.40 mmol) dropwise. The mixture was stirred at r.t. for 2 h, then carefully neutralised with saturated aqueous NaHCO₃ solution. EtOAc (10 mL) was added, and the layers were separated. The aqueous layer was extracted with EtOAc (2×10 mL). The combined organic layers were dried (Na₂SO₄), and concentrated in vacuo, to afford the title compound (mixture of two anti stereoisomers in 1:1 ratio, not distinguishable by LCMS or ¹H NMR) (38.8 mg, quantitative) as a yellow foam. LCMS (Method 1): [M+H]⁺ m/z 545.2, RT 1.25 minutes.

### Intermediate 87

### 1-(tert-Butoxycarbonyl)-4-hydroxy-4-methylpiperidine-2-carboxylic acid

To a solution of 1-(tert-butoxycarbonyl)-4-oxopiperidine-2-carboxylic acid (5.24 g, 20.9 mmol) in THF (40 mL) at −40° C. was added a 3M solution of methylmagnesium chloride in THF (21 mL, 63 mmol) dropwise. The mixture was stirred at r.t. overnight, then water was cautiously added until no more gas evolved. An equal volume of EtOAc was added with vigorous stirring. The layers were separated, and the aqueous layer was washed with EtOAc. The mixture was adjusted to pH 3 with concentrated HCl, then extracted twice with EtOAc. The combined organic phase was washed with brine, then dried over Na₂SO₄ and concentrated in vacuo, to give the title compound (3.68 g, 68%). 8H (400 MHz, 373K, DMSO-d₆) 11.78 (s, 1H), 4.48-4.42 (m, 1H), 3.72-3.64 (m, 1H), 3.37-3.26 (m, 1H), 2.14 (dt, J 13.8, 2.3 Hz, 1H), 1.62 (dd, J 13.8, 7.1 Hz, 1H), 1.53-1.47 (m, 1H), 1.43-1.38 (m, 10H), 1.14 (s, 3H). The OH proton signal was not observed.

### Intermediates 88 & 89

### tert-Butyl 2-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-hydroxy-4-methylpiperidine-1-carboxylate (syn isomers) (Intermediate 88)

### tert-Butyl 2-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-hydroxy-4-methylpiperidine-1-carboxylate (anti isomers) (Intermediate 89)

Intermediate 21 (480 mg, 1.27 mmol), Intermediate 86 (694 mg, 2.54 mmol), cesium carbonate (1.25 g, 3.82 mmol) and {Ir[dF(CF₃)ppy]₂(dtbpy)}PF₆ (15 mg, 0.013 mmol) were dissolved in DMF (25 mL). The solution was purged with nitrogen for 10 minutes, then irradiated in a Penn Photoreactor M2 system for 3 h (450 nm wavelength; fan=3000 rpm; stir=500 rpm; LED=100%). The same procedure was performed three times batch-wise, and the resulting reaction mixtures were combined. The combined mixture was partitioned between EtOAc and brine, and the layers were separated. The aqueous phase was extracted with EtOAc, then the combined organic phase was washed with brine, dried over Na₂SO₄ and concentrated in vacuo. Purification by flash column chromatography on silica, eluting with 20-60% EtOAc/DCM, gave Intermediate 88 (syn isomers) (550 mg, 25%) and Intermediate 89 (anti isomers) (255 mg, 11%).

Intermediate 88 (syn isomers): δ_{H} (400 MHz, DMSO-d₆) 9.49 (d, J 9.0 Hz, 1H), 8.65-8.60 (m, 1H), 8.24-8.19 (m, 1H), 5.49-5.25 (m, 3H), 5.23-5.14 (m, 1H), 4.07 (s, 1H), 3.87 (br d, J 12.8 Hz, 1H), 2.49-2.44 (m, 3H), 2.43-2.35 (m, 1H), 2.25-2.16 (m, 1H), 2.11-1.70 (m, 4H), 1.70-1.60 (m, 1H), 1.53-1.20 (m, 14H), 1.13 (s, 3H). LCMS (Method 1): [M+H]⁺ m/z 591.2, RT 1.40 minutes.

Intermediate 89 (anti isomers): δ_{H} (400 MHz, DMSO-d₆) 9.49 (dd, J 8.9, 1.9 Hz, 1H), 8.66 (s, 1H), 8.26 (s, 1H), 5.39 (br s, 1H), 5.20 (t, J 8.5 Hz, 1H), 5.00 (dd, J 7.9, 5.7 Hz, 1H), 4.65 (s, 1H), 3.70-3.59 (m, 1H), 3.57-3.46 (m, 1H), 2.47 (s, 3H), 2.24-2.17 (m, 1H), 2.09-1.69 (m, 3H), 1.66-1.55 (m, 3H), 1.47-1.24 (m, 5H), 1.20 (s, 9H), 1.03 (s, 3H). LCMS (Method 1): [M+H]⁺ m/z 591.2, RT 1.33 minutes.

### Intermediate 90

### N—{(S)-(4,4-Difluorocyclohexyl)[3-(4-hydroxy-4-methylpiperidin-2-yl)imidazo[1,2-b]-[1,2,4]triazin-6-yl]methyl}-4-methyl-1,2,5-oxadiazole-3-carboxamide hydrochloride (syn isomers)

Intermediate 88 (665 mg, 1.126 mmol) was dissolved in 4N HCl in 1,4-dioxane (10 mL). The mixture was stirred at r.t. for 1 h, then neutralised with saturated aqueous NaHCO₃ solution and diluted with EtOAc. The layers were separated, and the aqueous layer was re-extracted with EtOAc. The combined organic phase was dried over Na₂SO₄, and concentrated in vacuo, to give the title compound (pair of syn stereoisomers, assumed 1:1 ratio) (504 mg, 91%). LCMS (Method 1): [M+H]⁺ m/z 491.2, RT 1.09 minutes.

### Intermediate 91

### N—{(S)-(4,4-Difluorocyclohexyl)[3-(4-hydroxy-4-methylpiperidin-2-yl)imidazo[1,2-b]-[1,2,4]triazin-6-yl]methyl}-4-methyl-1,2,5-oxadiazole-3-carboxamide hydrochloride (anti isomers)

Intermediate 89 (270 mg, 0.457 mmol) was dissolved in 4N HCl in 1,4-dioxane (4.5 mL). The mixture was stirred at r.t. for 1 h, then neutralised with saturated aqueous NaHCO₃ solution and diluted with EtOAc. The layers were separated, and the aqueous layer was re-extracted with EtOAc. The combined organic phase was dried over Na₂SO₄, and concentrated in vacuo, to give the title compound (pair of anti stereoisomers, assumed 1:1 ratio) (224 mg, 99%). LCMS (Method 1): [M+H]⁺ m/z 491.2, RT 1.10 minutes.

### Intermediate 92

### tert-Butyl 4-[tert-butyl(dimethyl)silyl]oxypiperidine-1-carboxylate

To a solution of tert-butyl 4-hydroxypiperidine-1-carboxylate (4.99 g, 24.8 mmol) and DIPEA (6.70 mL, 38.3 mmol) in DCM (70 mL), cooled in an ice-water bath, was added tert-butyl(dimethyl)silyl trifluoromethanesulfonate (5.20 mL, 22.6 mmol). The solution was stirred with cooling for 1 h, then the bath was removed. The solution was allowed to reach r.t., then stirred for a total of 6 h. DCM (100 mL) and half-saturated aqueous Na₂CO₃ solution (100 mL) were added. The layers were separated, and the organic layer was washed with additional half-saturated aqueous Na₂CO₃ solution (50 mL), then dried over magnesium sulfate, filtered and concentrated under reduced pressure. The residue was purified by column chromatography, eluting with a gradient of EtOAc in heptane, to give the title compound (5.41 g, 76%). δ_{H} (400 MHz, CDCl₃) 3.86 (tt, J 7.0, 3.4 Hz, 1H), 3.61 (ddd, J 11.9, 7.5, 3.6 Hz, 2H), 3.24 (ddd, J 13.1, 7.6, 3.7 Hz, 2H), 1.74-1.63 (m, 2H), 1.53-1.45 (m, 2H), 1.45 (s, 9H), 0.88 (s, 9H), 0.05 (s, 6H).

### Intermediate 93

### 1-(tert-Butoxycarbonyl)-4-[tert-butyl(dimethyl)silyl]oxypiperidine-2-carboxylic acid (racemic)

To a solution of Intermediate 92 (5.41 g, 17.1 mmol) in diethyl ether (120 mL) at −78° C. under nitrogen was added TMEDA (4.6 mL, 30.9 mmol), then 1.3M sec-butyllithium in hexanes (24 mL, 30.9 mmol) was added dropwise over 10 minutes. The mixture was stirred for 2.5 h at −78° C., then solid CO₂ (five medium-sized pellets, approximately 10 g) was added. The mixture was stirred at −78° C. for 10 minutes, then allowed to warm to r.t. Aqueous HCl (1M, 100 mL) was carefully added. The organic layer was diluted with EtOAc (100 mL), then the layers were separated. The organic layer was washed with 1M aqueous HCl (50 mL), then dried over MgSO₄, filtered and concentrated under reduced pressure. The resulting material was purified by column chromatography, eluting with a gradient of EtOAc in heptane, to give the title compound (3.96 g, 57%). δ_{H} (500 MHz, DMSO-d₆) 12.14 (s, 1H), 4.40 (dd, J 45.1, 6.2 Hz, 1H), 4.04 (s, 1H), 3.71-3.60 (m, 1H), 3.30-3.14 (m, 1H), 2.20-2.10 (m, 1H), 1.86-1.76 (m, 1H), 1.67-1.44 (m, 2H), 1.43-1.31 (m, 9H), 0.83 (s, 9H), 0.00 (s, 6H).

### Intermediate 94

### O¹-tert-Butyl O²-(1,3-dioxoisoindolin-2-yl) 4-[tert-butyl(dimethyl)silyl]oxypiperidine-1,2-dicarboxylate (racemic)

To a solution of Intermediate 93 (89%, 1.65 g, 4.08 mmol) and N-hydroxyphthalimide (1.00 g, 6.13 mmol) in DCM (35 mL) was added EDCI·HCl (1:1) (1.17 g, 6.13 mmol). The cloudy mixture became a clear yellow solution and was stirred at r.t. for 2 h. Additional N-hydroxyphthalimide (0.67 g, 4.08 mmol) and EDCI·HCl (1:1) (0.78 g, 4.08 mmol) were added and stirring was continued at r.t. for 2 h. The solution was washed with water (50 mL) and passed through a hydrophobic frit, washing through with DCM. The resulting material was concentrated under reduced pressure and purified by column chromatography, eluting with a gradient of EtOAc in heptane, to give the title compound (1.65 g, 67%). δ_{H} (500 MHz, CDCl₃) 7.91-7.84 (m, 2H), 7.82-7.73 (m, 2H), 5.26-4.97 (m, 1H), 4.21-4.10 (m, 1H), 4.04-3.75 (m, 1H), 3.71-3.41 (m, 1H), 2.48-2.39 (m, 1H), 2.08-1.99 (m, 1H), 1.78-1.61 (m, 2H), 1.52 (s, 9H), 0.83 (s, 9H), 0.12-0.06 (m, 6H). LCMS (Method 19): [M−BOC+H]⁺ m/z 405.2, RT 2.82 minutes.

### Intermediate 95

### tert-Butyl 4-[tert-butyl(dimethyl)silyl]oxy-2-(6-{(S)-(4,4-difluorocyclohexyl)[(4-methyl-1,2,5-oxadiazole-3-carbonyl)amino]methyl}imidazo[1,2-b][1,2,4]triazin-3-yl)piperidine-1-carboxylate

To a solution of Intermediate 94 (84%, 1.53 g, 2.54 mmol), Intermediate 21 (800 mg, 2.12 mmol) and tris[2-phenylpyridinato-C₂,N]iridium(III) (28 mg, 42.4 μmol) in anhydrous DMSO (24 mL) was added TFA (243 μL, 3.18 mmol). The solution was purged by bubbling through nitrogen with stirring for 10 minutes, then sealed under nitrogen with parafilm. The reaction mixture was irradiated in a Penn M2 photoreactor (450 nm; LED 100%; stirring 50%; fan 100%) for 4 h at r.t. The mixture was diluted with EtOAc (50 mL), then washed with water (2×50 mL) and brine (20 mL). The resulting material was dried over MgSO₄, then filtered and concentrated under reduced pressure. The residue was purified by column chromatography, eluting with a gradient of EtOAc in heptane, to give the title compound (unresolved mixture of diastereomers) (970 mg, 37%). LCMS (Method 4): [M+H]⁺ m/z 691.4, RT 2.76 minutes (minor isomer); [M+H]⁺ m/z 691.4, RT 2.85 minutes (major isomer).

### Intermediate 96

### tert-Butyl 2-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-hydroxypiperidine-1-carboxylate

To a solution of Intermediate 95 (56%, 970 mg, 0.79 mmol) in THF (5 mL) was added 1M TBAF in THF (1.0 mL, 1.00 mmol). The solution was stirred at r.t. for 21 h. Additional 1M TBAF in THF (1 mL) was added, and stirring was continued for 26 h. Additional 1M TBAF in THF (0.5 mL) was added, and stirring was continued for 19 h. The reaction mixture was diluted with EtOAc (20 mL) and washed with half-saturated aqueous NaHCO₃ solution (2×20 mL), followed by brine (20 mL). The organic layer was dried over MgSO₄, then filtered and concentrated under reduced pressure. The residue was purified by column chromatography, eluting with a gradient of EtOAc in heptane, to give the title compound (unresolved mixture of isomers) (350 mg, 73%). 8H (400 MHz, CDCl₃) 8.47-8.33 (m, 1H), 7.92-7.82 (m, 1H), 7.81-7.73 (m, 1H), 5.88-5.50 (m, 1H), 5.33-5.21 (m, 1H), 4.32-3.91 (m, 2H), 3.30-2.67 (m, 2H), 2.65-2.55 (m, 3H), 2.48-2.11 (m, 3H), 2.12-1.86 (m, 3H), 1.86-1.31 (m, 16H). LCMS (Method 4): [M+H]⁺ m/z 577.2, RT 2.27 minutes.

### Intermediate 97

### N—{(S)-(4,4-Difluorocyclohexyl)[3-(4-hydroxypiperidin-2-yl)imidazo[1,2-b][1,2,4]triazin-6-yl]methyl}-4-methyl-1,2,5-oxadiazole-3-carboxamide hydrochloride

To Intermediate 96 (94%, 350 mg, 0.57 mmol) was added 4M HCl in 1,4-dioxane (10 mL). The solution was stirred at r.t. for 4 h, then the solvent was removed under reduced pressure and the crude material was left to stand for 16 h. The residue was dissolved in 4M HCl in 1,4-dioxane (10 mL) and stirred at r.t. for 2 h. The solvent was removed under reduced pressure to give the title compound (360 mg, 100%). LCMS (Method 4): [M+H]⁺ m/z 477.2, RT 1.76 minutes.

### Intermediate 98

### N—[(S)-(4,4-Difluorocyclohexyl)(imidazo[1,2-b][1,2,4]triazin-6-yl)methyl]-2-isopropyl-1,2,4-triazole-3-carboxamide

To a stirred solution of Intermediate 20 (9.40 g, 33.4 mmol), lithium 2-isopropyl-1,2,4-triazole-3-carboxylate (5.90 g, 36.6 mmol) and DIPEA (12 mL, 68.7 mmol) in anhydrous DMF (130 mL) was added HATU (15.00 g, 39.4 mmol) portionwise. The reaction mixture was stirred at r.t. for 16 h, then diluted with EtOAc (400 mL), washed with brine (4×200 mL), dried over anhydrous Na₂SO₄ and filtered. The solvent was removed in vacuo. The residue was purified using automated chromatography (Isolera 4, 350 g SFAR HC Duo column), eluting with a gradient of EtOAc in heptane (5-65%), to provide the title compound (13.90 g, 93%) as an orange solid. δ_{H} (500 MHz, DMSO-d₆) 8.99 (d, J 9.2 Hz, 1H), 8.65 (d, J 2.0 Hz, 1H), 8.57 (d, J 2.0 Hz, 1H), 8.38 (s, 1H), 8.10 (d, J 0.6 Hz, 1H), 5.53 (hept, J 6.6 Hz, 1H), 5.20 (t, J 8.7 Hz, 1H), 2.25-2.15 (m, 1H), 2.09-1.94 (m, 2H), 1.93-1.87 (m, 1H), 1.86-1.68 (m, 2H), 1.61 (d, J 13.6 Hz, 1H), 1.42 (d, J 6.6 Hz, 3H), 1.37 (d, J 6.6 Hz, 3H), 1.35-1.25 (m, 2H). LCMS (Method 2): [M+H]⁺ m z 405, RT 2.90 minutes.

### Intermediates 99 & 100

### tert-Butyl (2S,4S)-2-(6-{(S)-(4,4-difluorocyclohexyl)[(2-isopropyl-1,2,4-triazole-3-carbonyl)amino]methyl}imidazo[1,2-b][1,2,4]triazin-3-yl)-4-hydroxypiperidine-1-carboxylate

### tert-Butyl (2R,4S)-2-(6-{(S)-(4,4-difluorocyclohexyl)[(2-isopropyl-1,2,4-triazole-3-carbonyl)amino]methyl}imidazo[1,2-b][1,2,4]triazin-3-yl)-4-hydroxypiperidine-1-carboxylate

Into a screw-cap vial were introduced Intermediate 98 (450 mg, 1.11 mmol), (2R,4S)-1-(tert-butoxycarbonyl)-4-hydroxypiperidine-2-carboxylic acid (410 mg, 1.67 mmol), DMSO (11.1 mL), {Ir[dF(CF₃)ppy]₂(dtbpy)}PF₆ (25.0 mg, 0.022 mmol) and Cs₂CO₃ (544 mg, 1.67 mmol) sequentially. The vial was capped and the mixture was purged with N₂ for 10 minutes, then the cap was sealed with parafilm and the mixture was irradiated (450 nm) using an ‘integrated photoreactor’ (ACS Cent. Sci., 2017, 3, 647-653) (settings: Fan=1612 rpm; Stir=392 rpm; LED=100%) and stirred vigorously for 20 h. The mixture was diluted with EtOAc (20 mL) and washed with H₂O (2×10 mL). The combined organic layers were dried (Na₂SO₄) and concentrated in vacuo. Purification by flash chromatography, eluting with EtOAc/isohexane (0-100% gradient), then 5% MeOH/DCM, afforded the title compounds (mixture of two stereoisomers in approximately 3:1 anti:syn ratio) (237 mg, 35%) as an orange oil/foam. LCMS (Method 1): [M+H]⁺ m z 604.4, 604.2, RT 1.36 minutes.

### Intermediates 101 & 102

### N—[(S)-(4,4-Difluorocyclohexyl){3-[(2S,4S)-4-hydroxypiperidin-2-yl]imidazo[1,2-b]-[1,2,4]triazin-6-yl}methyl]-2-isopropyl-1,2,4-triazole-3-carboxamide hydrochloride

### N—[(S)-(4,4-Difluorocyclohexyl){3-[(2R,4S)-4-hydroxypiperidin-2-yl]imidazo[1,2-b]-[1,2,4]triazin-6-yl}methyl]-2-isopropyl-1,2,4-triazole-3-carboxamide hydrochloride

To Intermediates 99 and 100 (mixture of anti:syn stereoisomers in approximately 3:1 ratio) (237 mg, 0.39 mmol) was added 4N HCl in 1,4-dioxane (3.60 mL, 14.0 mmol) dropwise. The mixture was stirred at r.t. After 10 minutes, diethyl ether (25 mL) was added, resulting in precipitation. The mixture was stirred for 10 minutes, then filtered under suction. The sticky solid filter cake was washed with copious amounts of diethyl ether. The filtrate was discarded. The solid was re-dissolved in MeOH, and concentrated in vacuo, to afford the title compounds (mixture of anti:syn stereoisomers in approximately 3:1 ratio) (235 mg, quantitative) as an orange oil, which was utilised without further purification. LCMS (Method 1): [M+H]⁺ m/z 504.2, RT 1.09 minutes.

### Intermediate 103

### tert-Butyl 2-(6-{(S)-(4,4-difluorocyclohexyl)[(2-isopropyl-1,2,4-triazole-3-carbonyl)-amino]methyl}imidazo[1,2-b][1,2,4]triazin-3-yl)-4-hydroxy-4-methylpiperidine-1-carboxylate

To a mixture of Intermediate 87 (458 mg, 1.77 mmol), Intermediate 98 (353 mg, 0.804 mmol), {Ir[dF(CF₃)ppy]₂(dtbpy)}PF₆ (18 mg, 16.1 μmol) and Cs₂CO₃ (786 mg, 2.41 mmol) was added anhydrous DMF (16 mL). The mixture was degassed by bubbling through nitrogen whilst stirring for 10 minutes, then irradiated in a Penn M2 photoreactor (450 nm, LED 100%, stirring 100%, fan 100%) for 2×3 h at r.t. The reaction mixture was diluted with EtOAc (25 mL), washed with water (2×25 mL) and brine (25 mL), then dried over MgSO₄, filtered and concentrated under reduced pressure. The residue was taken up in DCM (10 mL), and manganese dioxide (1.40 g, 16.1 mmol) was added. The reaction mixture was stirred for 1 h. The suspension was filtered through Celite®, then concentrated under reduced pressure. The residue was purified by silica column chromatography, eluting with a gradient of EtOAc in heptane, followed by reverse-phase C18 column chromatography, eluting with a gradient of acetonitrile in water with 0.1% formic acid, to give the title compound (mixture of syn isomers) (87 mg, 16%). δ_{H} (500 MHz, CDCl₃) 8.36 (s, 1H), 8.29-8.11 (m, 1H), 8.10-7.67 (m, 3H), 5.79-5.70 (m, 1H), 5.69-5.35 (m, 1H), 5.24-5.13 (m, 1H), 4.10-3.89 (m, 1H), 3.28-3.05 (m, 1H), 2.85-2.70 (m, 1H), 2.26-2.19 (m, 1H), 2.18-2.10 (m, 1H), 1.99 (dd, J 14.2, 6.5 Hz, 2H), 1.80-1.57 (m, 4H), 1.55-1.33 (m, 19H), 1.31 (s, 3H). LCMS (Method 4): [M+H]⁺ m/z 618.02, RT 2.41 minutes.

### Intermediate 104

### N—{(S)-(4,4-Difluorocyclohexyl)[3-(4-hydroxy-4-methylpiperidin-2-yl)imidazo[1,2-b]-[1,2,4]triazin-6-yl]methyl}-2-isopropyl-1,2,4-triazole-3-carboxamide hydrochloride

A solution of Intermediate 103 (87 mg, 0.130 mmol) in 4M HCl in 1,4-dioxane (0.9 mL) was stirred for 15 minutes. The reaction mixture was concentrated under reduced pressure to afford the title compound (71 mg, 99%). LCMS (Method 4): [M+H]+m/z 518.2, RT 1.79 minutes.

### Intermediate 105

### O¹-tert-Butyl O⁴-methyl 4-(3,3,4,4-tetrafluoropyrrolidine-1-carbonyl)piperidine-1,4-dicarboxylate

To a stirred suspension of 1-(tert-butoxycarbonyl)-4-methoxycarbonylpiperidine-4-carboxylic acid (1.05 g, 3.65 mmol), 3,3,4,4-tetrafluoropyrrolidine hydrochloride (1:1) (0.72 g, 4.01 mmol) and HATU (1.81 g, 4.76 mmol) in DCM (20 mL) at r.t. was added DIPEA (2.0 mL, 11.7 mmol). The reaction mixture was stirred for 64 h, then diluted with DCM (50 mL) and quenched with saturated aqueous NaHCO₃ solution (50 mL). The phases were separated, and the aqueous phase was further extracted with DCM (2×50 mL). The combined organic layers were washed with brine (50 mL) and dried (frit), then filtered and concentrated in vacuo. The resulting crude material was purified by flash column chromatography, eluting with EtOAc/heptane (0-100% gradient), to afford the title compound (90% purity) (0.70 g, 42%) as a yellow solid. δ_{H} (500 MHz, DMSO-d₆) 4.19-4.00 (m, 4H), 3.72 (s, 3H), 3.49-3.24 (obs. m, 4H), 2.01-1.83 (m, 4H), 1.39 (s, 9H). LCMS (Method 30): [M−BOC+H]⁺ m/z 313, RT 4.00 minutes.

### Intermediate 106

### 1-(tert-Butoxycarbonyl)-4-(3,3,4,4-tetrafluoropyrrolidine-1-carbonyl)piperidine-4-carboxylic acid

To a stirred solution of Intermediate 105 (0.70 g, 1.52 mmol) in THF (15 mL) at r.t. was added 1M lithium hydroxide (4.6 mL, 4.55 mmol) in one portion. The reaction mixture was stirred for 18 h. Additional 1M lithium hydroxide (4.6 mL, 4.55 mmol) was added in one portion, and the reaction mixture was stirred for 3.5 h. Additional 1M lithium hydroxide (4.6 mL, 4.55 mmol) was added in one portion, and the reaction mixture was stirred for 1 h, then acidified to pH 2 with 1N HCl and extracted with EtOAc (3×30 mL). The combined organic phases were washed with water (30 mL) and brine (30 mL), then dried over MgSO₄, filtered and concentrated in vacuo, to afford the title compound (0.60 g, 99%) as a white solid. δ_{H} (500 MHz, DMSO-d₆) 14.66-12.36 (m, 1H), 4.19-4.00 (m, 4H), 3.52-3.09 (obs. m, 4H), 1.99-1.83 (m, 4H), 1.39 (s, 9H). LCMS (Method 30): [M−BOC+H]⁺ m/z 299, RT 3.33 minutes.

### Intermediate 107

### O¹-tert-Butyl O⁴-(1,3-dioxoisoindolin-2-yl) 4-(3,3,4,4-tetrafluoropyrrolidine-1-carbonyl)-piperidine-1,4-dicarboxylate

To a stirred suspension of Intermediate 106 (0.78 g, 1.95 mmol) and 2-hydroxy-1H-isoindole-1,3(2H)-dione (0.35 g, 2.13 mmol) in DCM (25 mL) at r.t. was added EDCI·HCl (0.41 g, 2.14 mmol). The resulting clear yellow solution was stirred under N₂ for 2 h, then the solvent was concentrated in vacuo. The residue was purified by flash column chromatography, eluting with EtOAc/heptane (0-100% gradient), to afford the title compound (90% purity) (0.80 g, 68%) as a white solid. δ_{H} (500 MHz, DMSO-d₆) 8.06-8.00 (m, 2H), 8.00-7.94 (m, 2H), 4.61-4.09 (m, 4H), 3.68-3.53 (m, 2H), 3.46-3.23 (m, 2H), 2.26-2.08 (m, 4H), 1.41 (s, 9H). LCMS (Method 30): [M-BOC+H]⁺ m/z 444, RT 3.98 minutes.

### Intermediate 108

### tert-Butyl 4-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-(3,3,4,4-tetrafluoropyrrolidine-1-carbonyl)piperidine-1-carboxylate

To a stirred solution of Intermediate 21 (275 mg, 0.73 mmol), Intermediate 107 (792 mg, 1.46 mmol) and {Ir[dF(CF₃)ppy]₂(dtbpy)}PF₆ (16.5 mg, 14.7 μmol) in anhydrous DMSO (24.5 mL) at r.t. was added TFA (84 mL, 1.20 mmol). The mixture was purged by bubbling through N2 whilst stirring for 10 minutes, then sealed under N₂ with parafilm and irradiated in a Penn M2 photoreactor (450 nm, LED 100%, stirring 50%, fan 100%) for 18 h. The reaction mixture was diluted with water (40 mL), quenched with saturated aqueous NaHCO₃ solution (40 mL) and extracted with EtOAc (5×40 mL). The combined organic layers were washed with water (40 mL) and brine (40 mL), then dried over MgSO₄, filtered and concentrated in vacuo. The resulting crude material was purified by flash column chromatography, eluting with EtOAc/heptane (0-100% gradient), to afford the title compound (77% purity) (374 mg, 50%) as a yellow-orange solid. δ_{H} (500 MHz, DMSO-d₆) 9.54 (d, J 9.0 Hz, 1H), 8.66 (s, 1H), 8.34 (s, 1H), 5.23 (t, J 8.6 Hz, 1H), 4.43-3.84 (m, 5H), 3.80-3.65 (m, 2H), 3.25-2.95 (m, 2H), 2.46 (s, 3H), 2.32-1.55 (m, 10H), 1.40 (s, 9H), 1.36-1.21 (m, 2H). LCMS (Method 31): [M+H]⁺ m/z 730, RT 1.03 minutes.

### Intermediate 109

### N—[(S)-(4,4-Difluorocyclohexyl){3-[4-(3,3,4,4-tetrafluoropyrrolidine-1-carbonyl)-piperidin-4-yl]imidazo[1,2-b][1,2,4]triazin-6-yl}methyl]-4-methyl-1,2,5-oxadiazole-3-carboxamide

To a stirred solution of Intermediate 108 (370 mg, 0.390 mmol) in DCM (15 mL) at r.t. was added TFA (3.3 mL). The solution was stirred for 2 h, then passed through a 20 g SCX-2 cartridge, washing with MeOH and eluting with 7N NH₃ in MeOH. The latter phase was concentrated in vacuo to give the title compound (90% purity) (285 mg, quantitative) as a yellow-orange oil, which solidified on standing. δ_{H} (500 MHz, DMSO-d₆) 9.53 (d, J 9.1 Hz, 1H), 8.61 (s, 1H), 8.34 (s, 1H), 5.23 (t, J 8.6 Hz, 1H), 4.23-3.80 (m, 4H), 2.94-2.85 (m, 2H), 2.84-2.73 (m, 2H), 2.47 (s, 3H), 2.27-2.17 (m, 3H), 2.07 (s, 4H), 1.96-1.87 (m, 1H), 1.87-1.71 (m, 2H), 1.70-1.60 (m, 2H), 1.51-1.25 (m, 2H). LCMS (Method 30): [M+H]⁺ m/z 630, RT 3.20 minutes.

### Intermediate 110

### O¹-tert-Butyl O⁴-methyl 4-[3-(trifluoromethyl)azetidine-1-carbonyl]piperidine-1,4-dicarboxylate

DIPEA (0.1 mL, 0.6 mmol) was added to a solution of 3-(trifluoromethyl)-azetidine hydrochloride (1.2 g, 7.1 mmol) and 1-(tert-butoxycarbonyl)-4-methoxy-carbonylpiperidine-4-carboxylic acid (1.9 g, 6.4 mmol) in DCM (25 mL), followed by HATU (95 mg, 0.25 mmol). The reaction mixture was left overnight at r.t., then diluted with DCM (25 mL) and washed with aqueous NH₄Cl solution (25 mL). The resulting material was passed through a hydrophobic frit and concentrated in vacuo. The residue was purified by flash chromatography (hexane/EtOAc, 1-50% gradient, on 25 g silica cartridge) to give the title compound (1.7 g, 67%) as a pale a yellow solid. LCMS (Method 1): [M-O^{t}Bu+H]⁺ m/z 339.0, RT 1.06 minutes.

### Intermediate 111

### Lithium 1-(tert-butoxycarbonyl)-4-[3-(trifluoromethyl)azetidine-1-carbonyl]piperidine-4-carboxylate

Lithium hydroxide monohydrate (138 mg, 3.33 mmol) was dissolved in water (1 mL) and added to a solution of Intermediate 110 (1 g, 2.54 mmol) in THF (5 mL). the mixture was stirred overnight. Additional lithium hydroxide monohydrate (30 mg) in water (˜0.5 mL) was added, and MeOH (1 mL) was also added to aid dissolution. The reaction mixture was stirred for a further 5 h, then evaporated in vacuo, azeotroping with MeOH and diethyl ether, to afford the title compound (985 mg, quantitative) as a white solid. LCMS (Method 1): [M-O^{t}Bu+H]⁺ m/z 325.0, RT 0.61 minutes.

### Intermediate 112

### O¹-tert-Butyl O⁴-(1,3-dioxoisoindolin-2-yl) 4-[3-(trifluoromethyl)azetidine-1-carbonyl]-piperidine-1,4-dicarboxylate

Intermediate 111 (1 g, 2.447 mmol) was added to a solution of N-hydroxyphthalimide (620 mg, 3.69 mmol) in DMF (10 mL), then EDCI·HCl (720 mg, 3.68 mmol) was added. After 18 h, additional N-hydroxyphthalimide (300 mg) and EDCI·HCl (300 mg) were added. The reaction mixture was left for a further 2 h, then diluted with DCM (25 mL) and washed with aqueous NH₄Cl solution (25 mL) and brine (2×20 mL). The resulting material was passed through a hydrophobic frit and evaporated in vacuo. The residue was purified by flash chromatography, eluting with hexane and EtOAc (1-33% gradient) on a 25 g silica column, to afford the title compound (780 mg, 55%) as an off-white solid. LCMS (Method 1): [M-BOC+H]⁺ m/z 426.2, RT 1.25 minutes.

### Intermediate 113

### tert-Butyl 4-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-[3-(trifluoromethyl)azetidine-1-carbonyl]piperidine-1-carboxylate

Intermediate 21 (125 mg, 0.331 mmol) was dissolved in DMSO (5 mL) and treated with Intermediate 112 (365 mg, 0.660 mmol) and TFA (0.030 mL, 0.36 mmol), then {Ir[dF(CF₃)ppy]₂(dtbpy)}PF₆ (6 mg, 0.0053 mmol). N2 was bubbled through, then the mixture was placed in a Merck Penn photoreactor and stirred for 7 h at 450 nm. The reaction mixture was separated between EtOAc (20 mL) and brine (20 mL). The organic layer was washed with brine (2×10 mL), then passed through a hydrophobic frit and concentrated in vacuo. The resulting yellow solid was purified by flash chromatography (hexane/EtOAc, 1-100% gradient, 25 g silica column) to afford the title compound (60 mg, 23%). LCMS (Method 8): [M+H]⁺ m/z 712.2, RT 2.71 minutes.

### Intermediate 114

### N—[(S)-(4,4-Difluorocyclohexyl)(3-{4-[3-(trifluoromethyl)azetidine-1-carbonyl]piperidin-4-yl}imidazo[1,2-b][1,2,4]triazin-6-yl)methyl]-4-methyl-1,2,5-oxadiazole-3-carboxamide

Intermediate 113 (70 mg, 0.049 mmol) was dissolved in DCM (4 mL) and treated with TFA (2 mL). The mixture was stirred for 3 h, then diluted with DCM (20 mL) and washed with water (10 mL) and 0.5M HCl (5 mL). The combined aqueous layers were basified with saturated aqueous NaHCO₃ solution and extracted with DCM (3×10 mL). The organic layers were passed through a hydrophobic frit, and evaporated in vacuo, to afford the title compound (47 mg, 92%). LCMS (Method 7): [M+H]⁺ m/z 612.4, RT 1.68 minutes.

### Intermediate 115

### O¹-tert-Butyl O⁴-methyl 4-(2,2-difluoropropylcarbamoyl)piperidine-1,4-dicarboxylate

To a solution of 1-(tert-butoxycarbonyl)-4-methoxycarbonylpiperidine-4-carboxylic acid (1.00 g, 3.48 mmol), 2,2-difluoropropylamine hydrochloride (572 mg, 4.18 mmol) and DIPEA (1.82 mL, 10.4 mmol) in DMF (17 mL) was added HATU (1.62 g, 4.18 mmol). The mixture was stirred at r.t. for 10 minutes, then diluted with water (100 mL) and extracted with EtOAc (3×50 mL). The combined organic extracts were passed through a phase separator and concentrated in vacuo. The crude material was purified by column chromatography, eluting with a gradient of 0-50% EtOAc in isohexane, to give the title compound (1.26 g, 99%) as a colourless oil. LCMS (Method 1): [M-BOC+H]⁺ m/z 265.2, RT 0.98 minutes.

### Intermediate 116

### 1-(tert-Butoxycarbonyl)-4-(2,2-difluoropropylcarbamoyl)piperidine-4-carboxylic acid

To a solution of Intermediate 115 (1.26 g, 3.45 mmol) in THF (14 mL) was added a solution of lithium hydroxide monohydrate (294 mg, 6.89 mmol) in water (3.5 mL). The reaction mixture was stirred at r.t. for 72 h. A second portion of lithium hydroxide monohydrate (294 mg, 6.89 mmol) was added, and the reaction mixture was stirred for 2 h, then acidified to pH 4 with 2.0M aqueous HCl (7 mL) and diluted with water (50 mL). The aqueous layer was extracted with EtOAc (3×50 mL). The combined organic extracts were passed through a phase separator, and concentrated in vacuo, to give the title compound (1.12 g, 93%) as a colourless amorphous solid. LCMS (Method 1): [M-BOC+H]⁺ m/z 251.2, RT 0.57 minutes.

### Intermediate 117

### O¹-tert-Butyl O⁴-(1,3-dioxoisoindolin-2-yl) 4-(2,2-difluoropropylcarbamoyl)piperidine-1,4-dicarboxylate

To a solution of Intermediate 116 (1.12 g, 3.20 mmol) and N-hydroxyphthalimide (591 mg, 3.52 mmol) in DMF (16 mL) was added EDCI·HCl (681 mg, 3.52 mmol). The mixture was stirred at r.t. for 1.5 h, then diluted with water (50 mL) and extracted with EtOAc (3×50 mL). The combined organic extracts were passed through a phase separator and concentrated in vacuo. The crude material was purified by column chromatography, eluting with a gradient of 0-100% EtOAc in isohexane, to give the title compound (1.22 g, 77%) as a colourless amorphous solid. LCMS (Method 1): [M-BOC+H]⁺ m/z 396.2, RT 1.19 minutes.

### Intermediate 118

### tert-Butyl 4-(6-{(S)-(4,4-difluorocyclohexyl)[(4-methyl-1,2,5-oxadiazole-3-carbonyl)-amino]methyl}imidazo[1,2-b][1,2,4]triazin-3-yl)-4-(2,2-difluoropropylcarbamoyl)-piperidine-1-carboxylate

N₂ gas was bubbled through a solution of Intermediate 21 (400 mg, 1.06 mmol), Intermediate 117 (1050 mg, 2.12 mmol), {Ir[dF(CF₃)ppy]₂(dtbpy)}PF₆ (24 mg, 0.02 mmol) and TFA (121 μL, 1.59 mmol) in DMSO (21 mL) for 5 minutes. The reaction mixture was placed under 450 nm irradiation at r.t. for 45 h, then quenched with saturated aqueous NaHCO₃ solution (100 mL) and extracted with EtOAc (3×50 mL). The combined organic extracts were passed through a phase separator and concentrated in vacuo. The crude material was purified by column chromatography, eluting with a gradient of 0-50% EtOAc in isohexane, to give the title compound (500 mg, 69%) as an orange amorphous solid. LCMS (Method 1): [M+H]⁺ m/z 682.4, RT 1.25 minutes.

### Intermediate 119

### N—[(S)-(4,4-difluorocyclohexyl){3-[4-(2,2-difluoropropylcarbamoyl)piperidin-4-yl]-imidazo[1,2-b][1,2,4]triazin-6-yl}methyl]-4-methyl-1,2,5-oxadiazole-3-carboxamide

To a solution of Intermediate 118 (500 mg, 0.73 mmol) in DCM (3.0 mL) was added TFA (1.0 mL). The mixture was stirred at r.t. for 3.5 h, then quenched with aqueous NaHCO₃ solution (50 mL). The aqueous layer was extracted with DCM (3×25 mL). The combined organic extracts were passed through a phase separator, and concentrated in vacuo, to give the title compound (316 mg, 74%) as a colourless amorphous solid. LCMS (Method 1): [M+H]⁺ m/z 582.4, RT 0.92 minutes.

### Examples 1 & 2

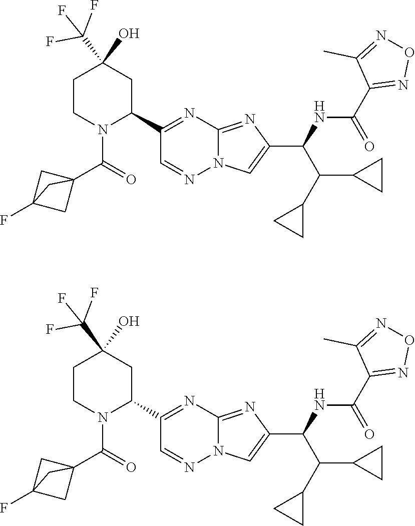 [SMILES: Cc1nonc1C(=O)N[C@H](c1cn2ncc([C@@H]3C[C@@](O)(C(F)(F)F)CCN3C(=O)C34CC(F)(C3)C4)nc2n1)C(C1CC1)C1CC1 ; Cc1nonc1C(=O)N[C@H](c1cn2ncc([C@H]3C[C@](O)(C(F)(F)F)CCN3C(=O)C34CC(F)(C3)C4)nc2n1)C(C1CC1)C1CC1]

### N-[(1S)-2,2-Dicyclopropyl-1-{3-[(2S,4R)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}ethyl]-4-methyl-1,2,5-oxadiazole-3-carboxamide

### N-[(1S)-2,2-Dicyclopropyl-1-{3-[(2R,4S)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}ethyl]-4-methyl-1,2,5-oxadiazole-3-carboxamide

To a solution of Intermediate 16 (mixture of two stereoisomers) (40.0 mg, 0.076 mmol), 4-methyl-1,2,5-oxadiazole-3-carboxylic acid (10.0 mg, 0.078 mmol) and DIPEA (0.06 mL, 0.30 mmol) in DMF (4 mL) at r.t. was added HATU (36.0 mg, 0.092 mmol) in one portion. The mixture was stirred for 15 minutes, then H₂O (10 mL) was added. The mixture was extracted with EtOAc (3×20 mL) and the combined organic extracts were washed with brine (20 mL), then dried (Na₂SO₄) and concentrated in vacuo. The residue was purified by flash chromatography, eluting with EtOAc/isohexane (0-75% gradient). The resulting material (35.0 mg) was subject to chiral purification (Method 9) to afford, after lyophilisation, the title compounds (Peak 1, 9.0 mg, 18.7% yield, >99% d.e.; and Peak 2, 9.0 mg, 18.7% yield, 82.9% d.e.).

Peak 1: δ_{H} (400 MHz, DMSO-d₆) 9.26-9.16 (m, 1H, major and minor rotamers), 8.77 (d, J 11.1 Hz, 1H), 8.30 (d, J 11.2 Hz, 1H), 6.63-5.84 (br s, 1H, and app. d, J 6.9 Hz, 1H, major rotamer), 5.61 (app. d, J 6.3 Hz, 1H, minor rotamer), 5.57-5.47 (m, 1H), 4.53-4.43 (m, 1H, minor rotamer), 4.14-4.02 (m, 1H, major rotamer), 3.82-3.69 (m, 1H, major rotamer), 3.24-3.11 (m, 1H, minor rotamer), 2.66-2.40 (2× obscured m, 4H, and obscured s, 3H), 2.32-2.10 (m, 4H), 1.81-1.67 (m, 2H), 1.03-0.90 (m, 1H), 0.86-0.66 (m, 2H), 0.47-0.34 (m, 2H), 0.33-0.16 (m, 4H), 0.13 to −0.06 (m, 2H). LCMS (Method 7): [M+H]⁺ m/z 633.4, RT 2.15 minutes. Chiral analysis (Method 10): RT 8.07 minutes.

Peak 2: δ_{H} (400 MHz, DMSO-d₆) 9.26-9.16 (m, 1H, major and minor rotamers), 8.76 (d, J 12.6 Hz, 1H), 8.29 (d, J 9.5 Hz, 1H), 6.10-5.86 (br. s, 1H, and app. d, J 6.8 Hz, 1H, major rotamer), 5.61 (app. d, J 6.3 Hz, 1H, minor rotamer), 5.56-5.46 (m, 1H), 4.53-4.43 (m, 1H, minor rotamer), 4.14-4.02 (m, 1H, major rotamer), 3.81-3.68 (m, 1H, major rotamer), 3.27-3.14 (m, 1H, minor rotamer), 2.66-2.40 (2× obscured m, 4H, and obscured s, 3H), 2.31-2.10 (m, 4H), 1.82-1.67 (m, 2H), 1.04-0.90 (m, 1H), 0.85-0.68 (m, 2H), 0.48-0.34 (m, 2H), 0.33-0.15 (m, 4H), 0.12 to −0.07 (m, 2H). LCMS (Method 7): [M+H]⁺ m/z 633.4, RT 2.15 minutes. Chiral analysis (Method 10): RT 9.31 minutes.

### Examples 3 & 4

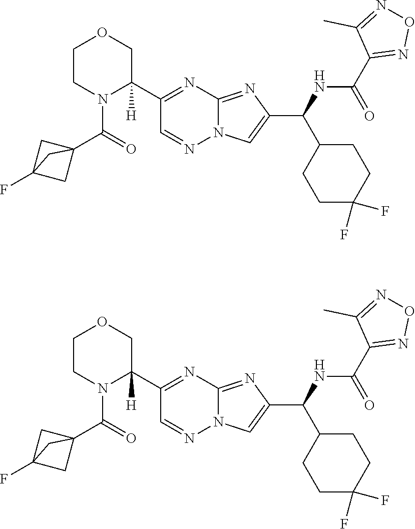 [SMILES: Cc1nonc1C(=O)N[C@H](c1cn2ncc([C@@H]3COCCN3C(=O)C34CC(F)(C3)C4)nc2n1)C1CCC(F)(F)CC1 ; Cc1nonc1C(=O)N[C@H](c1cn2ncc([C@H]3COCCN3C(=O)C34CC(F)(C3)C4)nc2n1)C1CCC(F)(F)CC1]

### N—[(S)-(4,4-Difluorocyclohexyl){3-[(3R)-4-(3-fluorobicyclo[1.1.1]pentane-1-carbonyl)-morpholin-3-yl]imidazo[1,2-b][1,2,4]triazin-6-yl}methyl]-4-methyl-1,2,5-oxadiazole-3-carboxamide

### N—[(S)-(4,4-Difluorocyclohexyl){3-[(3S)-4-(3-fluorobicyclo[1.1.1]pentane-1-carbonyl)-morpholin-3-yl]imidazo[1,2-b][1,2,4]triazin-6-yl}methyl]-4-methyl-1,2,5-oxadiazole-3-carboxamide

To a solution of Intermediate 26 (405 mg, 0.74 mmol), 4-methyl-1,2,5-oxadiazole-3-carboxylic acid (95.0 mg, 0.74 mmol) and DIPEA (0.52 mL, 3.00 mmol) in DMF (15 mL) at r.t. was added HATU (349 mg, 0.89 mmol) in one portion. The mixture was stirred for 10 minutes, then H₂O (25 mL) was added. The mixture was extracted with EtOAc (3×40 mL) and the combined organic extracts were washed with brine (80 mL), then dried (Na₂SO₄) and concentrated in vacuo. The residue was purified by flash chromatography, eluting with EtOAc/isohexane (0-75% gradient). The resulting material (244 mg) was subject to chiral purification (Method 11) to afford, after lyophilisation, the title compounds (Peak 1, 13.0 mg, 3% yield, >97.8% d.e.; and Peak 2, 13.0 mg, 3% yield, 89.3% d.e.).

Peak 1: δ_{H} (400 MHz, 373K, DMSO-d₆) 9.06 (d, J 8.4 Hz, 1H), 8.67 (s, 1H), 8.27 (s, 1H), 5.56-5.32 (br s, 1H), 5.27 (t, J 8.2 Hz, 1H), 4.53 (app. d, J 12.3 Hz, 1H), 4.11-3.93 (br s, 1H), 3.92-3.81 (m, 2H), 3.57 (app. t, J 11.1 Hz, 1H), 2.65-2.46 (obscured s, 3H), 2.46-2.37 (m, 7H), 2.31-2.18 (m, 1H), 2.16-1.91 (m, 3H), 1.91-1.68 (m, 3H), 1.56-1.42 (m, 1H), 1.42-1.30 (m, 1H). LCMS (Method 7): [M+H]⁺ m/z 575.2, RT 1.83 minutes. Chiral analysis (Method 12): RT 2.51 minutes.

Peak 2: δ_{H} (400 MHz, 373K, DMSO-d₆) 9.07 (d, J 8.4 Hz, 1H), 8.67 (s, 1H), 8.27 (s, 1H), 5.54-5.41 (br s, 1H), 5.27 (t, J 8.2 Hz, 1H), 4.53 (app. d, J 12.3 Hz, 1H), 4.11-3.92 (br s, 1H), 3.92-3.82 (m, 2H), 3.57 (app. t, J 11.0 Hz, 1H), 2.58-2.47 (obscured s, 3H), 2.47-2.37 (m, 7H), 2.32-2.18 (m, 1H), 2.15-1.91 (m, 3H), 1.91-1.68 (m, 3H), 1.56-1.42 (m, 1H), 1.42-1.31 (m, 1H). LCMS (Method 7): [M+H]⁺ m/z 575.2, RT 1.88 minutes. Chiral analysis (Method 12): RT 2.61 minutes.

### Examples 5 & 6

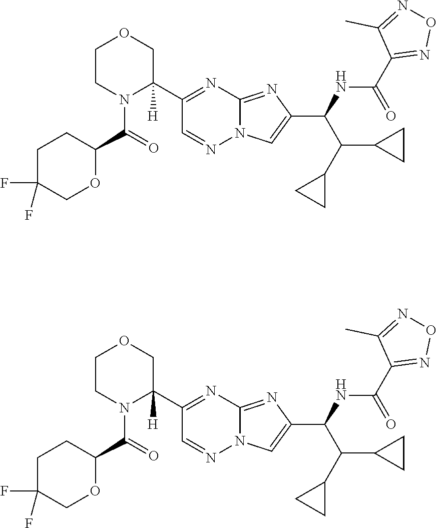 [SMILES: Cc1nonc1C(=O)N[C@H](c1cn2ncc([C@@H]3COCCN3C(=O)[C@@H]3CCC(F)(F)CO3)nc2n1)C(C1CC1)C1CC1 ; Cc1nonc1C(=O)N[C@H](c1cn2ncc([C@H]3COCCN3C(=O)[C@@H]3CCC(F)(F)CO3)nc2n1)C(C1CC1)C1CC1]

### N-[(1S)-2,2-Dicyclopropyl-1-(3-{(3R)-4-[(2S)-5,5-difluorotetrahydropyran-2-carbonyl]-morpholin-3-yl}imidazo[1,2-b][1,2,4]triazin-6-yl)ethyl]-4-methyl-1,2,5-oxadiazole-3-carboxamide

### N-[(1S)-2,2-Dicyclopropyl-1-(3-{(3S)-4-[(2S)-5,5-difluorotetrahydropyran-2-carbonyl]-morpholin-3-yl}imidazo[1,2-b][1,2,4]triazin-6-yl)ethyl]-4-methyl-1,2,5-oxadiazole-3-carboxamide

To a solution of Intermediate 30 (97.4 mg, 0.18 mmol), Intermediate 79 (29.0 mg, 0.17 mmol) and DIPEA (0.12 mL, 0.69 mmol) in DMF (5 mL) at r.t. was added HATU (83.0 mg, 0.21 mmol) in one portion. The mixture was stirred for 20 h, then H₂O (10 mL) was added. The mixture was extracted with EtOAc (3×20 mL) and the combined organic extracts were washed with brine (40 mL), then dried (Na₂SO₄) and concentrated in vacuo. The residue was purified by flash chromatography, eluting with EtOAc/isohexane (0-100% gradient). The resulting material (91.0 mg) was subject to chiral purification (Method 13) to afford, after lyophilisation, the title compounds (Peak 1, 26.0 mg, 25.1% yield, 98.0% d.e.; and Peak 2, 30.0 mg, 29.0% yield, 95.5% d.e.).

Peak 1: δ_{H} (400 MHz, 373K, DMSO-d₆) 8.74 (d, J 9.0 Hz, 1H), 8.70-8.57 (br s, 1H), 8.34-8.27 (m, 1H), 5.65-5.49 (m, 2H), 4.71-4.47 (m, 2H), 4.15-3.73 (m, 5H), 3.66-3.51 (m, 1H), 3.07-2.91 (obscured m, 1H), 2.64-2.45 (obscured s, 3H), 2.32-2.18 (m, 1H), 2.18-1.84 (m, 3H), 1.10 (td, J 8.9, 5.5 Hz, 1H), 0.89-0.75 (m, 2H), 0.51-0.38 (m, 2H), 0.38-0.30 (m, 2H), 0.30-0.22 (m, 2H), 0.21-0.07 (m, 2H). LCMS (Method 7): [M+H]+m/z 587.2, RT 2.10 minutes. Chiral analysis (Method 14): RT 4.46 minutes.

Peak 2: δ_{H} (400 MHz, 373K, DMSO-d₆) 8.73 (d, J 8.9 Hz, 1H), 8.65 (s, 1H), 8.28 (s, 1H), 6.95 (br s, 3H, ammonium adduct), 5.67-5.42 (m, 2H), 4.61 (dd, J 8.1, 4.0 Hz, 1H), 4.56 (app. d, J 12.5 Hz, 1H), 4.30-3.34 (m, 6H), 3.09-2.89 (obscured m, 1H), 2.66-2.42 (obscured s, 3H), 2.32-2.19 (m, 1H), 2.19-1.92 (m, 3H), 1.09 (td, J 9.0, 5.5 Hz, 1H), 0.94-0.73 (m, 2H), 0.52-0.39 (m, 2H), 0.39-0.30 (m, 2H), 0.30-0.22 (m, 2H), 0.20-0.06 (m, 2H). LCMS (Method 7): [M+H]⁺ m/z 587.4, RT 2.08 minutes. Chiral analysis (Method 14): RT 5.02 minutes.

### Examples 7 & 8

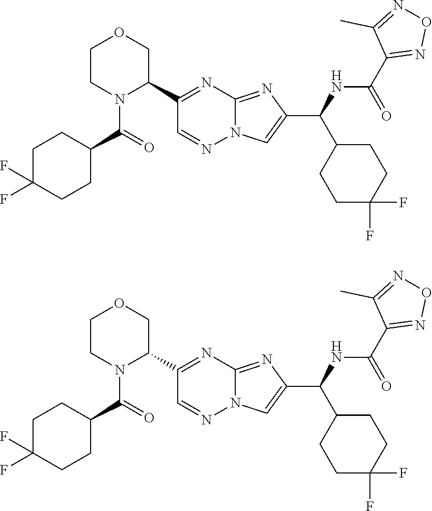

### N—[(S)-(4,4-Difluorocyclohexyl)(3-{(3R)-4-[(2S)-5,5-difluorotetrahydropyran-2-carbonyl]morpholin-3-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{(3S)-4-[(25)-5,5-difluorotetrahydropyran-2-carbonyl]morpholin-3-yl}imidazo[1,2-b][1,2,4]triazin-6-yl)methyl]-4-methyl-1,2,5-oxadiazole-3-carboxamide

To a solution of Intermediate 79 (147 mg, 0.88 mmol), Intermediate 24 (500 mg, 0.80 mmol) and DIPEA (420 μL, 2.41 mmol) in DCM (8 mL) was added HATU (366 mg, 0.96 mmol). The mixture was stirred at r.t for 4 h, then additional Intermediate 79 (20 mg, 0.12 mmol) was added, followed by HATU (40 mg, 0.11 mmol). The solution was stirred at r.t. for 2.5 h, then concentrated under reduced pressure. The residue was purified twice by column chromatography, eluting with a gradient of EtOAc in heptane, to give a first batch (single stereoisomer) (210 mg, 98% purity) as a solid, and a second batch (1:1 mixture of stereoisomers) (180 mg, 94% purity) as a solid. The first batch was subject to HPLC purification (reverse-phase HPLC: 5-95% acetonitrile/water (+0.1% formic acid), X-Bridge, 100×30 mm, 5 μm @ 40 mL/minute) to give one of the title compounds (Diastereomer 1, 92 mg, 19%) as a white solid. The second batch was further purified by reverse-phase HPLC (5-95% acetonitrile/water (+0.1% formic acid), X-Bridge, 100×30 mm, 5 μm @ 40 mL/minute) to give an additional crop of material consisting of the foregoing title compound (Diastereomer 1, 38 mg, 8%) and the other title compound (Diastereomer 2, 38 mg, 8%).

Diastereomer 1 (arbitrarily assigned as Example 7): δ_{H} (400 MHz, DMSO-d₆) 9.55-9.45 (m, 1H), 8.77-8.62 (m, 1H), 8.35-8.29 (m, 1H), 5.61-5.38 (m, 1H), 5.25-5.14 (m, 1H), 4.63-4.50 (m, 2H), 4.27-3.42 (m, 7H), 2.46 (s, 3H), 2.31-1.68 (m, 10H), 1.67-1.56 (m, 1H), 1.47-1.20 (m, 2H). LCMS (Method 2): [M+H]⁺ 611.3, RT 3.32 minutes.

Diastereomer 2 (arbitrarily assigned as Example 8): δ_{H} (400 MHz, DMSO-d₆) 9.61-9.48 (m, 1H), 8.93-8.58 (m, 1H), 8.37-8.28 (m, 1H), 5.65-5.43 (m, 1H), 5.26-5.13 (m, 1H), 4.76-4.23 (m, 2H), 4.17-3.02 (m, 7H), 2.46 (s, 3H), 2.31-1.68 (m, 10H), 1.67-1.56 (m, 1H), 1.46-1.25 (m, 2H). LCMS (Method 2): [M+H]⁺ 611.3, RT 3.32 minutes.

### Examples 9 & 10

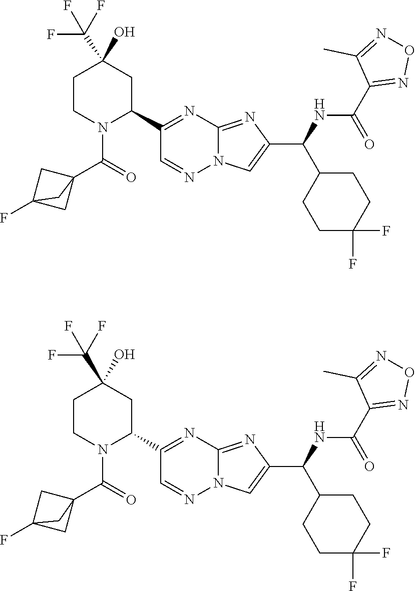 [SMILES: Cc1nonc1C(=O)N[C@H](c1cn2ncc([C@@H]3C[C@@](O)(C(F)(F)F)CCN3C(=O)C34CC(F)(C3)C4)nc2n1)C1CCC(F)(F)CC1 ; Cc1nonc1C(=O)N[C@H](c1cn2ncc([C@H]3C[C@](O)(C(F)(F)F)CCN3C(=O)C34CC(F)(C3)C4)nc2n1)C1CCC(F)(F)CC1]

### N—[(S)-(4,4-Difluorocyclohexyl){3-[(2S,4R)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-4-methyl-1,2,5-oxadiazole-3-carboxamide

### N—[(S)-(4,4-Difluorocyclohexyl){3-[(2R,4S)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-4-methyl-1,2,5-oxadiazole-3-carboxamide

To a solution of Intermediate 35 (60 mg, 0.099 mmol), 4-methyl-1,2,5-oxadiazole-3-carboxylic acid (15 mg, 0.12 mmol) and pyridine (0.032 mL, 0.40 mmol) in DCM (1.5 mL) was added T3P® (50 wt % in EtOAc) (176 μL, 0.30 mmol). The solution was stirred at r.t. for 16 h, then diluted with DCM (20 mL) and washed with saturated aqueous NaHCO₃ solution:water (1:1, 20 mL). The mixture was passed through a hydrophobic frit, washed through with DCM and concentrated under reduced pressure.

The residue was purified by column chromatography, eluting with a gradient of EtOAc in heptane. The resulting material was further purified by chiral HPLC (90:10 heptane:EtOH, Cellulose-4, 21.2×250 mm, 5 μm @ 9 mL/minute) to give the title compounds (Peak 1, 3.7 mg, 6%; and Peak 2, 5.7 mg, 8%).

Peak 1: δ_{H} (400 MHz, DMSO-d₆) 9.55-9.46 (m, 1H), 8.80-8.74 (m, 1H), 8.32-8.23 (m, 1H), 5.97-5.56 (m, 2H), 5.26-5.12 (m, 1H), 4.53-4.01 (m, 1H), 3.80-3.13 (m, 1H), 2.65-2.51 (m, 5H, obs. by DMSO), 2.48-2.44 (m, 3H), 2.32-1.52 (m, 12H), 1.47-1.19 (m, 2H). LCMS (Method 2): [M+H]⁺ 657.3, RT 3.57 minutes. Chiral analysis (Method: 90:10 heptane:EtOH, Cellulose-4, 4.6×250 mm, 5 μm @ 0.5 mL/minute): RT 20.92 minutes.

Peak 2: δ_{H} (400 MHz, DMSO-d₆) 9.56-9.44 (m, 1H), 8.81-8.73 (m, 1H), 8.33-8.23 (m, 1H), 5.96-5.57 (m, 2H), 5.24-5.12 (m, 1H), 4.53-4.02 (m, 1H), 3.81-3.11 (m, 1H), 2.64-2.51 (m, 5H, obs. by DMSO), 2.47-2.44 (m, 3H), 2.31-1.55 (m, 12H), 1.47-1.20 (m, 2H). LCMS (Method 2): [M+H]⁺ 657.3, RT 3.57 minutes. Chiral analysis (Method: 90:10 heptane:EtOH, Cellulose-4, 4.6×250 mm, 5 μm @ 0.5 mL/minute): RT 27.55 minutes.

### Examples 11 & 12

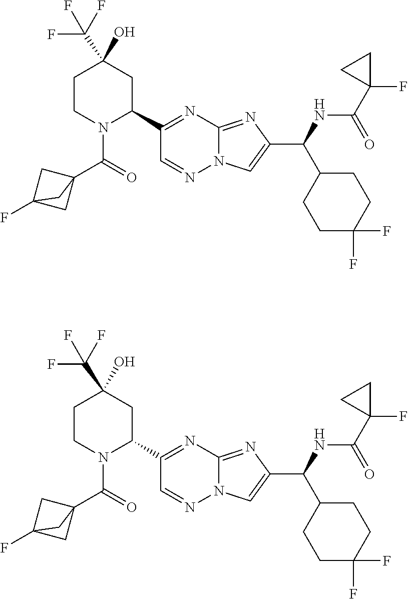 [SMILES: O=C(N[C@H](c1cn2ncc([C@@H]3C[C@@](O)(C(F)(F)F)CCN3C(=O)C34CC(F)(C3)C4)nc2n1)C1CCC(F)(F)CC1)C1(F)CC1 ; O=C(N[C@H](c1cn2ncc([C@H]3C[C@](O)(C(F)(F)F)CCN3C(=O)C34CC(F)(C3)C4)nc2n1)C1CCC(F)(F)CC1)C1(F)CC1]

### N—[(S)-(4,4-Difluorocyclohexyl){3-[(2S,4R)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-1-fluorocyclopropanecarboxamide

### N—[(S)-(4,4-Difluorocyclohexyl){3-[(2R,4S)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-1-fluorocyclopropanecarboxamide

To a solution of Intermediate 35 (60 mg, 0.099 mmol), 1-fluorocyclopropane-1-carboxylic acid (12 mg, 0.119 mmol) and pyridine (0.032 mL, 0.40 mmol) in DCM (1.5 mL) was added T3P® (50 wt % in EtOAc) (176 μL, 0.30 mmol). The solution was stirred at r.t. for 16 h, then diluted with DCM (20 mL) and washed with saturated aqueous Na₂CO₃ solution:water (1:1, 20 mL). The mixture was passed through a hydrophobic frit, washed through with DCM and concentrated under reduced pressure. The residue was purified by column chromatography, eluting with a gradient of EtOAc in heptane. The resulting material was further purified by chiral LC (Method: 90:10 heptane:EtOH, Cellulose-4, 21.2×250 mm, 5 μm @ 18 mL/minute) to give the title compounds (Peak 1, 6.3 mg, 10%; and Peak 2, 12 mg, 19%) as white solids.

Peak 1: δ_{H} (500 MHz, DMSO-d₆) 8.79-8.72 (m, 1H), 8.59-8.50 (m, 1H), 8.27-8.19 (m, 1H), 5.96-5.58 (m, 2H), 5.10-5.00 (m, 1H), 4.54-4.03 (m, 1H), 3.79-3.17 (m, 1H), 2.62-2.51 (m, 5H, obs. by DMSO), 2.33-1.85 (m, 7H), 1.85-1.65 (m, 4H), 1.53 (d, J 14.8 Hz, 1H), 1.39-1.09 (m, 6H). LCMS (Method 2): [M+H]⁺ 633.3, RT 3.36 minutes. Chiral analysis (Method: 90:10 heptane:EtOH, Cellulose-4, 4.6×250 mm, 5 μm @ 0.5 mL/minute): RT 20.78 minutes.

Peak 2: δ_{H} (500 MHz, DMSO-d₆) 8.79-8.72 (m, 1H), 8.62-8.50 (m, 1H), 8.28-8.19 (m, 1H), 5.99-5.59 (m, 2H), 5.12-4.98 (m, 1H), 4.53-4.02 (m, 1H), 3.81-3.17 (m, 1H), 2.63-2.51 (m, 5H, obs. by DMSO), 2.33-1.85 (m, 7H), 1.85-1.64 (m, 4H), 1.63-1.47 (m, 1H), 1.42-1.08 (m, 6H). LCMS (Method 2): [M+H]⁺ 633.3, RT 3.36 minutes. Chiral analysis (Method: 90:10 heptane:EtOH, Cellulose-4, 4.6×250 mm, 5 μm @ 0.5 mL/minute): RT 28.62 minutes.

### Example 13

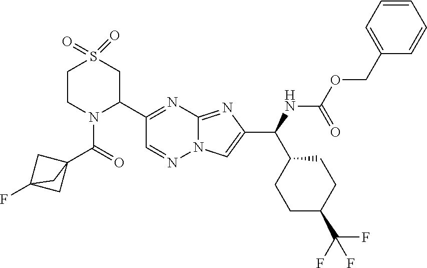 [SMILES: O=C(N[C@H](c1cn2ncc(C3CS(=O)(=O)CCN3C(=O)C34CC(F)(C3)C4)nc2n1)[C@H]1CC[C@H](C(F)(F)F)CC1)OCc1ccccc1]

### Benzyl N—[(S)-{3-[4-(3-fluorobicyclo[1.1.1]pentane-1-carbonyl)-1,1-dioxo-1,4-thiazinan-3-yl]imidazo[1,2-b][1,2,4]triazin-6-yl}[4-(trifluoromethyl)cyclohexyl]methyl]carbamate

To a solution of 3-fluorobicyclo[1.1.1]pentane-1-carboxylic acid (117 mg, 0.855 mmol) in anhydrous DMA (2 mL) was added DIPEA (113 μL, 0.765 mmol), followed by 2-chloro-1-methylpyridinium iodide (228 mg, 0.866 mmol). The resultant yellow suspension was stirred at ambient temperature for 10 minutes, then a solution of Intermediate 46 (170 mg, 0.300 mmol) in DMA (3 mL) was added. The reaction mixture was stirred at ambient temperature for 72 h, then partitioned between EtOAc (20 mL) and water (50 mL). The organic phases were separated, and the aqueous phase was extracted with EtOAc (2×30 mL). The combined organic extracts were washed with brine and dried over anhydrous Na₂SO₄, then filtered and concentrated in vacuo. The crude residue was purified by column chromatography on silica, eluting with EtOAc (0-100% in hexanes), to provide the title compound (120 mg, 59%) as a pale yellow solid. δ_{H} (400 MHz, DMSO-d₆) 8.91 (dd, J 43.4, 2.6 Hz, 1H), 8.31-8.02 (m, 1H), 7.79 (d, J 9.0 Hz, 1H), 7.56-6.97 (m, 5H), 6.42 (s, 1H), 5.24-4.80 (m, 2H), 4.65 (q, J 8.3 Hz, 1H), 4.52-4.13 (m, 2H), 4.03-3.69 (m, 2H), 3.41 (1H, m, obs by DMSO), 3.10 (d, J 13.2 Hz, 1H), 2.43-2.07 (m, 6H), 1.89 (m, 4H), 1.61 (d, J 14.1 Hz, 1H), 1.20 (m, 5H). LCMS (Method 7): [M+H]⁺ 679, RT 2.23 minutes.

### Examples 14 & 15

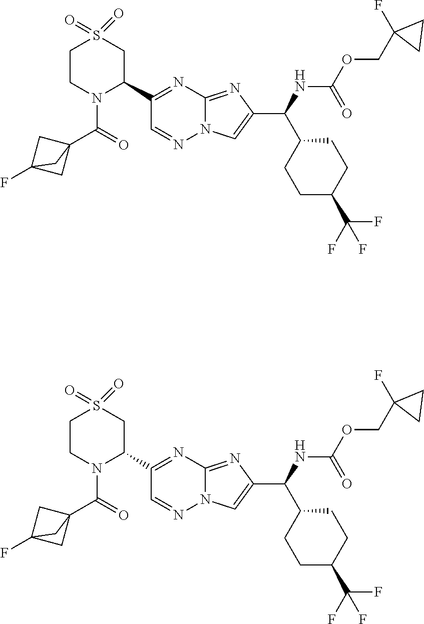 [SMILES: O=C(N[C@H](c1cn2ncc([C@@H]3CS(=O)(=O)CCN3C(=O)C34CC(F)(C3)C4)nc2n1)[C@H]1CC[C@H](C(F)(F)F)CC1)OCC1(F)CC1 ; O=C(N[C@H](c1cn2ncc([C@H]3CS(=O)(=O)CCN3C(=O)C34CC(F)(C3)C4)nc2n1)[C@H]1CC[C@H](C(F)(F)F)CC1)OCC1(F)CC1]

### 1-Fluoro-N—[(S)-{3-[(3R)-4-(3-fluorobicyclo[1.1.1]pentane-1-carbonyl)-1,1-dioxo-1,4-thiazinan-3-yl]imidazo[1,2-b][1,2,4]triazin-6-yl}[4-(trifluoromethyl)cyclohexyl]methyl]-cyclopropanecarboxamide

### 1-Fluoro-N—[(S)-{3-[(3S)-4-(3-fluorobicyclo[1.1.1]pentane-1-carbonyl)-1,1-dioxo-1,4-thiazinan-3-yl]imidazo[1,2-b][1,2,4]triazin-6-yl}[4-(trifluoromethyl)cyclohexyl]methyl]-cyclopropanecarboxamide

To a solution of Intermediate 47 (59 mg, 0.108 mmol) in anhydrous DMF (2 mL) were added 1-fluorocyclopropanecarboxylic acid (35 mg, 0.333 mmol), DIPEA (75 μL, 0.431 mmol) and HATU (83 mg, 0.212 mmol) sequentially. The resultant yellow solution was stirred at ambient temperature for 18 h, then diluted with EtOAc (20 mL) and water (20 mL). The organic phase was separated, and the aqueous phase was extracted with additional EtOAc (2×50 mL). The combined organic phases were washed with brine (2×100 mL) and dried over anhydrous Na₂SO₄, then filtered and concentrated in vacuo. The crude residue was purified by column chromatography on silica (gradient elution, 0-80% EtOAc in hexanes) and freeze-dried from acetonitrile/water. The resulting pale yellow solid was subject to chiral separation (Method 15) to yield the title compounds (Peak 1, 17 mg, 25% yield, 82% d.e.; and Peak 2, 15 mg, 22% yield, 85% d.e.).

Peak 1: δ_{H} (400 MHz, DMSO-d₆) 9.14-8.80 (m, 1H), 8.66-8.43 (m, 1H), 8.39-8.11 (m, 1H), 6.69-5.80 (m, 1H), 4.97 (m, 1.5H), 4.59-4.11 (m, 1.5H), 4.08-3.69 (m, 2H), 3.50-3.30 (1H, m, obs by DMSO), 3.10 (d, J 12.9 Hz, 1H), 2.45-2.13 (m, 5H), 2.11-1.71 (m, 4H), 1.69-1.47 (m, 1H), 1.43-0.93 (m, 10H). Mixture of rotamers in ˜3:2 ratio observed in ¹H NMR spectrum. LCMS (Method 7): [M+H]⁺ 631, RT 1.95 minutes. Chiral analysis (Method 16): RT 3.97 minutes.

Peak 2: δ_{H} (400 MHz, DMSO-d₆) 9.14-8.80 (m, 1H), 8.66-8.43 (m, 1H), 8.39-8.11 (m, 1H), 6.69-5.80 (m, 1H), 4.97 (m, 1.5H), 4.59-4.11 (m, 2H), 4.08-3.69 (m, 2H), 3.50-3.30 (1H, m, obs by DMSO), 3.10 (d, J 12.9 Hz, 1H), 2.45-2.13 (m, 5H), 2.11-1.71 (m, 4H), 1.69-1.47 (m, 1H), 1.43-0.93 (m, 10H). Mixture of rotamers in ˜3:2 ratio observed in ¹H NMR spectrum. LCMS (Method 7): [M+H]⁺ 631, RT 1.95 minutes. Chiral analysis (Method 16): RT 4.44 minutes.

### Example 16

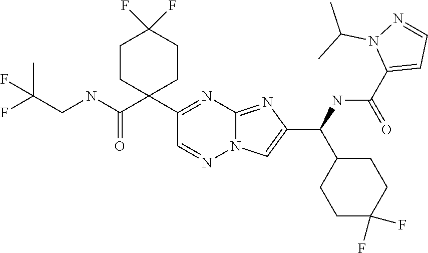 [SMILES: CC(C)n1nccc1C(=O)N[C@H](c1cn2ncc(C3(C(=O)NCC(C)(F)F)CCC(F)(F)CC3)nc2n1)C1CCC(F)(F)CC1]

### N—[(S)-(4,4-Difluorocyclohexyl){3-[1-(2,2-difluoropropylcarbamoyl)-4,4-difluoro-cyclohexyl]imidazo[1,2-b][1,2,4]triazin-6-yl}methyl]-2-isopropylpyrazole-3-carboxamide

To a solution containing Intermediate 54 (150 mg, 0.30 mmol), 1-isopropyl-1H-pyrazole-5-carboxylic acid (58 mg, 0.36 mmol) and DIPEA (0.16 mL, 0.92 mmol) in DMF (2 mL) was added HATU (140 mg, 0.36 mmol). The reaction mixture was stirred at r.t. for 1 h, then diluted with EtOAc (15 mL) and water (5 mL). The aqueous layer was extracted with EtOAc (3×15 mL) and the combined organic extracts were concentrated in vacuo. The crude material was purified by SFC preparative chromatography (Method 17) to give the title compound (41 mg, 21.5%) as a white solid. δ_{H} (400 MHz, DMSO-d₆) 8.82 (dd, J 9.0, 2.0 Hz, 1H), 8.69 (d, J 2.0 Hz, 1H), 8.30 (d, J 2.0 Hz, 1H), 8.17 (t, J 6.1 Hz, 1H), 7.50 (d, J 2.2 Hz, 1H), 6.96 (t, J 2.1 Hz, 1H), 5.39 (pd, J 6.6, 2.0 Hz, 1H), 5.21 (td, J 8.7, 2.0 Hz, 1H), 3.50 (dt, J 12.6, 9.5 Hz, 2H), 2.55 (s, 1H), 2.43-2.33 (m, 4H), 2.22 (d, J 10.3 Hz, 1H), 2.11-1.98 (m, 6H), 1.92 (d, J 13.1 Hz, 1H), 1.85-1.72 (m, 1H), 1.66 (d, J 13.2 Hz, 1H), 1.57-1.48 (m, 2H), 1.48-1.23 (m, 7H). LCMS (Method 7): [M+H]⁺ 643.4, RT 2.05 minutes.

### Example 17

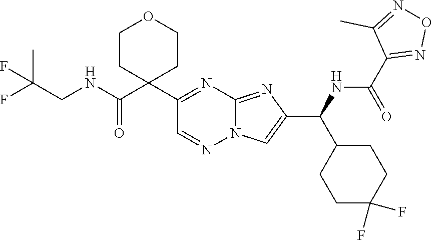 [SMILES: Cc1nonc1C(=O)N[C@H](c1cn2ncc(C3(C(=O)NCC(C)(F)F)CCOCC3)nc2n1)C1CCC(F)(F)CC1]

### N—[(S)-(4,4-Difluorocyclohexyl){3-[4-(2,2-difluoropropylcarbamoyl)tetrahydropyran-4-yl]imidazo[1,2-b][1,2,4]triazin-6-yl}methyl]-4-methyl-1,2,5-oxadiazole-3-carboxamide

DIPEA (33 μL, 0.186 mmol) was added to a stirred solution of Intermediate 61 (89%, 33 mg, 0.06 mmol), 4-methyl-1,2,5-oxadiazole-3-carboxylic acid (10 mg, 0.075 mmol) and HATU (35 mg, 0.0932 mmol) in DMF (2 mL) at r.t. The reaction mixture was stirred at r.t. for 45 minutes, then diluted with EtOAc (7 mL) and quenched with saturated aqueous NaHCO₃ solution (7 mL). The biphasic mixture was stirred at r.t. for 20 minutes. The layers were separated, and the aqueous phase was extracted with EtOAc (2×7 mL). The combined organic extracts were dried over MgSO₄, then filtered and concentrated in vacuo. The residue was purified by acidic open access preparative HPLC (Gilson 4, Standard Method; Column: Sunfire™ Prep. C18 10 μm OBDTM, 30×100 mm; Mobile Phase: 30-95% acetonitrile (0.1% formic acid) in water (0.1% formic acid) over 10 minutes; Flow Rate: 40 mL/minute; UV: 215 and 254 nm) to afford, after freeze-drying, the title compound (19 mg, 52%) as a white solid. δ_{H} (400 MHz, DMSO-d₆) 9.52 (d, J 9.0 Hz, 1H), 8.66 (s, 1H), 8.31 (s, 1H), 8.18 (t, J 6.2 Hz, 1H), 5.21 (t, J 8.5 Hz, 1H), 3.75-3.65 (m, 2H), 3.65-3.56 (m, 2H), 3.56-3.45 (m, 2H), 2.47 (s, 3H), 2.45-2.35 (m, 2H), 2.28-2.14 (m, 3H), 2.13-1.97 (m, 2H), 1.97-1.88 (m, 1H), 1.88-1.68 (m, 2H), 1.68-1.59 (m, 1H), 1.49 (t, J 19.0 Hz, 3H), 1.43-1.18 (m, 2H). LCMS (Method 2): [M+H]⁺ 583, RT 3.23 minutes.

### Examples 18 & 19

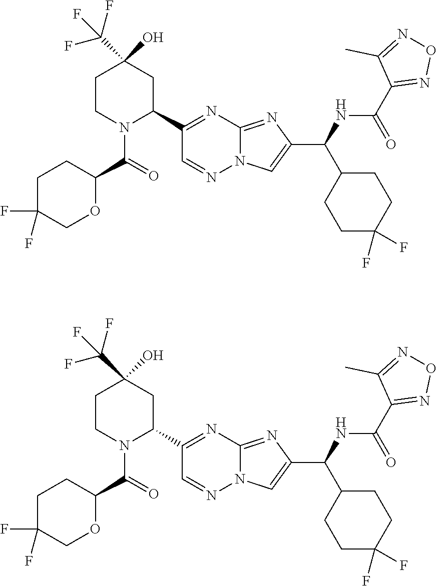 [SMILES: Cc1nonc1C(=O)N[C@H](c1cn2ncc([C@@H]3C[C@@](O)(C(F)(F)F)CCN3C(=O)[C@@H]3CCC(F)(F)CO3)nc2n1)C1CCC(F)(F)CC1 ; Cc1nonc1C(=O)N[C@H](c1cn2ncc([C@H]3C[C@](O)(C(F)(F)F)CCN3C(=O)[C@@H]3CCC(F)(F)CO3)nc2n1)C1CCC(F)(F)CC1]

### N—[(S)-(4,4-Difluorocyclohexyl)(3-{(2S,4R)-1-[(2S)-5,5-difluorotetrahydropyran-2-carbonyl]-4-hydroxy-4-(trifluoromethyl)piperidin-2-yl}imidazo[1,2-b][1,2,4]triazin-6-yl)-methyl]-4-methyl-1,2,5-oxadiazole-3-carboxamide

### N—[(S)-(4,4-Difluorocyclohexyl)(3-{(2R,4S)-1-[(2S)-5,5-difluorotetrahydropyran-2-carbonyl]-4-hydroxy-4-(trifluoromethyl)piperidin-2-yl}imidazo[1,2-b][1,2,4]triazin-6-yl)-methyl]-4-methyl-1,2,5-oxadiazole-3-carboxamide

To a solution of Intermediate 63 (two stereoisomers in 1:1 ratio) (85% purity) (82.0 mg, 0.14 mmol), 4-methyl-1,2,5-oxadiazole-3-carboxylic acid (18.0 mg, 0.14 mmol) and DIPEA (0.10 mL, 0.58 mmol) in DMF (5 mL) at r.t. was added HATU (67.0 mg, 0.17 mmol) in one portion. The mixture was stirred for 20 minutes, then H₂O (20 mL) was added. The mixture was extracted with EtOAc (3×20 mL). The combined organic extracts were washed with brine (40 mL), then dried (Na₂SO₄) and concentrated in vacuo. The residue was purified by flash chromatography, eluting with EtOAc/isohexane (0-100% gradient). Further chiral purification of the resulting white foam (77.0 mg) was performed by SFC (Lux Cellulose-1 250×21.2 mm, 5 μm column, flow rate 100 mL/minute, column temperature 40° C., eluting with a 3-40% MeOH (+0.1% NH₄+OH) method (ABPR 60 bar), using a 10 minute run time on a Waters Prep 150 fractionlynx system, in tandem with a Waters QDa mass spectrometer) to afford, after lyophilisation, the separated title compounds (Peak 1, 15.0 mg, 15.4% yield, 96.7% d.e.; and Peak 2, 14.0 mg, 14.4% yield, 95.7% d.e.).

Peak 1 (mixture of rotamers in 2.2:1 ratio by ¹H NMR): δ_{H} (400 MHz, DMSO-d₆) 9.51 (d, J 9.3 Hz, 1H, minor rotamer), 9.48 (d, J 9.0 Hz, 1H, major rotamer), 8.81 (s, 1H, major rotamer), 8.71 (s, 1H, minor rotamer), 8.28 (s, 1H), 5.94-5.89 (m, 1H, major rotamer), 6.11-5.81 (v br s, 1H), 5.65-5.60 (m, 1H, minor rotamer), 5.20 (obscured t, J 8.8 Hz, 1H, minor rotamer), 5.18 (t, J 8.7 Hz, 1H, major rotamer), 4.75-4.68 (m, 1H, major rotamer), 4.61-4.52 (m, 2H, 2× minor rotamers), 4.15-4.05 (m, 1H, major rotamer), 4.02-3.82 (m, 1H and 1H, major rotamer), 3.74-3.63 (m, 1H, major rotamer), 3.58-3.43 (m, 2H, 2× minor rotamers), 2.70-2.57 (obscured m, 1H), 2.47 (s, 3H), 2.32-1.57 (m, 14H), 1.48-1.21 (m, 2H). LCMS (Method 7): [M+H]⁺ m/z 693.2, RT 2.01 minutes. Chiral analysis (Method 10): RT 3.15 minutes.

Peak 2 (mixture of rotamers in 1.4:1 ratio by ¹H NMR): δ_{H} (400 MHz, DMSO-d₆) 9.51 (d, J 8.9 Hz, 1H, minor rotamer), 9.50 (d, J 8.9 Hz, 1H, major rotamer), 8.87 (s, 1H, minor rotamer), 8.71 (s, 1H, major rotamer), 8.28 (s, 1H, minor rotamer), 8.26 (s, 1H, major rotamer), 6.11-5.84 (v br s, 1H), 5.94 (d, J 6.9 Hz, 1H, major rotamer), 5.73 (d, J 6.2 Hz, 1H, minor rotamer), 5.19 (t, J 8.5 Hz, 2H, major and minor rotamers), 4.72-4.66 (m, 1H, major rotamer), 4.51-4.42 (m, 1H, minor rotamer), 4.33-4.25 (m, 1H, minor rotamer), 4.16-3.99 (m, 2H, 2× major rotamers), 3.98-3.69 (m, 1H and 2H, major and minor rotamers), 3.25-3.12 (m, 1H, minor rotamer), 2.79 (app d, J 14.4 Hz, 1H, minor rotamer), 2.62 (app d, J 14.2 Hz, 1H, major rotamer), 2.47 (s, 3H), 2.31-1.57 (m, 14H), 1.47-1.22 (m, 2H). LCMS (Method 7): [M+H]⁺ m/z 693.2, RT 2.02 minutes. Chiral analysis (Method 10): RT 3.74.

### Examples 20 & 21

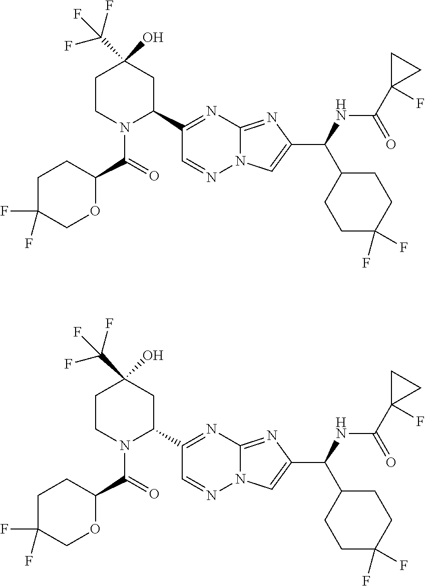 [SMILES: O=C([C@@H]1CCC(F)(F)CO1)N1CC[C@](O)(C(F)(F)F)C[C@H]1c1cnn2cc([C@@H](NC(=O)C3(F)CC3)C3CCC(F)(F)CC3)nc2n1 ; O=C([C@@H]1CCC(F)(F)CO1)N1CC[C@@](O)(C(F)(F)F)C[C@@H]1c1cnn2cc([C@@H](NC(=O)C3(F)CC3)C3CCC(F)(F)CC3)nc2n1]

### N—[(S)-(4,4-Difluorocyclohexyl)(3-{(2S,4R)-1-[(2S)-5,5-difluorotetrahydropyran-2-carbonyl]-4-hydroxy-4-(trifluoromethyl)piperidin-2-yl}imidazo[1,2-b][1,2,4]triazin-6-yl)-methyl]-1-fluorocyclopropanecarboxamide

### N—[(S)-(4,4-Difluorocyclohexyl)(3-{(2R,4S)-1-[(2S)-5,5-difluorotetrahydropyran-2-carbonyl]-4-hydroxy-4-(trifluoromethyl)piperidin-2-yl}imidazo[1,2-b][1,2,4]triazin-6-yl)-methyl]-1-fluorocyclopropanecarboxamide

To a solution of Intermediate 63 (two stereoisomers in 1:1 ratio) (85% purity) (80.0 mg, 0.14 mmol), 1-fluorocyclopropanecarboxylic acid (15.0 mg, 0.14 mmol) and DIPEA (0.10 mL, 0.58 mmol) in DMF (5 mL) at r.t. was added HATU (65.0 mg, 0.17 mmol) in one portion. The mixture was stirred for 20 minutes, then H₂O (20 mL) was added. The mixture was extracted with EtOAc (3×20 mL). The combined organic extracts were washed with brine (40 mL), then dried (Na₂SO₄) and concentrated in vacuo. The residue was purified by flash chromatography, eluting with EtOAc/isohexane (0-100% gradient). Further chiral purification of the resulting white foam (77 mg) was performed by SFC (Lux Cellulose-1 250×21.2 mm, 5 μm column, flow rate 100 mL/minute, column temperature 40° C., eluting with a 3-40% MeOH (+0.1% NH₄OH) method (ABPR 60 bar), using a 10 minute run time on a Waters Prep 150 fractionlynx system, in tandem with a Waters QDa mass spectrometer) to afford, after lyophilisation, the separated title compounds (Peak 1, 20.0 mg, 21.8% yield, 98.6% d.e.; and Peak 2, 17.0 mg, 18.5% yield, 98.5% d.e.).

Peak 1 (mixture of rotamers in 2.6:1 ratio by ¹H NMR): δ_{H} (400 MHz, DMSO-d₆) 8.81 (s, 1H, major rotamer), 8.70 (s, 1H, minor rotamer), 8.56 (d, J 9.0 Hz, minor rotamer), 8.53 (d, J 9.5 Hz, major rotamer), 8.24 (s, 1H), 5.92 (app d, J 6.8 Hz, 1H, major rotamer), 5.94-5.81 (v br s, 1H), 5.63 (app d, J 6.3 Hz, 1H, minor rotamer), 5.07 (obscured t, J 9.0 Hz, 1H, minor rotamer), 5.05 (t, J 8.9 Hz, 1H, major rotamer), 4.72 (t, J 6.4 Hz, 1H, major rotamer), 4.62-4.52 (m, 2H, 2× minor rotamers), 4.15-4.05 (m, 1H, major rotamer), 4.02-3.82 (m, 1H and 1H, major rotamer), 3.76-3.65 (m, 1H, major rotamer), 3.60-3.42 (m, 2H, 2× minor rotamers), 2.70-2.56 (obscured m, 1H), 2.32-1.63 (m, 13H), 1.62-1.49 (m, 1H), 1.40-1.11 (m, 6H). LCMS (Method 7): [M+H]⁺ m/z 669.2, RT 1.92 minutes. Chiral analysis (Method 10): RT 3.00 minutes.

Peak 2 (mixture of rotamers in 1.3:1 ratio by ¹H NMR): δ_{H} (400 MHz, DMSO-d₆) 8.86 (s, 1H, minor rotamer), 8.70 (s, 1H, major rotamer), 8.54 (app t, J 8.7 Hz, 2H, major and minor rotamers), 8.24 (s, 1H, minor rotamer), 8.23 (s, 1H, major rotamer), 5.97-5.91 (m, 1H, major rotamer), 5.92-5.85 (v br s, 1H), 5.76-5.70 (m, 1H, minor rotamer), 5.06 (t, J 8.5 Hz, 1H, major rotamer), 5.05 (t, J 8.9 Hz, 1H, minor rotamer), 4.73-4.66 (m, 1H, major rotamer), 4.52-4.42 (m, 1H, minor rotamer), 4.34-4.26 (m, 1H, minor rotamer), 4.17-4.00 (m, 2H, 2× major rotamers), 3.97-3.68 (m, 1H and 2H, major and minor rotamers), 3.27-3.16 (m, 1H, minor rotamer), 2.78 (app d, J 14.4 Hz, 1H, minor rotamer), 2.62 (app d, J 14.2 Hz, 1H, major rotamer), 2.32-1.63 (m, 13H), 1.62-1.50 (m, 1H), 1.42-1.09 (m, 6H). LCMS (Method 7): [M+H]⁺ m/z 669.2, RT 1.91 minutes. Chiral analysis (Method 10): 3.54 minutes.

### Examples 22 & 23

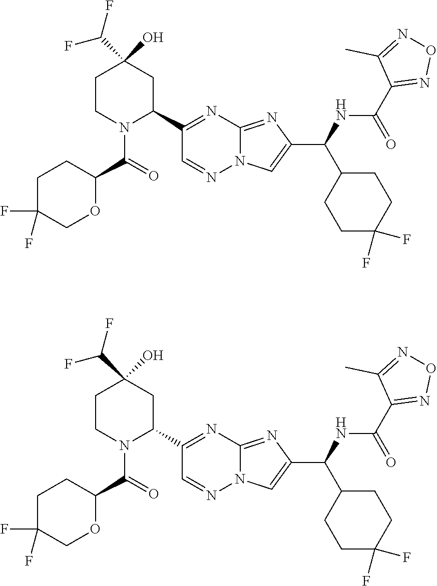 [SMILES: Cc1nonc1C(=O)N[C@H](c1cn2ncc([C@@H]3C[C@@](O)(C(F)F)CCN3C(=O)[C@@H]3CCC(F)(F)CO3)nc2n1)C1CCC(F)(F)CC1 ; Cc1nonc1C(=O)N[C@H](c1cn2ncc([C@H]3C[C@](O)(C(F)F)CCN3C(=O)[C@@H]3CCC(F)(F)CO3)nc2n1)C1CCC(F)(F)CC1]

### N—[(S)-(4,4-Difluorocyclohexyl)(3-{(2S,4R)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)-methyl]-4-methyl-1,2,5-oxadiazole-3-carboxamide

### N—[(S)-(4,4-Difluorocyclohexyl)(3-{(2R,4S)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)-methyl]-4-methyl-1,2,5-oxadiazole-3-carboxamide

To a solution of Intermediate 72 (two stereoisomers in 1:1 ratio) (124 mg, 0.22 mmol), 4-methyl-1,2,5-oxadiazole-3-carboxylic acid (28.0 mg, 0.22 mmol) and DIPEA (0.16 mL, 0.92 mmol) in DMF (5 mL) at r.t. was added HATU (104 mg, 0.27 mmol) in one portion. The mixture was stirred for 45 minutes, then H₂O (20 mL) was added. The mixture was extracted with EtOAc (3×20 mL). The combined organic extracts were washed with brine (40 mL), then dried (Na₂SO₄) and concentrated in vacuo. The residue was purified by flash chromatography, eluting with EtOAc/isohexane (0-100% gradient). Further chiral purification of the resulting white foam (140 mg) was performed by SFC (Lux Cellulose-1 250×21.2 mm, 5 μm column, flow rate 100 mL/minute, column temperature 40° C., eluting with a 3-40% MeOH (+0.1% NH+OH) method (ABPR 60 bar), using a 10 minute run time on a Waters Prep 150 fractionlynx system, in tandem with a Waters QDa mass spectrometer) to afford, after lyophilisation, the separated title compounds (Peak 1, 37.0 mg, 25% yield, 98.9% d.e.; and Peak 2, 27.0 mg, 18.2% yield, 94.2% d.e.).

Peak 1 (mixture of rotamers in 2.8:1 ratio by ¹H NMR): δ_{H} (400 MHz, DMSO-d₆) 9.50 (d, J 9.4 Hz, 1H, minor rotamer), 9.47 (d, J 8.9 Hz, 1H, major rotamer), 8.76 (s, 1H, major rotamer), 8.65 (s, 1H, minor rotamer), 8.26 (s, 1H), 5.90-5.84 (m, 1H, major rotamer), 5.78 (t, J 56.0 Hz, 1H, major rotamer), 5.75 (t, J 56.1 Hz, 1H, minor rotamer), 5.60-5.55 (m, 1H, minor rotamer), 5.26-5.07 (obscured v br s, 1H), 5.18 (app t, J 8.8 Hz, 2H, major and minor rotamers), 4.70 (app t, J 6.3 Hz, 1H, major rotamer), 4.61-4.44 (m, 2H, 2× minor rotamers), 4.11-3.80 (m, 1H and 2H, 2× major rotamers), 3.73-3.60 (m, 1H, major rotamer), 3.59-3.40 (m, 2H, 2× minor rotamers), 2.64-2.41 (obscured m, 1H), 2.47 (s, 3H), 2.31-1.51 (m, 14H), 1.47-1.21 (m, 2H). LCMS (Method 7): [M+H]⁺ m/z 675.2, RT 1.88 minutes. Chiral analysis (Method 10): RT 3.52 minutes.

Peak 2 (mixture of rotamers in 1.3:1 ratio by ¹H NMR): δ_{H} (400 MHz, DMSO-d₆) 9.51 (d, J 8.9 Hz, 1H, minor rotamer), 9.50 (d, J 9.0 Hz, 1H, major rotamer), 8.80 (s, 1H, minor rotamer), 8.65 (s, 1H, major rotamer), 8.27 (s, 1H, minor rotamer), 8.25 (s, 1H, major rotamer), 5.91-5.86 (m, 1H, major rotamer), 5.78 (t, J 55.7 Hz, 1H, minor rotamer), 5.77 (t, J 56.0 Hz, 1H, major rotamer), 5.69-5.65 (m, 1H, minor rotamer), 5.28-5.10 (obscured v br s, 1H), 5.19 (t, J 8.6 Hz, 1H, major rotamer), 5.19 (t, J 8.8 Hz, 1H, minor rotamer), 4.72-4.65 (m, 1H, major rotamer), 4.46-4.36 (m, 1H, minor rotamer), 4.31-4.23 (m, 1H, minor rotamer), 4.11-3.98 (m, 2H, 2× major rotamers), 3.94-3.67 (m, 1H and 2H, major and minor rotamers), 3.22-3.10 (m, 1H, minor rotamer), 2.67-2.42 (obscured m, 1H), 2.47 (s, 3H), 2.32-1.52 (m, 14H), 1.47-1.22 (m, 2H). LCMS (Method 7): [M+H]⁺ m/z 675.2, RT 1.89 minutes. Chiral analysis (Method 10): RT 4.04 minutes.

### Examples 24 & 25

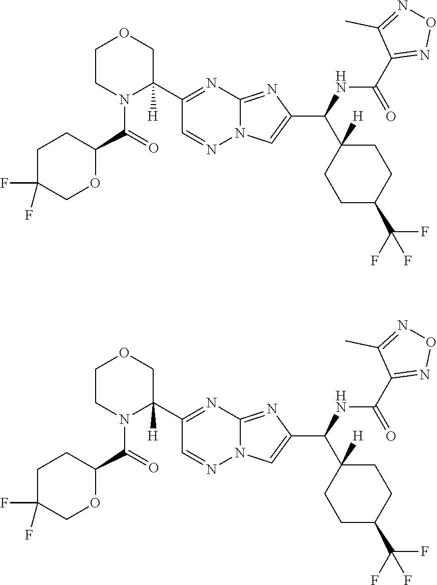 [SMILES: Cc1nonc1C(=O)N[C@H](c1cn2ncc([C@@H]3COCCN3C(=O)[C@@H]3CCC(F)(F)CO3)nc2n1)[C@H]1CC[C@H](C(F)(F)F)CC1 ; Cc1nonc1C(=O)N[C@H](c1cn2ncc([C@H]3COCCN3C(=O)[C@@H]3CCC(F)(F)CO3)nc2n1)[C@H]1CC[C@H](C(F)(F)F)CC1]

### N—{(S)-(3-{(3R)-4-[(2S)-5,5-Difluorotetrahydropyran-2-carbonyl]morpholin-3-yl}-imidazo[1,2-b][1,2,4]triazin-6-yl)[4-(trifluoromethyl)cyclohexyl]methyl}-4-methyl-1,2,5-oxadiazole-3-carboxamide

### N—{(S)-(3-{(3S)-4-[(2S)-5,5-Difluorotetrahydropyran-2-carbonyl]morpholin-3-yl}-imidazo[1,2-b][1,2,4]triazin-6-yl)[4-(trifluoromethyl)cyclohexyl]methyl}-4-methyl-1,2,5-oxadiazole-3-carboxamide

To a solution of Intermediate 76 (two stereoisomers in 1:1 ratio) (91.0 mg, 0.15 mmol), Intermediate 79 (25.0 mg, 0.15 mmol) and DIPEA (0.10 mL, 0.58 mmol) in DMF (5 mL) at r.t. was added HATU (71.0 mg, 0.18 mmol) in one portion. The mixture was stirred for 16 h, then H₂O (20 mL) was added. The mixture was extracted with EtOAc (3×20 mL). The combined organic extracts were washed with brine (40 mL), then dried (Na₂SO₄) and concentrated in vacuo. The residue was purified by flash chromatography, eluting with EtOAc/isohexane (0-100% gradient). Further chiral purification of the resulting white foam (57.0 mg) was performed (using a (R,R) Whelk-01 250×10 mm, 5 μm column, flow rate 4.7 mL/minute, column temperature ambient, eluting with a 40% EtOH:60% n-heptane (+0.1% diethylamine) isocratic method, using a 19 minute run time on a UV directed Agilent 1100/1200 hybrid system) to afford, after lyophilisation, the separated title compounds (Peak 1, 7.3 mg, 7.6% yield, >99% d.e.; and Peak 2, 12.0 mg, 12.5% yield, 99% d.e.).

Peak 1: δ_{H} (400 MHz, 373K, DMSO-d₆) 8.96 (d, J 8.8 Hz, 1H), 8.65 (s, 1H), 8.24 (s, 1H), 5.64-5.39 (v br s, 1H), 5.18 (t, J 8.2 Hz, 1H), 4.61 (dd, J 8.2, 4.0 Hz, 1H), 4.56 (app d, J 12.2 Hz, 1H), 4.12-3.44 (m, 6H), 3.07-2.93 (obscured m, 1H), 2.49 (s, 3H), 2.31-1.85 (m, 9H), 1.81-1.71 (m, 1H), 1.37-1.16 (m, 4H). LCMS (Method 7): [M+H]⁺ m/z 643.2, RT 2.07 minutes. Chiral analysis (Method 18): RT 3.75 minutes.

Peak 2: δ_{H} (400 MHz, 373K, DMSO-d₆) 8.98 (d, J 8.8 Hz, 1H), 8.74-8.53 (v br s, 1H), 8.25 (s, 1H), 5.61-5.50 (br s, 1H), 5.18 (t, J 8.2 Hz, 1H), 4.71-4.42 (m, 2H), 4.15-3.73 (m, 5H), 3.67-3.51 (m, 1H), 3.07-2.93 (obscured m, 1H), 2.49 (s, 3H), 2.31-1.85 (m, 9H), 1.81-1.70 (m, 1H), 1.38-1.13 (m, 4H). LCMS (Method 7): [M+H]⁺ m/z 643.2, RT 2.09 minutes. Chiral analysis (Method 18): RT 4.80 minutes.

### Examples 26 & 27

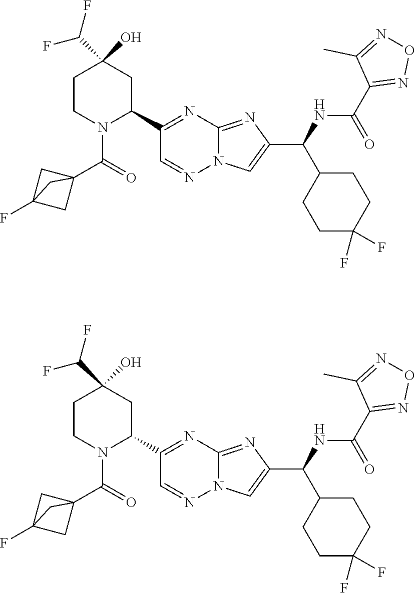 [SMILES: Cc1nonc1C(=O)N[C@H](c1cn2ncc([C@@H]3C[C@@](O)(C(F)F)CCN3C(=O)C34CC(F)(C3)C4)nc2n1)C1CCC(F)(F)CC1 ; Cc1nonc1C(=O)N[C@H](c1cn2ncc([C@H]3C[C@](O)(C(F)F)CCN3C(=O)C34CC(F)(C3)C4)nc2n1)C1CCC(F)(F)CC1]

### N—[(S)-(4,4-Difluorocyclohexyl){3-[(2S,4R)-4-(difluoromethyl)-1-(3-fluorobicyclo[1.1.1]-pentane-1-carbonyl)-4-hydroxypiperidin-2-yl]imidazo[1,2-b][1,2,4]triazin-6-yl}methyl]-4-methyl-1,2,5-oxadiazole-3-carboxamide

### N—[(S)-(4,4-Difluorocyclohexyl){3-[(2R,4S)-4-(difluoromethyl)-1-(3-fluorobicyclo[1.1.1]-pentane-1-carbonyl)-4-hydroxypiperidin-2-yl]imidazo[1,2-b][1,2,4]triazin-6-yl}methyl]-4-methyl-1,2,5-oxadiazole-3-carboxamide

To a solution of Intermediate 82 (mixture of two syn stereoisomers in 1:1 ratio) (303 mg, 0.54 mmol), 3-fluorobicyclo[1.1.1]pentane-1-carboxylic acid (81.0 mg, 0.59 mmol) and DIPEA (0.37 mL, 2.10 mmol) in DMF (10 mL) at r.t. was added HATU (253 mg, 0.65 mmol) in one portion. The mixture was stirred for 5 minutes, then H₂O (30 mL) was added. The mixture was extracted with EtOAc (3×30 mL), and the combined organic layers were washed with brine (40 mL), then dried (Na₂SO₄) and concentrated in vacuo. The residue was purified by flash chromatography, eluting with EtOAc/isohexane (0-100% gradient). The resulting white foam (184 mg) was subjected to chiral purification (Method 19) to afford, after lyophilisation, the title compounds (Peak 1, 44.0 mg, 12.8% yield, >99% d.e.; and Peak 2, 47.0 mg, 13.7% yield, 96.5% d.e.).

Peak 1 (mixture of rotamers in 1.6:1 ratio by ¹H NMR): δ_{H} (400 MHz, DMSO-d₆) 9.52 (d, J 9.0 Hz, 1H, major and minor rotamers), 8.73 (s, 1H, minor rotamer), 8.71 (s, 1H, major rotamer), 8.28 (s, 1H, minor rotamer), 8.26 (s, 1H, major rotamer), 5.93-5.87 (m, 1H, major rotamer), 5.76 (t, J 56.0 Hz, 1H, major and minor rotamers), 5.59-5.55 (m, 1H, minor rotamer), 5.25-5.08 (m, 2H, major and minor rotamers), 4.47-4.39 (m, 1H, minor rotamer), 4.06-3.97 (m, 1H, major rotamer), 3.78-3.65 (m, 1H, major rotamer), 3.22-3.10 (m, 1H, minor rotamer), 2.50-2.41 (obscured m, 1H, major and minor rotamers), 2.48 (s, 3H, minor rotamer), 2.47 (s, 3H, major rotamer), 2.32-2.16 (m, 7H, major and minor rotamers), 2.13-1.52 (m, 9H, major and minor rotamers), 1.47-1.21 (m, 2H, major and minor rotamers). LCMS (Method 7): [M+H]⁺ m/z 639.2, RT 1.90 minutes. Chiral analysis (Method 20): RT 2.83 minutes.

Peak 2 (mixture of rotamers in 1.6:1 ratio by ¹H NMR): δ_{H} (400 MHz, DMSO-d₆) 9.52 (d, J 8.9 Hz, 1H, minor rotamer), 9.50 (dd, J 8.9 Hz, 1H, major rotamer), 8.73 (s, 1H, minor rotamer), 8.71 (s, 1H, major rotamer), 8.29 (s, 1H, minor rotamer), 8.25 (s, 1H, major rotamer), 5.93-5.87 (m, 1H, major rotamer), 5.76 (t, J 56.4 Hz, 1H, major and minor rotamers), 5.60-5.55 (m, 1H, minor rotamer), 5.25-5.09 (m, 2H, major and minor rotamers), 4.47-4.39 (m, 1H, minor rotamer), 4.06-3.97 (m, 1H, major rotamer), 3.78-3.65 (m, 1H, major rotamer), 3.22-3.10 (m, 1H, minor rotamer), 2.50-2.41 (obscured m, 1H, major and minor rotamers), 2.47 (s, 3H, major and minor rotamers), 2.32-2.16 (m, 7H, major and minor rotamers), 2.13-1.52 (m, 9H, major and minor rotamers), 1.47-1.21 (m, 2H, major and minor rotamers). (Method 7): [M+H]⁺ m/z 639.2, RT 1.89 minutes. Chiral analysis (Method 20): RT 3.24 minutes.

### Examples 28 & 29

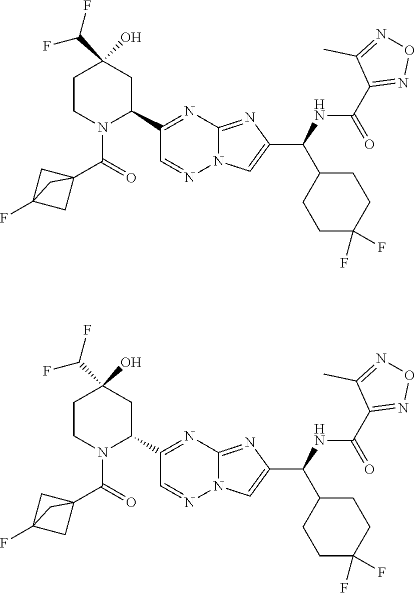 [SMILES: Cc1nonc1C(=O)N[C@H](c1cn2ncc([C@@H]3C[C@](O)(C(F)F)CCN3C(=O)C34CC(F)(C3)C4)nc2n1)C1CCC(F)(F)CC1 ; Cc1nonc1C(=O)N[C@H](c1cn2ncc([C@H]3C[C@@](O)(C(F)F)CCN3C(=O)C34CC(F)(C3)C4)nc2n1)C1CCC(F)(F)CC1]

### N—[(S)-(4,4-Difluorocyclohexyl){3-[(2S,4S)-4-(difluoromethyl)-1-(3-fluorobicyclo-[1.1.1]pentane-1-carbonyl)-4-hydroxypiperidin-2-yl]imidazo[1,2-b][1,2,4]triazin-6-yl}-methyl]-4-methyl-1,2,5-oxadiazole-3-carboxamide

### N—[(S)-(4,4-Difluorocyclohexyl){3-[(2R,4R)-4-(difluoromethyl)-1-(3-fluorobicyclo-[1.1.1]pentane-1-carbonyl)-4-hydroxypiperidin-2-yl]imidazo[1,2-b][1,2,4]triazin-6-yl}-methyl]-4-methyl-1,2,5-oxadiazole-3-carboxamide

To a solution of Intermediate 83 (mixture of two anti stereoisomers in 1:1 ratio) (93.4 mg, 0.17 mmol), 3-fluorobicyclo[1.1.1]pentane-1-carboxylic acid (25.0 mg, 0.18 mmol) and DIPEA (0.12 mL, 0.69 mmol) in DMF (5 mL) at r.t. was added HATU (78.0 mg, 0.20 mmol) in one portion. The mixture was stirred for 80 minutes, then H₂O (10 mL) was added. The mixture was extracted with EtOAc (3×10 mL), and the combined organic layers were washed with brine (20 mL), then dried (Na₂SO₄) and concentrated in vacuo. The residue was purified by flash chromatography, eluting with EtOAc/isohexane (0-100% gradient). The resulting white foam (42.0 mg) was subjected to chiral purification (Method 19) to afford, after lyophilisation, the title compounds (Peak 1, 10.0 mg, 9.4% yield, >99% d.e.; and Peak 2, 9.0 mg, 8.5% yield, 98.0% d.e.).

Peak 1: δ_{H} (400 MHz, DMSO-d₆) 9.48 (d, J 8.9 Hz, 1H), 8.60 (s, 1H), 8.25 (s, 1H), 5.83 (t, J 56.1 Hz, 1H), 5.64-5.55 (br s, 1H), 5.18 (app. t, J 8.5 Hz, 1H), 5.15-5.05 (m, 1H), 3.92-3.79 (m, 1H), 3.75-3.61 (m, 1H), 2.64-2.48 (obscured m, 1H), 2.47 (s, 3H), 2.45-2.35 (br s, 6H), 2.29-2.16 (m, 1H), 2.16-1.58 (m, 9H), 1.48-1.21 (m, 2H). LCMS (Method 7): [M+H]⁺ m/z 639.2, RT 1.89 minutes. Chiral analysis (Method 20): RT 3.16 minutes.

Peak 2: δ_{H} (400 MHz, DMSO-d₆) 9.48 (d, J 8.9 Hz, 1H), 8.60 (s, 1H), 8.25 (s, 1H), 5.83 (t, J 56.2 Hz, 1H), 5.66-5.55 (br s, 1H), 5.18 (app. t, J 8.5 Hz, 1H), 5.15-5.05 (m, 1H), 3.93-3.79 (m, 1H), 3.75-3.62 (m, 1H), 2.62-2.48 (obscured m, 1H), 2.47 (s, 3H), 2.45-2.35 (br s, 6H), 2.26-2.16 (m, 1H), 2.16-1.58 (m, 9H), 1.48-1.21 (m, 2H). LCMS (Method 7): [M+H]⁺ m/z 639.2, RT 1.89 minutes. Chiral analysis (Method 20) RT 3.59 minutes.

### Examples 30 & 31

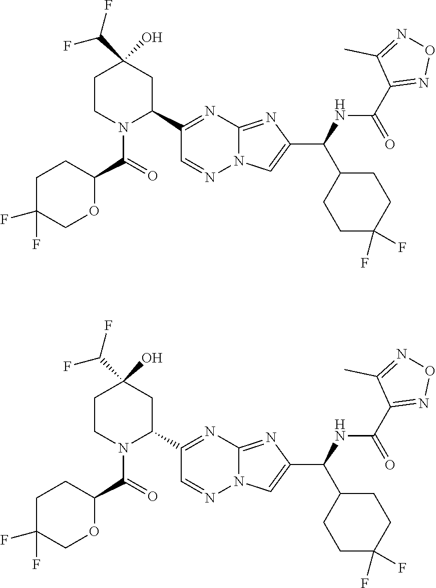 [SMILES: Cc1nonc1C(=O)N[C@H](c1cn2ncc([C@@H]3C[C@](O)(C(F)F)CCN3C(=O)[C@@H]3CCC(F)(F)CO3)nc2n1)C1CCC(F)(F)CC1 ; Cc1nonc1C(=O)N[C@H](c1cn2ncc([C@H]3C[C@@](O)(C(F)F)CCN3C(=O)[C@@H]3CCC(F)(F)CO3)nc2n1)C1CCC(F)(F)CC1]

### N—[(S)-(4,4-Difluorocyclohexyl)(3-{(2S,4S)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{(2R,4R)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide

To a solution of Intermediate 83 (mixture of two anti stereoisomers in 1:1 ratio) (73.7 mg, 0.13 mmol), Intermediate 79 and DIPEA (0.09 mL, 0.50 mmol) in DMF (5 mL) at r.t. was added HATU (62.0 mg, 0.16 mmol) in one portion. The mixture was stirred for 35 minutes, then H₂O (20 mL) was added. The mixture was extracted with EtOAc (3×20 mL), and the combined organic layers were washed with brine (40 mL), then dried (Na₂SO₄) and concentrated in vacuo. The residue was purified by flash chromatography, eluting with EtOAc/isohexane (0-100% gradient), to afford the crudely resolved anti diasteromers. Diastereomer 1 was subjected to chiral purification (Method 21) to yield Peak 1 (8 mg, 9%). Diastereomer 2 was subjected to chiral purification (Method 22) to yield Peak 2 (8 mg, 9%).

Peak 1: δ_{H} (400 MHz, DMSO-d₆) 9.48 (d, J 8.9 Hz, 1H), 8.64 (s, 1H), 8.26 (s, 1H), 5.83 (t, J 56.1 Hz, 1H), 5.64-5.54 (br s, 1H), 5.18 (t, J 8.6 Hz, 1H), 5.13 (dd, J 9.1, 7.6 Hz, 1H), 4.56 (t, J 6.2 Hz, 1H), 3.97-3.75 (m, 3H), 3.69-3.57 (m, 1H), 2.47 (s, 3H), 2.29-1.58 (m, 15H), 1.47-1.20 (m, 2H). LCMS (Method 7): [M+H]⁺ m/z 675.2, RT 1.90 minutes.

Peak 2: δ_{H} (400 MHz, DMSO-d₆) 9.48 (d, J 8.7 Hz, 1H), 8.58 (s, 1H), 8.26 (s, 1H), 6.03-5.54 (m, 2H), 5.19 (t, J 8.3 Hz, 1H), 5.15-5.07 (m, 1H), 4.54-4.45 (m, 1H), 4.00-3.63 (m, 4H), 2.47 (s, 3H), 2.29-1.54 (m, 15H), 1.48-1.22 (m, 2H). LCMS (Method 7): [M+H]⁺ m/z 675.2, RT 1.91 minutes.

### Examples 32 & 33

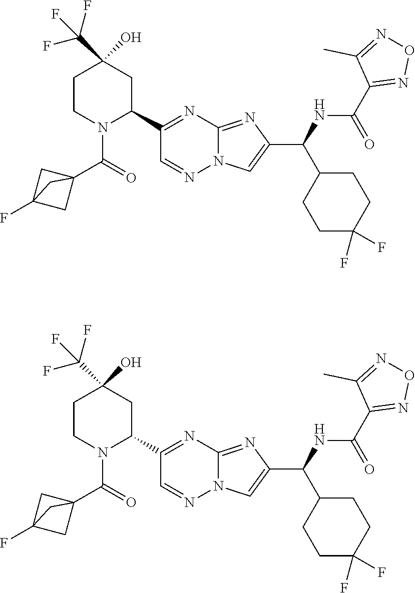 [SMILES: Cc1nonc1C(=O)N[C@H](c1cn2ncc([C@@H]3C[C@](O)(C(F)(F)F)CCN3C(=O)C34CC(F)(C3)C4)nc2n1)C1CCC(F)(F)CC1 ; Cc1nonc1C(=O)N[C@H](c1cn2ncc([C@H]3C[C@@](O)(C(F)(F)F)CCN3C(=O)C34CC(F)(C3)C4)nc2n1)C1CCC(F)(F)CC1]

### N—[(S)-(4,4-Difluorocyclohexyl){3-[(2S,4S)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-4-methyl-1,2,5-oxadiazole-3-carboxamide

### N—[(S)-(4,4-Difluorocyclohexyl){3-[(2R,4R)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-4-methyl-1,2,5-oxadiazole-3-carboxamide

To a solution of Intermediate 86 (mixture of two anti stereoisomers in 1:1 ratio) (38.8 mg, 0.067 mmol), 3-fluorobicyclo[1.1.1]pentane-1-carboxylic acid (10.0 mg, 0.073 mmol) and DIPEA (0.05 mL, 0.30 mmol) in DMF (5 mL) at r.t. was added HATU (32.0 mg, 0.08 mmol) in one portion. The mixture was stirred for 30 minutes, then H₂O (10 mL) was added. The mixture was extracted with EtOAc (3×10 mL), and the combined organic layers were washed with brine (20 mL), then dried (Na₂SO₄) and concentrated in vacuo. The residue was purified by flash chromatography, eluting with EtOAc/isohexane (0-100% gradient). The resulting white foam (35.0 mg) was subjected to chiral purification (Method 23) to afford, after lyophilisation the title compounds (Peak 1, 11.0 mg, 25.1% yield, >99% d.e.; and Peak 2, 9.0 mg, 20.6% yield, 92.6% d.e.).

Peak 1: δ_{H} (400 MHz, DMSO-d₆) 9.49 (d, J 8.9 Hz, 1H), 8.67-8.58 (br s, 1H), 8.30-8.21 (br s, 1H), 5.37-5.27 (br s, 1H), 5.18 (app. t, J 8.6 Hz, 1H), 5.17-5.08 (m, 1H), 3.95-3.81 (m, 1H), 3.81-3.68 (m, 1H), 2.47 (s, 3H), 2.45-2.36 (m, 6H), 2.35-2.14 (m, 3H), 2.12-1.68 (m, 7H), 1.68-1.59 (m, 1H), 1.48-1.22 (m, 2H). LCMS (Method 7): [M+H]⁺ m/z 657.2, RT 2.01 minutes. Chiral analysis (Method 24): RT 4.04 minutes.

Peak 2: δ_{H} (400 MHz, DMSO-d₆) 9.48 (d, J 8.9 Hz, 1H), 8.67-8.57 (br s, 1H), 8.32-8.22 (br s, 1H), 6.37-6.26 (br s, 1H), 5.18 (t, J 8.5 Hz, 1H), 5.17-5.08 (m, 1H), 3.96-3.81 (m, 1H), 3.81-3.67 (m, 1H), 2.47 (s, 3H), 2.45-2.36 (m, 6H), 2.35-2.14 (m, 3H), 2.13-1.68 (m, 7H), 1.67-1.57 (m, 1H), 1.47-1.21 (m, 2H). LCMS (Method 7): [M+H]⁺ m/z 657.2, RT 2.00 minutes. Chiral analysis (Method 24): RT 4.54 minutes.

### Examples 34 & 35

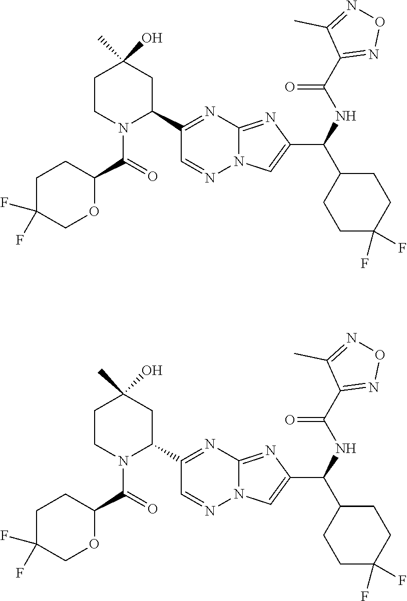 [SMILES: Cc1nonc1C(=O)N[C@H](c1cn2ncc([C@@H]3C[C@](C)(O)CCN3C(=O)[C@@H]3CCC(F)(F)CO3)nc2n1)C1CCC(F)(F)CC1 ; Cc1nonc1C(=O)N[C@H](c1cn2ncc([C@H]3C[C@@](C)(O)CCN3C(=O)[C@@H]3CCC(F)(F)CO3)nc2n1)C1CCC(F)(F)CC1]

### N—[(S)-(4,4-Difluorocyclohexyl)(3-{(2S,4R)-1-[(2S)-5,5-difluorotetrahydropyran-2-carbonyl]-4-hydroxy-4-methylpiperidin-2-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{(2R,4S)-1-[(2S)-5,5-difluorotetrahydropyran-2-carbonyl]-4-hydroxy-4-methylpiperidin-2-yl}imidazo[1,2-b][1,2,4]triazin-6-yl)methyl]-4-methyl-1,2,5-oxadiazole-3-carboxamide

To a solution of Intermediate 90 (277 mg, 0.473 mmol), Intermediate 79 (100 mg, 0.602 mmol) and DIPEA (220 μL, 1.3 mmol) in DMF (5 mL) was added HATU (240 mg, 0.612 mmol). The solution was stirred at r.t. overnight, then partitioned between EtOAc and brine. The aqueous phase was extracted twice with EtOAc, and the combined organic phase was washed with brine, then dried over Na₂SO₄ and concentrated in vacuo. Purification by flash column chromatography on silica, eluting with 30-100% EtOAc/isohexane, then chiral purification (Method 19), afforded, after lyophilisation, the title compounds (Peak 1, 45 mg, 15% yield, 100.0% d.e.; and Peak 2, 37 mg, 12% yield, 96.1% d.e.).

Peak 1: δ_{H} (400 MHz, 373K, DMSO-d₆) 9.03-8.96 (m, 1H), 8.62-8.58 (m, 1H), 8.16 (s, 1H), 5.74 (br s, 1H), 5.23 (t, J 8.3 Hz, 1H), 4.74-4.54 (m, 1H), 4.07-3.65 (m, 4H), 2.56-2.52 (m, 2H), 2.49 (s, 3H), 2.30-2.19 (m, 2H), 2.08-1.93 (m, 6H), 1.90-1.69 (m, 4H), 1.62-1.33 (m, 4H), 1.19 (s, 3H). LCMS (Method 7): [M+H]⁺ m/z 639.4, RT 1.85 minutes. Chiral analysis (Method 20): RT 3.35 minutes.

Peak 2: δ_{H} (400 MHz, 373K, DMSO-d₆) 9.02 (d, J 8.8 Hz, 1H), 8.69-8.45 (m, 1H), 8.16 (s, 1H), 5.69 (br s, 1H), 5.24 (t, J 8.3 Hz, 1H), 4.77-4.16 (m, 1H), 4.10-3.65 (m, 4H), 2.59-2.52 (m, 2H), 2.49 (s, 3H), 2.30-2.19 (m, 2H), 2.09-1.92 (m, 6H), 1.90-1.70 (m, 4H), 1.61-1.30 (m, 4H), 1.20 (s, 3H). LCMS (Method 7): [M+H]⁺ m/z 639.4, RT 1.86 minutes. Chiral analysis (Method 20): RT 3.74 minutes.

### Examples 36 & 37

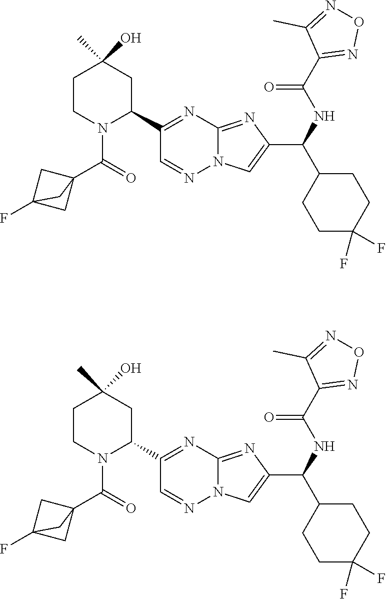 [SMILES: Cc1nonc1C(=O)N[C@H](c1cn2ncc([C@@H]3C[C@](C)(O)CCN3C(=O)C34CC(F)(C3)C4)nc2n1)C1CCC(F)(F)CC1 ; Cc1nonc1C(=O)N[C@H](c1cn2ncc([C@H]3C[C@@](C)(O)CCN3C(=O)C34CC(F)(C3)C4)nc2n1)C1CCC(F)(F)CC1]

### N—[(S)-(4,4-Difluorocyclohexyl){3-[(2S,4R)-1-(3-fluorobicyclo[1.1.1]pentane-1-carbonyl)-4-hydroxy-4-methylpiperidin-2-yl]imidazo[1,2-b][1,2,4]triazin-6-yl}methyl]-4-methyl-1,2,5-oxadiazole-3-carboxamide

### N—[(S)-(4,4-Difluorocyclohexyl){3-[(2R,4S)-1-(3-fluorobicyclo[1.1.1]pentane-1-carbonyl)-4-hydroxy-4-methylpiperidin-2-yl]imidazo[1,2-b][1,2,4]triazin-6-yl}methyl]-4-methyl-1,2,5-oxadiazole-3-carboxamide

To a solution of Intermediate 90 (220 mg, 0.376 mmol), 3-fluorobicyclo[1.1.1]-pentane-1-carboxylic acid (62 mg, 0.45 mmol) and DIPEA (160 μL, 0.94 mmol) in DMF (5 mL) was added HATU (177 mg, 0.452 mmol). The solution was stirred at r.t. overnight, then partitioned between EtOAc and brine. The aqueous phase was extracted twice with EtOAc, and the combined organic phase was washed with brine, then dried over Na₂SO₄ and concentrated in vacuo. Purification by flash column chromatography on silica, eluting with 20-100% EtOAc/isohexane, then chiral purification (Method 19), afforded, after lyophilisation, the title compounds (Peak 1, 18 mg, 8% yield, 100.0% d.e.; and Peak 2, 17 mg, 7% yield, 97.4% d.e.).

Peak 1: δ_{H} (400 MHz, 373K, DMSO-d₆) 9.03 (d, J 8.2 Hz, 1H), 8.58 (s, 1H), 8.16 (s, 1H), 5.68 (br s, 1H), 5.24 (t, J 8.1 Hz, 1H), 3.93 (s, 1H), 3.81-3.51 (m, 1H), 2.49 (s, 3H), 2.49-2.33 (m, 6H), 2.31-2.20 (m, 2H), 2.11-1.94 (m, 4H), 1.91-1.67 (m, 4H), 1.60-1.54 (m, 2H), 1.49-1.33 (m, 2H), 1.19 (s, 3H). LCMS (Method 7): [M+H]⁺ m/z 603.4, RT 1.85 minutes. Chiral analysis (Method 20): RT 3.41 minutes.

Peak 2: δ_{H} (400 MHz, 373K, DMSO-d₆) 9.02 (d, J 8.7 Hz, 1H), 8.58 (s, 1H), 8.16 (s, 1H), 5.66 (br s, 1H), 5.24 (t, J 7.9 Hz, 1H), 3.93 (s, 1H), 3.81-3.51 (m, 1H), 2.49 (s, 3H), 2.49-2.31 (m, 6H), 2.31-2.19 (m, 2H), 2.13-1.91 (m, 4H), 1.92-1.68 (m, 4H), 1.59-1.52 (m, 2H), 1.50-1.32 (m, 2H), 1.19 (s, 3H). LCMS (Method 7): [M+H]⁺ m/z 603.4, RT 1.85 minutes. Chiral analysis (Method 20): RT 3.83 minutes.

### Examples 38 & 39

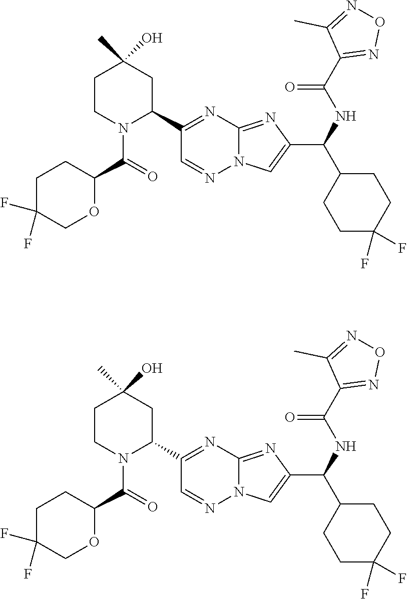 [SMILES: Cc1nonc1C(=O)N[C@H](c1cn2ncc([C@@H]3C[C@@](C)(O)CCN3C(=O)[C@@H]3CCC(F)(F)CO3)nc2n1)C1CCC(F)(F)CC1 ; Cc1nonc1C(=O)N[C@H](c1cn2ncc([C@H]3C[C@](C)(O)CCN3C(=O)[C@@H]3CCC(F)(F)CO3)nc2n1)C1CCC(F)(F)CC1]

### N—[(S)-(4,4-Difluorocyclohexyl)(3-{(2S,4S)-1-[(2S)-5,5-difluorotetrahydropyran-2-carbonyl]-4-hydroxy-4-methylpiperidin-2-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{(2R,4R)-1-[(2S)-5,5-difluorotetrahydropyran-2-carbonyl]-4-hydroxy-4-methylpiperidin-2-yl}imidazo[1,2-b][1,2,4]triazin-6-yl)methyl]-4-methyl-1,2,5-oxadiazole-3-carboxamide

To a solution of Intermediate 91 (100 mg, 0.123 mmol), Intermediate 79 (25 mg, 0.15 mmol) and DIPEA (54 μL, 0.31 mmol) in DMF (2.5 mL) was added HATU (60 mg, 0.15 mmol). The solution was stirred at r.t. overnight, then partitioned between EtOAc and brine. The aqueous phase was extracted twice with EtOAc, and the combined organic phase was washed with brine, then dried over Na₂SO₄ and concentrated in vacuo. Purification by flash column chromatography on silica, eluting with 50-100% EtOAc/isohexane, then chiral purification (Method 19), afforded, after lyophilisation, the title compounds (Peak 1, 4.6 mg, 6% yield, 100.0% d.e.; and Peak 2, 6.3 mg, 8% yield, 98.9% d.e.).

Peak 1: δ_{H} (400 MHz, 373K, DMSO-d₆) 9.06-8.99 (m, 1H), 8.57-8.52 (m, 1H), 8.19 (s, 1H), 5.41-5.33 (m, 1H), 5.25 (t, J 8.3 Hz, 1H), 4.52-4.46 (m, 1H), 4.35 (s, 1H), 4.04-3.72 (m, 4H), 3.60-3.50 (m, 1H), 2.63-2.57 (m, 2H), 2.49 (s, 3H), 2.29-2.16 (m, 3H), 2.13-1.65 (m, 8H), 1.54-1.35 (m, 3H), 1.09 (s, 3H). LCMS (Method 7): [M+H]⁺ m/z 639.2, RT 1.84 minutes. Chiral analysis (Method 20): RT 4.39 minutes.

Peak 2: δ_{H} (400 MHz, 373K, DMSO-d₆) 9.05-8.99 (m, 1H), 8.59-8.53 (m, 1H), 8.18 (s, 1H), 5.41-5.33 (m, 1H), 5.25 (t, J 8.3 Hz, 1H), 4.60-4.53 (m, 1H), 4.34 (s, 1H), 4.10-3.87 (m, 2H), 3.82-3.66 (m, 2H), 3.62-3.44 (m, 1H), 2.57-2.54 (m, 2H), 2.49 (s, 3H), 2.28-2.19 (m, 3H), 2.14-1.65 (m, 8H), 1.55-1.34 (m, 3H), 1.09 (s, 3H). LCMS (Method 7): [M+H]⁺ m/z 639.2, RT 1.82 minutes. Chiral analysis (Method 20): RT 4.70 minutes.

### Examples 40 & 41

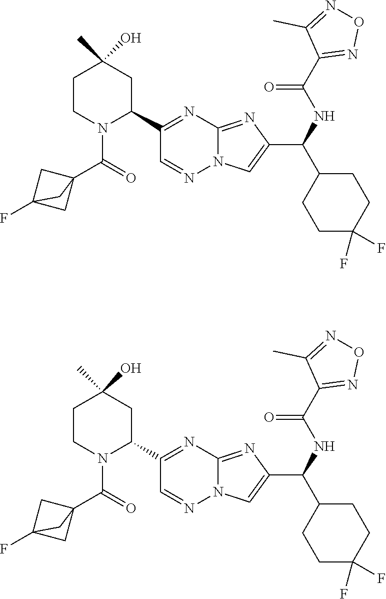 [SMILES: Cc1nonc1C(=O)N[C@H](c1cn2ncc([C@@H]3C[C@@](C)(O)CCN3C(=O)C34CC(F)(C3)C4)nc2n1)C1CCC(F)(F)CC1 ; Cc1nonc1C(=O)N[C@H](c1cn2ncc([C@H]3C[C@](C)(O)CCN3C(=O)C34CC(F)(C3)C4)nc2n1)C1CCC(F)(F)CC1]

### N—[(S)-(4,4-Difluorocyclohexyl){3-[(2S,4S)-1-(3-fluorobicyclo[1.1.1]pentane-1-carbonyl)-4-hydroxy-4-methylpiperidin-2-yl]imidazo[1,2-b][1,2,4]triazin-6-yl}methyl]-4-methyl-1,2,5-oxadiazole-3-carboxamide

### N—[(S)-(4,4-Difluorocyclohexyl){3-[(2R,4R)-1-(3-fluorobicyclo[1.1.1]pentane-1-carbonyl)-4-hydroxy-4-methylpiperidin-2-yl]imidazo[1,2-b][1,2,4]triazin-6-yl}methyl]-4-methyl-1,2,5-oxadiazole-3-carboxamide

To a solution of Intermediate 91 (100 mg, 0.123 mmol), 3-fluorobicyclo[1.1.1]-pentane-1-carboxylic acid (20 mg, 0.15 mmol) and DIPEA (54 μL, 0.31 mmol) in DMF (2.5 mL) was added HATU (60 mg, 0.15 mmol). The solution was stirred at r.t. overnight, then partitioned between EtOAc and brine. The aqueous phase was extracted twice with EtOAc, and the combined organic phase was washed with brine, then dried over Na₂SO₄ and concentrated in vacuo. Purification by flash column chromatography on silica, eluting with 50-100% EtOAc/isohexane, then chiral purification (Method 19), afforded, after lyophilisation, the title compounds (Peak 1, 6.1 mg, 8% yield, 100.0% d.e.; and Peak 2, 5.5 mg, 7% yield, 99.4% d.e.).

Peak 1: δ_{H} (400 MHz, 373K, DMSO-d₆) 9.05-8.99 (m, 1H), 8.55 (s, 1H), 8.19 (s, 1H), 5.33-5.20 (m, 2H), 4.35 (s, 1H), 3.98-3.88 (m, 1H), 3.60-3.48 (m, 1H), 2.49 (s, 3H), 2.43-2.34 (m, 6H), 2.28-2.16 (m, 2H), 2.11-2.02 (m, 3H), 1.98-1.92 (m, 1H), 1.87-1.65 (m, 5H), 1.53-1.43 (m, 1H), 1.41-1.31 (m, 1H), 1.10 (s, 3H). LCMS (Method 7): [M+H]⁺ m/z 603.4, RT 1.81 minutes. Chiral analysis (Method 20): RT 4.10 minutes.

Peak 2: δ_{H} (400 MHz, 373K, DMSO-d₆) 9.02 (d, J 9.0 Hz, 1H), 8.55 (s, 1H), 8.19 (s, 1H), 5.33-5.20 (m, 2H), 4.35 (s, 1H), 4.02-3.84 (m, 1H), 3.62-3.43 (m, 1H), 2.49 (s, 3H), 2.42-2.35 (m, 6H), 2.28-2.14 (m, 2H), 2.12-2.01 (m, 3H), 1.99-1.91 (m, 1H), 1.88-1.66 (m, 5H), 1.55-1.44 (m, 1H), 1.43-1.32 (m, 1H), 1.10 (s, 3H). LCMS (Method 7): [M+H]⁺ m/z 603.4, RT 1.82 minutes. Chiral analysis (Method 20): RT 5.52 minutes.

### Examples 42 to 45

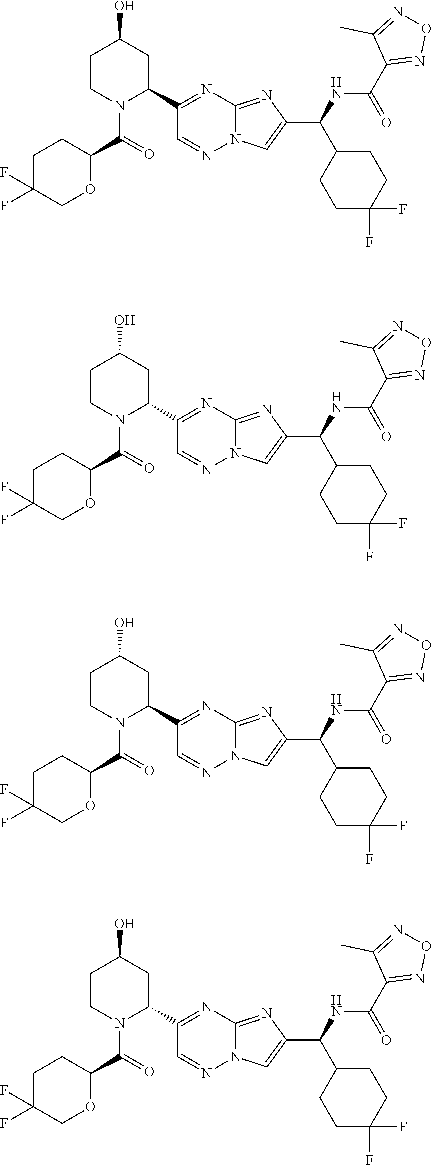 [SMILES: Cc1nonc1C(=O)N[C@H](c1cn2ncc([C@@H]3C[C@H](O)CCN3C(=O)[C@@H]3CCC(F)(F)CO3)nc2n1)C1CCC(F)(F)CC1 ; Cc1nonc1C(=O)N[C@H](c1cn2ncc([C@H]3C[C@@H](O)CCN3C(=O)[C@@H]3CCC(F)(F)CO3)nc2n1)C1CCC(F)(F)CC1 ; Cc1nonc1C(=O)N[C@H](c1cn2ncc([C@@H]3C[C@@H](O)CCN3C(=O)[C@@H]3CCC(F)(F)CO3)nc2n1)C1CCC(F)(F)CC1 ; Cc1nonc1C(=O)N[C@H](c1cn2ncc([C@H]3C[C@H](O)CCN3C(=O)[C@@H]3CCC(F)(F)CO3)nc2n1)C1CCC(F)(F)CC1]

### N—[(S)-(4,4-Difluorocyclohexyl)(3-{(2S,4R)-1-[(2S)-5,5-difluorotetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{(2R,4S)-1-[(2S)-5,5-difluorotetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{(2S,4S)-1-[(2S)-5,5-difluorotetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide

### N—[(S)-(4,4-Difluorocyclohexyl)(3-{(2R,4R)-1-[(2S)-5,5-difluorotetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide

To a solution of Intermediate 79 (123 mg, 0.74 mmol), DIPEA (0.50 mL, 2.86 mmol) and Intermediate 97 (81%, 360 mg, 0.57 mmol) in DCM (7 mL) was added HATU (346 mg, 0.91 mmol). The solution was stirred at r.t. for 2 h, then concentrated under reduced pressure. The residue was purified by column chromatography, eluting with a gradient of EtOAc in heptane, then with a gradient of MeOH in DCM. Further purification was carried out by reverse-phase HPLC (5-95% acetonitrile/water (+0.1% formic acid), X-Bridge, 30×100 mm, 5 μm @ 40 mL/minute). The resulting material was subjected to chiral purification using SFC (10% methanol:90% CO₂, Cellulose-4, 10×250 mm, 5 μm @ 18 mL/minute) to yield, after lyophilization, the title compounds (Peak 1, 8.2 mg, 2%; Peak 2, 7.5 mg, 2%; Peak 3, 1.9 mg, 1%; and Peak 4, 8.0 mg, 2%).

Peak 1 (arbitrarily assigned 2S,4R): δ_{H} (400 MHz, DMSO-d₆) 9.52-9.42 (m, 1H), 8.75-8.60 (m, 1H), 8.25-8.20 (m, 1H), 5.99-5.60 (m, 1H), 5.45-5.11 (m, 1H), 4.77-3.47 (m, 8H), 2.65-2.53 (m, 1H), 2.49-2.43 (m, 3H), 2.29-1.49 (m, 13H), 1.47-1.18 (m, 2H). LCMS (Method 2): [M+H]⁺ m/z 625.5, RT 3.19 minutes. Chiral analysis (Method 25): RT 3.92 minutes.

Peak 2 (arbitrarily assigned 2R,4S): δ_{H} (400 MHz, DMSO-d₆) 9.53-9.45 (m, 1H), 8.82-8.60 (m, 1H), 8.26-8.15 (m, 1H), 5.70-5.44 (m, 1H), 5.22-5.13 (m, 1H), 4.71-3.63 (m, 7H), 3.23-2.64 (m, 2H), 2.46 (s, 3H), 2.30-1.50 (m, 13H), 1.48-1.21 (m, 2H). LCMS (Method 2): [M+H]⁺ m/z 625.5, RT 3.21 minutes. Chiral analysis (Method 25): RT 4.62 minutes.

Peak 3 (arbitrarily assigned 2S,4S): δ_{H} (400 MHz, DMSO-d₆) 9.55-9.46 (m, 1H), 8.76-8.24 (m, 2H), 6.01-5.61 (m, 1H), 5.21 (t, J 8.5 Hz, 1H), 4.96-3.53 (m, 7H), 3.24-2.61 (m, 2H), 2.47 (s, 3H), 2.30-1.50 (m, 13H), 1.49-1.12 (m, 2H). LCMS (Method 2): [M+H]+m/z 625.5, RT 3.07 minutes. Chiral analysis (Method 25): RT 5.71 minutes.

Peak 4 (arbitrarily assigned 2R,4R): δ_{H} (400 MHz, DMSO-d₆) 9.55-9.46 (m, 1H), 8.54-8.48 (m, 1H), 8.32-8.25 (m, 1H), 6.01-5.52 (m, 1H), 5.26-5.14 (m, 1H), 4.91-3.59 (m, 7H), 3.17-2.63 (m, 2H), 2.47 (s, 3H), 2.31-1.48 (m, 13H), 1.48-1.16 (m, 2H). LCMS (Method 2): [M+H]⁺ m/z 625.5, RT 3.06 minutes. Chiral analysis (Method 25): RT 6.60 minutes.

### Examples 46 & 47

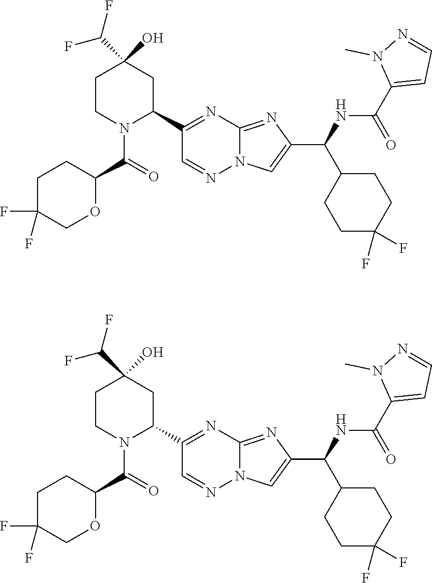 [SMILES: Cn1nccc1C(=O)N[C@H](c1cn2ncc([C@@H]3C[C@@](O)(C(F)F)CCN3C(=O)[C@@H]3CCC(F)(F)CO3)nc2n1)C1CCC(F)(F)CC1 ; Cn1nccc1C(=O)N[C@H](c1cn2ncc([C@H]3C[C@](O)(C(F)F)CCN3C(=O)[C@@H]3CCC(F)(F)CO3)nc2n1)C1CCC(F)(F)CC1]

### N—[(S)-(4,4-Difluorocyclohexyl)(3-{(2S,4R)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)-methyl]-2-methylpyrazole-3-carboxamide

### N—[(S)-(4,4-Difluorocyclohexyl)(3-[(2R,4S)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)-methyl]-2-methyl-pyrazole-3-carboxamide

To a solution of Intermediate 72 (1:1 ratio of two stereoisomers) (118.0 mg, 0.21 mmol), 1-methyl-1H-pyrazole-5-carboxylic acid (27.0 mg, 0.21 mmol) and DIPEA (0.15 mL, 0.86 mmol) in DMF (10 mL) at r.t. was added HATU (98.0 mg, 0.25 mmol) in one portion. The mixture was stirred for 35 minutes, then H₂O (20 mL) was added and the mixture was extracted with EtOAc (3×20 mL). The combined organic layers were washed with brine (40 mL), then dried (Na₂SO₄) and concentrated in vacuo. The residue was purified by flash chromatography, eluting with EtOAc/isohexane (0-100% gradient), then 10% MeOH/DCM. The resulting yellow solid (115.0 mg) was subjected to chiral purification (Method 26) to afford, after lyophilisation, the title compounds (Peak 1, 30.0 mg, 21.3%, >99% d.e.; and Peak 2, 30.0 mg, 21.3%, >99% d.e.) as separate diastereomers.

Peak 1 (arbitrarily assigned 2S,4R) (3.0:1 rotameric ratio by ¹H NMR at r.t.): δ_{H} (400 MHz, DMSO-d₆) 8.79 (d, J 8.9 Hz, 1H, minor rotamer), 8.77 (d, J 9.0 Hz, 1H, major rotamer), 8.74 (s, 1H, major rotamer), 8.64 (s, 1H, minor rotamer), 8.24 (s, 1H, major and minor rotamers), 7.46 (d, J 2.0 Hz, 1H, major and minor rotamers), 7.05 (d, J 2.2 Hz, 1H, minor rotamer), 7.04 (d, J 2.1 Hz, 1H, major rotamer), 5.90-5.84 (m, 1H, major rotamer), 5.78 (t, J 55.8 Hz, 1H, major rotamer), 5.74 (t, J 56.1 Hz, 1H, minor rotamer), 5.59-5.54 (m, 1H, minor rotamer), 5.22-5.04 (m, 2H, major and minor rotamers), 4.69 (app. t, J 6.4 Hz, 1H, major rotamer), 4.56 (dd, J 7.2, 4.4 Hz, 1H, minor rotamer), 4.53-4.45 (m, 1H, minor rotamer), 4.025 (s, 3H, minor rotamer), 4.019 (s, 3H, major rotamer), 4.09-3.98 (obscured m, 1H, major rotamer), 3.98-3.81 (m, 2H, 2× major rotamer), 3.71-3.60 (m, 1H, major rotamer), 3.60-3.41 (m, 2H, 2× minor rotamers), 3.37-3.25 (obscured m, 1H, minor rotamer), 2.31-1.51 (m, 15H, major and minor rotamers), 1.46-1.20 (m, 2H, major and minor rotamers). LCMS (Method 7): [M+H]⁺ m/z 673.2, RT 1.69 minutes. Chiral analysis (Method 27): RT 5.21 minutes.

Peak 2 (arbitrarily assigned 2R,4S) (1.1:1 rotameric ratio by ¹H NMR at r.t.): δ_{H} (400 MHz, DMSO-d₆) 8.80 (d, J 5.4 Hz, 1H, major rotamer), 8.78 (s, 1H, minor rotamer), 8.78 (d, J 5.8 Hz, 1H, minor rotamer), 8.64 (s, 1H, major rotamer), 8.24 (s, 1H, minor rotamer), 8.23 (s, 1H, major rotamer), 7.45 (d, J 2.0 Hz, 1H, major and minor rotamers), 7.05 (d, J 2.1 Hz, 1H, minor rotamer), 7.04 (d, J 2.1 Hz, 1H, major rotamer), 5.90-5.86 (m, 1H, major rotamer), 5.78 (t, J 55.9 Hz, 1H, minor rotamer), 5.77 (t, J 56.0 Hz, 1H, major rotamer), 5.69-5.63 (m, 1H, minor rotamer), 5.22-5.11 (m, 2H, major and minor rotamers), 4.68 (dd, J 8.6, 4.1 Hz, 1H, major rotamer), 4.45-4.35 (m, 1H, minor rotamer), 4.29-4.21 (m, 1H, minor rotamer), 4.10-3.97 (m, 2H, 2× major rotamers), 4.02 (s, 3H, major and minor rotamers), 3.94-3.67 (m, 2H, 2× minor rotamers), 3.22-3.11 (m, 1H, minor rotamer), 2.63 (app. d, J 14.3 Hz, 1H, minor rotamer), 2.56-2.43 (obscured m, 1H, major rotamer), 2.32-1.49 (m, 14H, major and minor rotamers), 1.46-1.20 (m, 2H, major and minor rotamers). LCMS (Method 7): [M+H]⁺ m/z 673.2, RT 1.69 minutes. Chiral analysis (Method 27): RT 5.59 minutes.

### Examples 48 & 49

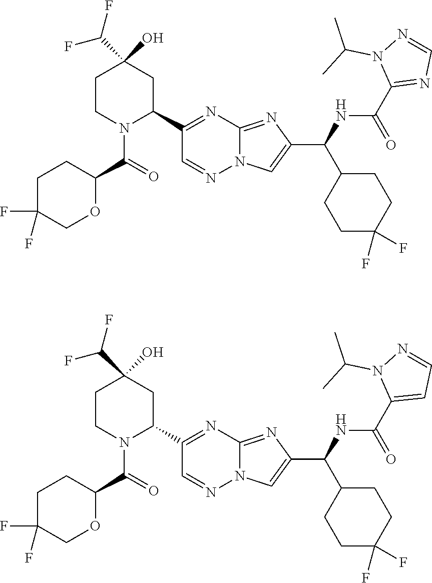 [SMILES: CC(C)n1ncnc1C(=O)N[C@H](c1cn2ncc([C@@H]3C[C@@](O)(C(F)F)CCN3C(=O)[C@@H]3CCC(F)(F)CO3)nc2n1)C1CCC(F)(F)CC1 ; CC(C)n1nccc1C(=O)N[C@H](c1cn2ncc([C@H]3C[C@](O)(C(F)F)CCN3C(=O)[C@@H]3CCC(F)(F)CO3)nc2n1)C1CCC(F)(F)CC1]

### N—[(S)-(4,4-Difluorocyclohexyl)(3-{(2S,4R)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)methyl]-2-isopropyl-1,2,4-triazole-3-carboxamide

### N—[(S)-(4,4-Difluorocyclohexyl)(3-{(2R,4S)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)methyl]-2-isopropyl-1,2,4-triazole-3-carboxamide

To a solution of Intermediate 72 (1:1 ratio of two stereoisomers) (118.0 mg, 0.21 mmol), lithium 1-(propan-2-yl)-1H-1,2,4-triazole-5-carboxylate (34.0 mg, 0.21 mmol) and DIPEA (0.15 mL, 0.86 mmol) in DMF (10 mL) at r.t. was added HATU (98.0 mg, 0.25 mmol) in one portion. The mixture was stirred for 35 minutes, then H₂O (20 mL) was added and the mixture was extracted with EtOAc (3×20 mL). The combined organic layers were washed with brine (40 mL), then dried (Na₂SO₄) and concentrated in vacuo. The residue was purified by flash chromatography, eluting with EtOAc/isohexane (0-100% gradient). The resulting yellow solid (130.0 mg) was subjected to chiral purification (Method 26) to afford, after lyophilisation, the title compounds (Peak 1, 38.0 mg, 25.9%, >99% d.e.; and Peak 2, 35.0 mg, 23.4%, >99% d.e.) as separate diastereomers.

Peak 1 (arbitrarily assigned 2S,4R) (2.4:1 rotameric ratio by ¹H NMR at r.t.): δ_{H} (400 MHz, DMSO-d₆) 8.94 (d, J 9.3 Hz, 1H, minor rotamer), 8.89 (d, J 9.2 Hz, 1H, major rotamer), 8.76 (s, 1H, major rotamer), 8.64 (s, 1H, minor rotamer), 8.292 (s, 1H, minor rotamer), 8.286 (s, 1H, major rotamer), 8.10 (s, 1H, major and minor rotamers), 5.90-5.85 (m, 1H, major rotamer), 5.78 (t, J 55.9 Hz, 1H, major rotamer), 5.74 (t, J 56.0 Hz, 1H, minor rotamer), 5.61-5.48 (sept., J 6.6 Hz, 1H, major and minor rotamers; and m, 1H, minor rotamer), 5.21-5.06 (m, 2H, major and minor rotamers), 4.72 (app. t, J 6.4 Hz, 1H, major rotamer), 4.56 (dd, J 7.4, 4.6 Hz, 1H, minor rotamer), 4.53-4.47 (m, 1H, minor rotamer), 4.10-4.00 (m, 1H, major rotamer), 4.00-3.82 (m, 2H, 2× major rotamers), 3.68 (td, J 13.1, 3.2 Hz, 1H, major rotamer), 3.59-3.41 (m, 2H, 2× minor rotamers), 3.39-3.25 (obscured m, 1H, minor rotamer), 2.31-1.52 (m, 15H, major and minor rotamers), 1.42 (d, J 6.7 Hz, 3H, major and minor rotamers), 1.38 (d, J 6.6 Hz, 3H, major and minor rotamers), 1.46-1.19 (m, 2H, major and minor rotamers). LCMS (Method 7): [M+H]⁺ m z 702.4, RT 1.96 minutes. Chiral analysis (Method 27): RT 4.50 minutes.

Peak 2 (arbitrarily assigned 2R,4S) (1.2:1 rotameric ratio by ¹H NMR at r.t.): δ_{H} (400 MHz, DMSO-d₆) 8.95 (d, J 9.2 Hz, 1H, major rotamer), 8.93 (d, J 9.1 Hz, 1H, minor rotamer), 8.80 (s, 1H, minor rotamer), 8.64 (s, 1H, major rotamer), 8.29 (s, 1H, minor rotamer), 8.28 (s, 1H, major rotamer), 8.10 (s, 1H, major and minor rotamers), 5.91-5.86 (m, 1H, major rotamer), 5.78 (t, J 55.9 Hz, 1H, minor rotamer), 5.77 (t, J 55.9 Hz, 1H, major rotamer), 5.69-5.64 (m, 1H, minor rotamer), 5.53 (sept., J 6.7 Hz, 1H, major and minor rotamers), 5.21-5.11 (m, 2H, major and minor rotamers), 4.69 (dd, J 8.4, 4.1 Hz, 1H, major rotamer), 4.47-4.37 (m, 1H, minor rotamer), 4.30-4.23 (m, 1H, minor rotamer), 4.11-4.00 (m, 2H, 2× major rotamers), 3.94-3.67 (m, 2H, major and minor rotamers), 3.23-3.12 (m, 1H, minor rotamer), 2.63 (app. d, J 14.3 Hz, 1H, minor rotamer), 2.58-2.44 (obscured m, 1H, major rotamer), 2.32-1.49 (m, 14H, major and minor rotamers), 1.42 (d, J 6.6 Hz, 3H, major and minor rotamers), 1.38 (d, J 6.6 Hz 3H, major and minor rotamers), 1.48-1.20 (m, 2H, major and minor rotamers). LCMS (Method 7): [M+H]⁺ m z 702.2, RT 1.96 minutes. Chiral analysis (Method 27): RT 4.95 minutes.

### Examples 50 & 51

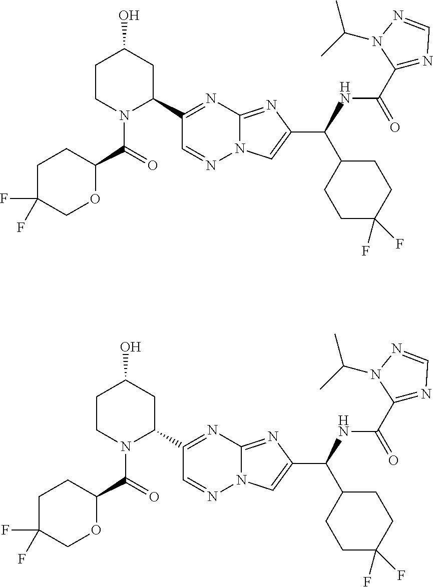

### N—[(S)-(4,4-Difluorocyclohexyl)(3-{(2S,4S)-1-[(2S)-5,5-difluorotetrahydropyran-2-carbonyl]-4-hydroxypiperidin-2-yl}imidazo[1,2-b][1,2,4]triazin-6-yl)methyl]-2-isopropyl-1,2,4-triazole-3-carboxamide

### N—[(S)-(4,4-Difluorocyclohexyl)(3-{(2R,4S)-1-[(2S)-5,5-difluorotetrahydropyran-2-carbonyl]-4-hydroxypiperidin-2-yl}imidazo[1,2-b][1,2,4]triazin-6-yl)methyl]-2-isopropyl-1,2,4-triazole-3-carboxamide

To a mixture of Intermediates 101 and 102 (anti:syn stereoisomers in ˜3:1 ratio) (212 mg, 0.39 mmol), Intermediate 79 (72.0 mg, 0.43 mmol) and DIPEA (0.27 mL, 1.60 mmol) in DMF (10 mL) at r.t. was added HATU (186.0 mg, 0.47 mmol) in one portion. The mixture was stirred for 35 minutes, then H₂O (20 mL) was added and the mixture was extracted with EtOAc (3×20 mL). The combined organic layers were washed with brine (40 mL), then dried (Na₂SO₄) and concentrated in vacuo. The residue was purified by flash chromatography, eluting with EtOAc/isohexane (0-100% gradient), then 5% MeOH/DCM. The resulting brown oil (233.0 mg) was subjected to chiral purification (Method 28) to afford, after lyophilisation, the title compounds (Peak 1, 28.8 mg, 11.3%, >99% d.e.; and Peak 2, 89.8 mg, 35.1%, >99% d.e.) as separate diastereomers.

Peak 1 (arbitrarily assigned 2S,4S) (1.3:1 rotameric ratio by ¹H NMR at r.t.): δ_{H} (400 MHz, DMSO-d₆) 8.93 (d, J 9.1 Hz, 1H, major rotamer), 8.91 (d, J 8.9 Hz, 1H, minor rotamer), 8.78 (s, 1H, minor rotamer), 8.62 (s, 1H, major rotamer), 8.264 (s, 1H, minor rotamer), 8.256 (s, 1H, major rotamer), 8.09 (s, 1H, major and minor rotamers), 5.70-5.63 (m, 1H, major rotamer), 5.53 (sept., J 6.6 Hz, 1H, major and minor rotamers), 5.58-5.48 (obscured m, 1H, minor rotamer), 5.15 (app. t, J 8.7 Hz, 1H, major and minor rotamers), 4.71-4.61 (m, 1H, major rotamer), 4.61-4.47 (m, 1H, major and minor rotamers), 4.31-4.18 (m, 2H, 2× minor rotamers), 4.08-3.95 (m, 1H, major and minor rotamer; and m, 1H, major rotamer), 3.94-3.78 (m, 2H, major and minor rotamers; and m, 1H, major rotamer), 3.21-3.08 (m, 1H, minor rotamer), 2.77-2.65 (m, 1H, minor rotamer), 2.31-1.48 (m, 14H, major and minor rotamers; and m, 1H, major rotamer), 1.42 (d, J 6.6 Hz, 3H, major and minor rotamers), 1.38 (d, J 6.6 Hz, 3H, major and minor rotamers), 1.46-1.20 (obscured m, 2H, major and minor rotamers). LCMS (Method 1): [M+H]⁺ m/z 652.4, RT 1.86 minutes. Chiral analysis (Method 29): RT 3.78 minutes.

Peak 2 (arbitrarily assigned 2R,4S) (2.2:1 rotameric ratio by ¹H NMR at r.t.): δ_{H} (400 MHz, DMSO-d₆) 8.96 (d, J 8.1 Hz, 1H, minor rotamer), 8.94 (d, J 9.0 Hz, 1H, major rotamer), 8.52 (s, 1H, major rotamer), 8.50 (s, 1H, minor rotamer), 8.33 (s, 1H, minor rotamer), 8.31 (s, 1H, major rotamer), 8.11 (s, 1H, major and minor rotamers), 5.94 (app. d, J 5.7 Hz, 1H, major rotamer), 5.59 (d, J 5.9 Hz, 1H, minor rotamer), 5.55 (sept., J 6.8 Hz, 1H, major and minor rotamers), 5.18 (t, J 8.7 Hz, 1H, minor rotamer), 5.17 (t, J 8.7 Hz, 1H, major rotamer), 4.87 (d, J 4.7 Hz, 1H, major and minor rotamers), 4.65-4.58 (m, 1H, major and minor rotamers), 4.47-4.48 (m, 1H, minor rotamer), 4.04-3.62 (m, 3H, major and minor rotamers; and m, 1H, major rotamer), 3.10 (app. t, J 13.1 Hz, 1H, major rotamer), 2.84-2.65 (m, 1H, major and minor rotamers; and m, 1H, minor rotamer), 2.32-1.48 (m, 14H, major and minor rotamers), 1.43 (d, J 6.6 Hz, 3H, major and minor rotamers), 1.38 (d, J 6.7 Hz, 3H, major and minor rotamers), 1.47-1.17 (obscured m, 2H, major and minor rotamers). LCMS (Method 1): [M+H]⁺ m/z 652.4, RT 1.86 minutes. Chiral analysis (Method 29): RT 4.66 minutes.

### Examples 52 & 53

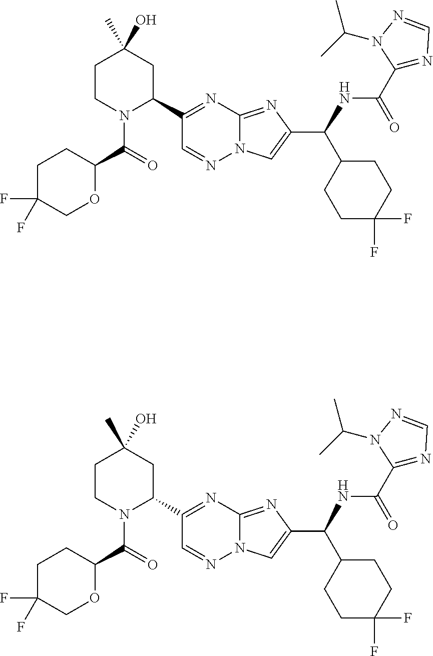 [SMILES: CC(C)n1ncnc1C(=O)N[C@H](c1cn2ncc([C@@H]3C[C@](C)(O)CCN3C(=O)[C@@H]3CCC(F)(F)CO3)nc2n1)C1CCC(F)(F)CC1 ; CC(C)n1ncnc1C(=O)N[C@H](c1cn2ncc([C@H]3C[C@@](C)(O)CCN3C(=O)[C@@H]3CCC(F)(F)CO3)nc2n1)C1CCC(F)(F)CC1]

### N—[(S)-(4,4-Difluorocyclohexyl)(3-{(2S,4R)-1-[(2S)-5,5-difluorotetrahydropyran-2-carbonyl]-4-hydroxy-4-methylpiperidin-2-yl}imidazo[1,2-b][1,2,4]triazin-6-yl)methyl]-2-isopropyl-1,2,4-triazole-3-carboxamide

### N—[(S)-(4,4-difluorocyclohexyl)(3-{(2R,4S)-1-[(2S)-5,5-difluorotetrahydropyran-2-carbonyl]-4-hydroxy-4-methylpiperidin-2-yl}imidazo[1,2-b][1,2,4]triazin-6-yl)methyl]-2-isopropyl-1,2,4-triazole-3-carboxamide

To a solution of Intermediate 79 (24 mg, 0.15 mmol), Intermediate 104 (71 mg, 0.13 mmol) and DIPEA (90 μL, 0.51 mmol) in DCM (2 mL) was added HATU (63 mg, 0.18 mmol). The reaction mixture was stirred at r.t. for 1 h, then quenched with water (2 mL) and extracted with DCM (3×2 mL). The combined organic layers were dried (MgSO₄), filtered and concentrated under reduced pressure. The residue was purified by reverse-phase HPLC (Method 32). The resulting material was subjected to chiral reverse-phase chromatography (Method 33) to afford, after lyophilisation, the title compounds (Peak 1, 7.1 mg, 8%; and Peak 2, 6.9 mg, 8%) as separate diastereomers.

Peak 1 (arbitrarily assigned 2S,4R): δ_{H} (500 MHz, CD₃OD) 8.70-8.45 (m, 1H), 8.12-8.03 (m, 1H), 7.95 (s, 1H), 5.96-5.56 (m, 2H), 5.21 (t, J 8.4 Hz, 1H), 4.67-4.38 (m, 1H), 4.29-3.98 (m, 2H), 3.89-3.60 (m, 2H), 2.78-2.58 (m, 1H), 2.43-1.89 (m, 9H), 1.86-1.57 (m, 5H), 1.54-1.35 (m, 8H), 1.27-1.23 (m, 3H). LCMS (Method 2): [M+H]⁺ m/z 666.1, RT 3.37 minutes. Chiral analysis (Method 34): RT 8.76 minutes.

Peak 2 (arbitrarily assigned 2R,4S): δ_{H} (500 MHz, CD₃OD) 8.66-8.45 (m, 1H), 8.15-8.05 (m, 1H), 7.96 (s, 1H), 5.94-5.56 (m, 2H), 5.22 (d, J 8.4 Hz, 1H), 4.72-4.52 (m, 1H), 4.11-3.92 (m, 2H), 3.89-3.65 (m, 2H), 2.75-2.50 (m, 1H), 2.39-1.93 (m, 9H), 1.91-1.56 (m, 5H), 1.58-1.34 (m, 8H), 1.29-1.12 (m, 3H). LCMS (Method 2): [M+H]⁺ m/z 666.1, RT 3.35 minutes. Chiral analysis (Method 34): RT 30.92 minutes.

### Example 54

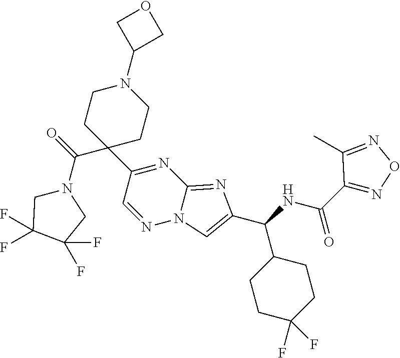 [SMILES: Cc1nonc1C(=O)N[C@H](c1cn2ncc(C3(C(=O)N4CC(F)(F)C(F)(F)C4)CCN(C4COC4)CC3)nc2n1)C1CCC(F)(F)CC1]

### N—[(S)-(4,4-Difluorocyclohexyl){3-[1-(oxetan-3-yl)-4-(3,3,4,4-tetrafluoropyrrolidine-1-carbonyl)piperidin-4-yl]imidazo[1,2-b][1,2,4]triazin-6-yl}methyl]-4-methyl-1,2,5-oxadiazole-3-carboxamide

To a stirred solution of Intermediate 109 (280 mg, 0.445 mmol) in anhydrous MeOH (6 mL) at r.t. was added oxetan-3-one (86 μL, 1.33 mmol), followed by AcOH (76 μL, 1.33 mmol). The solution was stirred for 10 minutes, then sodium cyanoborohydride (196 mg, 3.11 mmol) was added. The reaction mixture was stirred in a sealed vial at 50° C. for 4 h. Upon cooling to r.t., the material was diluted with EtOAc (30 mL) and washed with saturated aqueous NaHCO₃ solution (20 mL), water (20 mL) and brine (10 mL), then dried over Na₂SO₄, filtered and concentrated in vacuo. The resulting crude material was purified by flash column chromatography, eluting with methanol/DCM (0-5% gradient), followed by reverse-phase basic flash column chromatography, eluting with water/acetonitrile with 0.1% NH₄OH (10-100% gradient), to afford, after freeze-drying from acetonitrile/water, the title compound (178 mg, 57%) as an orange solid. δ_{H} (500 MHz, DMSO-d₆) 9.54 (d, J 9.1 Hz, 1H), 8.69 (s, 1H), 8.34 (s, 1H), 5.23 (t, J 8.6 Hz, 1H), 4.51 (t, J 6.5 Hz, 2H), 4.41 (t, J 6.1 Hz, 2H), 4.04 (s, 4H), 3.40-3.34 (m, 1H), 2.58-2.51 (m, 2H), 2.47 (s, 3H), 2.35-2.27 (m, 2H), 2.27-2.10 (m, 5H), 2.10-1.95 (m, 2H), 1.95-1.87 (m, 1H), 1.87-1.69 (m, 2H), 1.69-1.58 (m, 1H), 1.48-1.36 (m, 1H), 1.36-1.24 (m, 1H). LCMS (Method 2): [M+H]⁺ m/z 686, RT 2.34 minutes.

### Example 55

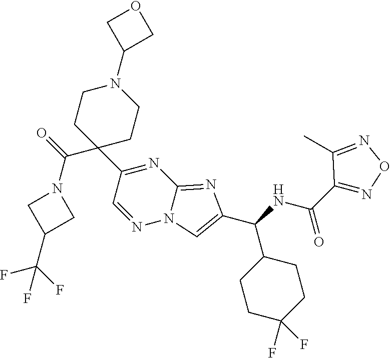 [SMILES: Cc1nonc1C(=O)N[C@H](c1cn2ncc(C3(C(=O)N4CC(C(F)(F)F)C4)CCN(C4COC4)CC3)nc2n1)C1CCC(F)(F)CC1]

### N—[(S)-(4,4-Difluorocyclohexyl)(3-{1-(oxetan-3-yl)-4-[3-(trifluoromethyl)azetidine-1-carbonyl]piperidin-4-yl}imidazo[1,2-b][1,2,4]triazin-6-yl)methyl]-4-methyl-1,2,5-oxadiazole-3-carboxamide

Intermediate 114 (20 mg, 0.033 mmol) was dissolved in MeOH (2 mL) and treated with oxetan-3-one (4 mg, 0.056 mmol). The mixture was stirred for 10 minutes, then sodium cyanoborohydride (4 mg, 0.060 mmol) was added. After 3 h, additional oxetan-3-one (4 mg, 0.056 mmol) and sodium cyanoborohydride (4 mg, 0.061 mmol) were added. The reaction mixture was diluted with DCM (20 mL), and washed with saturated aqueous NaHCO₃ solution (20 mL), brine (10 mL) and water (10 mL), then evaporated to dryness, to afford the title compound as a yellow solid. δ_{H} (400 MHz, DMSO-d₆) 9.53 (d, J 9.0 Hz, 1H), 8.69 (s, 1H), 8.34 (s, 1H), 5.23 (dd, J 8.5, 8.5 Hz, 1H), 4.50 (dd, J 6.4, 6.4 Hz, 2H), 4.40 (dd, J 6.1, 6.1 Hz, 2H), 4.29-3.74 (m, 4H), 3.41 (m, 1H), 2.48 (s, 3H), 2.42-1.53 (m, 15H), 1.53-1.15 (m, 3H). LCMS (Method 7): [M+H]⁺ m/z 668.4, RT 1.91 minutes.

### Example 56

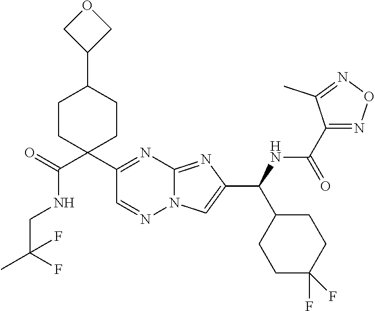 [SMILES: Cc1nonc1C(=O)N[C@H](c1cn2ncc(C3(C(=O)NCC(C)(F)F)CCC(C4COC4)CC3)nc2n1)C1CCC(F)(F)CC1]

### N—[(S)-(4,4-Difluorocyclohexyl){3-[4-(2,2-difluoropropylcarbamoyl)-1-(oxetan-3-yl)-piperidin-4-yl]imidazo[1,2-b][1,2,4]triazin-6-yl}methyl]-4-methyl-1,2,5-oxadiazole-3-carboxamide

To a solution of Intermediate 119 (316 mg, 0.54 mmol) in MeOH (5.4 mL) were added AcOH (94 μL, 1.63 mmol) and oxetan-3-one (106 μL, 1.63 mmol). The mixture was stirred for 5 minutes, then sodium cyanoborohydride (252 mg, 3.80 mmol) was added in one portion. The reaction mixture was allowed to stir for a further 16 h. Additional portions of oxetan-3-one (35 μL, 0.54 mmol), AcOH (31 μL, 0.54 mmol) and sodium cyanoborohydride (102 mg, 1.63 mmol) were added. The reaction mixture was stirred for 1 h, then quenched with saturated aqueous NaHCO₃ solution (1 mL) and concentrated in vacuo. The residue was diluted with saturated aqueous NaHCO₃ solution (50 mL) and extracted with EtOAc (3×50 mL). The combined organic extracts were passed through a phase separator and concentrated in vacuo. The crude material was purified by column chromatography, eluting with a gradient of 0-10% MeOH in DCM. The resultant material was further purified by preparative HPLC to give the title compound (191 mg, 55%) as a pale yellow amorphous solid. δ_{H} (400 MHz, DMSO-d₆) 9.06 (d, J 9.0 Hz, 1H), 8.62 (s, 1H), 8.25 (s, 1H), 7.89 (s, 1H), 5.28 (t, J 8.5 Hz, 1H), 4.58-4.49 (m, 4H), 3.54 (td, J 14.0, 6.0 Hz, 2H), 2.69-2.22 (m, 12H), 2.13-1.95 (m, 3H), 1.90-1.72 (m, 3H), 1.56-1.35 (m, 5H). One CH signal not resolved. LCMS (Method 7): [M+H]⁺ m/z 638.4, RT 1.80 minutes.

1. A compound of formula (I) or an N-oxide thereof, or a pharmaceutically acceptable salt thereof: 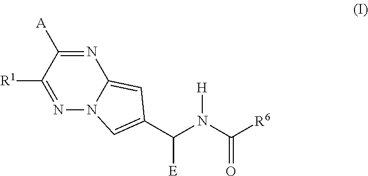
wherein
E represents a group of formula (Ea), (Eb), (Ec), (Ed) or (Ee): 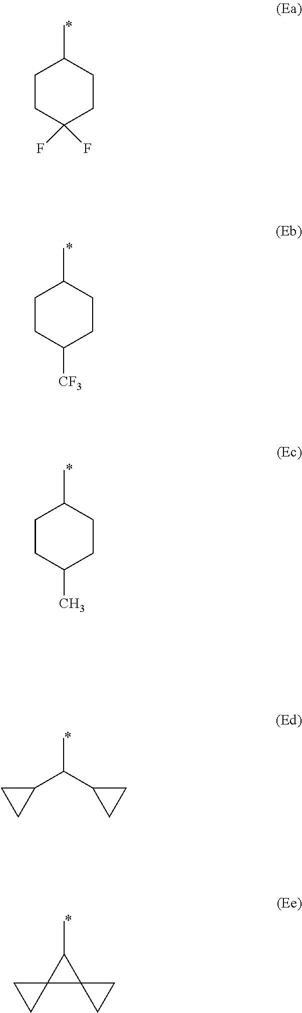
in which the asterisk (*) represents the point of attachment to the remainder of the molecule;
A represents a group of formula (Aa), (Ab), (Ac), (Ad) or (Ae): 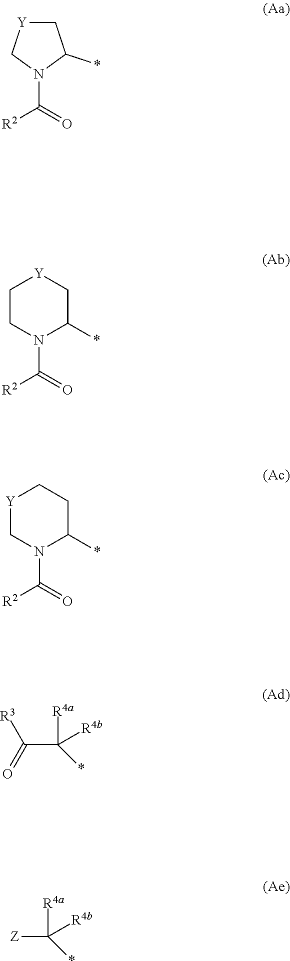
in which the asterisk (*) represents the point of attachment to the remainder of the molecule;
Y represents —O—, —N(R⁷)—, —C(R^{5a})(R^{5b})—, —S—, —S(O)—, —S(O)₂— or —S(O)(N—R⁸)—;
Z represents heteroaryl, which group may be optionally substituted by one or more substituents;
R¹ represents hydrogen, fluoro, chloro, methyl, difluoromethyl or trifluoromethyl;
R² represents —OR^{2a}; or R² represents C₃₋₉ cycloalkyl, C₄₋₁₂ bicycloalkyl, C₃₋₇ heterocycloalkyl or C₄₋₉ heterobicycloalkyl, any of which groups may be optionally substituted by one or more substituents;
R^{2a} represents C₁₋₆ alkyl; or R^{2a} represents C₃₋₉ cycloalkyl, which group may be optionally substituted by one or more substituents;
R³ represents —NR^{3a}R^{3b}; or R³ represents a group of formula (Wa): 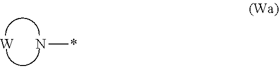
in which the asterisk (*) represents the point of attachment to the remainder of the molecule;
W represents the residue of an optionally substituted saturated monocyclic ring containing 3 to 6 carbon atoms, one nitrogen atom, and 0, 1, 2 or 3 additional heteroatoms independently selected from N, O and S, but containing no more than one O or S atom; or
W represents the residue of an optionally substituted saturated bicyclic ring system containing 4 to 10 carbon atoms, one nitrogen atom, and 0, 1, 2 or 3 additional heteroatoms independently selected from N, O and S, but containing no more than one O or S atom; or
W represents the residue of an optionally substituted saturated spirocyclic ring system containing 5 to 10 carbon atoms, one nitrogen atom, and 0, 1, 2 or 3 additional heteroatoms independently selected from N, O and S, but containing no more than one O or S atom;
R^{3a} represents hydrogen or C₁₋₆ alkyl;
R^{3b} represents C₁₋₆ alkyl, C₃₋₇ cycloalkyl, C₃₋₇ cycloalkyl(C₁₋₆)alkyl, C₄₋₁₂ bicycloalkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents;
R^{4a} represents hydrogen, fluoro or hydroxy; or R^{4a} represents C₁₋₆ alkyl, which group may be optionally substituted by one or more substituents; and
R^{4b} represents hydrogen, fluoro or C₁₋₆ alkyl; or
R^{4a} and R^{4b}, when taken together with the carbon atom to which they are both attached, represent C₃₋₉ cycloalkyl or C₃₋₇ heterocycloalkyl, either of which groups may be optionally substituted by one or more substituents;
R^{5a} represents hydrogen, fluoro, methyl, difluoromethyl or trifluoromethyl; and
R^{5b} represents hydrogen, fluoro, methyl or hydroxy; or
R^{5a} and R^{5b}, when taken together with the carbon atom to which they are both attached, represent cyclopropyl;
R⁶ represents —OR^{6a} or —NR^{6b}R^{6c}—; or R⁶ represents C₁₋₆ alkyl, C₃₋₉ cycloalkyl, C₃₋₉ cycloalkyl(C₁₋₆)alkyl, aryl, aryl(C₁₋₆)alkyl, C₃₋₇ heterocycloalkyl, C₃₋₇ heterocycloalkyl-(C₁₋₆)alkyl, heteroaryl or heteroaryl(C₁₋₆)alkyl, any of which groups may be optionally substituted by one or more substituents;
R^{6a} represents C₁₋₆ alkyl, C₃₋₉ cycloalkyl or aryl(C₁₋₆)alkyl, any of which groups may be optionally substituted by one or more substituents;
R^{6b} represents hydrogen or C₁₋₆ alkyl; and
R^{6c} represents hydrogen or C₁₋₆ alkyl; or
R^{6b} and R^{6c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents;
R⁷ represents —COR^{7a}, —CO₂R^{7a} or —SO₂R^{7b}; or R⁷ represents hydrogen; or R⁷ represents C₁₋₆ alkyl or C₃₋₉ cycloalkyl, either of which groups may be optionally substituted by one or more fluorine atoms;
R^{7a} represents C₁₋₆ alkyl, optionally substituted by one or more fluorine atoms;
R^{7b} represents C₁₋₆ alkyl; and
R⁸ represents C₁₋₆ alkyl.

2. A compound as claimed in claim 1 wherein E represents a group of formula (Ea), (Eb) or (Ed) as defined in claim 1.

3. A compound as claimed in claim 1 wherein R⁶ represents C₃₋₉ cycloalkyl, aryl or heteroaryl, any of which groups may be optionally substituted by one or more substituents.

4. A compound as claimed in claim 1 represented by formula (IIA-1) or an N-oxide thereof, or a pharmaceutically acceptable salt thereof: 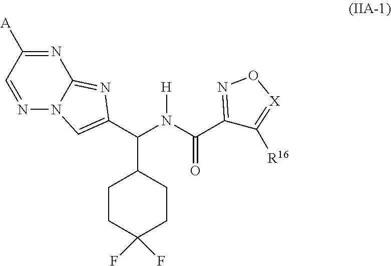
wherein
X represents CH or N; and
R¹⁶ represents methyl, ethyl, isopropyl or cyclopropyl.

5. A compound as claimed in claim 1 represented by formula (IIA-2) or an N-oxide thereof, or a pharmaceutically acceptable salt thereof: 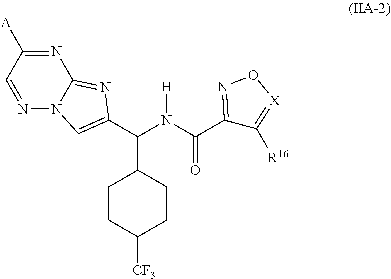
wherein
X represents CH or N; and
R¹⁶ represents methyl, ethyl, isopropyl or cyclopropyl.

6. A compound as claimed in claim 1 represented by formula (IIA-3) or an N-oxide thereof, or a pharmaceutically acceptable salt thereof: 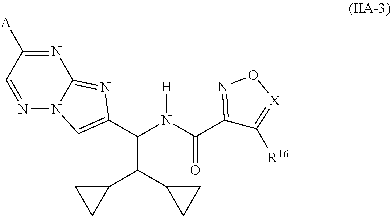
wherein
X represents CH or N; and
R¹⁶ represents methyl, ethyl, isopropyl or cyclopropyl.

7. A compound as claimed in claim 1 represented by formula (IIB-1) or an N-oxide thereof, or a pharmaceutically acceptable salt thereof: 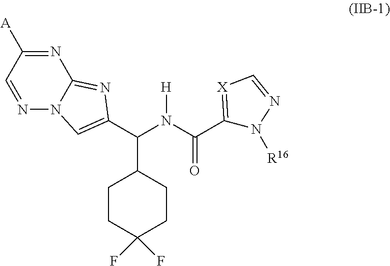
wherein
X represents CH or N; and
R¹⁶ represents methyl, ethyl, isopropyl or cyclopropyl are as defined in claim 4.

8. A compound as claimed in claim 1 represented by formula (IIC-1) or an N-oxide thereof, or a pharmaceutically acceptable salt thereof: 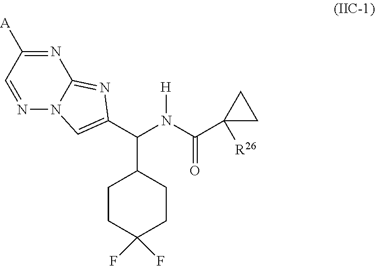
wherein
R²⁶ represents fluoro or trifluoromethyl.

9. A compound as claimed in claim 1 represented by formula (IIC-2) or an N-oxide thereof, or a pharmaceutically acceptable salt thereof: 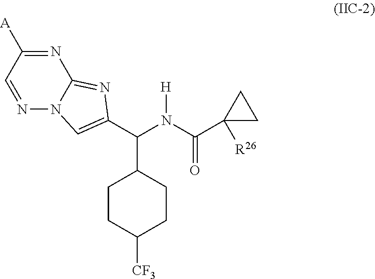
wherein
R²⁶ represents fluoro or trifluoromethyl.

10. A compound according to claim 1 wherein A represents a group of formula (Ab) or (Ad).

11. A compound which is
N-[(1S)-2,2-Dicyclopropyl-1-{3-[(2S,4R)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}ethyl]-4-methyl-1,2,5-oxadiazole-3-carboxamide;
N-[(1S)-2,2-Dicyclopropyl-1-{3-[(2R,4S)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}ethyl]-4-methyl-1,2,5-oxadiazole-3-carboxamide;
N—[(S)-(4,4-Difluorocyclohexyl){3-[(3R)-4-(3-fluorobicyclo[1.1.1]pentane-1-carbonyl)-morpholin-3-yl]imidazo[1,2-b][1,2,4]triazin-6-yl}methyl]-4-methyl-1,2,5-oxadiazole-3-carboxamide;
N—[(S)-(4,4-Difluorocyclohexyl){3-[(3S)-4-(3-fluorobicyclo[1.1.1]pentane-1-carbonyl)-morpholin-3-yl]imidazo[1,2-b][1,2,4]triazin-6-yl}methyl]-4-methyl-1,2,5-oxadiazole-3-carboxamide;
N-[(1S)-2,2-Dicyclopropyl-1-(3-{(3R)-4-[(2S)-5,5-difluorotetrahydropyran-2-carbonyl]-morpholin-3-yl}imidazo[1,2-b][1,2,4]triazin-6-yl)ethyl]-4-methyl-1,2,5-oxadiazole-3-carboxamide;
N-[(1S)-2,2-Dicyclopropyl-1-(3-{(3S)-4-[(2S)-5,5-difluorotetrahydropyran-2-carbonyl]-morpholin-3-yl}imidazo[1,2-b][1,2,4]triazin-6-yl)ethyl]-4-methyl-1,2,5-oxadiazole-3-carboxamide;
N—[(S)-(4,4-Difluorocyclohexyl)(3-{(3R)-4-[(2S)-5,5-difluorotetrahydropyran-2-carbonyl]morpholin-3-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(3S)-4-[(2S)-5,5-difluorotetrahydropyran-2-carbonyl]morpholin-3-yl}imidazo[1,2-b][1,2,4]triazin-6-yl)methyl]-4-methyl-1,2,5-oxadiazole-3-carboxamide;
N—[(S)-(4,4-Difluorocyclohexyl){3-[(2S,4R)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-4-methyl-1,2,5-oxadiazole-3-carboxamide;
N—[(S)-(4,4-Difluorocyclohexyl){3-[(2R,4S)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-4-methyl-1,2,5-oxadiazole-3-carboxamide;
N—[(S)-(4,4-Difluorocyclohexyl){3-[(2S,4R)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-1-fluorocyclopropanecarboxamide;
N—[(S)-(4,4-Difluorocyclohexyl){3-[(2R,4S)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-1-fluorocyclopropanecarboxamide;
Benzyl N—[(S)-{3-[4-(3-fluorobicyclo[1.1.1]pentane-1-carbonyl)-1,1-dioxo-1,4-thiazinan-3-yl]imidazo[1,2-b][1,2,4]triazin-6-yl}[4-(trifluoromethyl)cyclohexyl]methyl]carbamate;
1-Fluoro-N—[(S)-{3-[(3R)-4-(3-fluorobicyclo[1.1.1]pentane-1-carbonyl)-1,1-dioxo-1,4-thiazinan-3-yl]imidazo[1,2-b][1,2,4]triazin-6-yl}[4-(trifluoromethyl)cyclohexyl]methyl]-cyclopropanecarboxamide;
1-Fluoro-N—[(S)-{3-[(3S)-4-(3-fluorobicyclo[1.1.1]pentane-1-carbonyl)-1,1-dioxo-1,4-thiazinan-3-yl]imidazo[1,2-b][1,2,4]triazin-6-yl}[4-(trifluoromethyl)cyclohexyl]methyl]-cyclopropanecarboxamide;
N—[(S)-(4,4-Difluorocyclohexyl){3-[1-(2,2-difluoropropylcarbamoyl)-4,4-difluoro-cyclohexyl]imidazo[1,2-b][1,2,4]triazin-6-yl}methyl]-2-isopropylpyrazole-3-carboxamide;
N—[(S)-(4,4-Difluorocyclohexyl){3-[4-(2,2-difluoropropylcarbamoyl)tetrahydropyran-4-yl]imidazo[1,2-b][1,2,4]triazin-6-yl}methyl]-4-methyl-1,2,5-oxadiazole-3-carboxamide;
N—[(S)-(4,4-Difluorocyclohexyl)(3-{(2S,4R)-1-[(2S)-5,5-difluorotetrahydropyran-2-carbonyl]-4-hydroxy-4-(trifluoromethyl)piperidin-2-yl}imidazo[1,2-b][1,2,4]triazin-6-yl)-methyl]-4-methyl-1,2,5-oxadiazole-3-carboxamide;
N—[(S)-(4,4-Difluorocyclohexyl)(3-{(2R,4S)-1-[(2S)-5,5-difluorotetrahydropyran-2-carbonyl]-4-hydroxy-4-(trifluoromethyl)piperidin-2-yl}imidazo[1,2-b][1,2,4]triazin-6-yl)-methyl]-4-methyl-1,2,5-oxadiazole-3-carboxamide;
N—[(S)-(4,4-Difluorocyclohexyl)(3-{(2S,4R)-1-[(2S)-5,5-difluorotetrahydropyran-2-carbonyl]-4-hydroxy-4-(trifluoromethyl)piperidin-2-yl}imidazo[1,2-b][1,2,4]triazin-6-yl)-methyl]-1-fluorocyclopropanecarboxamide;
N—[(S)-(4,4-Difluorocyclohexyl)(3-{(2R,4S)-1-[(2S)-5,5-difluorotetrahydropyran-2-carbonyl]-4-hydroxy-4-(trifluoromethyl)piperidin-2-yl}imidazo[1,2-b][1,2,4]triazin-6-yl)-methyl]-1-fluorocyclopropanecarboxamide;
N—[(S)-(4,4-Difluorocyclohexyl)(3-{(2S,4R)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)-methyl]-4-methyl-1,2,5-oxadiazole-3-carboxamide;
N—[(S)-(4,4-Difluorocyclohexyl)(3-{(2R,4S)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)-methyl]-4-methyl-1,2,5-oxadiazole-3-carboxamide;
N—{(S)-(3-{(3R)-4-[(2S)-5,5-Difluorotetrahydropyran-2-carbonyl]morpholin-3-yl}-imidazo[1,2-b][1,2,4]triazin-6-yl)[4-(trifluoromethyl)cyclohexyl]methyl}-4-methyl-1,2,5-oxadiazole-3-carboxamide;
N—{(S)-(3-{(3S)-4-[(2S)-5,5-Difluorotetrahydropyran-2-carbonyl]morpholin-3-yl}-imidazo[1,2-b][1,2,4]triazin-6-yl)[4-(trifluoromethyl)cyclohexyl]methyl}-4-methyl-1,2,5-oxadiazole-3-carboxamide;
N—[(S)-(4,4-Difluorocyclohexyl){3-[(2S,4R)-4-(difluoromethyl)-1-(3-fluorobicyclo[1.1.1]-pentane-1-carbonyl)-4-hydroxypiperidin-2-yl]imidazo[1,2-b][1,2,4]triazin-6-yl}methyl]-4-methyl-1,2,5-oxadiazole-3-carboxamide;
N—[(S)-(4,4-Difluorocyclohexyl){3-[(2R,4S)-4-(difluoromethyl)-1-(3-fluorobicyclo[1.1.1]-pentane-1-carbonyl)-4-hydroxypiperidin-2-yl]imidazo[1,2-b][1,2,4]triazin-6-yl}methyl]-4-methyl-1,2,5-oxadiazole-3-carboxamide;
N—[(S)-(4,4-Difluorocyclohexyl){3-[(2S,4S)-4-(difluoromethyl)-1-(3-fluorobicyclo-[1.1.1]pentane-1-carbonyl)-4-hydroxypiperidin-2-yl]imidazo[1,2-b][1,2,4]triazin-6-yl}-methyl]-4-methyl-1,2,5-oxadiazole-3-carboxamide;
N—[(S)-(4,4-Difluorocyclohexyl){3-[(2R,4R)-4-(difluoromethyl)-1-(3-fluorobicyclo-[1.1.1]pentane-1-carbonyl)-4-hydroxypiperidin-2-yl]imidazo[1,2-b][1,2,4]triazin-6-yl}-methyl]-4-methyl-1,2,5-oxadiazole-3-carboxamide;
N—[(S)-(4,4-Difluorocyclohexyl)(3-{(2S,4S)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(2R,4R)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide;
N—[(S)-(4,4-Difluorocyclohexyl){3-[(2S,4S)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-4-methyl-1,2,5-oxadiazole-3-carboxamide;
N—[(S)-(4,4-Difluorocyclohexyl){3-[(2R,4R)-1-(3-fluorobicyclo[1.1.1]pentane-1-carbonyl)-4-hydroxy-4-(trifluoromethyl)piperidin-2-yl]imidazo[1,2-b][1,2,4]triazin-6-yl}-methyl]-4-methyl-1,2,5-oxadiazole-3-carboxamide;
N—[(S)-(4,4-Difluorocyclohexyl)(3-{(2S,4R)-1-[(2S)-5,5-difluorotetrahydropyran-2-carbonyl]-4-hydroxy-4-methylpiperidin-2-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(2R,4S)-1-[(2S)-5,5-difluorotetrahydropyran-2-carbonyl]-4-hydroxy-4-methylpiperidin-2-yl}imidazo[1,2-b][1,2,4]triazin-6-yl)methyl]-4-methyl-1,2,5-oxadiazole-3-carboxamide;
N—[(S)-(4,4-Difluorocyclohexyl){3-[(2S,4R)-1-(3-fluorobicyclo[1.1.1]pentane-1-carbonyl)-4-hydroxy-4-methylpiperidin-2-yl]imidazo[1,2-b][1,2,4]triazin-6-yl}methyl]-4-methyl-1,2,5-oxadiazole-3-carboxamide;
N—[(S)-(4,4-Difluorocyclohexyl){3-[(2R,4S)-1-(3-fluorobicyclo[1.1.1]pentane-1-carbonyl)-4-hydroxy-4-methylpiperidin-2-yl]imidazo[1,2-b][1,2,4]triazin-6-yl}methyl]-4-methyl-1,2,5-oxadiazole-3-carboxamide;
N—[(S)-(4,4-Difluorocyclohexyl)(3-{(2S,4S)-1-[(2S)-5,5-difluorotetrahydropyran-2-carbonyl]-4-hydroxy-4-methylpiperidin-2-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(2R,4R)-1-[(2S)-5,5-difluorotetrahydropyran-2-carbonyl]-4-hydroxy-4-methylpiperidin-2-yl}imidazo[1,2-b][1,2,4]triazin-6-yl)methyl]-4-methyl-1,2,5-oxadiazole-3-carboxamide;
N—[(S)-(4,4-Difluorocyclohexyl){3-[(2S,4S)-1-(3-fluorobicyclo[1.1.1]pentane-1-carbonyl)-4-hydroxy-4-methylpiperidin-2-yl]imidazo[1,2-b][1,2,4]triazin-6-yl}methyl]-4-methyl-1,2,5-oxadiazole-3-carboxamide;
N—[(S)-(4,4-Difluorocyclohexyl){3-[(2R,4R)-1-(3-fluorobicyclo[1.1.1]pentane-1-carbonyl)-4-hydroxy-4-methylpiperidin-2-yl]imidazo[1,2-b][1,2,4]triazin-6-yl}methyl]-4-methyl-1,2,5-oxadiazole-3-carboxamide;
N—[(S)-(4,4-Difluorocyclohexyl)(3-{(2S,4R)-1-[(2S)-5,5-difluorotetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(2R,4S)-1-[(2S)-5,5-difluorotetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(2S,4S)-1-[(2S)-5,5-difluorotetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(2R,4R)-1-[(2S)-5,5-difluorotetrahydropyran-2-carbonyl]-4-hydroxypiperidin-2-yl}imidazo[1,2-b][1,2,4]triazin-6-yl)methyl]-4-methyl-1,2,5-oxadiazole-3-carboxamide;
N—[(S)-(4,4-Difluorocyclohexyl)(3-{(2S,4R)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)-methyl]-2-methylpyrazole-3-carboxamide;
N—[(S)-(4,4-Difluorocyclohexyl)(3-{(2R,4S)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)-methyl]-2-methyl-pyrazole-3-carboxamide;
N—[(S)-(4,4-Difluorocyclohexyl)(3-{(2S,4R)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)methyl]-2-isopropyl-1,2,4-triazole-3-carboxamide;
N—[(S)-(4,4-Difluorocyclohexyl)(3-{(2R,4S)-4-(difluoromethyl)-1-[(2S)-5,5-difluoro-tetrahydropyran-2-carbonyl]-4-hydroxypiperidin-2-yl}imidazo[1,2-b][1,2,4]triazin-6-yl)methyl]-2-isopropyl-1,2,4-triazole-3-carboxamide;
N—[(S)-(4,4-Difluorocyclohexyl)(3-{(2S,4S)-1-[(2S)-5,5-difluorotetrahydropyran-2-carbonyl]-4-hydroxypiperidin-2-yl}imidazo[1,2-b][1,2,4]triazin-6-yl)methyl]-2-isopropyl-1,2,4-triazole-3-carboxamide;
N—[(S)-(4,4-Difluorocyclohexyl)(3-{(2R,4S)-1-[(2S)-5,5-difluorotetrahydropyran-2-carbonyl]-4-hydroxypiperidin-2-yl}imidazo[1,2-b][1,2,4]triazin-6-yl)methyl]-2-isopropyl-1,2,4-triazole-3-carboxamide;
N—[(S)-(4,4-Difluorocyclohexyl)(3-{(2S,4R)-1-[(2S)-5,5-difluorotetrahydropyran-2-carbonyl]-4-hydroxy-4-methylpiperidin-2-yl}imidazo[1,2-b][1,2,4]triazin-6-yl)methyl]-2-isopropyl-1,2,4-triazole-3-carboxamide;
N—[(S)-(4,4-difluorocyclohexyl)(3-{(2R,4S)-1-[(2S)-5,5-difluorotetrahydropyran-2-carbonyl]-4-hydroxy-4-methylpiperidin-2-yl}imidazo[1,2-b][1,2,4]triazin-6-yl)methyl]-2-isopropyl-1,2,4-triazole-3-carboxamide;
N—[(S)-(4,4-Difluorocyclohexyl){3-[1-(oxetan-3-yl)-4-(3,3,4,4-tetrafluoropyrrolidine-1-carbonyl)piperidin-4-yl]imidazo[1,2-b][1,2,4]triazin-6-yl}methyl]-4-methyl-1,2,5-oxadiazole-3-carboxamide;
N—[(S)-(4,4-Difluorocyclohexyl)(3-{1-(oxetan-3-yl)-4-[3-(trifluoromethyl)azetidine-1-carbonyl]piperidin-4-yl}imidazo[1,2-b][1,2,4]triazin-6-yl)methyl]-4-methyl-1,2,5-oxadiazole-3-carboxamide;
N—(S)-(4,4-Difluorocyclohexyl){3-[4-(2,2-difluoropropylcarbamoyl)-1-(oxetan-3-yl)-piperidin-4-yl]imidazo[1,2-b][1,2,4]triazin-6-yl}methyl]-4-methyl-1,2,5-oxadiazole-3-carboxamide; or
a pharmaceutically acceptable salt thereof.

12. (canceled)

13. (canceled)

14. (canceled)

15. A pharmaceutical composition comprising a compound of formula (I) as defined in claim 1 or an N-oxide thereof, or a pharmaceutically acceptable salt thereof, in association with a pharmaceutically acceptable carrier.

16. (canceled)

17. (canceled)

18. A method for the treatment and/or prevention of disorders for which the administration of a modulator of IL-17 function is indicated which comprises administering to a patient in need of such treatment an effective amount of a compound of formula (I) as defined in claim 1 or an N-oxide thereof, or a pharmaceutically acceptable salt thereof.

19. A method for the treatment and/or prevention of an inflammatory or autoimmune disorder, which comprises administering to a patient in need of such treatment an effective amount of a compound of formula (I) as defined in claim 1 or an N-oxide thereof, or a pharmaceutically acceptable salt thereof.
